SEQUENCE LISTING (293 sequences)

SEQ ID NO: 1 - DNA, 396 bp, Homo sapiens
  misc_feature 303, 370, 377, 382: n = A,T,C or G
caacctcactagtaaatgaaagaaatattgtaatttgtatttgatctgctgggtctttggagtcagaactggttttatcagcagtttgatcttctgaggtctggtatgtagtttgctggcccacagaaccttcacgtgtattcacagcctcaatgccataaggaaactcttttagaagttctgacagctggtcatgtaggtataagacaggtgccttatcactgtggatttcatttcttgcaggatcttggggagtatagttgctggatgcatctatttcctgagggtaaatatcctcctggncgacgcggccgctcgagtctagagggcccgtttaaacccgctgatcagcctcgactgtgccttctanttgccanccatntgttgtttgcccct

SEQ ID NO: 2 - DNA, 396 bp, Homo sapiens
cgaccaaaaagtaaactccaagtgaacatcaaatcaaatctaatccttttggccacatgactggttgttctttatctcatagttacaatgaatcatataaactgtagactgccactaccacgatacttctgtgacacagaaggaatgtcctatttgcctatctatctgaggaatgttaaatagagaaaaatagattataaaacaacctggaggtcacaggattctgagataatccctctgttaaaaaacatctgaacagcaaatgtccaatctgtaataaaatagttaaaggtccaagtcaagtccacttctacttggctggcccagcacaagaaatctaacagcactttgtaatcattttgcttttctaattttcccggaggacatgggccattg

SEQ ID NO: 3 - DNA, 396 bp, Homo sapiens
  misc_feature 22, 28, 29, 30, 33, 36, 41, 43, 45, 46, 53, 56, 58,61, 64, 69, 70, 74, 75, 78, 83, 84, 85, 102, 143, 335: n = A,T,C or G
cgcccttttttttttttttttnattggnnnaantcnctttnantnnaaaaacntgnanggnaancccanncccnnggnaccannnccaggagttgggtgganactgagtggggtttgtgtgggtgagggggcatctactcctnttgcaacaagccaaaagtagaacagcctaaggaaaagtgacctgccttggagccttagtccctcccttagggccccctcagcctaccctatccaagtctgaggctatggaagtctccctcctagttcactagcaggttccccatcttttccaggctgcccctagcactccacgtttttctgaaaaaatctanacaggccctttttgggtacctaaaacccagctgaggttgtgagcttgtaaggtaaagcaag

SEQ ID NO: 4 - DNA, 396 bp, Homo sapiens
  misc_feature 13, 15, 21, 27, 34, 37, 41, 57, 58, 59, 63, 64, 71, 72,77, 78, 83, 87, 93, 170, 207, 210, 308, 379, 382, 389, 391,392, 393, 395: n = A,T,C or G
gaccaatccttgncncactancaaaangaccccnctnaccnccaggaactgaacctnnntgtnnacctccnnctgcnnagccntatntccaanatcacccaccgtatccactgggaatctgccagcctcctgcgatcagaagagaccaatcgaaaatgagggtttcacantcacagctgaaggaaaaggccaaggcaccttgtcggnggngacaatgtaccatgctaaggccaaagatcaactcacctgtaataaattcgacctcaaggtcaccataaaaccagcaccggaacagaaaaagaggcctnaggatgcccaagaaacacttttgatcctttgaaaactgtaccaaggtaccggggggagacccaggaaaggnccnttatgtntnnntnt

SEQ ID NO: 5 - DNA, 396 bp, Homo sapiens
  misc_feature 135, 172, 343, 348, 354, 395: n = A,T,C or G
gacgccggagctgccgcgccagtcgcctagcaggtcctctaccggcttattcctgtgccggatcttcatcggcacaggggccactgagacgtttctgcctccctctttcttcctccgctctttctcttccctctngtttagtttgcctgggagcttgaaaggagaaagcacnggggtcgccccaaaccctttctgcttctgcccatcacaagtgccactaccgccatgggcctcactatctcctccctcttctcccgactatttggcaagaagcagatgcgcattttgatggttggattggatgctgctggcaagacaaccattcttgataaactgaaagtanggganataagnaccaccatttctaccattgggtttaatgggggaaacagtana

SEQ ID NO: 6 - DNA, 396 bp, Homo sapiens
  misc_feature 212: n = A,T,C or G
acgggaggcgccgggaagtcgacggcgccggcggctcctgcaggaggccactgtctgcagctcccgtgaagatgtccactccagacccacccctgggcggaactcctcggccaggtccttccccgggccctgcccttcccctggagccatgctgggccctagcccgggtccctcgccgggctccgcccacagcatgatggggcccagcccangggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccc

SEQ ID NO: 7 - DNA, 396 bp, Homo sapiens
acccgagagtcgtcggggtttcctgcttcaacagtgcttggacggaacccggcgctcgttccccaccccggccggccgcccatagccagccctccgtcacctcttcaccgcaccctcggactgccccaaggcccccgccgccgctccagcgccgcgcagccaccgccgccgccgccgcctctccttagtcgccgccatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgc

SEQ ID NO: 8 - DNA, 396 bp, Homo sapiens
cgacaacaaggttaataccttagttcttaacattttttttctttatgtgtagtgttttcatgctaccttggtaggaaacttatttacaaaccatattaaaaggctaatttaaatataaataatataaagtgctctgaataaagcagaaatatattacagttcattccacagaaagcatccaaaccacccaaatgaccaaggcatatatagtatttggaggaatcaggggtttggaaggagtagggaggagaatgaaggaaaatgcaaccagcatgattatagtgtgttcatttagataaaagtagaaggcacaggagaggtagcaaaggccaggcttttctttggttttcttcaaacataggtgaaaaaaacactgccattcacaagtcaaggaac

SEQ ID NO: 9 - DNA, 396 bp, Homo sapiens
  misc_feature 321, 344: n = A,T,C or G
tcgacatcgcggcaactttttgcggattgttcttgcttccaggctttgcgctgcaaatccagtgctaccagtgtgaagaattccagctgaacaacgactgctcctcccccgagttcattgtgaattgcacggtgaacgttcaagacatgtgtcagaaagaagtgatggagcaaagtgccgggatcatgtaccgcaagtcctgtgcatcatcagcggcctgtctcatcgcctctgccgggtaccagtccttctgctccccagggaaactgaactcagtttgcatcagctgctgcaacacccctctttgtaacgggccaaggnccaaaaaaaggggaaagttctgncctcggccctcaggccagggctccgcaccaccatcctgttcctcaaattagc

SEQ ID NO: 10 - DNA, 396 bp, Homo sapiens
  misc_feature 115, 116, 117, 130, 138, 142, 143, 144, 145, 146, 153,157, 158, 159, 160, 164, 175, 176, 177, 178, 179, 183, 187, 197,198, 202, 203, 204, 205, 206, 211, 212, 213, 215, 216, 217,220, 221, 222, 226, 231, 234, 236, 237, 245, 246, 247: n = A,T,C or G
  misc_feature 250, 255, 264, 266, 267, 268, 269, 270, 271, 272, 279,284, 297, 303, 304, 305, 308, 315, 317, 318, 319, 320, 321, 322,323, 333, 334, 337, 338, 342, 343, 368, 372, 374, 380, 381,391, 395: n = A,T,C or G
ccttttttttttttttttttttttttttttttttttttttttttttttttttttttttttttttttttttttttttttttttttttttttttttttttttttttaaaaaaaaaannnttttttttttttnaaaaaaangggnnnnnttttttncccnnnngggnggggggggggnnnnntttnaaanaaaaaaaccnnaaannnnnggggnnnannnaannncccnccccnaancnntaaaaaannnggnaaaanagggggggnannnnnnnggggggnaaaanttttttttttttnaagggnnnggnaaaaaantnnnnnnntttttttttnnaanngggnnaaaaaaaaaaaaaaaaaattttttngggntnaggggnngggggaaaancccna

SEQ ID NO: 11 - DNA, 396 bp, Homo sapiens
agaacacaggtgtcgtgaaaactacccctaaaagccaaaatgggaaaggaaaagactcatatcaacattgtcgtcattggacacgtagattcgggcaagtccaccactactggccatctgatctataaatgcggtggcatcgacaaaagaaccattgaaaaatttgagaaggaggctgctgagatgggaaagggctccttcaagtatgcctgggtcttggataaactgaaagctgagcgtgaacgtggtatcaccattgatatctccttgtggaaatttgagaccagcaagtactatgtgactatcattgatgccccaggacacagagactttatcaaaaacatgattacagggacatctcaggctgactgtgctgtcctgattgttgctgctggt

SEQ ID NO: 12 - DNA, 396 bp, Homo sapiens
cgaaaacctttaaaccccggtcatccggacatcccaacgcatgctcctggagctcacagccttctgtggtgtcatttctgaaacaagggcgtggatccctcaaccaagaagaatgtttatgtcttcaagtgacctgtactgcttggggactattggagaaaataaggtggagtcctacttgtttaaaaaatatgtatctaagaatgttctagggcactctgggaacctataaaggcaggtatttcgggccctcctcttcaggaatcttcctgaagacatggcccagtcgaaggcccaggatggcttttgctgcggccccgtggggtaggagggacagagagacagggagagtcagcctccacattcagaggcatcacaagtaatggcacaattctt

SEQ ID NO: 13 - DNA, 396 bp, Homo sapiens
accacaggctggccacaagaagcgctggagtgtgctggcggctgcaggcctacggggcctggtccggctgctgcacgtgcgtgccggcttctgctgcggggtcatccgagcccacaagaaggccatcgccaccctgtgcttcagccccgcccacgagacccatctcttcacggcctcctatgacaagcggatcatcctctgggacatcggggtgcccaaccaggactacgaattccaggccagccagctgctcacactggacaccacctctatccccctgcgcctctgccctgtcgcctcctgcccggacgcccgcctgctggccggctgcgagggcggctgctgctgctgggacgtgcggctggaccagccccaaaagaggagggtgtgtgaagt

SEQ ID NO: 14 - DNA, 396 bp, Homo sapiens
acggcgtcctcgtggaagtgacatcgtctttaaaccctgcgtggcaatccctgacgcaccgccgtgatgcccagggaagacagggcgacctggaagtccaactacttccttaagatcatccaactattggatgattatccgaaatgtttcattgtgggagcagacaatgtgggctccaagcagatgcagcagatccgcatgtcccttcgcgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactgagatcagggacatgttgctggccaataaggtgccagctgct

SEQ ID NO: 15 - DNA, 396 bp, Homo sapiens
  misc_feature 333: n = A,T,C or G
accgcgcgggcacagggtgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgcttgtcaacggggtgaaacagataaagactctcatagaaaaaacaaacgaagagcgcaagacactgctcagcaacctagaagaagccaagaagaagaaagaggatgccctaaatgagaccagggaatcanagacaaagctgaaggagctcccaggagtgtgcaatgagaccatgatggccctctgggaagagt

SEQ ID NO: 16 - DNA, 396 bp, Homo sapiens
  misc_feature 114, 121, 122, 123, 127, 134, 136, 138, 140, 141, 142,143, 144, 148, 163, 166, 172, 173, 174, 176, 177, 183, 184, 185,187, 195, 196, 198, 199, 202, 203, 206, 213, 214, 215, 216,217, 218, 219, 223, 225, 226, 227, 229, 230, 236, 238: n = A,T,C or G
  misc_feature 239, 252, 256, 257, 261, 262, 268, 269, 273, 278, 280,288, 289, 290, 292, 293, 303, 312, 325, 327, 333, 335, 336, 341,342, 347, 354, 359, 365, 371, 383, 384, 386, 388, 391: n = A,T,C or G
tttttttttttttttttttttttttttttttttttttttttttttttttttttttttttttttttttttttttttttttttttttttttttttttttttttttttttttttttnggggggnnnaaanttttttntnanannnnngggnaaaaaaaaaaaaaanaangggggnnntnnggcccnnnanaaaaaaanngnnaannaanccccccnnnnnnncccncnnntnnggaaanannaaaaccccccccngggnngggnnaaaaanncccnggggnantttttatnnnanncccccccccnggggggggnggaaaaaaaaaantncccccnannaaaannggggnccccccnttttncaaaangggggnccgggccccccnnantnttnggggg

SEQ ID NO: 17 - DNA, 396 bp, Homo sapiens
accacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaactaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtac

SEQ ID NO: 18 - DNA, 396 bp, Homo sapiens
  misc_feature 51, 54, 66, 81, 86, 98, 106, 111, 117, 124, 129, 133,135, 150, 151, 154, 159, 161, 172, 179, 181, 183, 185, 220, 223,229, 238, 258, 259, 264, 282, 289, 292, 294, 299, 303, 311,315, 329, 343, 349, 351, 353, 361, 369, 370, 389, 392: n = A,T,C or G
  misc_feature 396: n = A,T,C or G
tttttttttttttttttttttttttttttttttttttttttttttttttantcnaaaggggaaggnccctttttattaaanttggncattttactttncttttttnaaaangctaanaaaaaanttttntttntncttaaaaaaaccctnnatntcacnancaaaaaaaacnattcccncntncnttttgtgataaaaaaaaaggcaatggaattcaacntancctaanaaaactttncctgggaggaaaaaaaattnntccgngggaaacacttggggctntccaaantgnanccatnctangaggaccntctntaagatttccaaangaaaccccttcctnccaaangnantaccccgntgcctacnncccataaaaaaaacctcanccntaan

SEQ ID NO: 19 - DNA, 396 bp, Homo sapiens
  misc_feature 47, 69, 75, 80, 83, 87, 88, 90, 92, 102, 104, 108, 116,121, 130, 138, 139, 142, 153, 156, 158, 162, 165, 166, 180, 192,193, 195, 201, 224, 226, 232, 235, 237, 241, 248, 251, 253,256, 269, 272, 274, 277, 284, 287, 290, 292, 297: n = A,T,C or G
  misc_feature 299, 305, 306, 315, 323, 324, 326, 332, 351, 368, 377,380, 383, 387, 392: n = A,T,C or G
ttttttttttttttttttttttttttttttttttttttttttttttntggtctgggcttttattttacnaaaaanctaanggnaaanntncnttaaactaantngaanacaaagtnttaangaaaaaggnctgggggnntcntttacaaaaanggncngggncanntttgggcttaaaanttcaaaaagggnncntcaaangggtttgcatttgcatgtttcancnctaaancgnangaanaaacccnggngnccnctgggaaaagttnttnanctnccaaaanatnaantntttgnancagggnntttttgggnaaaaaaannanttccanaaactttccatcccctggntttgggttcggccttgngttttcggnatnatntccnttaangggg

SEQ ID NO: 20 - DNA, 396 bp, Homo sapiens
  misc_feature 29, 43, 49, 53, 55, 75, 81, 100, 110, 111, 125, 129,160, 162, 168, 246, 277: n = A,T,C or G
ttttttttttttttttttttttttttctnaacaaaccctgttnttgggngggngngggtataatactaagttganatgatntcatttacgggggaaggcnctttgtgaannaggccttatttctnttgncctttcgtacagggaggaatttgaagtaaananaaaccnacctggattactccggtctgaactcaaatcacgtaggactttaatcgttgaacaaacaaacctttaatagcggctgcnccattgggatgtcctgatccaacatcgaggncgtaaaccctattgttgatatggactctaaaaataggattgcgctgttatccctagggtaacttgttcccgtggtcaaagttattggatcaattgagtataagtagttcgctttgactg

SEQ ID NO: 21 - DNA, 396 bp, Homo sapiens
  misc_feature 6, 9, 18, 23, 37, 43, 48, 55, 65, 73, 75, 103, 110, 117,123, 125, 134, 153, 182, 195, 202, 205, 213, 216, 223, 239,249, 276, 293, 294, 302, 307, 344, 356, 359, 369, 374, 381,392: n = A,T,C or G
acatanatnttatactancattnaccatctcacttgnaggaanactantatatcnctcacacctnatatcctncntactatgcctagaaggaataatactatngctgttnattatanctactntnataaccctnaacacccactccctcttanccaatattgtgcctattgccatactagtntttgccgcctgcnaagcagnggngggcctanccntactagnctcaatctccaacacntatggcctanactacgtacataacctaaacctactcnaatgctaaaactaatcnncccaacanttatnttactaccactgacatgactttccaaaaaacacatantttgaatcaacncanccacccacancctanttattancatcatccccntact

SEQ ID NO: 22 - DNA, 396 bp, Homo sapiens
  misc_feature 17, 244: n = A,T,C or G
ttttttttttttttganaaaagccggcataaagcacttttattgcaataataaaacttgagactcataaatggtgctgggggaagggtgcagcaacgatttctcaccaaatcactacacaggacagcaaaggggtgagaaggggctgagggaggaaaagccaggaaactgagatcagcagagggagccaagcatcaaaaaacaggagatgctgaagctgcgatgaccagcatcattttcttaanagaacattcaaggatttgtcatgatggctgggctttcactgggtgttaagtctacaaacagcaccttcaattgaaactgtcaattaaagttcttaagatttaggaagtggtggagcttggaaagttatgagattacaaaattcctgaaagtc

SEQ ID NO: 23 - DNA, 396 bp, Homo sapiens
acaaaggcggttccaagctaaggaattccatcagtgcttttttcgcagccaccaaatttagcaggcctgtgaggttttcatatcctgaagagatgtattttaaagctttttttttttaatgaaaaaatgtcagacacacacaaaagtagaatagtaccatggagtccccacgtacccagcctgcagcttcaacagttaccacatttgccaaccggagagactgccaaggcaggaaaaagccctggaaagcccacggcccctttttcccttgggtcagaggccttagagctggctgccaaagcagccaaccaaaggggcagctcagctccttcgtggcaccagcagtgttcctgatgcagttgaagagttgatgtctttgacaacatacggacactg

SEQ ID NO: 24 - DNA, 396 bp, Homo sapiens
  misc_feature 313, 337, 340, 350, 351, 352, 353, 354, 355, 356, 366,376, 377, 378, 382, 384, 385, 387, 389, 390, 392, 393, 396: n = A,T,C or G
cgactatcctctcagattcttatctggcactaatttataactattatattatcagagactatgtagcaatatatcagtgcacaggcgcatcccaggcctgtacagatgtatgtctacacgtaagtataaatgaatttgcataccaggttttacacttgcatctctaatagagattaaaaacaacaaattggcctcttcctaagtatattaatatcatttatccttacattttatgcctccccctaaattaatgactgagttggtggaaagcggctaggttttattcatactgttttttgttctcaacttcaanagtaatctacctctgaaaaatttntantttaatattnnnnnnnaggaatttgngccactttannncttncnntntnntnnccn

SEQ ID NO: 25 - DNA, 396 bp, Homo sapiens
  misc_feature 90, 125, 136, 278, 299, 301, 305, 344, 347, 353, 355,356, 357, 359, 360, 361, 365, 369, 378, 380, 381, 382, 383, 384,385, 386, 391, 392, 393, 395, 396: n = A,T,C or G
ttttttttttttttttttttgtcttttaaaaaatataaaagtgttattattttaaaacatcaagcattacagactgtaaaatcaattaanaactttctgtatatgaggacaaaaatacatttaanacatatacaanaagatgctttttcctgagtagaatgcaaacttttatattaagcttctttgaattttcaaaatgtaaaataccaaggctttttcacatcagacaaaaatcaggaatgttcaccttcacatccaaaaagaaaaaaaaaaaaaanccaattttcaagttgaagttnancaanaatgatgtaaaatctgaaaaaagtggccaaaattttaanttncaacanannngnnncagntttnatggatctntnnnnnnncttcnnntnn

SEQ ID NO: 26 - DNA, 396 bp, Homo sapiens
  misc_feature 313, 314, 316, 318, 321, 343, 344, 352, 353, 356, 363,366, 370, 372, 373, 374, 375, 377, 378, 379, 383, 384, 385, 386,387, 391, 393, 394, 395, 396: n = A,T,C or G
gacgctcccccctccccccgagcgccgctccggctgcaccgcgctcgctccgagtttcaggctcgtgctaagctagcgccgtcgtcgtctcccttcagtcgccatcatgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtacccgaaagcacagtaatcactgnngncnatnttgtcatgaaccatcacctgcnngaaacaannttnacaaaanaancctncnnnnannncctnnnnnattncnnnn

SEQ ID NO: 27 - DNA, 396 bp, Homo sapiens
  misc_feature 49, 61, 66, 73, 75, 99, 102, 103, 105, 107, 120, 124,126, 129, 138, 139, 141, 147, 155, 157, 162, 165, 175, 187, 191,193, 198, 207, 217, 218, 220, 221, 223, 226, 231, 232, 245,257, 259, 260, 263, 266, 271, 287, 305, 306, 307, 308: n = A,T,C or G
  misc_feature 321, 330, 332, 335, 342, 343, 344, 345, 349, 350, 351,352, 354, 355, 356, 357, 365, 366, 367, 370, 371, 372, 373, 374,375, 376, 377, 378, 379, 380, 381, 382, 383, 386, 387, 388,389, 391, 392, 393, 394, 395, 396: n = A,T,C or G
tttttttttttttttttttttttttttttttttttttttttggctaaantttatgtatacnggttnttcaaangngggggaggggggggggcatccatntanncncnccaggtttatggngggntnttntactattannanttttcncttcaaancnaaggnttntcaaatcatnaaaattattaanattncngctgntaaaaaaangaatgaaccnncnnanganagganntttcatggggggnatgcatcggggnannccnaanaaccncggggccattcccganaggcccaaaaaatgtttnnnnaaaaagggtaaanttacccccntnaantttatannnnaaannnnannnnagcccaannnttnnnnnnnnnnnnnnccnnnnannnnnn

SEQ ID NO: 28 - DNA, 396 bp, Homo sapiens
  misc_feature 278, 283, 298, 309, 326, 331, 338, 351, 355, 356, 357,358, 360, 371, 377, 378, 383, 386, 387, 391, 393, 394, 395: n = A,T,C or G
cgacctttttttttttttttatagatgaaagagggtttatttattaatatatgatagccttggctcaaaaaagacaaatgagggctcaaaaaggaattacagtaactttaaaaaatatattaaacatatccaagatcctaaatatattattctccccaaaagctagctgcttccaaacttgatttgatattttgcatgttttccctacgttgcttggtaaatatatttgcttctcctttctgcaatcgacgtctgacagctgatttttgctgttttgncaacntgacgtttcaccttntgtttcaccanttctggaggaattgttnaacancttacancactgccttgaanaaannnnangcctcaaaagntcttgnnctatnctnnttcntnnnt

SEQ ID NO: 29 - DNA, 396 bp, Homo sapiens
  misc_feature 329, 334, 361, 386, 390: n = A,T,C or G
gacttgctcatttagagtttgcaggaggctccatactaggttcagtctgaaagaaatctcctaatggtgctatagagagggaggtaacagaaagactcttttagggcatttttctgactcatgaaaagagcacagaaaaggatgtttggcaatttgtcttttaagtcttaaccttgctaatgtgaatactgggaaagtgatttttttctcactcgtttttgttgctccattgtaaagggcggaggtcagtcttagtggccttgagagttgcttttggcatttaaatattctaagagaattaactgtatttcctgtcacctattcactantgcangaaatatacttgctccaaataagtcantatgagaagtcactgtcaatgaaanttgntttgtt

SEQ ID NO: 30 - DNA, 396 bp, Homo sapiens
  misc_feature 28, 83, 126, 138, 254, 275, 298, 310, 311, 353, 363,374, 379, 393: n = A,T,C or G
tttttttttttttttttttgaaatttanaaacaaattttatttaagatctgaaatacaattcctaaaatatcaacttttccanaaaaccgtggctacacaataatgcattgcctctatcatgttanaacgtgcattanactcaaatacaaaaaccatgaaacaaatcaccatccttcaacaatttgagcaaagatagaatgcctaagaacaacatagatggacttgcagaggatgggctgttttacttcaagcnccataaaaaaaaaaaagagcncaaatgcattgggttttcaggtntatacattaagnngaacctttggcactaggaatcagggcgttttgtcacatagcnttaacacatnttaaaaaattntgtantgtcaaagggatangaa

SEQ ID NO: 31 - DNA, 396 bp, Homo sapiens
  misc_feature 285, 287, 350, 362, 365, 377, 378, 382, 388, 390, 393: n = A,T,C or G
gacgggccagggccatctggaaagggaactcggcttttccagaacgtggtggatcatctgtcgggtgtgtggtgaacacgttcagttcatcagggcctacgctccgggaaggggcccccagctgtggctctgccatgccgggctgtgtttgcagctgtccgagtctccatccgcctttagaaaaccagccacttcttttcataagcactgacagggcccagcccacagccacaggtgcgatcagtgcctcacgcaggcaaatgcactgaaacccaggggcacacncncgcagagtgaacagtgagttcccccgacagcccacgacagccaggactgccctccccaccccnccccgaccccangancacggcacacanntcancctctnanctngct

SEQ ID NO: 32 - DNA, 396 bp, Homo sapiens
  misc_feature 341: n = A,T,C or G
cgactggcctcataccttgtctacacagtccctgcacagggttcctaacctgtggttagtaaagaatgtcactttctaacaggtctggaagctccgagtttatcttgggaactcaagaggagaggatcacccagttcacaggtatttgaggatacaaacccattgctgggctcggctttaaaagtcttatctgaaattccttgtgaaacagagtttcatcaaagccaatccaaaaggcctatgtaaaaataaccattcttgctgcactttatgcaaataatcaggccaaatataagactacagtttatttacaatttgtttttaccaaaaatgaggactanagagaaaaatggtgctccaaagcttatcatacatttgtcattaagtcctagtctc

SEQ ID NO: 33 - DNA, 396 bp, Homo sapiens
  misc_feature 121, 122, 124, 125, 126, 128, 130, 131, 132, 133, 134,136, 137, 153, 154, 155, 156, 157, 158, 159, 168, 169, 170, 171,172, 173, 174, 175, 176, 177, 178, 179, 184, 185, 192, 197,199, 200, 202, 204, 205, 208, 209, 210, 211, 214, 215: n = A,T,C or G
  misc_feature 216, 217, 218, 222, 227, 228, 229, 233, 234, 241, 242,244, 245, 246, 247, 248, 249, 252, 260, 261, 262, 263, 264, 265,270, 272, 273, 274, 275, 279, 282, 284, 288, 290, 291, 292,293, 294, 299, 300, 301, 302, 303, 306, 313, 314, 319: n = A,T,C or G
  misc_feature 327, 328, 330, 331, 332, 333, 334, 335, 343, 349, 350,351, 352, 355, 360, 369, 370, 371, 375, 379, 387, 388, 390, 391,392, 393, 394, 395, 396: n = A,T,C or G
ccttttttttttttttttttttttttttttttttttttttttttttttttttttttttttttttttttttttttttttttttttttttttttttttttttttttttttttttttttttttnngnnntntnnnnnannaaaaaaaaaaaaaaannnnnnnaaaaaaaannnnnnnnnnnnttttnnggggggnttttnanngnannttnnnnttnnnnnaaanccccnnngggnnggggggnntnnnnnnggnaaaaaaannnnnnggggncnnnngggnccncncccnannnnnaaaannnnnggnttttttnnttttnaaaaaaanngnnnnnnaacaaaantttttnnnnaanttttngggggaaannncccntttntttttttnnannnnnnn

SEQ ID NO: 34 - DNA, 396 bp, Homo sapiens
  misc_feature 8, 60, 72, 123, 128, 155, 172, 198, 207, 246, 305, 325,348, 349, 369, 371, 380, 393, 394: n = A,T,C or G
acggaccnagctggaggagctgggtgtggggtgcgttgggctggtggggaggcctagttngggtgcaagtangtctgattgagcttgtgttgtgctgaagggacagccctgggtctagggganagagnccctgagtgtgagacccaccttccccngtcccagcccctcccanttcccccagggacggccacttcctgntccccgacncaaccatggctgaagaacaaccgcaggtcgaattgttcntgaaggctggcagtgatggggccaagattgggaactgcccattctcccacagactgttnatggtactgtggctcaaggnagtcaccttcaatgttaccaccnntgacaccaaaaggcggaccnanacagtgcanaagctgtgcccanngg

SEQ ID NO: 35 - DNA, 396 bp, Homo sapiens
tcgaccaaaatcaaatctggcactcacaagccctggccgacccccaatgggttttaccactccccctctagaccctgtcttgcaaaatcctctccctagccagctagtattttctgggctaaagactgtacaaccagttcctccattttatagaagtttactcactccaggggaaatggtgagtcctccaacctccctttcaaccagtcccatcattccaaccagtggtaccatagagcagcaccccccgccaccctctgagccagtagtgccagcagtgatgatggccacccatgagcccagtgctgacctggcacccaagaaaaagcccaggaagtcaagcatgcctgtgaagattgagaaggaaattattgataccgccgatgagtttgatga

SEQ ID NO: 36 - DNA, 396 bp, Homo sapiens
tcgacgggaagagcctgctacggtggactgtgagactcagtgcactgtcctcctcccagcgaccccacgctggaccccctgccggaccctccacccttcggcccccaagcttcccaggggcttcctttggactggactgtccctgctcatccattctcctgccacccccagacctcctcagctccaggttgccacctcctctcgccagagtgatgaggtcccggcttctgctctccgtggcccatctgcccacaattcgggagaccacggaggagatgctgcttgggggtcctggacaggagcccccaccctctcctagcctggatgactacgtgaggtctatatctcgactggcacagcccacctctgtgctggacaaggccacggcccagggcc

SEQ ID NO: 37 - DNA, 396 bp, Homo sapiens
  misc_feature 376: n = A,T,C or G
cgacggtgtcagcaactggccatgccacagcacataaagattacagtgacaagaaaaacattgtttgaggattcctttcaacagataatgagcttcagtccccaagatctgcgaagacgtttgtgggtgatttttccaggagaagaaggtttagattatggaggtgtagcaagagaatggttctttcttttgtcacatgaagtgttgaacccaatgtattgcctgtttgaatatgcagggaaggataactactgcttgcagataaaccccgcttcttacatcaatccagatcacctgaaatattttcgttttattggcagatttattgccatggctctgttccatgggaaaattcatagacacgggtttttctttnccattctataagcgtatctt

SEQ ID NO: 38 - DNA, 396 bp, Homo sapiens
cgaccaaaatgataaatagctttaagaatgtgctaatgataaatgattacatgtcaatttaatgtacttaatgtttaataccttatttgaataattacctgaagaatatattttttagtactgcatttcattgattctaagttgcactttttacccccatactgttaacatatctgaaatcagaatgtgtcttacaatcagtgatcgtttaacattgtgacaaagtttaatggacagttttttcccatatgtatatataaaataatgtgttttacaatcagtggcttagattcagtgaaatacagtaattcattcaattatgatagtatctttacagacattttaaaaataagttatttttatatgctaatattctatgttcaagtggaatttgga

SEQ ID NO: 39 - DNA, 396 bp, Homo sapiens
tcgaccaagaatagatgctgactgtactcctcccaggcgccccttccccctccaatcccaccaaccctcagagccacccctaaagagatactttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcaagtgggacagggcatggccaggtggccactccagacccctggcttttcactgctggctgccttagaacctttcttacattagcagtttgctttgtatgcactttgtttttttctttgggtcttgttttttttttccacttagaaattg

SEQ ID NO: 40 - DNA, 396 bp, Homo sapiens
  misc_feature 200, 375: n = A,T,C or G
ttttttttttttttgttatttagtttttatttcataatcataaacttaactctgcaatccagctaggcatgggagggaacaaggaaaacatggaacccaaagggaactgcagcgagagcacaaagattctaggatactgcgagcaaatggggtggaggggtgctctcctgagctacagaaggaatgatctggtggttaanataaaacacaagtcaaacttattcgagttgtccacagtcagcaatggtgatcttcttgctggtcttgccattcctggacccaaagcgctccatggcctccacaatattcatgccttctttcactttgccaaacaccacatgcttgccatccaaccactcagtcttggcagtgcanatgaaaaactgggaaccattt

SEQ ID NO: 41 - DNA, 396 bp, Homo sapiens
  misc_feature 288: n = A,T,C or G
tcgacctcttgtgtagtcacttctgattctgacaatcaatcaatcaatggcctagagcactgactgttaacacaaacgtcactagcaaagtagcaacagctttaagtctaaatacaaagctgttctgtgtgagaattttttaaaaggctacttgtataataacccttgtcatttttaatgtacaaaacgctattaagtggcttagaatttgaacatttgtggtctttatttactttgcttcgtgtgtgggcaaagcaacatcttccctaaatatatattacccaaagnaaaagcaagaagccagattaggtttttgacaaaacaaacaggccaaaagggggctgacctggagcagagcatggtgagaggcaaggcatgagagggcaagtttgttgt

SEQ ID NO: 42 - DNA, 396 bp, Homo sapiens
  misc_feature 65, 68, 69, 71, 72, 75, 77, 79, 82, 85, 86, 87, 89, 90,97, 98, 105, 107, 109, 112, 117, 121, 122, 124, 126, 149, 152,153, 155, 157, 161, 163, 167, 168, 169, 174, 177, 178, 179,180, 186, 188, 192, 201, 202, 207, 208, 215, 217, 220: n = A,T,C or G
  misc_feature 225, 230, 242, 243, 247, 250, 259, 263, 271, 272, 279,284, 295, 298, 299, 308, 309, 312, 323, 342, 348, 351, 363, 366,370, 386, 390, 392: n = A,T,C or G
ctttttttttttttttttttttttttttttttttttttttttttttttttttttttttttaaaanccnnannaanananggnaannnannaaaaaanncaaaccncntntanaaaangccnntntnagggggggggttcaaaaccaaanggnngntnggangnaaannnaaaanttnnnngggggnanaaanaaaaagggnngaaanntgacccnanaangaccngaaancccgggaaaccnngggntanaaaaaaagntganccctaaanncccccgnaaaangggggaagggnaannccaaatccnntgngggttgggggnggggaaaaaaaaaaccccnaaaaantgnaaaaaaccgggnttnaaanatttgggttcgggggntttntnttaa

SEQ ID NO: 43 - DNA, 396 bp, Homo sapiens
  misc_feature 108, 195, 213, 279, 287, 349: n = A,T,C or G
ttttttttttttttgcttcactgctttatttttgaaatcacaagcaattcaaagtgatcatcattgaggcttctgttaaaagttcttccaaagttgcccagttttaanattaaacaatattgcactttaagatgaactaacttttgggattctcttcaaagaaggaaagtattgctccatctgtgcttttcttanactaaaagcatactgcanaaaactctattttaaaaatcaacactgcagggtacagtaacatagtaaagtacctgcctattttanaatcctanagaacatttcattgtaagaaactagcccattatttaagtgtccacagtatttttcatttcantggtccaagatgccaaggtttccaaacacaatcttgttctctaatac

SEQ ID NO: 44 - DNA, 396 bp, Homo sapiens
gacctagttttacctcttaaatatctctgttcccttctaagttgtttgctgtgttttcttcagagcaagaaggttatattttttaaaatttacttagtaatgcacattcaaaacacacatcaagtcttcaggataaagttcaaaaccgctgtcatggccccatgtgatctctccctcccctacccctctatcatttagtttcttctgcgcaagccactctggcttcctttcagttttgtggttcccgtttttagctagttcagtggttttcaatgggcatttcttgcctttttttttctaaacgacaaatagaaatacatcttctttattatcctccaaatccaattcagaggtaatatgctccacctacacacaattttagaaataaattaaaaa

SEQ ID NO: 45 - DNA, 396 bp, Homo sapiens
  misc_feature 18, 19, 22, 39, 40, 43, 62, 84, 90, 99, 103, 104, 105,117, 120, 123, 128, 134, 139, 141, 142, 143, 144, 145, 182, 187,207, 218, 219, 242, 247, 257, 260, 263, 272, 276, 277, 279,284, 288, 294, 296, 297, 305, 310, 314, 319, 320, 322: n = A,T,C or G
  misc_feature 364, 366, 376, 378, 381, 387, 388, 396: n = A,T,C or G
ttttttttttttttaaannttntaaatttttaatgaaannganttagaacaatgtattattnacatgtaaataaaaaaagagancataanccccatatnctcnnnaaaggaaggganacngcnggccntttatnagaanannnnncatataagaccccattaagaagaatctggatctaaanacttncaaacaggagttcacagtangtgaacagcannccctaatcccactgatgtgatgnttcanataaaatcancancgntgatcgggnatcnnancaatntgancggaanannactgctcnatatntttnagganncngatgtggtcattttttacaaagataatggccacacccttccngnccgaatcgancnganctcccnnttctgtgn

SEQ ID NO: 46 - DNA, 396 bp, Homo sapiens
  misc_feature 24, 105, 144, 188, 190, 214, 317, 369, 371, 378: n = A,T,C or G
tttttttttttttttttttctganacagagtctcattctgttgcctaggctggattgcagtggtgccatctcggctcactgcaacctccgcctcctgggttccanaaattctcctgcctcagcctcccgggtagctgggactanaggcacacgccaccacgccaggctaatttttatatttttagtananatggcgtttcaccatgttgaccanactgatctcgaactcccgacctcgtgatccacccacctcggcctcccaaagtgctgggattacaggcgtgaaaccaccaggcccggcctgaaatatctatttnttttcagattatttttaaaattccatttgatgaatcttttaaagtgagctananaaagtgngtgtgtacatgcacacac

SEQ ID NO: 47 - DNA, 396 bp, Homo sapiens
  misc_feature 290: n = A,T,C or G
tttttttttttttttttgctgttgccaactgtttattcagggccctgaacgggtggtgcgtggacatgcaacacactcgggcccacagcagcgtgaccggccgctcccaagccccgggcgcacaaccacagccaggagcagcccctgccaccactgggccaccgtccagggccccacaggaccagccgaaggtgccccgggccgaggccagctgggtcaggtgtacccctagcctggggttgagtgaggagcggcacccccagtatcctgtgtaccccaagttgcccagnaggccgagggggccttgggctccatctgcactggccaccccgtgccaagcatcacagctgcgtgagcaggtttgtgtgtgagcgtgtggcggggcctggttgtccc

SEQ ID NO: 48 - DNA, 396 bp, Homo sapiens
  misc_feature 393, 396: n = A,T,C or G
ctgggcctgtgccgaagggtctgggcagatcttccaaagatgtacaaaatgtagaaattgccctcaagcaaatgcaaagatgctcaacacccttagtcatcaagaaaatgcaaatggaatccacagagagatactgcacactgacaaagatggtcgtattactaaaggtgaataaccagcgcggggggcacgtggagtcactggaacatttgtgcaatgctggtgggaatgtcaacccgtgcggccctctggaataagcctggcagctcctccaagagttacccgtgtgacccagcaattccactcctagctccacccacaggaattgaaagcaaagacgcaaacagatgcctgtgcaccaaagttcacggcagcatccttcgccatagtggnaan

SEQ ID NO: 49 - DNA, 396 bp, Homo sapiens
  misc_feature 32, 40, 44, 64, 70, 83, 87, 92, 104, 115, 118, 125,127, 130, 137, 155, 168, 171, 173, 175, 192, 201, 206, 208, 218,219, 235, 247, 249, 256, 259, 260, 269, 297, 306, 310, 320,321, 328, 331, 345, 356, 381, 389, 395: n = A,T,C or G
accccaaaatgggaaaggaaaagactcatatnaacattgncgtnattggacacgtacattcggncaagtncaccactactggncatntgatntataaatgcggnggcatcgacanaanaaccatngnaanatttganaaggaggctgctgatatnggaaagggctccntcnantntgcctgggtcttggatnaactgaaanctgancntgaacgtggnntcaccattgatatctncttgtggaaatntnagaccancanntactatgtnactatcattgatgccccaggacacaganactttatcnaaancatgattacnnggacatntanagctgactgtgctngcctgattgtngctgctggtgttggtgaatttgaanctggtatntccaana

SEQ ID NO: 50 - DNA, 396 bp, Homo sapiens
cgacttcttgctggtgggtggggcagtttggtttagtgttatactttggtctaagtatttgagttaaactgcttttttgctaatgagtgggctggttgttagcaggtttgtttttcctgctgttgattgttactagtggcattaacttttagaatttgggctggtgagattaattttttttaatatcccagctagagatatggcctttaactgacctaaagaggtgtgttgtgatttaattttttcccgttcctttttcttcagtaaacccaacaatagtctaaccttaaaaattgagttgatgtccttataggtcactacccctaaataaacctgaagcaggtgttttctcttggacatactaaaaaatacctaaaaggaagcttagatgggctg

SEQ ID NO: 51 - DNA, 396 bp, Homo sapiens
  misc_feature 18, 52, 59, 148, 267, 321, 332: n = A,T,C or G
ttttttttttttcagcgnggatttattttatttcattttttactctcaaganaaagaanagttactattgcaggaacagacatttttttaaaaagcgaaactcctgacacccttaaaacagaaaacattgttattcacataataatgnggggctctgtctctgccgacaggggctgggttcgggcattagctgtgccgtcgacaatagccccattcaccccattcataaatgctgctgctacaggaagggaacagcggctctcccanagagggatccaccctggaacacgagtcacctccaaagagctgcgactgtttganaatctgccaanaggaaaaccactcaatgggacctggataacccaggcccgggagtcatagcaggatgtggtactt

SEQ ID NO: 52 - DNA, 396 bp, Homo sapiens
  misc_feature 81, 189: n = A,T,C or G
acctcgctaagtgttcgctacgcggggctaccggatcggtcggaaatggcagaggtggaggagacactgaagcgactgcanagccagaagggagtgcagggaatcatcgtcgtgaacacagaaggcattcccatcaagagcaccatggacaaccccaccaccacccagtatgccagcctcatgcacagnttcatcctgaaggcacggagcaccgtgcgtgacatcgacccccagaacgatctcaccttccttcgaattcgctccaagaaaaatgaaattatggttgcaccagataaagactatttcctgattgtgattcagaatccaaccgaataagccactctcttggctccctgtgtcattccttaatttaatgccccccaagaatgttaatgt

SEQ ID NO: 53 - DNA, 396 bp, Homo sapiens
  misc_feature 224, 225, 228, 235, 240, 246, 257, 266, 274, 279, 281,282, 283, 285, 287, 288, 290, 291, 292, 293, 294, 295, 296, 297,300, 301, 303, 307, 311, 313, 314, 317, 318, 319, 320, 321,323, 324, 328, 329, 330, 336, 337, 338, 339, 340, 341: n = A,T,C or G
  misc_feature 342, 343, 344, 345, 346, 347, 348, 349, 350, 351, 352,356, 357, 358, 359, 362, 363, 364, 365, 366, 367, 373, 380, 381,382, 385, 387, 388, 389, 390, 392: n = A,T,C or G
ttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttannttnttttttnttttncctttnttttaattcanaaaaagaanaagaaaanataanannnancnnannnnnnnnatnntncttnatantnnttnnnnnanngggnnngcgagnnnnnnnnnnnnnnnnntctnnnnttnnnnnncttgcnccccttnnnttngnnnnangcaa

SEQ ID NO: 54 - DNA, 396 bp, Homo sapiens
  misc_feature 367: n = A,T,C or G
ctcttggggctgctgggactcgcgtcggttggcgactcccggacgtaggtagtttgttgggccgggttctgaggccttgcttctctttacttttccactctaggccacgatgccgcagtaccagacctgggaggagttcagccgcgctgccgagaagctttacctcgctgaccctatgaaggcacgtgtggttctcaaatataggcattctgatgggaacttgtgtgttaaagtaacagatgatttagtttgtttggtgtataaaacagaccaagctcaagatgtaaagaagattgagaaattccacagtcaactaatgcgacttatggtagccaaggaagcccgcaatgttaccatggaaactgantgaatggtttgaaatgaagactttgtcgt

SEQ ID NO: 55 - DNA, 396 bp, Homo sapiens
cgacggtttgccgccagaacacaggtgtcgtgaaaactacccctaaaagccaaaatgggaaaggaaaagactcatatcaacattgtcgtcattggacacgtagattcgggcaagtccaccactactggccatctgatctataaatgcggtggcatcgacaaaagaaccattgaaaaatttgagaaggaggctgctgagatgggaaagggctccttcaagtatgcctgggtcttggataaactgaaagctgagcgtgaacgtggtatcaccattgatatctccttgtggaaatttgagaccagcaagtactatgtgactatcattgatgccccaggacacagagactttatcaaaaacatgattacagggacatctcaggctgactgtgctgtcctg

SEQ ID NO: 56 - DNA, 396 bp, Homo sapiens
  misc_feature 134, 145, 255, 279, 337, 344, 369: n = A,T,C or G
ttttttttttttttttctcatttaacttttttaatgggtctcaaaattctgtgacaaatttttggtcaagttgtttccattaaaaagtactgattttaaaaactaataacttaaaactgccacacgcaaaaaanaaaaccaaagnggtccacaaaacattctcctttccttctgaaggttttacgatgcattgttatcattaaccagtcttttactactaaacttaaatggccaattgaaacaaacagttctganaccgttcttccaccactgattaanagtggggtggcaggtattagggataatattcatttagccttctgagctttctgggcanacttggngaccttgccagctccagcagccttnttgtccactgctttgatgacacccacc

SEQ ID NO: 57 - DNA, 396 bp, Homo sapiens
  misc_feature 52, 57, 58, 61, 72, 75, 77, 84, 87, 88, 93, 100, 101,111, 117, 119, 121, 131, 132, 133, 134, 142, 143, 154, 156, 159,167, 168, 170, 175, 176, 182, 183, 185, 186, 190, 192, 194,198, 199, 200, 209, 212, 217, 218, 220, 232, 235, 253: n = A,T,C or G
  misc_feature 255, 257, 258, 260, 262, 263, 270, 271, 273, 277, 280,281, 284, 285, 289, 296, 297, 298, 303, 305, 307, 309, 310, 317,322, 324, 337, 338, 342, 344, 346, 347, 349, 351, 356, 358,366, 368, 371, 377, 380, 388, 389, 393, 396: n = A,T,C or G
cctttttttttttttttttttttttttttttttttttttttttttttttttnaaaannttntttttgcaaanccnancaaaaanggnnggaangaaaaannggaaaaattntttttncntntttgggaacnnnnagcccttnntttgaaaaaangnggncttaaaanngntgaannaaaggnnanncccngntncttnnntttaaaaanaanggggnngnttttttttaaanaanattttttttttccctaanancnncnanntgaaacnngncccnacnnctnncttnaaagggnnnaaatnanangnnaaaaaanccctnancccccccccttanntttncnannananaaagncnttttgggncntgnaaaaanaancctttttnntgcnttn

SEQ ID NO: 58 - DNA, 396 bp, Homo sapiens
cgacctcaaatatgccttattttgcacaaaagactgccaaggacatgaccagcagctggctacagcctcgatttatatttctgtttgtggtgaactgattttttttaaaccaaagtttagaaagaggtttttgaaatgcctatggtttctttgaatggtaaacttgagcatcttttcactttccagtagtcagcaaagagcagtttgaattttcttgtcgcttcctatcaaaatattcagagactcgagcacagcacccagacttcatgcgcccgtggaatgctcaccacatgttggtcgaagcggccgaccactgactttgtgacttaggcggctgtgttgcctatgtagagaacacgcttcacccccactccccgtacagtgcgcacaggcttt

SEQ ID NO: 59 - DNA, 396 bp, Homo sapiens
  misc_feature 25, 45, 116, 178, 198, 211, 225, 235, 253, 266, 281,324, 367, 377, 389: n = A,T,C or G
cttttttttttttttttttttcagnggaaaataacttttattganaccccaccaactgcaaaatctgttcctggcattaagctccttcttcctttgcaattcggtctttcttcagnggtcccatgaatgctttcttctcctccatggtctggaagcggccatggccaaacttggaggnggtgtcaatgaacttaaggncaatcttctccanagcccgccgcttcntctgcaccancaaggacttgcggagggngagcacccgcttnttggttcccaccacncagcctttcagcatgacaaagtcattggtcacttcaccatagnggacaaagccacccaaagggttgatgctccttggcaaataggncatagtcacnggaggcattgtncttgatc

SEQ ID NO: 60 - DNA, 396 bp, Homo sapiens
acctcagctctcggcgcacggcccagcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggg

SEQ ID NO: 61 - DNA, 396 bp, Homo sapiens
tagcttgtcggggacggtaaccgggacccggtgtctgctcctgtcgccttcgcctcctaatccctagccactatgcgtgagtgcatctccatccacgttggccaggctggtgtccagattggcaatgcctgctgggagctctactgcctggaacacggcatccagcccgatggccagatgccaagtgacaagaccattgggggaggagatgactccttcaacaccttcttcagtgagacgggcgctggcaagcacgtgccccgggctgtgtttgtagacttggaacccacagtcattgatgaagttcgcactggcacctaccgccagctcttccaccctgagcagctcatcacaggcaaggaagatgctgccaataactatgcccgagggcactac

SEQ ID NO: 62 - DNA, 396 bp, Homo sapiens
  misc_feature 261, 269, 313, 333, 346, 354, 359, 390, 394, 395, 396: n = A,T,C or G
tcgacgtttcctaaagaaaaccactctttgatcatggctctctctgccagaattgtgtgcactctgtaacatctttgtggtagtcctgttttcctaataactttgttactgtgctgtgaaagattacagatttgaacatgtagtgtacgtgctgttgagttgtgaactggtgggccgtatgtaacagctgaccaacgtgaagatactggtacttgatagcctcttaaggaaaatttgcttccaaattttaagctggaaagncactggantaactttaaaaaagaattacaatacatggctttttagaatttcnttacgtatgttaagatttgngtacaaattgaantgtctgtnctgancctcaaccaataaaatctcagtttatgaaanaaannn

SEQ ID NO: 63 - DNA, 396 bp, Homo sapiens
  misc_feature 3, 11, 16, 18, 23, 26, 30, 34, 37, 50, 51, 60, 61, 62,63, 64, 75, 82, 83, 84, 85, 87, 89, 93, 94, 97, 98, 99, 118,119, 120, 122, 134, 136, 138, 139, 141, 144, 145, 147, 152,156, 187, 188, 193, 195, 204, 211, 214, 216, 222, 226: n = A,T,C or G
  misc_feature 228, 235, 242, 258, 264, 265, 269, 275, 294, 298, 301,307, 316, 326, 334, 335, 339, 340, 343, 350, 351, 355, 373, 378,390: n = A,T,C or G
ttntttttttnttttntnttttntcnttgnttgnacngaacccggcgctnnttccccacnnnnnacggccgcccntattcannnntncntcanntannnaccgcaccctcggactgcnnntngggccccgccgncnanncnccnncncccanttcnccgccgccgccgccgcctttttttattggcnnccatnanaaccggggncacctcncangngcgccnaaantnggggcangactcanagggggccatcaaccnccaagnncaanctgganctctacaaacggcctacgntttntgnccatgngggtagggntttacccgcnatgatgannatgnnaanaactttnncaanccctttattaaccaatgnggtgnggagacggaacntggtta

SEQ ID NO: 64 - DNA, 396 bp, Homo sapiens
  misc_feature 175, 177, 340, 393: n = A,T,C or G
tcgacgtcggggtttcctgcttcaacagtgcttggacggaacccggcgctcgttccccaccccggccggccgcccatagccagccctccgtcacctcttcaccgcaccctcggactgccccaaggcccccgccgccgctccagcgccgcgcagccaccgccgccgccgccgcctntncttagtcgccgccatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaanaactttgccaaatactttcttcccaatctcatgaggagaaggaacatgctganaaa

SEQ ID NO: 65 - DNA, 396 bp, Homo sapiens
  misc_feature 26, 56, 103, 122, 145, 151, 154, 187, 189, 203, 224,256, 273, 305, 344: n = A,T,C or G
tttttttttttttttttttttttttnaccaataatgcttttattttccacatcaanattaatttatatgttagttttagtacaagtactaaaatgtatacttnttgccctaatagctaaggnatacataagcttcaccatacatnttgcanccncctgtctgtcctatgtcattgttataaatgtananattttaggaaactnttttattcaacctgggacatntatactgtaggagttagcactgacctgatgtnttatttaaaagtaatgnatattacctttacatatattccttatatattnaaacgtatttccatgttatccagcttaaaatcacatggnggttaaaagcatgagttctgagtcaaatctggactgaaatcctgatgctccc

SEQ ID NO: 66 - DNA, 396 bp, Homo sapiens
tcgacttttttttttccaggacattgtcataattttttattatgtatcaaattgtcttcaatataagttacaacttgattaaagttgatagacatttgtatctatttaaagacaaaaaaattcttttatgtacaatatcttgtctagagtctagcaaatatagtacctttcattgcaggatttctgcttaatataacaagcaaaaacaaacaactgaaaaaatataaaccaaagcaaaccaaaccccccgctcaactacaaatgtcaatattgaatgaagcattaaaagacaaacataaagtaacttcagcttttatctagcaatgcagaatgaatactaaaattagtggcaaaaaaacaaacaacaaacaacaaacaaaacaaaacaaacaaaca

SEQ ID NO: 67 - DNA, 396 bp, Homo sapiens
acgcttttgtccttcattttaactgttatgtcatactgttatgttgacatatttctttataagagaatagaggcaaaagtatagaactgaggatcatttgtatttttgagttggaaattatgaaacttcaccatattatgatcatacatattttgaagaacagactgaccaaagctcacctgttttttgtgttaggtgctttggctgaacttgattccagcccccttttccctttggtgttgtgtatgtctcttcatttcctctcaaatcttcaactcttgccccatgtctccttggcagcaggatgctggcatctgtgtagtcctcatactgtttactgataacccacaaattcattttcatggcagacctaagctcagaccctgccttgtcctg

SEQ ID NO: 68 - DNA, 396 bp, Homo sapiens
acctgagtcctgtcctttctctctccccggacagcatgagcttcaccactcgctccaccttctccaccaactaccggtccctgggctctgtccaggcgcccagctacggcgcccggccggtcagcagcgcggccagcgtctatgcaggcgctgggggctctggttcccggatctccgtgtcccgctccaccagcttcaggggcggcatggggtccgggggcctggccaccgggatagccgggggtctggcaggaatgggaggcatccagaacgagaaggagaccatgcaaagcctgaacgaccgcctggcctcttacctggacagagtgaggagcctggagaccgagaaccggaggctggagagcaaaatccgggagcacttggagaagaagggac

SEQ ID NO: 69 - DNA, 396 bp, Homo sapiens
  misc_feature 1, 4, 6, 8, 9, 11, 18, 19, 36, 53, 60, 64, 79, 84, 92,94, 97, 105, 114, 120, 123, 127, 129, 134, 137, 138, 139, 142,143, 147, 149, 151, 152, 156, 158, 167, 170, 172, 180, 182,184, 187, 188, 189, 194, 197, 201, 209, 212, 218, 219: n = A,T,C or G
  misc_feature 220, 222, 223, 225, 228, 229, 230, 232, 233, 236, 242,244, 247, 250, 251, 253, 256, 257, 259, 261, 270, 271, 274, 277,278, 279, 282, 284, 288, 289, 296, 298, 300, 310, 315, 316,320, 321, 324, 328, 330, 331, 334, 336, 340, 347, 350: n = A,T,C or G
  misc_feature 352, 353, 355, 359, 361, 362, 364, 367, 370, 372, 374,376, 382, 388, 390, 394, 396: n = A,T,C or G
ntcncngnngntgtggtnntttttttaatttttatnttttctttttttttctngctagcncttnctttttttggaattncggtncctttttntntcnattttttngacaaaaanaacctnttntttnanaccanagnnnggnncacncntnnaatntnccccttttncgntngggagctncncnttnnncgccnacntcantcgagacngtncttttnnntnnancannntnngtncgttgncngcnttnntncannantnttccctatnnacntgnnntcncncatnnttggacnancncctagccttnccatnntttnnttntttntnnatnancctngaaaacntcngnntnttcncnncnttnccncncncnccttcntatgtncnatgncn

SEQ ID NO: 70 - DNA, 396 bp, Homo sapiens
  misc_feature 15, 38, 57, 59, 63, 64, 65, 66, 68, 78, 79, 84, 87, 90,97, 114, 115, 127, 128, 141, 143, 145, 151, 159, 168, 169, 172,173, 176, 178, 197, 198, 207, 209, 211, 215, 220, 221, 223,225, 228, 240, 248, 249, 260, 262, 263, 273, 283, 287: n = A,T,C or G
  misc_feature 294, 304, 314, 334, 339, 340, 348, 362, 367, 376, 382,384, 386, 395: n = A,T,C or G
ttttttttttttttnttttttttttttttttttttttnttttttttttttttttttntncaannnntnaacttttaannggccnccngcnccccaanggggaccctgcttttgnnggctaaatgccnnaaaactttggggnantnggtatnaaaccccnctttgcccnncannttncnggggggggggggtttttgnnggggaacangnanaacnttttnncnanggnatcaccaaaaanaaagcccnnccctttttccnanngggggggggnggggggaaantcancccccanattgaccttnatttcaaaanggggcttataatcctgggcntgganncttccctntacccgggggttgnccacnttttattanaggggnangnggatccccnt

SEQ ID NO: 71 - DNA, 396 bp, Homo sapiens
  misc_feature 15, 21, 30, 33, 35, 36, 42, 43, 44, 45, 46, 51, 56, 58,59, 63, 70, 77, 81, 88, 94, 95, 96, 97, 101, 102, 109, 114,118, 119, 120, 124, 131, 132, 133, 134, 135, 141, 142, 143,144, 145, 146, 148, 149, 154, 158, 162, 164, 166, 172: n = A,T,C or G
  misc_feature 177, 179, 181, 184, 185, 213, 216, 218, 219, 222, 223,224, 230, 231, 240, 241, 242, 245, 247, 251, 252, 255, 258, 259,261, 264, 268, 269, 272, 276, 285, 288, 289, 291, 292, 293,297, 299, 300, 307, 312, 315, 316, 317, 325, 329, 334: n = A,T,C or G
  misc_feature 340, 341, 347, 350, 354, 355, 357, 360, 361, 367, 368,370, 371, 376, 377, 378, 387, 393, 394: n = A,T,C or G
gcatctagagggccngtttantctagaggnccngnntaaacnnnnncatcnacctncnntgcncctgctngttgccncccntctgtgncttgcnnnncccnngagcgtnccttnaccnnngaangtgcctnnnnnactgannnnnncnnataanatgngganantncgtcgncattntntnatnnggggtgatgctattctggggggtggggnggngnnatnnnatactnnggggacgtnnnatnangagnnatntcnngnttntctnntgntttntggggggcnatnngnnntctntnnggactcntcgcncannnatcaatancttnattcngtgtanngtccgnccntagnncngcnngtactnnanngttgnnntcattactnttcgtnngg

SEQ ID NO: 72 - DNA, 396 bp, Homo sapiens
  misc_feature 2, 23, 27, 34, 35, 36, 37, 39, 41, 45, 55, 56, 59, 61,88, 92, 96, 97, 98, 101, 103, 104, 106, 108, 111, 114, 115,121, 128, 129, 131, 159, 170, 191, 202, 227, 233, 235, 240,262, 268, 271, 272, 280, 281, 303, 304, 305, 311, 316, 317: n = A,T,C or G
  misc_feature 321, 324, 336, 344, 345, 353, 360, 362, 363, 364, 365,366, 370, 373, 389, 391, 392, 394, 395: n = A,T,C or G
tntttttttttttctaaaacatnactntttattnnnnangntttntgaacctctnngcntnatggtgagagtttgtctgattaataanaatnggannnttnannanangcntgnncgcaangatggcnncnctgtatatcccaccatcccattacactntgaaccttttntttgattaataaaaggaaggnatgcggggaanggggaaagagaatgcttgaacattnccatgngnccttngacaaactttccaatggaggcnggaacnaannaccaccanncaactcccctttttgtaatttnnnaacttncaacnnctanctntttattttggcntccctggnngaaacagnctgtatnannnnnaagnccntgagaacatccctggntnncnna

SEQ ID NO: 73 - DNA, 396 bp, Homo sapiens
  misc_feature 1, 7, 9, 14, 23, 35, 38, 44, 48, 50, 61, 74, 76, 79, 80,85, 86, 91, 95, 101, 109, 112, 113, 117, 118, 121, 122,127, 129, 132, 137, 141, 146, 214, 234, 243, 251, 266, 296,305, 306, 336: n = A,T,C or G
ntcaacntngactnctgtgaggnatggtgctgggngcntatgcngtgngnttttggatacnaccttatggacantngcnntcccnnggaangatnataatncttactgnagnnactnnaannttccntntcnaaaangttnaaaancattggatgtgccacaatgatgacagtttatttgctactcttgagtgctataatgatgaagatcttanccaccattatcttaactgangcacccaanatggtganttggggaacatatanagtacacctaagttcacatgaagttgtttnttcccaggnnctaaagagcaagcctaactcaagccattgncacacaggtgagacacctctattttgtacttctcacttttaagggattagaaaatagcca

SEQ ID NO: 74 - DNA, 396 bp, Homo sapiens
  misc_feature 22, 118: n = A,T,C or G
cctttttttttttttttactgngaatatatactttttatttagtcatttttgtttacaattgaaactctgggaattcaaaattaacatccttgcccgtgagcttcttatagacaccanaaaaagtttcaaccttgtgttccacattgttctgctgtgctttgtccaaatgaacctttatgagccggctgccatctagtttgacgcggattctcttgcccacaatttcgcttgggaagaccaagtcctcaaggatggcatcgtgcacagctgtcagagtacggctcctgggacgcttttgcttattttttgtacggctttttcgagttggcttaggcagaattctcctctgagcgataaagacgacatgcttcccactgaactttttctccaattcg

SEQ ID NO: 75 - DNA, 396 bp, Homo sapiens
  misc_feature 14, 38, 41, 43, 47, 53, 73, 75, 78, 83, 96, 112, 113,117, 124, 127, 146, 160, 167, 169, 176, 177, 178, 179, 194, 197,198, 209, 210, 220, 222, 226, 227, 231, 238, 241, 244, 258,259, 260, 270, 271, 274, 288, 301, 302, 305, 307, 316: n = A,T,C or G
  misc_feature 319, 328, 339, 344, 347, 354, 359, 364, 367, 369, 370,371, 373, 374, 381, 384, 387, 388: n = A,T,C or G
tttttttttttttntttttttttttttttttttttttnaantntaangggganggccccttttttttaaactngnccnttttnctttccttttttnaaaaggaaaaaaaaanntttntttttcnttnaaaaaccctttttcccacnaacaaaaaaaaccnttccccntnccttttnnnnaaaaaaaaggggctnggnntttccccttanncaaaaaaccntntccnngggnaaaaaanttntcnccgggggggaaacnnntgggggtgtnnccnaaatttgggggccntcggaaggggggnnccncncctaaagangtntttcaaaanaaaaacccccntcctnttntaaaaanaaaanaaaanaangnnngnnttttttntcnttnnccccccaa

SEQ ID NO: 76 - DNA, 396 bp, Homo sapiens
  misc_feature 87, 94, 102, 108, 138, 139, 143, 144, 145, 146, 151,152, 158, 168, 170, 171, 187, 204, 206, 224, 261, 262, 267, 268,270, 287, 305, 306, 313, 315, 319, 320, 330, 331, 333, 342,344, 348, 349, 356, 358, 360, 362, 368, 374, 376, 381: n = A,T,C or G
  misc_feature 390: n = A,T,C or G
acattcttcagaaatacagtgatgaaaattcattttgaaactcaaatattttcattttggatattctcctgtttttattaaaccagngattacncctggccntccctntaaatgttctaggaaggcatgtctgttgtnntttnnnnaaaannaaattntttttttttngnnaaaccccaaatcccantttatcaggaagttagncnaatgaaatggaaattggntaatggacaaaagctagcttgtaaaaaggaccacccnnccacnngnctttacccccttggttngttgggggaaaaaccatnnttaaccntntggnnaaaattgggnncntaaagtttncntggnnaacagtncntncngtattnaattgncnttatnggaaaatcngggatt

SEQ ID NO: 77 - DNA, 396 bp, Homo sapiens
  misc_feature 63, 66, 81, 83, 89, 107, 115, 118, 147, 151, 190, 232,275, 288, 294, 304, 323, 332, 369, 392: n = A,T,C or G
tttttttttttttttttttttttttttttttatcaacatttatatgctttattgaaagttganaanggcaacagttaaatncngggacnccttacaattgtgtaaanaacatgcncanaaacatatgcatataactactatacaggngatntgcaaaaacccctactgggaaatccatttcattagttanaactgagcatttttcaaagtattcaaccagctcaattgaaanacttcagtgaacaaggatttacttcagcgtattcagcagctanatttcaaattacncaaagngagtaactgngccaaattcttaaaatttntttaggggnggtttttggcatgtaccagtttttatgtaaatctatntataaaagtccacacctcctcanacag

SEQ ID NO: 78 - DNA, 396 bp, Homo sapiens
  misc_feature 8, 14, 16, 20, 26, 28, 36, 38, 39, 40, 51, 52, 55, 57,58, 67, 71, 114, 120, 132, 138, 142, 159, 165, 169, 172, 174,175, 183, 187, 195, 197, 198, 200, 202, 206, 209, 243, 259,260, 267, 283, 292, 305, 311, 315, 317, 319, 323, 324: n = A,T,C or G
  misc_feature 331, 333, 334, 338, 343, 348, 353, 355, 357, 366, 376,388: n = A,T,C or G
agctggcnaaaggngnatgngctgcnangcgattangnnnggtaacgtcannggntnnccagtgcangacnttgtaaaacgacggccacatgaattgtaatacgactcactatngggcgnattgggccgtgnaggatngtgntcacactcgaatgtatnctggcngatncananngctttatngctnttgacggngnntnanccanctngggctttagggggtatcccctcgcccctgcttcnttgatttgcacgggcnnctccganttccttcataataccngacgcttcnatcccctagctcngacctntcantntnttcnntgggttntnnccgntcacngcttncccgnangntataatctnggctcctttngggatccattantctttact

SEQ ID NO: 79 - DNA, 396 bp, Homo sapiens
  misc_feature 116, 153, 189, 194, 210, 218, 241, 270, 272, 288, 291,304, 324, 325, 329, 333, 334, 338, 340, 342, 366, 372, 377, 384,396: n = A,T,C or G
caccaaccaaaacctggcgccgttggcatcgtagagtgaacacaacccaaaaacgatacgccatctgttctgccctggctgcctcagccctaccagcactggtcatgtctaaaggncatcgtattgaggaagttcctgaacttcctttggtangttgaagataaagctgaaggctacaagaagaccaangaagntgttttgctccttaanaaacttanacgcctggaatgatatcaaaaangctatgcctctcagcgaatgagactgganangcaaaatgagaaaccntcnccgcatccagcgnaggggccgtgcatctctatnntgangatnntggnancnttcaaggccttcagaacctccctngaaatnctctnctttaangaaccaaactgn

SEQ ID NO: 80 - DNA, 396 bp, Homo sapiens
  misc_feature 312, 319, 353, 383: n = A,T,C or G
tgtacataggcatcttattcactgcaccctgtcacacccagcaccccccgccccgcacattatttgaaagactgggaatttaatggttagggacagtaaatctacttctttttccagggacgactgtcccctctaaagttaaagtcaatacaagaaaactgtctatttttagcctaaagtaaaggctgtgaagaaaattcattttacattgggtagacagtaaaaaacaagtaaaataacttgacatgagcacctttagatccttcccttcatggggctttgggcccagaatgacctttgaggcctgtaaanggattgnaatttcctataagctgtatagtggagggattggngggtcatttgagtaagccctccaagatacnttcaatacctggg

SEQ ID NO: 81 - DNA, 396 bp, Homo sapiens
  misc_feature 240, 286, 361, 364, 374, 375, 379, 380, 381, 387: n = A,T,C or G
gcagctgaagttcagcaggtgctgaatcgattctcctcggcccctctcattccacttccaacccctcccattattccagtactacctcagcaatttgtgccccctacaaatgttagagactgtatacgccttcgaggtcttccctatgcagccacaattgaggacatcctgcatttcctgggggagttcgccacagatattcgtactcatggggttcacatggttttgaatcaccagggnccgccatcaggagatgcctttatccagatgaagtctgcggacagancatttatggctgcacagaagtggcataaaaaaaacatgaaggacagatatgttgaagttttcagtgtcagctganganagaacattgnngtannngggggnactttaaat

SEQ ID NO: 82 - DNA, 396 bp, Homo sapiens
  misc_feature 220, 251, 297, 301, 309, 349, 395: n = A,T,C or G
gactcagaaatgtcagtctcatgaagttcaaaagatcgagaatgtttgctatcttggtggagcagccgcagccaagcaagtaacttgtaaaatgaggaatgccatcacccctcgagtgtccatcccacataacttggggttagagcacaagcgttcccaggaactactcaccttaccatcttggccgtttcatttgcttccaccagttctggaaagaganggcctagaagttcaaaaaaaaagtaggaaangtgcttttggagaaaatcacctgctcctcagaactgggcttacaanctgngaagtacnctatgtgccacctaatcctcatatatgacctcaagagacnccaataagcatatttccaccacggaatgaccagtgctttgggtaana

SEQ ID NO: 83 - DNA, 396 bp, Homo sapiens
  misc_feature 13, 372, 379, 393: n = A,T,C or G
tttgatttaaganatttattatttttttaaaaaaagcaacttccagggttgtcattgtacaggttttgcccagtctcctatagcatggtatagtgataactgattttttataacaatgactcagaggcattgaagatccataactatcttctgaattatcacagaaagaagaaagttagaagagtttaatgttaagtgtattaaaaatcatattctaattcttttaatttggttatctgagtatgataatataggagagctcagataacaaggaaaaggcattggggtaagaacactccttcccacaggatggcattaacagactttttctgcatatgctttatatagttgccaactaattcaccttttacncagcttnatttttttttactnggg

SEQ ID NO: 84 - DNA, 396 bp, Homo sapiens
  misc_feature 61, 232, 254, 270, 271, 286, 354, 356, 368, 374, 389,394: n = A,T,C or G
tttttacagcaatttttttttattgatgtttaacctgtatacaaccatacccattttaagngtacagacaaatgaattttgacaaattcattcactcatctaatcatcactataaccatgatacagatttttatcactccaaaagtccatcctgtgctcttttcaagtccatcctcctcatctgataccccaagccaccattgttttgctttctggaactacagttttgggnttttagaatttcatatatggtngaatcataccatttgnnatttggggctgacgnctttcctccaataatggatttgagaattatctacattttgcatggatcctgggttatttataccaacnangggttattatgnaaaatnggaccacaatttggnggcanta

SEQ ID NO: 85 - DNA, 396 bp, Homo sapiens
  misc_feature 293, 305, 306, 317, 347, 357, 372, 377, 386, 391: n = A,T,C or G
cagtgaccgtgctcctacccagctctgctccacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgcaacgcgcaggacttctgcacggacctggccgctccagtgccaacttcattccacggcactgcatctcgaccanccggacttgcannggttggggaanccgcccttgtttctccgtggcccatctaanaccaaacccntcaccttttcggagnccccncccctccgntgggnttact

SEQ ID NO: 86 - DNA, 396 bp, Homo sapiens
  misc_feature 5, 6, 28, 50, 58, 90, 108, 110, 118, 145, 154, 194, 244,285, 292, 300, 312, 315, 342, 344, 346, 359, 374, 378, 380,396: n = A,T,C or G
ttttnnactgaatgtttaatacatttgnaggaacagaagaaatgcagtanggattaanattttataattagacattaatgtaacagatgnttcatttttcaaagaagntncccccttntccctatctttttttaatcttccttanagcaataantagtaattactatatttgtggacaagctgctccactgtgntggacagtaattattaaatctttatgtttcacatcattattaccttccanaattctaccttcatttccctgcacaggttcactggactggntcacaancaaattgnactccactcaantanaagagcccaaagaaattagagtaacgncnantcctatgaattanagacccaaagatttnaggngntgattagaaacataan

SEQ ID NO: 87 - DNA, 396 bp, Homo sapiens
  misc_feature 231, 277, 285, 296, 341, 351, 372, 377, 380: n = A,T,C or G
atggaggcgctggggaagctgaagcagttcgatgcctaccccaagactttggaggacttccgggtcaagacctgcgggggcgccaccgtgaccattgtcagtggccttctcatgctgctactgttcctgtccgagctgcagtattacctcaccacggaggtgcatcctgagctctacgtggacaagtcgcggggagataaactgaagatcaacatcgatgtactttttccncacatgccttgtgcctatctgagtattgatgccatggatgtggccngagaacancagctggatgnggaacacaacctgtttaagccaccactagataaagatgcatcccngtgagctcanagctgagcggcatgagcttgngaaantcnaggtgaccgggtttga

SEQ ID NO: 88 - DNA, 396 bp, Homo sapiens
  misc_feature 246, 266, 301, 328, 347, 349, 368, 370, 371, 374, 379,387, 391: n = A,T,C or G
tccagagcagagtcagccagcatgaccgagcgccgcgtccccttctcgctcctgcggggccccagctgggaccccttccgcgactggtacccgcatagccgctcttcgaccaggccttcgggctgccccggctgccggaggagtggtcgcagtggttaggcggcagcagctggccaggctacgtgcgccccctgccccccgccgcatcgagagccccgcagtggccgcgcccgctacagccgcgcngctcagccggcaactcacancggggctcggagatccgggacactgcggaccgctngcgcgtgccctggatgtcaccactttngcccggacaactgacggtnanacaaggatggggggtgganannccngtaanccaagaangggnaggac

SEQ ID NO: 89 - DNA, 396 bp, Homo sapiens
  misc_feature 37, 76, 230, 295, 306, 333, 346, 370, 376, 377, 395: n = A,T,C or G
gagagaacagtaaacatccagccttagcatctctcangagtactgcagatcttcattagctatattcacatggagnaatgctattcaacctatttctcttatcaaaactaattttgtattctttgaccaatgttcctaaattcactctgcttctctatctcaatctttttcccctttctcatctttcctccttttttcagtttctaactttcactggttctttggaatgntttttctttcatctcttttcttttacattttggggtgtcccctctcttttcttaccctctttctncatccttcttnttcttttgaattggctgccctttatcntctcatctgctgncatcttcatttctcctccctcctntttccnntcattctactctctcccnt

SEQ ID NO: 90 - DNA, 396 bp, Homo sapiens
  misc_feature 82, 110, 115, 120, 121, 125, 126, 129, 131, 140, 141,144, 145, 146, 148, 149, 150, 153, 154, 157, 158, 160, 161, 163,164, 166, 170, 172, 173, 174, 175, 179, 182, 184, 189, 193,194, 195, 200, 206, 213, 215, 217, 218, 219, 220, 227: n = A,T,C or G
  misc_feature 228, 231, 233, 236, 241, 247, 248, 249, 250, 254, 259,262, 269, 273, 274, 275, 280, 281, 282, 286, 287, 289, 293, 294,301, 302, 304, 309, 311, 318, 319, 324, 325, 330, 331, 333,334, 336, 337, 341, 342, 343, 344, 349, 352, 353, 358: n = A,T,C or G
  misc_feature 361, 365, 367, 373, 377, 381, 385, 386, 387, 392: n = A,T,C or G
gggcgccggcgcgcccccccacccccgccccacgtctcgtcgcgcgcgcgtccgctgggggcggggagcggtcgggccggcngcggtcggccggcggcagggtggtgcgntttcnttttnnattnnccncnttcttcttnnttnnncnnnctnntanncnntnncnttcncnnnntttnctntntcttnaccnnnttttntaatcntcttctncntnnnntctcttnnatntnttncttanttcctnnnntttnttctntcntttctcncctnnntctcnnnctcnncnctcnncattttnntnttttntnccttctnntcttnnttctnntnntnntttnnnnttctnttnntcatnttncctntnttactntcancttntatnnncctcntttt

SEQ ID NO: 91 - DNA, 396 bp, Homo sapiens
  misc_feature 1, 3, 8, 9, 16, 17, 18, 21, 22, 32, 33, 45, 50, 63, 64,68, 75, 82, 92, 95, 98, 102, 106, 108, 110, 111, 116, 121, 135,151, 154, 158, 162, 167, 170, 176, 181, 185, 187, 209, 212,215, 225, 231, 245, 257, 278, 283, 288, 290, 292, 293: n = A,T,C or G
  misc_feature 312, 324, 326, 330, 331, 333, 334, 344, 345, 349, 351,352, 357, 358, 382, 384, 390, 392: n = A,T,C or G
ntntcctnnatttttnnntcnncttttttttnnaatttttctttnttttntttataaaaatcnncacntaaaacngcggaanaggggatttnttnttngggngtancncnnggccncaaanaaccccaaaaatancccaaaatgcacaggnccngggnaaangaccnacntgggtnttttntttntnaacaaggggggttttaaagggnatnggnatcaaagggnataaantttaaacctttganaaattttttaanaggcttgccccccactttggnccccnccccncngnngggatccaattttttttcnttggggctcccngncccnnannttccgggttnntggncnntcctnntttttttttttttgccttcacccntnccattncntttt

SEQ ID NO: 92 - DNA, 396 bp, Homo sapiens
  misc_feature 3, 7, 8, 9, 11, 31, 149, 152, 221, 233, 259, 263, 264,265, 266, 274, 278, 279, 283, 286, 294, 302, 307, 309, 310, 311,314, 316, 320, 343, 351, 363, 372, 377, 386, 393: n = A,T,C or G
ctntttnnntntttttttccccatcatccanaaatgggttttattctcagccgagggacagcaggactggtaaaaactgtcaggccacacggttgcctgcacagcacccccatgcttggtagggggtgggagggatggcgggggctggntgnccacaggccgggcatgacaaggaggctcactggaggtggcacactttggagtgggatgtcgggggacancttctttggtanttgggccacaagattcccaaggatancacnnnnactgattnccannctanagncaagcggntggccatntgtangnnnttntntatntgactatttatagatttttatanaacagggnaagggcataccncaaaagggnccaantttttaccnccgggcnccc

SEQ ID NO: 93 - DNA, 396 bp, Homo sapiens
  misc_feature 290, 304, 313, 320, 325, 333, 337, 348, 351: n = A,T,C or G
gctgccacagatctgttcctttgtccgtttttgggatccacaggccctatgtatttgaagggaaatgtgtatggctcagatcctttttgaaacatatcatacaggttgcagtcctgacccaagaacagttttaatggaccactatgagcccagttacataaagaaaaaggagtgctacccatgttctcatccttcagaagaatcctgcgaacggagcttcagtaatatatcgtggcttcacatgtgaggaagctacttaacactagttactctcacaatgaaggacctgnaatgaaaaatctgnttctaaccnagtcctntttanattttagngcanatccagaccancgncggtgctcgagtaattctttcatgggacctttggaaaactttcag

SEQ ID NO: 94 - DNA, 396 bp, Homo sapiens
  misc_feature 115, 204, 205, 243, 266, 276, 316, 319, 355, 357, 364: n = A,T,C or G
tgccttaaccagtctctcaagtgatgagacagtgaagtaaaattgagtgcactaaacgaataagattctgaggaagtcttatcttctgcagtgagtatggcccaatgctttctgnggctaaacagatgtaatgggaagaaataaaagcctacgtgttggtaaatccaacagcaagggagatttttgaatcataataactcatanngtgctatctgtcagtgatgccctcagagctcttgctgntagctggcagctgacgcttctangatagttagnttggaaatggtcttcataataactacacaaggaaagtcanccnccgggcttatgaggaattggacttaataaatttagngngcttccnacctaaaatatatcttttggaagtaaaattta

SEQ ID NO: 95 - DNA, 396 bp, Homo sapiens
  misc_feature 11, 16, 31, 36, 42, 49, 53, 56, 57, 60, 67, 70, 84, 89,91, 92, 99, 105, 106, 112, 120, 121, 125, 127, 128, 133, 137,141, 151, 152, 153, 154, 155, 162, 166, 167, 168, 174, 177,179, 186, 188, 194, 195, 199, 203, 205, 213, 217, 221: n = A,T,C or G
  misc_feature 227, 232, 235, 236, 240, 242, 260, 261, 265, 266, 291,297, 318, 325, 330, 339, 348, 351, 352, 354, 356, 362, 364, 372,380, 392, 395, 396: n = A,T,C or G
cctcccacccncttanttcatgagattcganaatgncacttntgtgctntttnctnnttntattctnacnatttctttcttggngcggnannaatcccntttttnngggcgnctctcccnncttntnntttcntggngctntcccttttcnnnnnaaacttntacnnngtttanaantntttctgnangggggnntccnaaanantttttccncctncctnattccnctctnaannctcncnaattgtttcccccccccnntagnntattttttctaaaaaattaactccnacgganaaaattttccctaaaatttcncctccanatttngaaaaaacncgcccgganctnntntncgaatntnaatttttnaaaaaaanttattttcatcnggnn

SEQ ID NO: 96 - DNA, 396 bp, Homo sapiens
  misc_feature 161, 193, 253, 259, 281, 288, 299, 309, 318, 319, 335,340, 344, 352, 355, 356, 387, 396: n = A,T,C or G
cctgggtaccaaatttctttatttgaaggaatggtacaaatcaaagaacttaagtggatgttttggacaacttatagaaaaggtaaaggaaaccccaacatgcatgcactgccttggcgaccagggaagtcaccccacggctatggggaaattagcccgangcttaactttcattatcactgcttccaagggngtgcttggcaaaaaaatattccgccaaccaaatcgggcgctccatcttgcccagttggtnccgggnccccaattcttggatgctttcncctcttnttccggaatgngctcatgaantcccccaannggggcattttgccagnggccntttngccattcnagnnggcctgatccattttttccaatgtaatgccncttcattgn

SEQ ID NO: 97 - DNA, 396 bp, Homo sapiens
  misc_feature 13, 15, 16, 19, 23, 31, 38, 39, 41, 45, 68, 94, 95, 100,119, 131, 133, 141, 144, 164, 171, 182, 186, 190, 191, 195,196, 198, 213, 229, 231, 235, 239, 247, 257, 265, 269, 272,278, 279, 286, 289, 291, 306, 309, 310, 312, 317, 320: n = A,T,C or G
  misc_feature 321, 327, 328, 337, 340, 343, 351, 360, 361, 368, 375,381, 385, 386, 387, 388: n = A,T,C or G
ctcaccctcctcntnnttntcanaatattgngaacttnntnctgntcgaatcactggcattaaaggancactagctaatggcactaaatttacnnactanggaaacttttttataatantgcaaaaacatntnaaaaagantgnagttcgcccatttctgcttnggaaganctcttcacttntaancccnnatgnngncctttgggtcaaaanctccgcgattattacngngttncccnctatttgnccttcctttntccccaangccncanatttcnnaactttnccntnaaatgcctttatttnatnncntttcnacnncttaannttccctttnaanaangatccctncttcaaatnntttcccngttcctngcattncccnnnnatttctct

SEQ ID NO: 98 - DNA, 396 bp, Homo sapiens
  misc_feature 130, 202, 285, 296, 299, 308, 314, 321, 322, 336, 373: n = A,T,C or G
acagggacaatgaagcctttgaagtgccagtctatgaagaggccgtggtgggactagaatcccagtgccgcccccaagagttggaccaaccaccccctacagcactgttgtgatacccccagcacctgangaggaacaacctaccatccagaggggccaggaaaagccaaactggaacagaggcgaatggctcagaggggtncatggccaagaaggaagccctggaagaacttcaatcaccttcggtttcgggaccaccggcttgtgtccctgttctgactgcanaacttggcgcngtnccccattanaacctntgactcnncccttgctataagnctgttttggcccctgatgatgatagggtttttatgangacacttgggcacccccttaatg

SEQ ID NO: 99 - DNA, 396 bp, Homo sapiens
  misc_feature 1, 4, 13, 15, 26, 31, 43, 46, 48, 52, 54, 55, 60, 62,68, 72, 93, 112, 118, 119, 122, 131, 132, 133, 134, 145, 147,152, 157, 163, 164, 186, 190, 225, 231, 239, 246, 247, 250,255, 262, 285, 314, 316, 319, 325, 332, 339, 343, 345: n = A,T,C or G
  misc_feature 348, 351, 352, 355, 357, 361, 370, 387: n = A,T,C or G
nttntttttccgncnaaagggcaagngtttncatctttcctgnccncncaananngggtntntgtgcntttnttttttcccaaaacccgggtnggggacaccttttgagganccactnntcntccggggcnnnnttttagaaggngnctaanaagcntcttgnngggggaaaaacatctttttgcncccnacatacccccaaggggggggggtgtctgggagganactaangacttttnttttttnnccncaaanaactganggcccccattgctccccccccantctttaaaaaaccccttcaatttccttgncnggnaaaaanggttggnaaaaaangagngngcntcnnttncntttnatggaaggnaaaaggtttttggttgnaaaaccccg

SEQ ID NO: 100 - DNA, 396 bp, Homo sapiens
  misc_feature 229, 286, 303, 312, 334, 335, 348, 350, 357, 364, 371,395: n = A,T,C or G
ctaacacggtgaaaccctgtctctactaaaaatacaaaaaaattagccaggcgtggtggcgggcacctgtagtcccagctgctcaggaagctgaggcaggagaatggcgtgaacccagaaggcggagcttgcagtgagctgagatcgtgtcagtgcactccagcctgggcgacagagcgagactcccgctcaaaaaaaaaaaaaaaaagagaaaagaaaaagctgcagngagctgggaatgggccctatcccctccttggggatcaatgagaccccttttcaaaanaaaaaaaaaaataatgngattttggnaacatatggcactggtgcttcnnggaattctgtttntnggcatgnccccctntgactgnggaaaaatccagcaggaggcccana

SEQ ID NO: 101 - DNA, 396 bp, Homo sapiens
  misc_feature 93, 99, 100, 111, 168, 172, 174, 199, 209, 216, 218,219, 227, 242, 243, 269, 272, 297, 300, 301, 308, 315, 317, 323,331, 341, 344, 348, 357, 359, 363, 364, 366, 376, 379, 386,389, 392: n = A,T,C or G
agttataactcaacagttcatttatatgctgttcatttaacagttcatttaaacagttcattataactgtttaaaaatatatatgcttatagncaaaanntgttgtggcgnagttgttgccgcttatagctgagcattatttcttaaattcttgaatgttcttttggngggntnctaaaaccgtatatgatccattttnatgggaaacngaattcntnncattatcncaccttggaaatacnnaacgtgggggaaaaaaatcattcccnccntccaaaactatacttcttttatctngannttcttgntcctgcncnggtttngaatatanctgggcaaanggntttnccaaatccntntacnntnctttgggaantancggcaantcntcncttt

SEQ ID NO: 102 - DNA, 396 bp, Homo sapiens
  misc_feature 17, 93, 136, 183, 317: n = A,T,C or G
actatacataagaacangctcacatgggaggctggaggtgggtacccagctgctgtggaacgggtatggacaggtcataaacctagagtcagngtcctgttggcctagcccatttcagcaccctgccacttggagnggacccctctactcttcttagcgcctaccctcatacctatctccctnctcccatctcctacggactggcgccaaatggctttcctgccaattttgggatcttctctggctctccagcctgcttactcctctatttttaaagggccaaacaaatcccttctctttctcaaacacagtaatgnggcactgaccctaccacacctcatgaagggggcttgttgcttttatttgggcccgatctggggggggcaaaatattttg

SEQ ID NO: 103 - DNA, 396 bp, Homo sapiens
  misc_feature 91, 174, 176, 188, 201, 214, 254, 277, 299, 325, 349,355, 365, 372, 390: n = A,T,C or G
ttgtgttgggactgctgataggaagatgtcttcaggaaatgctaaaattgggcaccctgccccaacttcaaagccacagctggtatgccanatggtcaggttaaagatatcaacctgctgactacaaaggaaaatatggtggggtcttcttttaccctcttgacttccctttgngngccccccgagancattgctttccgngatagggcaaaanaaattaaaaaacttaactggccagtgaatggggcttctgnggatctccttctggcattacatnggcaatccctaaaaaacaagangactgggacccataacattcttttgnatcaaccgaagcccccattgttangatatngggcttaaangctgatnaagcatctcgtccgggcnttttat

SEQ ID NO: 104 - DNA, 396 bp, Homo sapiens
  misc_feature 32, 53, 86, 141, 154, 156, 181, 182, 197, 204, 219, 224,226, 229, 232, 245, 253, 260, 262, 271, 273, 276, 292, 301,303, 305, 321, 325, 332, 343, 352, 382, 392: n = A,T,C or G
aagggagggcgcgccaagaccttcccactcgngcacactgggggcgccgacangacgcaacccagtccaacttggatacccttggntttagttctcggacacttcttttatctctccgtcgcaacttgtcaagttctcaanactgtctctctgngntatcttttttcttcgctgctcttcnncccccgacgtatttntcaaaangtctgcaattgttgnatacntnganctncaccactgttacnaggtcatnaatttcncntcaactctntnccncttgttccctgatatntcggccggngncnccaattctgtattttnctcntcaacgntctcacttttncctcctccnggccactttctccccttccttattccggcnttgtttgccnccat

SEQ ID NO: 105 - DNA, 396 bp, Homo sapiens
  misc_feature 57, 306, 356, 388, 391: n = A,T,C or G
tcaatagccagccagtgttcatttttatccttgagcttttagtaaaaacttcctggntttatttttagtcattgggtcatacagcactaaagtctgctatttatggaaactaacttttttgtttttaatccaggccaacatgtatgtaaattaaatttttagataattgattatctctttgtactacttgagatttgattatgagatgtgcatattgctttgggaagagctcgaggaaggaaataattctctcctttggtttgaacctcaactagataaaccctaggaattgttaactgcacaagnattttcattccacaaaacctgaggcagctcttttgccagagcgttcctgnacccccccaccccacttgccttgggtctttanaangagcc

SEQ ID NO: 106 - DNA, 396 bp, Homo sapiens
gctgtgtagcacactgagtgacgcaatcaatgtttactcgaacagaatgcatttcttcactccgaagccaaatgacaaataaagtccaaaggcattttctcctgtgctgaccaaccaaataatatgtatagacacacacacatatgcacacacacacacacacacccacagagagagagctgcaagagcatggaattcatgtgtttaaagataatcctttccatgtgaagtttaaaattactatatatttgctgatggctagattgagagaataaaagacagtaacctttctcttcaaagataaaatgaaaagcaattgctcttttcttcctaaaaaatgcaaaagatttacattgctgccaaatcatttcaactgaaaagaacagtattgctttg

SEQ ID NO: 107 - DNA, 396 bp, Homo sapiens
  misc_feature 12, 210, 257, 261, 271, 302, 311, 314, 318, 368, 374,385, 389, 396: n = A,T,C or G
ttcacagaacanggtggtttattatttcaatagcaaagagctgaaaaatgtcgggtcccataaaggagcagaacctgacccagagcctgcagtacatttccaccccacaggggtgcaggctgggccaggcagggccaaaggcagcagaaatgggagtaagagactgtgcccactgagaagctctgctgggtgtgggcaggtgggcatganatgatgatgatgtagtgtaaggaccaggtaggcaaaacctgtcaggnttgntgaatgtcanagtggatccaaaaggctgagggggtcgtcanaaggccggnggncccncccttgcccgtatgggccttcaaaaagtatgcttgctcatccgttgtttnccccanggagctgccangganaaggctn

SEQ ID NO: 108 - DNA, 396 bp, Homo sapiens
  misc_feature 280, 281, 286, 305, 311, 313, 323, 326, 327, 340, 352,356, 363, 369, 378, 388, 392: n = A,T,C or G
gcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgcttctcacgtgttttggagaaaannatcanccctgcaatcactttttgnaactgncnttgattttcngcnnccaagctatatcnaatatcgtctgngtanaaaatgncctggncttttgaangaatacatgngtgntgct

SEQ ID NO: 109 - DNA, 396 bp, Homo sapiens
  misc_feature 237, 279, 284, 291, 305, 307, 308, 313, 326, 343, 351,366, 376, 392, 394, 395: n = A,T,C or G
ggccgtaggcagccatggcgcccagcccggaatggcatggtcttgaagccccacttccacaaggactggcagcggcgcgtggccacgtggttcaaccagccggcccggaagatccgcagacgtaaggcccggcaagccaaggcgcgccgcatcgctccgcgccccgcgtcgggtcccatccggcccatcgtgcgctgcccacggttcggtaccacacgaagggcgcgccggcgcggnttcagcctggaggagctcagggtggccggatttacaagaagnggccngacatcngtattcttgggatncnngaagnggaacaagtcacngagtccttgcagccacntcagcggntgatgacaccgttcnaactcatctnttcccaagaaacctcngnnc

SEQ ID NO: 110 - DNA, 396 bp, Homo sapiens
  misc_feature 1, 2, 12, 13, 16, 18, 29, 39, 60, 66, 70, 86, 90, 104,121, 122, 127, 128, 146, 165, 171, 172, 173, 176, 188, 189, 193,195, 205, 210, 211, 224, 226, 227, 231, 233, 240, 243, 244,248, 249, 255, 257, 258, 260, 266, 268, 272, 273, 275: n = A,T,C or G
  misc_feature 278, 280, 287, 292, 294, 303, 308, 312, 315, 320, 322,332, 333, 334, 335, 345, 347, 351, 363, 364, 369, 371, 372, 379,381, 382, 386, 391, 393: n = A,T,C or G
nntgggctcctnncantnataataaaccngactcatacnccacaaggagatgaacaggantatgtncatnctgacgcggaaacagngcanggagctgaggaggngccaagatgagacctannggccnnggtgggcgcattcccggnggagggggccactaaggantacgannntcnagcggctcttgnnggcngncctcctcacncctgnntattcgattgtcncnnatgncntcctatnatnntcannattctntnntnatctcntntacnncntcncnttcatgnttacngntccctctcnttctnaccnttntctgnanctcctttctnnnnctttcatctntnttcngctttctttctnnaatcntnntttaacntnntctnctttntnatt

SEQ ID NO: 111 - DNA, 396 bp, Homo sapiens
  misc_feature 4, 7, 11, 16, 19, 25, 26, 30, 33, 39, 54, 60, 69, 75,81, 99, 102, 130, 132, 143, 154, 156, 166, 180, 182, 188, 190,192, 194, 198, 201, 226, 242, 253, 261, 264, 295, 305, 313,315, 320, 323, 325, 330, 334, 337, 340, 344, 348, 349: n = A,T,C or G
  misc_feature 351, 352, 357, 358, 359, 361, 362, 381, 387, 388, 389,394: n = A,T,C or G
taangancatnctggnttntgcctnnccgnctnattgantgttaaaggcaattntgtggntgtcccagngaatgncggctnattttctttccacattgngcncattcactcctcccactcttggcatgtngngacataagcanggtacataatngnaaaaatctgnatttctgatgccanangggtanancntnttgnatntcattccattgatatacagccactnttttatttttgatcancggccttcggntcactgcncanggtacttgacctcagtgtcactattatgggntttggtttcnctcttttncnggccnttntntttcncacnttncancttncttnntnnaaaannnanncactctctcttgctctctngatacnnngtctnaa

SEQ ID NO: 112 - DNA, 396 bp, Homo sapiens
  misc_feature 172, 186, 378, 380, 382, 388: n = A,T,C or G
tcaacgtcaccaattactgccatttagcccacgagctgcgtctcagctgcatggagaggaaaaaggtccagattcgaagcatggatccctccgccttggcaagcgaccgatttaacctcatactggcagataccaacagtgaccggctcttcacagtgaacgatgttaaagntggaggctccaagnatggtatcatcaacctgcaaagtctgaagacccctacgctcaaggtgttcatgcacgaaaacctctacttcaccaaccggaaggtgaattcggggggctgggcctcgctgaatcacttggattccacattctgctatgcctcatgggactcgcagaacttcaggctggccaccctgctcccaccatcactgntngncaatantcacccag

SEQ ID NO: 113 - DNA, 396 bp, Homo sapiens
  misc_feature 1, 2, 3, 4, 7, 8, 9, 10, 11, 65, 273, 279, 280, 289,321, 338, 380: n = A,T,C or G
nnnnttnnnnnggagccttaatttcagagttttattgtattgcactaaaggaacagcaggatggntatacaattttctctcattcagttttgaaaatctgtagtacctgcaaattcttaagaatacctttaccaccagattagaacagtaagcataataaccaatttcttaataagtaatgtcttacaaataaaaacacatttaaaatagctttaaatgcattcttcacaagtaattcagcatatattttatatcatggttacttatgcttangaattnnagcaggatntttattcttttgatggaaatatgggaaaactntattcatgcatatacanggataatattcagcgaagggaaaatcccgtttttattttggnaatgattcatatataa

SEQ ID NO: 114 - DNA, 396 bp, Homo sapiens
  misc_feature 40, 82, 114, 116, 146, 164, 166, 174, 185, 212, 215,219, 224, 236, 242, 254, 258, 263, 270, 286, 299, 308, 327, 328,329, 345, 363, 378, 382, 385: n = A,T,C or G
aaatgggacaacgtgattcttttgttttaaataaatactnagaacacggacttggctcctacaagcatttggactctaaggnttagaactggagagtcttacccatgggccccncncagggacgccacggttccctcccaccccgngatcaagacacggaatcngntggcgatngttggatcgcnatgtgccccttatctatagccttcccnggncatntacangcaggatgcggntggganaactacaactgnaatntctcnaacggtnatggtccccaccgatnaagattctacctngtcttttcntcccctggagtgtgagtgnnngaggaagaagcccttnccttacatcaccttttgnacttctgaacaagancaanacnatggccccccc

SEQ ID NO: 115 - DNA, 396 bp, Homo sapiens
  misc_feature 277, 297, 321, 341, 381, 391: n = A,T,C or G
ccgcctggttcggcccgcctgcctccactcctgcctctaccatgtccatcagggtgacccagaagtcctacaaggtgtccacctctggcccccgggccttcagcagccgctcctacacgagtgggcccggttcccgcatcagctcctcgagcttctcccgagtgggcagcagcaactttcgcggtggcctggcggcggctatggtggggccagcggcatgggaggcatcacccgcagttacggcaaccagagcctgctgagccccttgcctggaggnggaccccaacatcaagccgngcgcacccaggaaaaggagcagancaagaccctcaacaacaagnttgcttcttcatagacaagggaccggtccttgaacagcanaacaagatgntggag

SEQ ID NO: 116 - DNA, 396 bp, Homo sapiens
  misc_feature 267, 290, 343, 351, 376: n = A,T,C or G
atctcagtttactagctaagtgactttgggcaagggatttaacctctcgtccctcagtttcctcctatgtaaaatgacaaggataatagtaccaacccaatgtagattaaatgagtttacgaagtgttagaatagtgcttggcacattagtgctttacaactgctattttgattgttgttgtgggctctctcaaatgcattgtctctagatgccagtgacccaggtcaaaatttacctttaaccaagctgcatgtttcccagactgntgcacagtcctctaccctgaganaaagcttccacccaaggatacttttactttctgctggaaaactgatgagcaanggcaacangggacacttatcgccaactggaaangagaaattcttccttttgct

SEQ ID NO: 117 - DNA, 396 bp, Homo sapiens
  misc_feature 228, 267, 318, 331, 357, 368, 376: n = A,T,C or G
aaacattttttaataaaattcctatagaaagctcagtcatagggcaaatactcagttctctttcccatatcaccgaggattgagagctcccaatattctttggagaataagcagtagttttgctggatgttgccaggactcagagagatcacccatttacacattcaaaccagtagttcctattgcacatattaacattacttgcccctagcaccctaaatatatggnacctcaacaaataacttaaagatttccgtggggcgcganaccatttcaatttgaactaatatccttgaaaaaaatcacattattacaagntttaataaatacnggaagaagagctggcatttttctaanatctgaattcngacttggntttattccataaatacggtt

SEQ ID NO: 118 - DNA, 396 bp, Homo sapiens
  misc_feature 4, 5, 12, 14, 15, 16, 24, 59, 80, 87, 225, 280, 286,287, 295, 297, 298, 337, 349, 362, 375, 387, 394: n = A,T,C or G
accnncacctgntnnnttttaacnattacaacttctttatatggcagtttttactgggngcctaacactctctttactgnctcaagnggaagtccaaacaaatttcatttttgtagtaaaaaatctttatttccaaaatgatttgttagccaaaagaactataaaccacctaacaagactttggaagaaagagacttgatgcttcttataaattccccattgcanacaaaaaataacaatccaacaagagcatggtacccattcttaccattaacctggntttaannctccaaancnngatttaaaaatgaccccactgggcccaatccaacatganacctaggggggnttgccttgattangaatcccccttanggactttatctnggctganaa

SEQ ID NO: 119 - DNA, 396 bp, Homo sapiens
  misc_feature 251, 281, 298, 301, 308, 326, 332, 337, 351, 358, 362,388, 394: n = A,T,C or G
atggccagctcactttaaataccacctcaagactcatcgaaatgaccgctccttcatctgtcctgcagaaggttgtgggaaaagcttctatgtgctgcagaggctgaaggtgcacatgaggacccacaatggagagaagccctttatgtgccatgagtctggctgtggtaagcagtttactacagctggaaacctgaagaaccaccggcgcatccacacaggagagaaacctttcctttgtgaagcccaangatgtggccgtcctttgctgagtattctancttcgaaaacatctggnggntactcangagagaaagcctcattantgccantctgngggaaaaccttctntcagagnggangcaggaatgtgcatattaaaaagctnccttgnac

SEQ ID NO: 120 - DNA, 396 bp, Homo sapiens
  misc_feature 261, 263, 265, 272, 273, 288, 308, 310, 330, 379: n = A,T,C or G
catgggtcagtcggtcctgagagttcgaagagggcacattcccaaagacattcccagtcatgaaatgtagaagactggaaaattaagacattatgtaaaggtagatatggcttttagagttacattatgcttggcatgaataaggtgccaggaaaacagtttaaaattatacatcagcatacagactgctgttagaaggtatgggatcatattaagataatctgcagctctactacgcatttattgttaattgagttacanangncattcannactgagtttataganccatattgctctatctctgngnagaacatttgattccattgngaagaatgcagtttaaaatatctgaatgccatctagatgtattgtaccnaaaggggaaaaataaca

SEQ ID NO: 121 - DNA, 396 bp, Homo sapiens
  misc_feature 77, 125, 130, 142, 155, 162, 166, 176, 204, 227, 242,243, 245, 246, 249, 251, 252, 265, 279, 306, 310, 314, 336, 341,354, 367, 382, 385, 390, 395: n = A,T,C or G
ttttttttttttttttttaaaatcaagttatgtttaataaacattaataaatgtttacttaaaagggttaataaacntttactacatggcaaattattttagctagaatgcttttggcttcaagncatanaaaccagattcnaatgcccttaaanaattttnaaanatccattganggggataactgtaatccccaaggggaanagggttgggtatgacaggtacanggggccagcccagtnntnncananncagactcttaccntctttctgctgtgncaccctcaggcattggctccattctcngggntgcncatgggaagatggctttggacntaacnacacccttttgtncacgtaaaggccngatgcagggtcaaanagnttccnccatnt

SEQ ID NO: 122 - DNA, 396 bp, Homo sapiens
gtcgacatggctgccctctgggctcccagaacccacaacatgaaagaaatggtgctacccagctcaagcctgggcctttgaatccggacacaaaaccctctagcttggaaatgaatatgctgcactttacaaccactgcactacctgactcaggaatcggctctggaaggtgaagctagaggaaccagacctcatcagcccaacatcaaagacaccatcggaacagcagcgcccgcagcacccaccccgcaccggcgactccatcttcatggccaccccctgcggtggacggttgaccaccagccaccacatcatcccagagctgagctcctccagcgggatgacgccgtccccaccacctccctcttcttctttttcatccttctgtctctttgt

SEQ ID NO: 123 - DNA, 396 bp, Homo sapiens
  misc_feature 74, 94, 142, 149, 194, 219, 233, 279, 316, 335, 368: n = A,T,C or G
gccctttttttttttttttttttcctagtgccaggtttattccctcacatgggtggttcacatacacagcacanaggcacgggcaccatggganagggcagcactcctgccttctgaggggatcttggcctcacggtgtaanaaggganaggatggtttctcttctgccctcactagggcctagggaacccagnagcaaatcccaccacgccttccatntctcagccaagganaagccaccttggtgacgtttagttccaaccattatagtaagtgganaagggattggcctggtcccaaccattacagggtgaanatataaacagtaaaggaanatacagtttggatgaggccacaggaaggagcanatgacaccatcaaaagcatatgcaggga

SEQ ID NO: 124 - DNA, 396 bp, Homo sapiens
gaccattgccccagacctggaagatataacattcagttcccaccatctgattaaaacaacttcctcccttacagagcatacaacagagggggcacccggggaggagagcacatactgtgttccaatttcacgcttttaattctcatttgttctcacaccaacagtgtgaagtgcgtggtataatctccatttcaaaaccaaggaagcagcctcagagtggtcgagtgacacacctcacgcaggctgagtccagagcttgtgctcctcttgattcctggtttgactcagttccaggcctgatcttgcctgtctggctcagggtcaaagacagaatggtggagtgtagcctccacctgatattcaggctactcattcagtcccaaatatgtattttcc

SEQ ID NO: 125 - DNA, 396 bp, Homo sapiens
  misc_feature 43, 88, 91, 94, 139, 141, 150, 163, 193, 202, 212, 215,222, 238, 253, 256, 286, 297, 331, 343, 350, 360, 376, 385, 396: n = A,T,C or G
ccctttttttttttttttttttttttttttttttttactttgnaacaaaaatttattaggattaagtcaaattaaaaaacttcatgcnccnccncttgtcatatttacctgaaatgacaaagttatacttagcttgagngnaaaacttgngccccaaaaattntgtttggaaagcaaaaaaataattgatgcncatagcagngggcctgatnccnccacagngaatgttgtttaaggnctaacaaacaggggncancaaagcatacattacttttaagctttgggnccaaggaaaangtcattccctacctccttcaaaagcaaactcatnatagcctgggcncctaggnctggagcctnttttttcgagtctaanatgaacatntggatttcaan

SEQ ID NO: 126 - DNA, 396 bp, Homo sapiens
cgcgtcgactcgcaagtggaatgtgacgtccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccacgacctggacacgctggggctacggctacagggcggcatccccaacggctacctggtcctagacctcagcatgcaagaggccctctcggggacgccctgcctcctaggacctggacctgttctcaccgtcctggcactgctcctagcctc

SEQ ID NO: 127 - DNA, 396 bp, Homo sapiens
  misc_feature 15: n = A,T,C or G
ttttttttttttggnggtaaaatgcaaatgttttaaaatatgtttattttgtatgttttacaatgaatacttcagcaaagaaaataattataatttcaaaatgcaatccctggatttgataaatatcctttataatcgattacactaatcaatatctagaaatatacatagacaaagttagctaatgaataaaataagtaaaatgactacataaactcaatttcagggatgagggatcatgcatgatcagttaagtcactctgccactttttaaaataatacgattcacatttgcttcaatcacataaacattcattgcaggagttacacggctaatcattgaaaattatgatctttgttagcttaaaagaaaattcagtttaatacaaagacatt

SEQ ID NO: 128 - DNA, 396 bp, Homo sapiens
  misc_feature 220, 244, 351, 384: n = A,T,C or G
gccctttttttttttttttaaaggcaaataaaataagtttattgggatgtaaccccatcataaattgaggagcatccatacaggcaagctataaaatctggaaaatttaaatcaaattaaattctgcttttaaaaaggtgccttaagttaaccaagcattttgataacacattcaaatttaatatataaaaatagatgtatcctggaagatataatgaanaacatgccatgtgtataaattcanaatacgctttttacacaaagaactacaaaaagttacaaagacagccttcaggaaccacacttaggaaaagtgagccgagcagccttcacgcaaagcctccttcaaanaagtctcacaaagactccagaaccagccgagtntgtgaaaaagga

SEQ ID NO: 129 - DNA, 396 bp, Homo sapiens
  misc_feature 104, 164, 177, 204, 217, 234, 273, 312, 350, 353, 370: n = A,T,C or G
gcccttttttttttttttttttttactcagacaggcaatatttgctcacatttattctcttgcatcgtaaatagtagccaactcacaaaaataaagtatacaanaatgtaatattttttaaaataagattaacagtgtaagaaggaaaatctcaaaaaaagcanatagacaatgtanaaaattgaaatgaaatcccacagtaanaaaaaaaaaacanaaaagtgcctatttaanaattatgctacatgtggaacttaactagaccattttaanaaagaccaatttctaatgcaaattttctgaggttttcanattttatttttaaaatatgttatagctacatgttgtcnacncggccgctcgagtctanagggcccgtttaaacccgctgatcag

SEQ ID NO: 130 - DNA, 396 bp, Homo sapiens
  misc_feature 23, 24, 26, 32, 56, 191, 286, 355: n = A,T,C or G
cgcccttttttttttttttttanngnacgtgnctttatttctggatgatataaaanaaaaaacttaaaaaacaccccaaaccaaacaccaatggatccccaaagcgatgtgactccctcttcccacccggataaatagagacttctgtatgtcagtctaccctcccgcccccataaccccctctgctatanacatactctgggtatatattactctactcggcaatagacatctcccgaaaatagaattcctgccctgacacctgactcttccctggccgcatcanaccacccgccactgtagcacactggtgtccttgccccctgtggtcagggccatgctgtcatcccacaanaaggccacatttgtcacatggctgctgtgtccaccgtactt

SEQ ID NO: 131 - DNA, 396 bp, Homo sapiens
  misc_feature 49, 68, 69, 83, 88, 93, 136, 140, 154, 158, 166, 167,168, 170, 172, 173, 187, 226, 239, 241, 247, 257, 259, 271, 293,301, 318, 334, 336, 342, 344, 357, 377, 384: n = A,T,C or G
gcccttttttttttttttttttttttttttttcagtttacacaaaaacnctttaattgacagtatacnnttttccaaaatatnttttngtaanaaaatgcaataattattaactatagtttttacaaacaagtttntcantaaattccagtgtncttnaaaccccnnncnannaaaacatatatgancccccagttcctgggcaaactgttgaacattcactgcanacaaaaagaccancnccaaanagtcatctgngncctccatgctgngtttgcaccaaacctgagggancagctagngaccgtgacaaaagctntgctacagttttactntngccctntntgcctcccccatnatgtttccttggtccctcantcctgtnggagtaagttcc

SEQ ID NO: 132 - DNA, 396 bp, Homo sapiens
  misc_feature 69: n = A,T,C or G
cgcgtcgaccgcggccgtagcagccgggctggtcctgctgcgagccggcggcccggagtggggcggcgntatgtaccttccacattgagtattcagaaagaagtgatctgaactctgaccattctttatggatacattaagtcaaatataagagtctgactacttgacacactggctcggtgagttctgctttttctttttaatataaatttattatgttggtaaatttagcttttggcttttcactttgctctcatgatataagaaaatgtaggttttctctttcagtttgaattttcctattcagtaaaacaacatgctagaaaacaaacttttggaaaggcattgtaactattttttcaaatagaaccataataacaagtcttgtcttaccct

SEQ ID NO: 133 - DNA, 396 bp, Homo sapiens
  misc_feature 1, 17, 18, 20, 21, 25, 26, 30, 31, 40, 44, 45, 46, 51,52, 66, 67, 68, 74, 89, 109, 122, 166, 193, 214, 218, 266, 269,291, 307, 315, 348, 375, 378, 379, 386, 393: n = A,T,C or G
ntattacccctcctggnnanntggnnatannctgcaaggngatnnncccgnngaacttcactgatnnnccaatnaaaactgctttaaanctgactgcacatatgaattntaatacttacttngcgggaggggtggggcagggacagcaagggggaggattgggaanacaatagacaggcatgctggggatgcngcgggctctatggcttctgangcgnaaagaaccagctggggctctagggggtatccccacgcgccctgtagcngcncattaaacgcggcgggtgtggnggttacttcgcaaagngaccgatncacttgccagcgccctagctgcccgctcctttngctttcttcccttcctttctcgccacnttnnccggctntccccgncaa

SEQ ID NO: 134 - DNA, 396 bp, Homo sapiens
  misc_feature 133, 144, 221, 229, 302, 358: n = A,T,C or G
ttttttttttttctgctttttatatgtttaaaaatctctcattctattgctgctttatttaaagaaagattactttcttccctacaagatctttattaattgtaaagggaaaatgaataactttacaatgganacacctggcanacaccatcttaaccaaagcttgaagttaacataaccagtaatagaactgatcaatatcttgtgcctcctgatatggngtactaanaaaaacacaacatcatgccatgatagtcttgccaaaagtgcataacctaaatctaatcataaggaaacattanacaaactcaaattgaaggacattctacaaagtgccctgtattaaggaattattcanagtaaaggagacttaaaagacatggcaacaatgcagta

SEQ ID NO: 135 - DNA, 396 bp, Homo sapiens
gcgtcgacgctggcagagccacaccccaagtgcctgtgcccagagggcttcagtcagctgctcactcctccagggcacttttaggaaagggtttttagctagtgtttttcctcgcttttaatgacctcagccccgcctgcagtggctagaagccagcaggtgcccatgtgctactgacaagtgcctcagcttccccccggcccgggtcaggccgtgggagccgctattatctgcgttctctgccaaagactcgtgggggccatcacacctgccctgtgcagcggagccggaccaggctcttgtgtcctcactcaggtttgcttcccctgtgcccactgctgtatgatctgggggccaccaccctgtgccggtggcctctgggctgcctcccgtggt

SEQ ID NO: 136 - DNA, 396 bp, Homo sapiens
  misc_feature 18, 185, 188, 191, 193, 396: n = A,T,C or G
ttatgcttccggctcgtntgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgcggncgntcnantctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacan

SEQ ID NO: 137 - DNA, 396 bp, Homo sapiens
  misc_feature 156, 216: n = A,T,C or G
ttttttttttttctgctttgtacttgagtttatttcacaaaaccacggagaaagatactgaaatggagctctttccagcctccaagcaaggaggccccagcagccagtctccagccccttgagccctttttgttaggcccacacccaaaagagganaaccagtgtgtgcgcgaaggtacatggcaaggcacttttgaaaacatcccagtttaccgnggtgaaattgaacttactctgaaacagatgaaaagggacatgcaaaattgctgagcacatggaggtgtttgttagtaggtgaaaatcatgtcctgggtataacccagcttctccaggttagggtgagccgccgtctggatcagtggtggcgggccacacaccaggatgagcgtggacttc

SEQ ID NO: 138 - DNA, 396 bp, Homo sapiens
  misc_feature 69, 136, 265, 272: n = A,T,C or G
ccctttttttttttttttacaaatgagaaaaatgtttattaagaaaacaatttagcagctctcctttanaattttacagactaaagcacaacccgaaggcaattacagtttcaatcattaacacactacttaaggngcttgcttactctacaactggaaagttgctgaagtttgtgacatgccactgtaaatgtaagtattattaaaaattacaaattgtttggtgattattttgatgacctcttgagcagcagctccccccaanaatgcancaatggtatgtggctcaccagctccatatcggcaaaattcgtggacataatcatctttcaccattacagataaaccatattcctgaaggaagccagtgagacaagacttcaactttcctatatc

SEQ ID NO: 139 - DNA, 396 bp, Homo sapiens
  misc_feature 51, 105, 126, 147, 210, 212, 236, 241, 258, 263, 348: n = A,T,C or G
ccgcccttttttttttttttttcacaaaagcactttttatttgaggcaaanagaagtcttgctgaaaggattccagttccaagcagtcaaaactcaaccgttagnggcactattttgacctggtanattttgcttctctttggtcanaaaagggtattcaggttgtactttccccagcagggtaaaaagaagggcaaagcaaactggaananacttctactctactgacagggctnttganatccaacatcaagctanacacnccctcgctggccactctacaggttgctgtcccactgctgagtgacacaggccatactacatttgcaaggaaaaaaatgaggcaanaaacacaggtataggtcacttggggacgagcaggcaaccacagcttca

SEQ ID NO: 140 - DNA, 396 bp, Homo sapiens
  misc_feature 50, 60, 63, 100, 133, 135, 172, 183, 190, 196, 220, 240,262, 266, 273, 278, 293, 327, 332, 341, 348, 355, 380, 391: n = A,T,C or G
tttttttttttttttttttttttttttctcatttaacttttttaatgggnctcaaaattntgngacaaatttttggtcaagttgtttccattaaaaagtnctgattttaaaaactaataacttaaaactgccncncccaaaaaaaaaaaccaaaggggtccacaaaacattntcctttccttntgaaggntttacnatgcattgttatcattaaccagtnttttactactaaacttaaanggccaattgaaacaaacagttntganaccgttnttccnccactgattaaaagngggggggcaggtattagggataatattcatttanccttntgagctttntgggcanacttggngaccttgccagctccagcagccttnttgtccactgntttga

SEQ ID NO: 141 - DNA, 396 bp, Homo sapiens
acgccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccc

SEQ ID NO: 142 - DNA, 396 bp, Homo sapiens
acgcaggagaggaagcccagcctgttctaccagagaacttgcccaggtcagaggtctgcgtagaagcccttttctgagcatcctctcctctcctcacacctgccactgtcctctgcgttgctgtcgaattaaatcttgcatcaccatggtgcacttctgtggcctactcaccctccaccgggagccagtgccgctgaagagtatctctgtgagcgtgaacatttacgagtttgtggctggtgtgtctgcaactttgaactacgagaatgaggagaaagttcctttggaggccttctttgtgttccccatggatgaagactctgctgtttacagctttgaggccttggtggatgggaagaaaattgtagcagaattacaagacaagatgaaggcccg

SEQ ID NO: 143 - DNA, 396 bp, Homo sapiens
  misc_feature 19, 48, 69, 122, 183, 227, 332, 390: n = A,T,C or G
tttttttttttttccatanaaaataggatttattttcacatttaaggngaacacaaatccatgttccanaaatgttttatgcataacacatcatgagtagattgaatttctttaacacacanaaaaatcaaagcctaccaggaaatgcttccctccggagcacaggagcttacaggccacttntgttagcaacacaggaattcacattgtctaggcacagctcaagngaggtttgttcccaggttcaactgctcctacccccatgggccctcctcaaaaacgacagcagcaaaccaacaggcttcacagtaaccaggaggaaagatctcagngggggaaccttcacaaaagccctgagttgtgtttcaaaagccaagctctggggtctgnggcctg

SEQ ID NO: 144 - DNA, 396 bp, Homo sapiens
  misc_feature 221, 331: n = A,T,C or G
tttttttttttttcgctctttggtctgacaagaaaagagttttaggtgtgtgaagtagggtgggaaaaaaggtcagtttcaaattcagtaacatatggtaacactaagttaggctgctgcattcttttctttgggtacttaagccagctggcacttccactttgtaaccaattatattatgatcaacaactaatcagttagttcctcagcttcaactgaanagttcctgattacctgatgaaggacatacttgctctggcttcaattagcatgctgtcaagcatccctctccatgcttaacatggcaacacaaaacccaagagtccttctntttttttcattagccatgaataaacactcacaaaggggaagagtagacactgcttttagtaaacg

SEQ ID NO: 145 - DNA, 396 bp, Homo sapiens
  misc_feature 45, 56, 61, 63, 120, 122, 147, 151, 158, 259, 262, 274,339, 345, 353: n = A,T,C or G
tttttttttttttttttcaatggatccgttagctttactactaanatcttgctganatcananaagggcttctgggcaggctgagcactgggggtgtgcaacatggtaactctgaataananaaaccctgagttttactgggcaaanaaanaacaagnggtaggtatgatttctgaacctggaaatagcgaaaatgaaggaaattccaaaagcgcgtatttccaaataatgacaggccagcaagaggacaccaaacctntanaaagaggtattntttcttccagctactgatggctttggcatcccacaggcacattcctttggccttcaggatcttanatgcanatgtgganagtcaagaggtaggctgactctgagtcttcagctaaattcttt

SEQ ID NO: 146 - DNA, 396 bp, Homo sapiens
  misc_feature 120, 130, 176, 180, 185, 208, 238, 254, 259, 261, 275,285, 296, 347: n = A,T,C or G
ttttttttttttttcattagcaaggaaggatttattttttcttttgaggggagggcggaacagccgggatttttggaacactacctttgtctttcactttgttgtttgtgtgttaacacnaataaatcanaagcgactttaaatctcccttcgcaggactgtcttcacgtatcagngcanacaanaaaacagtggctttacaaaaaanatgttcaagtaggctgcactttgcctctgngggtgaggcacactgngggananacaaggtcccctgnaaccagaggngggaaggacanagctggctgactccctgctctcccgcattctctcctccatgtgttttgaanagggaagcaacatgttgaggtctgatcatttctacccagggaacctgtt

SEQ ID NO: 147 - DNA, 396 bp, Homo sapiens
acggggaagccaagtgaccgtagtctcatcagacatgagggaatgggtggctccagagaaagcagacatcattgtcagtgagcttctgggctcatttgctgacaatgaattgtcgcctgagtgcctggatggagcccagcacttcctaaaagatgatggtgtgagcatccccggggagtacacttcctttctggctcccatctcttcctccaagctgtacaatgaggtccgagcctgtagggagaaggaccgtgaccctgaggcccagtttgagatgccttatgtggtacggctgcacaacttccaccagctctctgcaccccagccctgtttcaccttcagccatcccaacagagatcctatgattgacaacaaccgctattgcaccttggaatt

SEQ ID NO: 148 - DNA, 396 bp, Homo sapiens
acgtcccatgattgttccagaccatgactcttcctggttgtgggtttgttacagagcaggagaagcagaggttatgacagttatgcagactttccccctcctttttctcttttctcttccccttgcttttccactgtttcttcctgctgccacctgggccttgaattcctgggctgtgaagacatgtagcagctgcagggtttaccacacgtgggagggcagcccagtactgtccctctgccttccccactttgagaatatggcagcccctttcattcctggcttggggtaggggagaccattgaagtagaagcctcaaagcagacttttccctttactgtgtgtactccaggacgaagaaggaagatcatgcttgatacttagattggttttccc

SEQ ID NO: 149 - DNA, 396 bp, Homo sapiens
  misc_feature 214, 295: n = A,T,C or G
tttttttttttttaaagagtcacattttattcaatgcctatttgtacatgttactagcaataaactcttttatctttaattttgagaagttttacaaatacagcaaagcagaatgactaatagagccggtaaccaggacacagatttggaaaaataggtctaattggttgttacactgtgtttatgtcatacatttcgcttatttttatcaaanaaaaatcagaatttataaaatgttaattaaaaggaaaacattctgagtaaatttagtcccgtgtttcttcctccaaatctntttgttctacactaacaggtcaggataagtatggatggggaggctggaaaaagggcatccttccccatgcggtccccagagccaccctctccaagcaggac

SEQ ID NO: 150 - DNA, 396 bp, Homo sapiens
acgcctctcttcagttggcacccaaacatctggattggcaaatcagtggcaagaagttccagcatctggacttttcagaattgatcttaagtctactgtcatttccagatgcattattttacaactgtatccttggaaatatatttctagggagaatattattgaagaaaatgttaatagcctgagtcaaatttcagcagacttaccagcatttgtatcagtggtagcaaatgaagccaaactgtatcttgaaaaacctgttgttcctttaaatatgatgttgccacaagctgcattggagactcattgcagtaatatttccaatgtgccacctacaagagagatacttcaagtctttcttactgatgtacacatgaaggaagtaattcagcagtt

SEQ ID NO: 151 - DNA, 396 bp, Homo sapiens
  misc_feature 146, 299, 332: n = A,T,C or G
acaaaatgcccagcctacagagtctgagaaggaaatttataatcaggtgaatgtagtattaaaagatgcagaaggcatcttggaggacttgcagtcatacagaggagctggccacgaaatacgagaggcaatccagcatccagcanatgagaagttgcaagagaaggcatggggtgcagttgttccactagtaggcaaattaaagaaattttacgaattttctcagaggttagaagcagcattaagaggtcttctgggagccttaacaagtaccccatattctcccacccagcatctanagcgagagcaggctcttgctaaacagtttgcanaaattcttcatttcacactccggtttgatgaactcaagatgacaaatcctgccatacagaatga

SEQ ID NO: 152 - DNA, 396 bp, Homo sapiens
  misc_feature 249: n = A,T,C or G
acgcagcgctcggcttcctggtaattcttcacctcttttctcagctccctgcagcatgggtgctgggccctccttgctgctcgccgccctcctgctgcttctctccggcgacggcgccgtgcgctgcgacacacctgccaactgcacctatcttgacctgctgggcacctgggtcttccaggtgggctccagcggttcccagcgcgatgtcaactgctcggttatgggaccacaagaaaaaaaagtagnggtgtaccttcagaagctggatacagcatatgatgaccttggcaattctggccatttcaccatcatttacaaccaaggctttgagattgtgttgaatgactacaagtggtttgccttttttaagtataaagaagagggcagcaaggt

SEQ ID NO: 153 - DNA, 396 bp, Homo sapiens
ccagagacaacttcgcggtgtggtgaactctctgaggaaaaacacgtgcgtggcaacaagtgactgagacctagaaatccaagcgttggaggtcctgaggccagcctaagtcgcttcaaaatggaacgaaggcgtttgcggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatctt

SEQ ID NO: 154 - DNA, 396 bp, Homo sapiens
  misc_feature 42, 45, 59, 82: n = A,T,C or G
acagcaaacctcctcacagcccactggtcctcaagaggggcnacntcttcacacatcancacaactacgcattgcctccctncactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggact

SEQ ID NO: 155 - DNA, 396 bp, Homo sapiens
  misc_feature 15, 17, 202, 280, 339: n = A,T,C or G
tttttttttttgaananacaggtctttaatgtacggagtctcacaaggcacaaacaccctcaccaggaccaaataaataactccacggttgcaggaaggcgcggtctggggaggatgcggcatctgagctctcccagggctggtgggcgagccgggggtctgcagtctgtgaggggcctcctgggtgtgtccgggcctctanagcgggtccagtctccaggatggggatcgctcactcactctccgagtcggagtagtccgccacgagggaggagccganactgcaggggtgccgcgtgtcgggggtgtcagctgcctcctgggaggagcctgctggcnacaggggcttgtcctgacggctcccttcctgccccctcgggctgctgcacttggggg

SEQ ID NO: 156 - DNA, 396 bp, Homo sapiens
  misc_feature 11, 30, 32, 37, 309, 332: n = A,T,C or G
gaaggggggcngggcaggggcggaatgtananattantgccatgattgaagatttaagaaacgtgagattcaggattttcaccacatccccatttagttagcttgctcgtttggctggtgcaaatgccagatggattatgaacaatgacagtaaattaatgcaacataatcaggtaatgatgccaagcgtatctggtgttccaggtattgtacctttaccggaacaaatcagtaaatccacaatccctggcacctgttaggcagctattaacctagtaaatgctcccccatcccatctcaatcagcaangacaatcaaaaacatttgctttnagtggcaggaacactggtacatttttacttgctccaagggctgtgccaacgctccctctctctg

SEQ ID NO: 157 - DNA, 396 bp, Homo sapiens
  misc_feature 121, 202, 204, 255, 314, 332, 368: n = A,T,C or G
tttttttttttttttggggaatgtaaatcttttattaaaacagttgtctttccacagtagtaaagctttggcacatacagtataaaaaataatcacccaccataattataccaaattcctnttatcaactgcatactaagtgttttcaatacaattttttccgtataaaaatactgggaaaaattgataaataacaggtaananaaagatatttctaggcaattactaggatcatttggaaaaagtgagtactgnggatatttaaaatatcacagtaacaagatcatgcttgttcctacagtattgcgggccanacacttaagtgaaagcanaagtgtttgggtgactttcctacttaaaattttggncatatcatttcaaaacatttgcatcttg

SEQ ID NO: 158 - DNA, 396 bp, Homo sapiens
tttccgaagacgggcagcttcagagaagaggattattcgggagattgctggtgtggcccatagactctttggcatagactctttcgcaggcagccactctgagtgtggccagttctataaccatccccaaactagctggagcctgatggataggaacgggtagtctgtcctcttccccataaaaatgttccaaaaagttatctccagagagagtcccttatgaagacagttgccaagctgtattctcattctttaaaccaatacccaggtcagggctagttcacactagcactgttagggacatggtgtggctagaaatgaattgagtgtgacttctccctacaaccccaggcccagggataggaggaggcagaggggtgcctggagtttctgcac

SEQ ID NO: 159 - DNA, 396 bp, Homo sapiens
tccgcgcgttgggaggtgtagcgcggctctgaacgcgctgagggccgttgagtgtcgcaggcggcgagggcgcgagtgaggagcagacccaggcatcgcgcgccgagaaggccgggcgtccccacactgaaggtccggaaaggcgacttccgggggctttggcacctggcggaccctcccggagcgtcggcacctgaacgcgaggcgctccattgcgcgtgcgcgttgaggggcttcccgcacctgatcgcgagaccccaacggctggtggcgtcgcctgcgcgtctcggctgagctggccatggcgcagctgtgcgggctgaggcggagccgggcgtttctcgccctgctgggatcgctgctcctctctggggtcctggcggccgaccgagaacg

SEQ ID NO: 160 - DNA, 396 bp, Homo sapiens
  misc_feature 96, 102, 122, 124, 129, 146, 148, 184, 189, 196, 205,208, 229, 246, 259, 261, 269, 272, 281, 297, 305, 308, 327, 331,337, 338, 339, 343, 346, 354, 366, 367, 369, 378, 379, 380,381, 391, 395: n = A,T,C or G
ggaaaccttctcaactaagagaacatcatttctggcaaactatttttgttagctcacaatatatgtcgtacactctacaatgtaaatagcactganccacancttacagaaggtaaaaagangnataanaacttcctttacaaaananttcctgttgttcttaatactccccattgcttatganaattntctatangtctctcangantgttcgcacccatttcttttntaacttctactaaaaanccatttacattgnanagtgtacnacntatatttgngagctaacaaaaaatngttttccnganatgatgttcttttagtttnaganggttcnnncaanttnctactccngcccgccactgnncnccacatttnnnnaattacaccncacng

SEQ ID NO: 161 - DNA, 396 bp, Homo sapiens
  misc_feature 271, 273, 325, 364: n = A,T,C or G
tttttgtttgattatttttattataatgaaattaaacttatgactattacagtatgctcagcttaaaacatttatgagtactgcaaggactaacagaaacaggaaaaatcctactaaaaatatttgttgatgggaaatcattgtgaaagcaaacctccaaatattcatttgtaagccataagaggataagcacaaccatatgggaggagataaccagtctctcccttcatatatattcttttttatttcttggtataccttcccaaaacananacattcaacagtagttagaatggccatctcccaacattttaaaaaaactgcnccccccaatgggtgaacaaagtaaagagtagtaacctanagttcagctgagtaagccactgtggagcctta

SEQ ID NO: 162 - DNA, 396 bp, Homo sapiens
  misc_feature 33, 38, 51, 62, 71, 72, 88, 97, 98, 100, 106, 142, 155,160, 161, 163, 168, 170, 174, 183, 190, 194, 203, 214, 216, 231,232, 241, 242, 252, 258, 260, 264, 265, 267, 276, 278, 282,287, 289, 292, 295, 297, 301, 311, 319, 322, 325: n = A,T,C or G
  misc_feature 330, 337, 341, 342, 347, 348, 354, 356, 361, 367, 368,375, 379, 385, 391, 394, 395: n = A,T,C or G
ttttttttttttttttttttttttttttttttnggggnccaaatttttttntttgaaggaangggacaaannaaaaaacttaaggggntgttttggnncnacttanaaaaaagggaaaggaaaccccaacatgcatgccctnccttggggaccanggaanncnccccncnggtntggggaaantaacccnaggnttaactttnattatcactgncncccagggggggcttnnaaaaaaaannttcccccaanccaaantngggnncncccattttncncaanttggncnccnggncncccnattttttgangggtttcnccngcncattnagggaangggnntcaannaaaccncncaaangggggnnatttttntcangggccnatttgngcnnt

SEQ ID NO: 163 - DNA, 396 bp, Homo sapiens
cactgtccggctctaacacagctattaagtgctacctgcctctcaggcactctcctcgcccagtttctgaggtcagacgagtgtctgcgatgtcttcccgcactctattcccccagcctctttctgctttcatgctcagcacatcatcttcctaggcagtctcttccccaaagtctcaccttttcttccaatagaaaattccgcttgacctttggtgcactgcccacttcccagctccactggcccaagtctgagccggaggcccttgttttgggggcggggggagagttggatgtgattgcccttgaagaacaaggctgacctgagaggttcctggcgccctgaggtggctcagcacctgcccagggtaggcctggcatgaggggttaggtcagc

SEQ ID NO: 164 - DNA, 396 bp, Homo sapiens
gacacgcggcggtgtcctgtgttggccatggccgactacctgattagtgggggcacgtcctacgtgccagacgacggactcacagcacagcagctcttcaactgcggagacggcctcacctacaatgactttctcattctccctgggtacatcgacttcactgcagaccaggtggacctgacttctgctctgaccaagaaaatcactcttaagaccccactggtttcctctcccatggacacagtcacagaggctgggatggccatagcaatggcgcttacaggcggtattggcttcatccaccacaactgtacacctgaattccaggccaatgaagttcggaaagtgaagaaatatgaacagggattcatcacagaccctgtggtcctcagcccc

SEQ ID NO: 165 - DNA, 396 bp, Homo sapiens
  misc_feature 29, 33, 55, 57, 65, 77, 82, 87, 98, 101, 103, 114, 118,124, 169, 171, 173, 183, 186, 188, 216, 219, 227, 230, 242, 243,245, 252, 265, 273, 290, 296, 321, 324, 332, 338, 340, 342,345, 359, 372, 380: n = A,T,C or G
ttttttttttttttttttttttttttcangggncactgaggctttttattttgancncaaaaccnccggggatctancctgnggccnccccggaaatnacncnaggctcacatnactntaaacncttgggggaaagggaggcaaaaaaaacaatgacttgggccaattncncnactgcaaagntananctgccaacagggctccagggagcttggnttntgtaaaanttntaaggaagcggnncnaactccncggggggggggcnctaactancagggacccctgcaagngttggncgggggcctcaacctgcctgagctnacncaaggggnggggtntntntanccaacaggggaccnaagggcttgcctncccacagnttacttggccaagggg

SEQ ID NO: 166 - DNA, 396 bp, Homo sapiens
  misc_feature 151, 255: n = A,T,C or G
ttttttcaaattcagagcatttttattaaaagaacaaaatattaaggcacaaaatacatcaatttttcaaatgaaaacccttcaaacggttatgtcctacattcaacgaaacttcttccaaattacggaataatttaactttttaaaatanaaaaatacaagttcttaaatgcctaaaatttctccccaaataaatgttttcttagttttaatgaagtctcttcatgcagtactgagctccaatattataatgtncacttccttaaaaatctagttttgccacttatatacattcaatatgtttaaccagtatattaaccagtatattaaccaatatgttaaacttcttttaagtataaggcttggtattttgtattgcttattgcatgctttgat

SEQ ID NO: 167 - DNA, 396 bp, Homo sapiens
tggcggcagcggcggtggcggtggctgagcagaggacccggcgggcggcctcgcgggtcaggacacaatgtttgcacgaggactgaagaggaaatgtgttggccacgaggaagacgtggagggagccctggccggcttgaagacagtgtcctcatacagcctgcagcggcagtcgctcctggacatgtctctggtgaagttgcagctttgccacatgcttgtggagcccaatctgtgccgctcagtcctcattgccaacacggtccggcagatccaagaggagatgacgcaggatgggacgtggcgcacagtggcaccccaggctgcagagcgggcgccgctcgaccgcttggtctccacggagatcctgtgccgtgcagcgtgggggcaagaggg

SEQ ID NO: 168 - DNA, 396 bp, Homo sapiens
taggatggtaagagtattataaggattggtacaaggcatgatgagtccttttgcttttaggcttttgacttctggttttagactttctttagcttctgttgttagacaacattgtgcaagcttggtttttataagtttgcatggattaaactgaacttaatgaaattgtccctccccccaaattctcagcacaatttttaggcccacaaggagtcaagcacctcaaggagatcttcagtttgaacttggtgtagacacagggatactgatgaatcaatattcaaattagctgttacctacttaagaaagagaggagaccttggggatttcgaggaagggttcataagggagattttagctgagaaataccatttgcacagtcaatcacttctgacc

SEQ ID NO: 169 - DNA, 396 bp, Homo sapiens
  misc_feature 16, 58, 76, 84, 99, 111, 114, 124, 136, 140, 161, 167,184, 189, 204, 206, 210, 228, 230, 232, 243, 275, 277, 289, 301,303, 312, 319, 321, 323, 325, 333, 345, 349, 355, 359, 364,365, 372, 375, 377, 379, 383, 387, 389, 394, 396: n = A,T,C or G
tttttttttttttcanaattaaattctttaatacaaaatgcttttttttttttaaaanatatctgtatttctttgncgttgttnaaaaataaatatgtnctacggaatatntcnaaaaactgcnctaaaaacaaanacgngatgttaatatcttttccccncaattnttacggataaacagtanccccnataaataaatgatancnaatnttaaaattaaaaaagganananatttagtatgnaaaattctctattttttcttggtttggttttncntataaaaaacanaatagcaatgtntnttttatcanaatcccntntntncctaaacntttttttttttntttncccccnaatncaagnngccaaanatntntntagnatgnanatgtntn

SEQ ID NO: 170 - DNA, 396 bp, Homo sapiens
tgagaagtaccatgccgcttctgcagaggaacaggcaaccatcgaacgcaacccctacaccatcttccatcaagcactgaaaaactgtgagcctatgattgggctggtacccatcctcaagggaggccgtttctaccaggtccctgtacccctacccgaccggcgtcgccgcttcctagccatgaagtggatgatcactgagtgccgggataaaaagcaccagcggacactgatgccggagaagctgtcacacaagctgctggaggctttccataaccagggccccgtgatcaagaggaagcatgacttgcacaagatggcagaggccaaccgtgccctggcccactaccgctggtggtagagtctccaggaggagcccagggccctctgcgcaag

SEQ ID NO: 171 - DNA, 396 bp, Homo sapiens
  misc_feature 133, 224, 260, 264, 268, 279, 283, 317, 322, 338, 360,370, 371, 378: n = A,T,C or G
ggtcctcgtcgtggtgagcgcagccactcaggctggtcctgggggtggggctgtaggggaaagtgctaaagccgctgagtgaagtaagaactctgctagagaggaaaatgggcttgctttcatcatcatcctnctcagctggtggggtcaagtgggaagttctgtcactgggatctggttcagtgtctcaagaccttgccccaccacggaaagcctttttcacntaccccaaaggacttggagagatgttagaagatggntctnaaanattcctctgcnaatntgtttttagctatcaagtggcttccccccttaancaggnaaaacatgatcagcangttgctcggatggaaaaactancttggtttgnnaaaaaanctggaggcttgacaatgg

SEQ ID NO: 172 - DNA, 396 bp, Homo sapiens
  misc_feature 239, 242, 244, 246, 249, 257, 260, 314, 329, 355, 372,378, 385, 387, 388, 395: n = A,T,C or G
agccttgggccaccctcttggagcatctggctgtcgaattcttgtgaccctgttacacacactggagagaatgggcagaagtcgtggtgttgcagccctgtgcattgggggtgggatgggaatagcaatgtgtgttcagagagaatgaattgcttaaactttgaacaacctcaatttctttttaaactaataaagtactaggttgcaatatgtgaaaaaaaaaaaaaaagggcggccgntcnantntanagggcccnttnaaacccgttgatcaacctcgactgtgccttctagttgccagccatctgttgttngcccctcccccgtgnctttcttgaccttgaaaggggccccncccctgtctttcctaanaaaaangaagaantnnccttccnt

SEQ ID NO: 173 - DNA, 396 bp, Homo sapiens
  misc_feature 209, 210, 232, 244, 270, 275, 284, 341, 343, 349, 359,364, 368, 376, 380, 382, 388, 389, 390, 392: n = A,T,C or G
aagcatgtggatatgtttagctacgtttactcacagccagcgaactgacattaaaataactaacaaacagattcttttatgtgatgctggaactcttgacagctataattattattcagaaatgactttttgaaagtaaaagcagcataaagaatttgtcacaggaaggctgtctcagataaattatggtaaaattttgcaggggacannctttttaagacttgcacaattnccggatcctgcnctgactttggaaaaggcatatatgtnctagnggcatgganaatgccccatactcatgcatgcaaattaaacaaccaagtttgaatctttttgggggngngctatnctttaacccngtacnggcnttattatntaangnccctgnnncntgtg

SEQ ID NO: 174 - DNA, 924 bp, Homo sapiens
cctgacgacccggcgacggcgacgtctcttttgactaaaagacagtgtccagtgctccagcctaggagtctacggggaccgcctcccgcgccgccaccatgcccaacttctctggcaactggaaaatcatccgatcggaaaacttcgaggaattgctcaaagtgctgggggtgaatgtgatgctgaggaagattgctgtggctgcagcgtccaagccagcagtggagatcaaacaggagggagacactttctacatcaaaacctccaccaccgtgcgcaccacagagattaacttcaaggttggggaggagtttgaggagcagactgtggatgggaggccctgtaagagcctggtgaaatgggagagtgagaataaaatggtctgtgagcagaagctcctgaagggagagggccccaagacctcgtggaccagagaactgaccaacgatggggaactgatcctgaccatgacggcggatgacgttgtgtgcaccagggtctacgtccgagagtgagtggccacaggtagaaccgcggccgaagcccaccactggccatgctcaccgccctgcttcactgccccctccgtcccaccccctccttctaggatagcgctccccttaccccagtcacttctgggggtcactgggatgcctcttgcagggtcttgctttctttgacctcttctctcctcccctacaccaacaaagaggaatggctgcaagagcccagatcacccattccgggttcactccccgcctccccaagtcagcagtcctagccccaaaccagcccagagcagggtctctctaaaggggacttgagggcctgagcaggaaagactggccctctagcttctaccctttgtccctgtagcctatacagtttagaatatttatttgttaattttattaaaatgcttta

SEQ ID NO: 175 - DNA, 3321 bp, Homo sapiens
atgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaagctggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagcacaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaagacaccaaatctggctgaatgaaataaattggtgataagtggaaaaaagagaaaaaccaatgattcataacaatgtatgtgaaagtgtaaaatagaatgttactttggaatgactataaacattaaaagagactggagcat

SEQ ID NO: 176 - DNA, 487 bp, Homo sapiens
gaaatactttctgtcttattaaaattaataaattattggtctttacaagacttggatacattacagcagacatggaaatataattttaaaaaatttctctccaacctccttcaaattcagtcaccactgttatattaccttctccaggaaccctccagtggggaaggctgcgatattagatttccttgtatgcaaagtttttgttgaaagctgtgctcagaggaggtgagaggagaggaaggagaaaactgcatcataactttacagaattgaatctagagtcttccccgaaaagcccagaaacttctctgcagtatctggcttgtccatctggtctaaggtggctgcttcttccccagccatgagtcagtttgtgcccatgaataatacacgacctgttatttccatgactgctttactgtatttttaaggtcaatatactgtacatttgataataaaataatattctcccaaaaaaaaaaaaaaa

SEQ ID NO: 177 - DNA, 3999 bp, Homo sapiens
caagattccacatttgatggggtgactgacaaacccatcttagactgctgtgcctgcggaactgccaagtacagactcacattttatgggaattggtccgagaagacacacccaaaggattaccctcgtcgggccaaccactggtctgcgatcatcggaggatcccactccaagaattatgtactgtgggaatatggaggatatgccagcgaaggcgtcaaacaagttgcagaattgggctcacccgtgaaaatggaggaagaaattcgacaacagagtgatgaggtcctcaccgtcatcaaagccaaagcccaatggccagcctggcagcctctcaacgtgagagcagcaccttcagctgaattttccgtggacagaacgcgccatttaatgtccttcctgaccatgatgggccctagtcccgactggaacgtaggcttatctgcagaagatctgtgcaccaaggaatgtggctgggtccagaaggtggtgcaagacctgattccctgggacgctggcaccgacagcggggtgacctatgagtcacccaacaaacccaccattccccaggagaaaatccggcccctgaccagcctggaccatcctcagagtcctttctatgacccagagggtgggtccatcactcaagtagccagagttgtcatcgagagaatcgcacggaagggtgaacaatgcaatattgtacctgacaatgtcgatgatattgtagctgacctggctccagaagagaaagatgaagatgacacccctgaaacctgcatctactccaactggtccccatggtccgcctgcagctcctccacctgtgacaaaggcaagaggatgcgacagcgcatgctgaaagcacagctggacctcagcgtcccctgccctgacacccaggacttccagccctgcatgggccctggctgcagtgacgaagacggctccacctgcaccatgtccgagtggatcacctggtcgccctgcagcatctcctgcggcatgggcatgaggtcccgggagaggtatgtgaagcagttcccggaggacggctccgtgtgcacgctgcccactgaggaaacggagaagtgcacggtcaacgaggagtgctctcccagcagctgcctgatgaccgagtggggcgagtgggacgagtgcagcgccacctgcggcatgggcatgaagaagcggcaccgcatgatcaagatgaaccccgcagatggctccatgtgcaaagccgagacatcacaggcagagaagtgcatgatgccagagtgccacaccatcccatgcttgctgtccccatggtccgagtggagtgactgcagcgtgacctgcgggaagggcatgcgaacccgacagcggatgctcaagtctctggcagaacttggagactgcaatgaggatctggagcaggtggagaagtgcatgctccctgaatgccccattgactgtgagctcaccgagtggtcccagtggtcggaatgtaacaagtcatgtgggaaaggccacgtgattcgaacccggatgatccaaatggagcctcagtttggaggtgcaccctgcccagagactgtgcagcgaaaaaagtgccgcatccgaaaatgccttcgaaatccatccatccaaaagctacgctggagggaggcccgagagagccggcggagtgagcagctgaaggaagagtctgaaggggagcagttcccaggttgtaggatgcgcccatggacggcctggtcagaatgcaccaaactgtgcggaggtggaattcaggaacgttacatgactgtaaagaagagattcaaaagctcccagtttaccagctgcaaagacaagaaggagatcagagcatgcaatgttcatccttgttagcaagggtacgagttccccagggctgcactctagattccagagtcaccaatggctggattatttgcttgtttaagacaatttaaattgtgtacgctagttttcatttttgcagtgtggttcgcccagtagtcttgtggatgccagagacatcctttctgaatacttcttgatgggtacaggctgagtggggcgccctcacctccagccagcctcttcctgcagaggagtagtgtcagccaccttgtactaagctgaaacatgtccctctggagcttccacctggccagggaggacggagactttgacctactccacatggagaggcaaccatgtctggaagtgactatgcctgagtcccagggtgcggcaggtaggaaacattcacagatgaagacagcagattccccacattctcatctttggcctgttcaatgaaaccattgtttgcccatctcttcttagtggaactttaggtctcttttcaagtctcctcagtcatcaatagttcctggggaaaaacagagctggtagacttgaagaggagcattgatgttgggtggcttttgttctttcactgagaaattcggaatacatttgtctcacccctgatattggttcctgatgcccccccaacaaaaataaataaataaattatggctgctttatttaaatataaggtagctagtttttacacctgagataaataataagcttagagtgtatttttcccttgcttttgggggttcagaggagtatgtacaattcttctgggaagccagccttctgaactttttggtactaaatccttattggaaccaagacaaaggaagcaaaattggtctctttagagaccaatttgcctaaattttaaaatcttcctacacacatctagacgttcaagtttgcaaatcagtttttagcaagaaaacatttttgctatacaaacattttgctaagtctgcccaaagcccccccaatgcattccttcaacaaaatacaatctctgtactttaaagttattttagtcatgaaattttatatgcagagagaaaaagttaccgagacagaaaacaaatctaagggaaaggaatattatgggattaagctgagcaagcaattctggtggaaagtcaaacctgtcagtgctccacaccagggctgtggtcctcccagacatgcataggaatggccacaggtttacactgccttcccagcaattataagcacaccagattcagggagactgaccaccaagggatagtgtaaaaggacattttctcagttgggtccatcagcagtttttcttcctgcatttattgttgaaaactattgtttcatttcttcttttataggccttattactgcttaatccaaatgtgtaccattggtgagacacatacaatgctctgaatacactacgaatttgtattaaacacatcagaatatttccaaatacaacatagtatagtcctgaatatgtacttttaacacaagagagactattcaataaaaactcactgggtctttcatgtctttaagctaagtaagtgttcagaaggttcttttttatattgtcctccacctccatcattttcaataaaagatagggcttttgctcccttgttcttggagggaccattattacatctctgaactacctttgtatccaacatgttttaaatccttaaatgaattgctttctcccaaaaaaagcacaatataaagaaacacaagatttaattatttttctacttggggggaaaaaagtcctcatgtagaagcacccacttttgcaatgttgttctaagctatctatctaactctcagcccatgataaagttccttaagctggtgattcctaatcaaggacaagccaccctagtgtctcatgtttgtatttggtcccagttgggtacattttaaaatcctgattttggagacttaaaaccaggttaatggctaagaatgggtaacatgactcttgttggattgttattttttgtttgcaatggggaatttataagaagcatcaagtctctttcttaccaaagtcttgttaggtggtttatagttcttttggctaacaaatcattttggaaataaagattttttactacaaaaatg

SEQ ID NO: 178 - DNA, 1069 bp, Homo sapiens
aaaaaagatgaataaatgaataagagagatgaataaacaaatttacattacatgtgatagttatcatggtatggccttcatgacaagatggatgagaatatcactgataggatattagccttctttcatatctttatattgaaatatgggctttacttcaatttgaaggtctttcatgaacaataaaagagagtagaaggactgtctgagaaggcaggagacatataaaacagatgactgaaagactgactagctcctggaaagggaaacatttggaacatccagagtaagggcaaatgggcttctaccagcacaacaaagagcctccaggtggcaacatggaagcaggttatcagagaaaataaatgtgcaaattccttatttacaatgactcacttaaccccacaaacatgtttcactgctgccttccccagttgtcgcttatgtactgttgttacctttcagttacatgcctttgatcctaaaattctctacttttggtgccttatcagttctttgcaatctgcctgtggttatcagcacttaaagcacaattttgaaggggaaaaaaatgataatcaccttagtcccaaagaaataatttgtcaaactgccttattagtattaaaaacagacacactgaatgaagtagcatgatacgcatatatcctactcagtatcattggccttttatcaaatggggaaactatacttttgtattacatagttttagaaatcgaaagttagagactctttataagtaatgtcaaggaacagtaatttaaaaacaaagttctaacaaatatattgtttgcttaatcacaatgccctcaacttgtatttgaataactaaataggacatgtcttccttggagctgtgggcattagttcagaagcactacctgcatcttaattttcaaaacttaagttttattagcaaatcctcttctctgtaagacttagctatgaagtggtatattttttccaaatatttttctgaaaacatttgttgttgtaactgcacaataaaagtccagttgcaattaaaaaaaaaaaaaaaaaaaaaaaa

SEQ ID NO: 179 - DNA, 1817 bp, Homo sapiens
tgctattctgccaaaagacaatttctagagtagttttgaatgggttgatttcccccactcccacaaactctgaagccagtgtctagcttactaaaaaaagagttgtatataatatttaagatgctgagtatttcataggaaagctgaatgctgctgtaaagtgctctttaagtcttttttttttttaatccccttctaatgaatgaaactaggggaatttcaggggacagagatgggatttgttgtatgataaactgtatgtagtttttagtctttctgttttgagaagcagtggttggggcatttttaagatggctggctactcttgttttccctcatgataataaatttgtcataactcagtaacatgaacttgcccctagaggtagttgttaataattttgaaatattaaggtcttgccaagcttctgatgattcacacctgtactactgattattaagcaggacagactgagctttctgttgcaaataccttggaggagaaagtaatttctaaatatacagagaggtaacttgactatatatgttgcatcctgtgcctcccttcatattaatatttgataaagattttaatttatgtaaaacttctaaagcagaatcaaagctcctcttggggaaatggcaagtctttaggataggcaagaccctgtatgaatagtaccaaagcattaccgcatggtagagaacacactcgattaaaaatgttaagctatctgaaaaataaaatgtgcaagtcttcaggatggcacaaaacaaaggttaatgcttcttggggcacatttcttagagggcttgctgagtgtgtaaatataatcgacttttgtttgtgttacatgacttctgtgacttcattgaaaatctgcacaattcagtttcagctctggattacttcagttgacctttgtgaaggtttttatctgtgtagaatgggtgtttgacttgttttagcctattaaatttttattttctttcactctgtattaaaagtaaaacttactaaaagaaaagaggtttgtgttcacattaaatggttttggtttggcttcttttagtcaggctttctgaacattgagatatcctgaacttagagctcttcaatcctaagattttcatgaaaagcctctcacttgaacccaaaccagagtactcttactgcctcttttctaaatgttcaggaaaagcattgccagttcagtcttttcaaaatgagggagaaacatttgcctgccttgtaataacaagactcagtgcttattttttaaactgcattttaaaaattggatagtataataacaataaggagtaagccaccttttataggcaccctgtagttttatagttcttaatctaaacattttatatttccttcttttggaaaaaacctacatgctacaagccaccatatgcacagactatacagtgagttgagttggctctcccacagtctttgaggtgaattacaaaagtccagccattatcatcctcctgagttatttgaaatgattttttttgtacattttggctgcagtattggtggtagaatatactataatatggatcatctctacttctgtatttatttatttattactagacctcaaccacagtcttctttttccccttccacctctctttgcctgtaggatgtactgtatgtagtcatgcactttgtattaatatattagaaatctacagatctgttttgtactttttatactgttggatacttataatcaaaacttttactagggtattgaataaatctagtcttactagaaaaaaaaaaaaaaaaaaaa

SEQ ID NO: 180 - DNA, 2382 bp, Homo sapiens
acttttattggaagcagcagccacatccctgcatgatttgcattgcaatacaaccataaccgggcagccactcctgagtgataaccagtataacataaacgtagcagcctcaatttttgcctttatgacgacagcttgttatggttgcagtttgggtctggctttacgaagatggcgaccgtaacactccttagaaactggcagtcgtatgttagtttcacttgtctactttatatgtctgatcaatttggataccattttgtccagatgcaaaaacattccaaaagtaatgtgtttagtagagagagactctaagctcaagttctggtttatttcatggatggaatgttaattttattatgatattaaagaaatggccttttattttacatctctcccctttttccctttccccctttattttcctccttttctttctgaaagtttccttttatgtccataaaatacaaatatattgttcataaaaaattagtatcccttttgtttggttgctgagtcacctgaaccttaattttaattggtaattacagcccctaaaaaaaacacatttcaaataggcttcccactaaactctatattttagtgtaaaccaggaattggcacactttttttagaatgggccagatggtaaatatttatgcttcacggtccatacagtctctgtcacaactattcagttctgctagtatagcgtgaaagcagctatacacaatacagaaatgaatgagtgtggttatgttctaataaaacttatttataaaaacaaggggaggctgggtttagcctgtgggccatagtttgtcaaccactggtgtaaaaccttagttatatatgatctgcattttcttgaactgatcattgaaaacttataaacctaacagaaaagccacataatatttagtgtcattatgcaataatcacattgcctttgtgttaatagtcaaatacttacctttggagaatacttacctttggaggaatgtataaaatttctcaggcagagtcctggatataggaaaaagtaatttatgaagtaaacttcagttgcttaatcaaactaatgatagtctaacaactgagcaagatcctcatctgagagtgcttaaaatgggatccccagagaccattaaccaatactggaactggtatctagctactgatgtcttactttgagtttatttatgcttcagaatacagttgtttgccctgtgcatgaatatacccatatttgtgtgtggatatgtgaagcttttccaaatagagctctcagaagaattaagtttttacttctaattattttgcattactttgagttaaatttgaatagagtattaaatataaagttgtagattcttatgtgtttttgtattagcccagacatctgtaatgtttttgcactggtgacagacaaaatctgttttaaaatcatatccagcacaaaaactatttctggctgaatagcacagaaaagtattttaacctacctgtagagatcctcgtcatggaaaggtgccaaactgttttgaatggaaggacaagtaagagtgaggccacagttcccaccacacgagggcttttgtattgttctactttttcagccctttactttctggctgaagcatccccttggagtgccatgtataagttgggctattagagttcatggaacatagaacaaccatgaatgagtggcatgatccgtgcttaatgatcaagtgttacttatctaataatcctctagaaagaaccctgttagatcttggtttgtgataaaaatataaagacagaagacatgaggaaaaacaaaaggtttgaggaaatcaggcatatgactttatacttaacatcagatcttttctataatatcctactactttggttttcctagctccataccacacacctaaacctgtattatgaattacatattacaaagtcataaatgtgccatatggatatacagtacattctagttggaatcgtttactctgctagaatttaggtgtgagattttttgtttcccaggtatagcaggcttatgtttggtggcattaaattggtttctttaaaatgctttggtggcacttttgtaaacagattgcttctagattgttacaaaccaagcctaagacacatctgtgaatacttagatttgtagcttaatcacattctagacttgtgagttgaatgacaaagcagttgaacaaaaattatggcatttaagaatttaacatgtcttagctgtaaaaatgagaaagtgttggttggttttaaaatctggtaactccatgatgaaaagaaatttattttatacgtgttatgtctctaataaagtattcatttgataaaaaaaaaaaaaaa

SEQ ID NO: 181 - DNA, 2377 bp, Homo sapiens
atctttatgcaagacaagagtcagccatcagacactgaaatatattatgatagattatgaagaattttctctgtagaattatattcttcctggaacctggtagagtagattagactcaaaggctttttcttccttttcttactcctgttttttccactcactcttcccaagagatttcctaaagcttcaagcttaataagcctaatagtgaaaaataactgaatttaatggtataatgaagttcttcatttccagacatctttaattgatcttaaagctcatttgagtctttgcccctgaacaaagacagacccattaaaatctaagaattctaaattttcacaactgtttgagcttcttttcattttgaaggatttggaatatatatgttttcataaaagtatcaagtgaaatatagttacatgggagctcaatcatgtgcagattgcattctgttatgttgactcaatatttaatttacaactatccttatttatattgacctcaagaactccattttatgcaatgcagaccactgagatatagctaacattctttcaaataattttccttttcttttataattcctctatagcaaatttttatgtataactgattatacatatccatatttatatttcattgattccaagacatcactttttcaatttaacatctctgaaattgtgacatttcttgcaactgttggcacttcagatgcagtgtttaaaattatgcttgaataaatattacactaatccaactttacctaaatgtttatgcatctaggcaaattttgttttcttataaagatttgagagcccatttatgacaaaatatgaaggcgaaatttaaggacaactgagtcacgcacaactcaacatggagcctaactgattatcagctcagatcccgcatatcttgagtttacaaaagctctttcaggtccccatttatactttacgtgagtgcgaatgatttcagcaaaccctaacttaactaacaagaatgggtaggtatgtctacgtttcattaacaaatttttattatttttattctattatatgagatccttttatattatcatctcacttttaaacaaaattaactggaaaaatattacatggaactgtcatagttaggttttgcagcatcttacatgtcttgtatcaatggcaggagaaaaatatgataaaaacaatcagtgctgtgaaaaacaactttcttctagagtcctcttactttttattcttctttatcatttgtgggtttttcccccttggctctcactttaacttcaagcttatgtaacgactgttataaaactgcatatttaaattatttgaattatatgaaataattgttcagctatctgggcagctgttaatgtaaacctgagagtaataacactactcttttatctacctggaatacttttctgcataaaatttatctttgtaagctaactctattaatcaggtttcttctagcctctgcaacctacttcagttagaattgtctaatactgctctattaatcaggtttctaccctctacaacctacttcagttaaaattgtctaatacagcaatatttaaaaaaaaaacactgcaattgtcaaggatggaaaatgtgtgatttgtgtaaacaatttttaccaactttacattttcctacagataaatgtgaaattttgataagaagtctacgcaatgacaagtacggtacataaattttattaagaatattgagtataaagtactttaattctaaattataagaaaatatacatttgcacatattaatatagaaattcattttgtgtatatttaacatagcttttaaactattttacattagctacttcattatggtttcttgaacttctgaaaaaaattagaaatgtattaaacttatcagtaacataaaaacttattttgtttcacctaacgaatactgcgtttgtaaaaataaatttaatatagaatatatttttaaattaaatatttgaatataaaatagctctaagaaagaagcaaattatcactgaacatatttcttattatttctggctttgaattatacgtaacttaaattgtcttaaatgatacagaatattggagaatatgatactttcacataatatactatgaacctgttcatataactctgattgactactaacttctgttttatgtatttattaaagagctgacactgtagtttgtggtgagatgtttatttttctaacagagcttataacagttaggacaaggcatttaattaatgcatcattctgtttagtagtaggtgttaatcaatatgaaattctctgttttaaaataaaaatgtaaaaatctaaaaaaaaaaaaaaaaaa

SEQ ID NO: 182 - DNA, 1370 bp, Homo sapiens
tgtgagcatggtattttgtctcggaagaaaaaaatatgggtcaggcgcaaagtaagcccaccccactgggaactatgttaaaaaaaaatttcaagatttaagggagattacggtgttactatgacaccagaaaaacttagaactttgtgtgaaatagactggctaacattagaggtgggttggctatcagaagaaagcctggagaggtcccttgtttcaaaggtatggcacaaggtaacctgtaagccaaagcacccggaccagtttctatacatagacagttacagctggtttagaccccttccccctctccccacagtagttaagagaacagcagcataagcagctggcagaggcaaggaaagaccagcagagagaaaaaaaggccatctataccaattttaagttaatttagactgaacaagggcttattaatagcaaaggataattgaaatcacaaacttataagggtttcaacaaaagtgaagtttgctaaaagttaacagtgtaacatgtattatggtaacttctaatcttgtggccttagacagtctagtcaaaacacataaagaaagtttgctttaaaaaaacaatggttatcttcaaaaataaaggggagaggcagaatttatataaaaagagttatatgataaattcttgtcctgaaataaattaactggttgtttaaagaaaagaatgtttgtaataagtcaaaaagttaaaacatgtttaaaaaattgtctgcaaaagtcataaaagaaaaaattttattaaaaaaattttaagcaaaaaatgttgtataatttaaaagtaataaggcctcctgtgtactattaagacagatgcaaattcctggttgaaatggatcaaatattccatctgcacattaaacaaaagcaattgttatgcttgtgcacatggcaggccagaggccctgattgtcccccttccactaaggtggtcctctagtcgaccaggcgtggactgcatggtagctcttttccaggattctacagcctggagtaataagtcatgccaagctctctctgctatatcccaaagtctctgcgggtcagcccccaagggccatgcagcttctgtctcccaacactaagttcacttcgtgtctctcacggcagagaggaaacttagtattccttggagacctgaagggatgcagtgagcttaagaattttcaagagcttatcaatcagtcagcccttgttcatccccgagtggatgtgtggtggtattgtggtggacctttactgggcactctgccaaataactagtgtggcacttgtgctttagtccatttggctatccctttcaccctggcatttcatcaaccaaaaaaaaaaaaaaaaaaa

SEQ ID NO: 183 - DNA, 2060 bp, Homo sapiens
  misc_feature 2003: n = A,T,C or G
gtttcaggggaggagacaaggtttcttgtttgccgtatatgctcctgcagagaagaggaagtgaccgtggaggccatctggccctgtgttttgatatggcaaaattaatgaatgcaatcagaagacctttgagcaagaaagtaccctggaacaacccaatttggactgcaagtattagttgggtcttccaggtgcctctcacagcagcagtcatggcagcagtgactctagccatgtccatgaccaactgctgcataacaaatagccccgagactcagcagcttacaacagggtccccagcccacagactggcactggtccatggcttgttaggaacctgactgcgcagcagaaggtgagtgagcattactgcctgagctctgcctcctgtcagatcatcaggggcattagattctcataggagcgtgaaccctattgcaaaccgcgcatgcgaaggatgtacgttgcgtgctccttatgagaatctaactaatgcctgatgatttgaggtggggcagtttcatccccaaaccatctctctcccttcatgtccatggaaaaattgtcttctacaaaaccagtccgtggtgccaaaaaggttggagactgctggtttacaaccgcaatgaacattcatcatcccacacagtgtcagagggtcgggaacacgggtgccctgcctgtgtgcttccggttccagatttctcagtgggttgtgatcaaggtatcagcggaggccgtattcatctgcaagcttgaccaggaatagaagagccacttcatgggtggctcactcagatgccagcaggtcagtgctggtggctggcaggcagcctcagctcctcacctcatggatctctcctgagcacagttttcctgtccttacaacctggtagctggcttctccagagcaggtgactcaggagaggacaaggtgagagcccagcaccttatggtctagtctcagaagtcacacgccatcatttctgcaatgtcattttggggttccaggtcagctgtatcactgtgggaggtgagtatatagatgtcctagaccattcaggctgctatgacagaacaccatgaactgagtggctcatgaacaacagaaatttcccacagttctgtaggctgggaaatccaagatcaaggtggcagcaggttcagcgtctgctaagctcctgcttttcatggattgcatcttctcactgtgtcctcacgtgatggacagagcaaatgagctctcaggcactagtcccagccatgaggactctgctttcatgactcatcactccgcaaaggcccacctccatcagaagacagctgctaactgcagctgccatcctccaagacgggagacacagaattgggggacatatacattgagatctgaaaggcctggacagcaacaggtggggatcgtgggggcatcttggagggtggctgccgcagtaacatttctgacccatgctttctgcttgcactcatctcctgcctttgatcttcattatctcargcagtccccacaacgactgtatctaggagttcattttaccctcattttacagatgaaacgtctcagagggtaatgtgcttgcccagtgtctcacaaatgcaaagtcactgaggtaggatttcaacctaggtccaatcatctctgcagcattaggggttcaccattgccatagacttaactgtgtcccccaaaatttgtatgttgaagccctaccagcctcccccccccaatgtgctgatgtttggagaaagggcctttgggaggtaattaggtttagatgagatcatgagggtgggactctcataatggcattaatgccatcaggtgaagagataccagagaccttgtgtcctctctctctgcaatgtgaggacacagtgagaaggcagctgtctgcaagctgggaagagagtactgaccaggaacttaatcagagggcatcttgatcttggacttcccagcctccagaactctgaaaagttaatgnctattatttaagccacgcagtctatggaattttgttagagccaaccccaagcttact

SEQ ID NO: 184 - DNA, 3079 bp, Homo sapiens
ggcacaaagttgggggccgcgaagatgaggctgtccccggcgcccctgaagctgagccggactccggcactgctggccctggcgctgcccctggccgcggcgctggccttctccgacgagaccctggacaaagtgcccaagtcagagggctactgtagccgtatcctgcgcgcccagggcacgcggcgcgagggctacaccgagttcagcctccgcgtggagggcgaccccgacttctacaagccgggaaccagctaccgcgtaacactttcagctgctcctccctcctacttcagaggattcacattaattgccctcagagagaacagagagggtgataaggaagaagaccatgctgggaccttccagatcatagacgaagaagaaactcagtttatgagcaattgccctgttgcagtcactgaaagcactccacggaggaggacccggatccaggtgttttggatagcaccaccagcgggaacaggctgcgtgattctgaaggccagcatcgtacaaaaacgcattatttattttcaagatgagggctctctgaccaagaaactttgtgaacaagattccacatttgatggggtgactgacaaacccatcttagactgctgtgcctgcggaactgccaagtacagactcacattttatgggaattggtccgagaagacacacccaaaggattaccctcgtcgggccaaccactggtctgcgatcatcggaggatcccactccaagaattatgtactgtgggaatatggaggatatgccagcgaaggcgtcaaacaagttgcagaattgggctcacccgtgaaaatggaggaagaaattcgacaacagagtgatgaggtcctcaccgtcatcaaagccaaagcccaatggccagcctggcagcctctcaacgtgagagcagcaccttcagctgaattttccgtggacagaacgcgccatttaatgtccttcctgaccatgatgggccctagtcccgactggaacgtaggcttatctgcagaagatctgtgcaccaaggaatgtggctgggtccagaaggtggtgcaagacctgattccctgggacgctggcaccgacagcggggtgacctatgagtcacccaacaaacccaccattccccaggagaaaatccggcccctgaccagcctggaccatcctcagagtcctttctatgacccagagggtgggtccatcactcaagtagccagagttgtcatcgagagaatcgcacggaagggtgaacaatgcaatattgtacctgacaatgtcgatgatattgtagctgacctggctccagaagagaaagatgaagatgacacccctgaaacctgcatctactccaactggtccccatggtccgcctgcagctcctccacctgtgacaaaggcaagaggatgcgacagcgcatgctgaaagcacagctggacctcagcgtcccctgccctgacacccaggacttccagccctgcatgggccctggctgcagtgacgaagacggctccacctgcaccatgtccgagtggatcacctggtcgccctgcagcatctcctgcggcatgggcatgaggtcccgggagaggtatgtgaagcagttcccggaggacggctccgtgtgcacgctgcccactgaggaaatggagaagtgcacggtcaacgaggagtgctctcccagcagctgcctgatgaccgagtggggcgagtgggacgagtgcagcgccacctgcggcatgggcatgaagaagcggcaccgcatgatcaagatgaaccccgcagatggctccatgtgcaaagccgagacatcacaggcagagaagtgcatgatgccagagtgccacaccatcccatgcttgctgtccccatggtccgagtggagtgactgcagcgtgacctgcgggaagggcatgcgaacccgacagcggatgctcaagtctctggcagaacttggagactgcaatgaggatctggagcaggtggagaagtgcatgctccctgaatgccccattgactgtgagctcaccgagtggtcccagtggtcggaatgtaacaagtcatgtgggaaaggccacgtgattcgaacccggatgatccaaatggagcctcagtttggaggtgcaccctgcccagagactgtgcagcgaaaaaagtgccgcatccgaaaatgccttcgaaatccatccatccaaaagccacgctggagggaggcccgagagagccggcggagtgagcagctgaaggaagagtctgaaggggagcagttcccaggttgtaggatgcgcccatggacggcctggtcagaatgcaccaaactgtgcggaggtggaattcaggaacgttacatgactgtaaagaagagattcaaaagctcccagtttaccagctgcaaagacaagaaggagatcagagcatgcaatgttcatccttgttagcaagggtacgagttccccagggctgcactctagattccagagtcaccaatggctggattatttgcttgtttaagacaatttaaattgtgtacgctagttttcatttttgcagtgtggttcgcccagtagtcttgtggatgccagagacatcctttctgaatacttcttgatgggtacaggctgagtggggcgccctcacctccagccagcctcttcctgcagaggagtagtgtcagccaccttgtactaagctgaaacatgtccctctggagcttccacctggccagggaggacggagactttgacctactccacatggagaggcaaccatgtctggaagtgactatgcctgagtcccagggtgcggcaggtaggaaacattcacagatgaagacagcagattccccacattctcatctttggcctgttcaatgaaaccattgtttgcccatctcttcttagtggaactttaggtctcttttcaagtctcctcagtcatcaatagttcctggggaaaaacagagctggtagacttgaagaggagcattgatgttgggtggcttttgttctttcactgagaaattcggaatacatttgtctcacccctgatattggttcctgatgccccagc

SEQ ID NO: 185 - DNA, 3000 bp, Homo sapiens
gtttcaggggaggagacaaggtttcttgtttgccgtatatgctcctgcagagaagaggaagtgaccgtggaggccatctggccctgtgttttgatatggcaaaattaatgaatgcaatcagaagacctttgagcaagaaagtaccctggaacaacccaatttggactgcaagtattagttgggtcttccaggtgcctctcacagcagcagtcatggcagcagtgactctagccatgtccatgaccaactgctgcataacaaatagccccgagactcagcagcttacaacagggtccccagcccacagactggcactggtccatggcttgttaggaacctgactgcgcagcagaaggtgagtgagcattactgcctgagctctgcctcctgtcagatcatcaggggcattagattctcataggagcgtgaaccctattgcaaaccgcgcatgcgaaggatgtacgttgcgtgctccttatgagaatctaactaatgcctgatgatttgaggtggggcagtttcatccccaaaccatctctctcccttcatgtccatggaaaaattgtcttctacaaaaccagtccgtggtgccaaaaaggttggagactgctggtttacaaccgcaatgaacattcatcatcccacacagtgtcagagggtcgggaacacgggtgccctgcctgtgtgcttccggttccagatttctcagtgggttgtgatcaaggtatcagcggaggccgtattcatctgcaagcttgaccaggaatagaagagccacttcatgggtggctcactcagatgccagcaggtcagtgctggtggctggcaggcagcctcagctcctcacctcatggatctctcctgagcacagttttcctgtccttacaacctggtagctggcttctccagagcaggtgactcaggagaggacaaggtgagagccacagcaccttatggtctagtctcagaagtcacacgccatcatttctgcaatgtcattttggggttccaggtcagctgtatcactgtgggaggtgagtatatagatgtcctagaccattcaggctgctatgacagaacaccatgaactgagtggctcatgaacaacagaaatttcccacagttctgtaggctgggaaatccaagatcaaggtggcagcaggttcagcgtctgctaagctcctgcttttcatggattgcatcttctcactgtgtcctcacgtgatggacagagcaaatgagctctcaggcactagtcccagccatgaggactctgctttcatgactcatcactccgcaaaggcccacctccatcagaagacagctgctaactgcagctgccatcctccaagacgggagacacagaattgggggacatatacattgagatctgaaaggcctggacagcaacaggtggggatcgtgggggcatcttggagggtggctgccgcagtaacatttctgacccatgctttctgcttgcactcatctcctgcctttgatcttcattatctcaggcagtccccacaacgactgtatctaggagttcattttaccctcattttacagatgaaacgtctcagagggtaatgtgcttgcccagtgtctcacaaatgcaaagtcactgaggtaggatttcaacctaggtccaatcatctctgcagcattaggggttcaccattgccatagacttaactgtgtcccccaaaatttgtatgttgaagccctaccagcctcccccccccaatgtgctgatgtttggagaaagggcctttgggaggtaattaggtttagatgagatcatgagggtgggactctcataatggcattaatgccatcaggtgaagagataccagagaccttgtgtcctctctctctgcaatgtgaggacacagtgagaaggcagctgtctgcaagctgggaagagagtactgaccaggaacttaatcagagggcatcttgatcttggacttcccagcctccagaactctgaaaagttaatgtctattatttaagccacgcagtctatggaattttgttagagccaacccaagcttactaagataatcagtatgctgcactttctataaatgtaatttttacatttataaaaacaaaacaagagatttgctgctctataacaactgtacctacattgtagatggaataacaaatctacatacagatttagtaatctctatgtagatatagaacatagtgtatctaatagagacatagtgtctgtggtctgatgttaattttaggaattagccgtcactgattgggccttgtccaggtattcttctcccttgtcctggctctgtaacctagttatccttgtctttgctaacccataaccaactattgtatcaggactattatgccactacagatgatgcagtttgggtttactgtttctcaccatttagacaatacttcatcaaatatatttctgtatgactttagtgatatcagtttttgattcattcctgcatagatctgggcaaattgtagaccttaggaggtgtattcaccatccagttctctggaactgcttatgacatttttctctgagctttcttgtcccaaaaggagccttcctaaaatagtctttaagtgcctttaaaaagagaaagagaaattaagagaaaaaaaaccccaaactcattcctttactctgatgtgacagtcctcccaggacactgcagtggcctgagttttgctgttaatttcattcacttatgtttgggctatgtaaattctgcctagagctggaatgtcattatgtaaagaaatattttttgtttatattctttaatagtaccagtaatgtatatcttattcagcttcgagaatataattgggttgtttataaaaaccacacatcatcaaactcacattgtaacgattatttcacttttcaaaaaaaatggcattagaaaaacttgaatgatgttagttatcttaaagaagtgtgtactatgtttaaaaaaaaaaaaa

SEQ ID NO: 186 - protein, 807 aa, Homo sapiens [sequence omitted]

SEQ ID NO: 187 - DNA, 892 bp, Homo sapiens
tttattgatgtttcaacaggcacttattcaaataagttatatatttgaaaacagccatggtaagcatccttggcttctcacccattcctcatgtggcatgctttctagactttaaaatgaggtaccctgaatagcactaagtgctctgtaagctcaaggaatctgtgcagtgctacaaagcccacaggcagagaaagaactcctcaagtgcttgtggtcagagactaggttccatatgaggcacacctatgatgaaggtcttcacctccagaaggtgacactgttcagagatcctcatttcctggagagtgggagaaaatccctcctttgggaaatcccttttcccagcagcagagcccacctcattgcttagtgatcatttggaaggcactgagagccttcaggggctgacagcagagaaatgaaaatgagtacagttcagatggtggaagaagcatggcagtgacatcttccatgctctttttctcagtgtctgcaactccaaagatcaaggccataacccaggagaccatcaacggaagattagttctttgtcaagtgaatgaaatccaaaagcacgcatgagaccaatgaaagtttccgcctgttgtaaaatctattttcccccaaggaaagtccttgcacagacaccagtgagtgagttctaaaagatacccttggaattatcagactcagaaacttttattttttttttctgtaacagtctcaccagacttctcataatgctcttaatatattgcacttttctaatcaaagtgcgagtttatgagggtaaagctctactttcctactgcagccttcagattctcatcattttgcatctattttgtagccaataaaactccgcactagcaaaaaaaaaaaaaaaaaa

SEQ ID NO: 188 - DNA, 1448 bp, Homo sapiens
  misc_feature 1124: n = A,T,C or G
tgtgactcacatttcttttactgtgacacaataatgtgatcctaaaactggcttatccttgagtgtttacaactcaaacaactttttgaatgcagtagttttttttttttaaaaacaaacttttatgtcaaattttttttcttagaagtagtcttcattattataaatttgtacaccaaaaggccatggggaactttgtgcaagtacctcatcgctgagcaaatggagcttgctatgttttaatttcagaaaatttcctcatatacgtagtgtgtagaatcaagtcttttaataattcattttttcttcataatatttactcaaagttaagcttaaaaataagttttatcttaaaatcatatttgaagacagtaagacagtaaactattttaggaagtcaacccccattgcactctgtggcagttattctggtaaaaataggcaaaagtgacctgaatctacaatggtgtcccaaagtaaccaagtaagagagattgtaaatgataaaccgagctttaaaggataaagtgttaataaagaaaggaagctgggcacatgtcaaaaagggagatcgaaatgttaggtaatcatttagaaaggacagaaaatatttaaagtggctcataggtaatgaatatttctgacttagatgtaaatccatctggaatctttacatcctttgccagctgaaacaagaaagtgaagggacaatgatatttcatggtcagtttattttgtaagagacagaagaaattatatctatacattaccttgtagcagcagtacctggaagccccagcccgtcacagaagtgtggaggggggctcctgactagacaatttccctagcccttgtgatttgaagcatgaaagttctggcaggttatgagcagcactagggataaagtatggttttattttggtgtaatttaggtttttcaacaaagcccttgtctaaaataaaaggcattattggaaatatttgaaaactagaaaatgatggataaaagggctgataagaaaatttctgactgtcagtagaagtgagataagatcctcagaggaaacagtaagaagggataatcattaagatagtaaaacaggcaaagcagaatcacatgtgcncacacacatacacatgtaaacattggaatgcataagttttaatattttagcgctatcagtttctaaatgcattaattactaactgccctctcccaagattcatttagttcaaacagtatccgtaaactaggaataatgccacatgcattcaatgggatcttttaagtactcttcagtttgttccaagaaatgtgcctactgaaatcaaattaatttgtattcaatgtgtacttcaagactgctaattgtttcatctgaaagcctacaatgaatcattgttcamccttgaaaaataaaattttgtaaatcaaaaaaaaaaaaaaa

SEQ ID NO: 189 - DNA, 460 bp, Homo sapiens
ttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatccttcaaaataaaaagtaatgacttaaaaaaaaaaaaaaaa

SEQ ID NO: 190 - DNA, 481 bp, Homo sapiens
aggtggtggaagaaactgtggcacgaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctga

SEQ ID NO: 191 - DNA, 489 bp, Homo sapiens
  misc_feature 312, 455: n = A,T,C or G
atataaattagactaagtgttttcaaataaatctaaatcttcagcatgatgtgttgtgtataattggagtagatattaattaagtcccctgtataatgttttgtaattttgcaaaacatatcttgagttgtttaaacagtcaaaatgtttgatattttataccagcttatgagctcaaagtactacagcaaagcctagcctgcatatcattcacccaaaacaaagtaatagcgcctcttttattattttgactgaatgttttatggaattgaaagaaacatacgttcttttcaagacttcctcatgaatctntcaattataggaaaagttattgtgataaaataggaacagctgaaagattgattaatgaactattgttaattcttcctattttaatgaatgacattgaactgaattttttgtctgttaaatgaacttgatagctaataaaaagncaactagccatcaaaaaaaaaaaaaaaaaaaaaa

SEQ ID NO: 192 - DNA, 516 bp, Homo sapiens
acttcaaagccagctgaaggaaagaggaagtgctagagagagcccccttcagtgtgcttctgacttttacggacttggcttgttagaaggctgaaagatgatggcaggaatgaaaatccagcttgtatgcatgctactcctggctttcagctcctggagtctgtgctcagattcagaagaggaaatgaaagcattagaagcagatttcttgaccaatatgcatacatcaaagattagtaaagcacatgttccctcttggaagatgactctgctaaatgtttgcagtcttgtaaataatttgaacagcccagctgaggaaacaggagaagttcatgaagaggagcttgttgcaagaaggaacttcttactgctttagatggctttagcttggaagcaatgttgacaatataccagctccacaaaatctgtcacagcagggcttttcaacactgggagttaatccaggaagatattcttgatactggaaatgacaaaaatggaaaggaagaagtcata

SEQ ID NO: 193 - DNA, 1409 bp, Homo sapiens
tgattcttttccaaaacttttagccatagggtcttttatagacagggatagtaaaatgaaaattgagaaatataagatgaaaaggaatggtaaaaatatcttttagggggcttttaattggtgatctgaaatcttgggagaagctgttcttttcaggcctgaggtgctcttgactgtcgcctgcgcactgtgtaccccgagcaacattctaagggtgtgctttcgccttggctaactcctttgacctcattcttcatatagtagtctaggaaaaagttgcaggtaatttaaactgtctagtggtacatagtaactgaatttctattcctatgagaaatgagaattatttatttgccatcaacacattttatactttgcatctccaaatttattgcggcgagacttgtccattgtgaaagttagagaacattatgtttgtatcatttctttcataaaacctcaagagcatttttaagcccttttcatcagacccagtgaaaactaaggatagatgttttttaactggaggtctcctgataaggagaacacaatccaccattgtcatttaagtaataagacaggaaattgaccttgacgctttcttgttaaatagatttaacaggaacatctgcacatcttttttccttgtgcactatttgtttaattgcagtggattaatacagcaagagtgccacattataactaggcaattatccattcttcaagacttagttattgtcacactaattgatcgtttaaggcataagatggtctagcattaggaacatgtgaagctaatctgctcaaaaagatcaacaaattaatattgttgctgatatttgcataattggctgcaattatttaatgtttaattgggttgatcaaatgagattcagcaattcacaagtgcattaatataaacagaactggggcacttaaaatgataatgattaacttatattgcatgttctcttcctttcacttttttcagtgtctacatttcagaccgagtttgtcagcttttttgaaaacacatcagtagaaaccaagattttaaaatgaagtgtcaagacgaaggcaaaacctgagcagttcctaaaaagatttgctgttagaaattttctttgtggcagtcatttattaaggattcaactcgtgatacaccaaaagaagagttgacttcagagatgtgttccatgctctctagcacaggaatgaataaatttataacacctgctttagcctttgttttcaaaagcacaaaggaaaagtgaaagggaaagagaaacaagtgactgagaagtcttgttaaggaatcaggttttttctacctggtaaacattctctattcttttctcaaaagattgttgtaagaaaaaatgtaagmcaaaaaaaaaaaaaaaaaaaaa

SEQ ID NO: 194 - DNA, 441 bp, Homo sapiens
cagatttcggtagccatctccctccaaatatgtctctttctgctttcttagtgcccattatttccccttctcctttcttctgtcactgccatctccttcttggtcttcccattgttctttaactggccgtaatgtggaattgatatttacattttgatacggtttttttcttggcctgtgtacgggattgcctcatttcctgctctgaattttaaaattagatattaaagctgtcatatggtttcctcacaaaagtcaacaaagtccaaacaaaaatagtttgccgttttactttcatccattgaaaaaggaaattgtgcctcttgcagcctaggcaaaggacatttagtactatcgattctttccaccctcacgatgacttgcggttctctctgtagaaaagggatggcctaagaaatacaactaaaaaaaaaaaaaaaa

SEQ ID NO: 195 - DNA, 707 bp, Homo sapiens
cagaaaaatatttggaaaaaatataccacttcatagctaagtcttacagagaagaggatttgctaataaaacttaagttttgaaaattaagatgcaggtagagcttctgaactaatgcccacagctccaaggaagacatgtcctatttagttattcaaatacaagttgagggcattgtgattaagcaaacaatatatttgttagaactttgtttttaaattactgttccttgacattacttataaagagtctctaactttcgatttctaaaactatgtaatacaaaagtatagtttccccatttgataaaaggccaatgatactgagtaggatatatgcgtatcatgctacttcattcagtgtgtctgtttttaatactaataaggcagtttgacagaaattatttctttgggactaaggtgattatcatttttttccccttcaaaattgtgctttaagtgctgataaccacaggcagattgcaaagaactgataaggcaacaaaagtagagaattttaggatcaaaggcatgtaactgaaaggtaacaacagtacataagcgacaactggggaaggcagcagtgaaacatgtttgtggggttaagtgagtcattgtaaataaggaatttgcacatttattttctgtcgacgcggccgccactgtgctggatatctgcagaattccaccacactggactagtggatc

SEQ ID NO: 196 - DNA, 552 bp, Homo sapiens
  misc_feature 61, 129, 189, 222, 241, 278, 324, 338, 363, 408, 415,463, 483: n = A,T,C or G
tggccagccagcctgatgtggatggcttccttggggtggtgcttccctcaagcccgaattngtggacatcatcaatgccaaacaatgagccccatccattttccctacccttcctgccaagccagggantaagcagcccagaagcccagtaactgccctttccctgcatatgcttttgatggtgtcatntgctccttcctgtggcctcatccaaactgtatnttcctttactgtttatatnttcaccctgtaatggttgggaccaggccaatcccttntccacttactataatggttggaactaaacgtcaccaaggtggcttntccttggctgaganatggaaggcgtggtgggatttgctnctgggttccctaggccctagtgagggcagaagagaaaccatcctntcccttnttacaccgtgaggccaagatcccctcagaaggcaggagtgctgccctntcccatggtgcccgtgcctntgtgctgtgtatgtgaaccacccatgtgagggaataaacctggcactaggaaaaaaaaaaaaaaaaaaa

SEQ ID NO: 197 - DNA, 449 bp, Homo sapiens
  misc_feature 56, 58, 76: n = A,T,C or G
ctccagagacaacttcgcggtgtggtgaactctctgaggaaaaacacgtgcgtggnancaagtgactgagacctanaaatccaagcgttggaggtcctgaggccagcctaagtcgcttcaaaatggaacgaaggcgtttgcggggttccattcagagccgatacatcagcatgagtgtgtggacaagcccacggagacttgtggagctggcagggcagagcctgctgaaggatgaggccctggccattgcccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgc

SEQ ID NO: 198 - DNA, 606 bp, Homo sapiens
tgagtttgcccccttacccccatcccagtgaatatttgcaattcctaaagacgtgttttgattgtcacacctgggtggggaacatgctactggcatctaatgcatagagggcagtaatgctgctaaacatctttcaacgcacaggacagagccccacaaaagagaattatctagccccaaatgtccataacactgctgttgagaaaacctaccgcaggatcttactgggcttcataggtaagcttgcctttgttctggcttctgtagatatataaaataaagacactgcccagtccctccctcaacgtcccgagccagggctcaaggcaattccaataacagtagaatgaacactaaatattgatttcaaaatctcagcaactagaagaatgaccaaccatcctggttggcctgggactgtcctagttttagcattgaaagtttcaggttccaggaaagccctcaggcctgggctgctggtcaccctagcagctgagggactcttcaatacagaattagtctttgtgcactggagatgaatatactttaatttgtaacatgtgaaaacatctataaacatctactgaagcctgttcttgtctgcac

SEQ ID NO: 199 - DNA, 369 bp, Homo sapiens
  misc_feature 29, 345: n = A,T,C or G
ggcaactttttgcggattgttcttgcttncaggctttgcgctgcaaatccagtgctaccagtgtgaagaattccagctgaacaacgactgctcctcccccgagttcattgtgaattgcacggtgaacgttcaagacatgtgtcagaaagaagtgatggagcaaagtgccgggatcatgtaccgcaagtcctgtgcatcatcagcggcctgtctcatcgcctctgccgggtaccagtccttctgctccccagggaaactgaactcagtttgcatcagctgctgcaacacccctctttgtaacgggccaaggcccaagaaaaggggaagttctgcctcggccctcangccatggctccgcaccaccatcct

SEQ ID NO: 200 - protein, 55 aa, Homo sapiens [sequence omitted]

SEQ ID NO: 201 - protein, 67 aa, Homo sapiens
MQLLSPNTKFTSCLSRQRGNLVFLGDLKGCSELKNFQELINQSALVHPRVDVWWYCGGPLLGTLPNN

SEQ ID NO: 202 - protein, 73 aa, Homo sapiens
MTPEKLRTLCEIDWLTLEVGWLSEESLERSLVSKVWHKVTCKPKHPDQFLYIDSYSWFRPLPPLPTVVKRTAA

SEQ ID NO: 203 - DNA, 2008 bp, Homo sapiens
ctccagagacaacttcgcggtgtggtgaactctctgaggaaaaacacgtgcgtggtaacaagtgactgagacctagaaatccaagcgttggaggtcctgaggccagcctaagtcgcttcaaaatggaacgaaggcgtttgcggggttccattcagagccgatacatcagcatgagtgtgtggacaagcccacggagacttgtggagctggcagggcagagcctgctgaaggatgaggccctggccattgcccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaactagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttggggataggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaaaaaaaaaaaaa

SEQ ID NO: 204 - DNA, 923 bp, Homo sapiens
tgagtttgcccccttacccccatcccagtgaatatttgcaattcctaaagacgtgttttgattgtcacacctgggtggggaacatgctactggcatctaatgcatagagggcagtaatgctgctaaacatctttcaacgcacaggacagagccccacaaaagagaattatctagccccaaatgtccataacactgctgttgagaaaacctaccgcaggatcttactgggcttcataggtaagcttgcctttgttctggcttctgtagatatataaaataaagacactgcccagtccctccctcaacgtcccgagccagggctcaaggcaattccaataacagtagaatgaacactaaatattgatttcaaaatctcagcaactagaagaatgaccaaccatcctggttggcctgggactgtcctagttttagcattgaaagtttcaggttccaggaaagccctcaggcctgggctgctggtcaccctagcagctgagggactcttcaatacagaattagtctttgtgcactggagatgaatatactttaatttgtaacatgtgaaaacatctataaacatctactgaagcctgttctgtctgcaccgacattttcattgagtacggattcttcctaccagatacagctgctctacaactttcgagggctggtataaaactagcttttacctatttttaaaaattacatgaatagtaaaaacttggattaacccagtattcgggtattttcaatttccttgggagcttagaggacggacaaataaaaagattatttcaacatcaaatatatgctattgtttacatatgaagataaccacatatatgtataaattcaccgttactttttagcaatactataaaatccaacagaaaaaaatagcatttactaaaaaaaaaaaaaaa

SEQ ID NO: 205 - DNA, 1619 bp, Homo sapiens
ggcaactttttgcggattgttcttgcttccaggctttgcgctgcaaatccagtgctaccagtgtgaagaattccagctgaacaacgactgctcctcccccgagttcattgtgaattgcacggtgaacgttcaagacatgtgtcagaaagaagtgatggagcaaagtgccgggatcatgtaccgcaagtcctgtgcatcatcagcggcctgtctcatcgcctctgccgggtaccagtccttctgctccccagggaaactgaactcagtttgcatcagctgctgcaacacccctctttgtaacgggccaaggcccaagaaaaggggaagttctgcctcggccctcaggccagggctccgcaccaccatcctgttcctcaaattagccctcttctcggcacactgctgaagctgaaggagatgccaccccctcctgcattgttcttccagccctcgcccccaaccccccacctccctgagtgagtttcttctgggtgtccttttattctgggtagggagcgggagtccgtgttctcttttgttcctgtgcaaataatgaaagagctcggtaaagcattctgaataaattcagcctgactgaattttcagtatgtacttgaaggaaggaggtggagtgaaagttcacccccatgtctgtgtaaccggagtcaaggccaggctggcagagtcagtccttagaagtcactgaggtgggcatctgccttttgtaaagcctccagtgtccattccatccctgatgggggcatagtttgagactgcagagtgagagtgacgttttcttagggctggagggccagttcccactcaaggctccctcgcttgacattcaaacttcatgctcctgaaaaccattctctgcagcagaattggctggtttcgcgcctgagttgggctctagtgactcgagactcaatgactgggacttagactggggctcggcctcgctctgaaaagtgcttaagaaaatcttctcagttctccttgcagaggactggcgccgggacgcgaagagcaacgggcgctgcacaaagcgggcgctgtcggtggtggagtgcgcatgtacgcgcaggcgcttctcgtggttggcgtgctgcagcgacaggcggcagcacagcaccttgcacgaacacccgccgaaactgctgcgaggacaccgtgtacaggagcgggttgatgaccgagctgaggtagaaaaacgtctccgagaaggggaggaggatcatgtacgcccggaagtaggacctcgtccagtcgtgcttgggtttggccgcagccatgatcctccgaatctggttgggcatccagcatacggccaatgtcacaacaatcagccctgggcagacacgagcaggagggagagacagagaaaagaaaaacacagcatgagaacacagtaaatgaataaaaccataaaatatttagcccctctgttctgtgcttactggccaggaaatggtaccaatttttcagtgttggacttgacagcttcttttgccacaagcaagagagaatttaacactgtttcaaacccgggggagttggctgtgttaaagaaagaccattaaatgctttagacagtgtaaaaaaaaaaaaaaaaaaaa

SEQ ID NO: 206 - DNA, 2364 bp, Homo sapiens
atgcagcatcaccaccatcaccacttctccgacgagaccctggacaaagtgcccaagtcagagggctactgtagccgtatcctgcgcgcccagggcacgcggcgcgagggctacaccgagttcagcctccgcgtggagggcgaccccgacttctacaagccgggaaccagctaccgcgtaacactttcagctgctcctccctcctacttcagaggattcacattaattgccctcagagagaacagagagggtgataaggaagaagaccatgctgggaccttccagatcatagacgaagaagaaactcagtttatgagcaattgccctgttgcagtcactgaaagcactccacggaggaggacccggatccaggtgttttggatagcaccaccagcgggaacaggctgcgtgattctgaaggccagcatcgtacaaaaacgcattatttattttcaagatgagggctctctgaccaagaaactttgtgaacaagattccacatttgatggggtgactgacaaacccatcttagactgctgtgcctgcggaactgccaagtacagactcacattttatgggaattggtccgagaagacacacccaaaggattaccctcgtcgggccaaccactggtctgcgatcatcggaggatcccactccaagaattatgtactgtgggaatatggaggatatgccagcgaaggcgtcaaacaagttgcagaattgggctcacccgtgaaaatggaggaagaaattcgacaacagagtgatgaggtcctcaccgtcatcaaagccaaagcccagtggccagcctggcagcctctcaacgtgagagcagcaccttcagctgaattttccgtggacagaacgcgccatttaatgtccttcctgaccatgatgggccctagtcccgactggaacgtaggcttatctgcagaagatctgtgcaccaaggaatgtggctgggtccagaaggtggtgcaagacctgattccctgggacgctggcaccgacagcggggtgacctatgagtcacccaacaaacccaccattccccaggagaaaatccggcccctgaccagcctggaccatcctcagagtcctttctatgacccagagggtgggtccatcactcaagtagccagagttgtcatcgagagaatcgcacggaagggtgaacaatgcaatattgtacctgacaatgtcgatgatattgtagctgacctggctccagaagagaaagatgaagatgacacccctgaaacctgcatctactccaactggtccccatggtccgcctgcagctcctccacctgtgacaaaggcaagaggatgcgacagcgcatgctgaaagcacagctggacctcagcgtcccctgccctgacacccaggacttccagccctgcatgggccctggctgcagtgacgaagacggctccacctgcaccatgtccgagtggatcacctggtcgccctgcagcatctcctgcggcatgggcatgaggtcccgggagaggtatgtgaagcagttcccggaggacggctccgtgtgcacgctgcccactgaggaaacggagaagtgcacggtcaacgaggagtgctctcccagcagctgcctgatgaccgagtggggcgagtgggacgagtgcagcgccacctgcggcatgggcatgaagaagcggcaccgcatgatcaagatgaaccccgcagatggctccatgtgcaaagccgagacatcacaggcagagaagtgcatgatgccagagtgccacaccatcccatgcttgctgtccccatggtccgagtggagtgactgcagcgtgacctgcgggaagggcatgcgaacccgacagcggatgctcaagtctctggcagaacttggagactgcaatgaggatctggagcaggtggagaagtgcatgctccctgaatgccccattgactgtgagctcaccgagtggtcccagtggtcggaatgtaacaagtcatgtgggaaaggccacgtgattcgaacccggatgatccaaatggagcctcagtttggaggtgcaccctgcccagagactgtgcagcgaaaaaagtgccgcatccgaaaatgccttcgaaatccatccatccaaaagctacgctggagggaggcccgagagagccggcggagtgagcagctgaaggaagagtctgaaggggagcagttcccaggttgtaggatgcgcccatggacggcctggtcagaatgcaccaaactgtgcggaggtggaattcaggaacgttacatgactgtaaagaagagattcaaaagctcccagtttaccagctgcaaagacaagaaggagatcagagcatgcaatgttcatccttgttag

SEQ ID NO: 207 - protein, 787 aa, Homo sapiens
MQHHHHHHFSDETLDKVPKSEGYCSRILRAQGTRREGYTEFSLRVEGDPDFYKPGTSYRVTLSAAPPSYFRGFTLIALRENREGDKEEDHAGTFQIIDEEETQFMSNCPVAVTESTPRRRTRIQVFWIAPPAGTGCVILKASIVQKRIIYFQDEGSLTKKLCEQDSTFDGVTDKPILDCCACGTAKYRLTFYGNWSEKTHPKDYPRRANHWSAIIGGSHSKNYVLWEYGGYASEGVKQVAELGSPVKMEEEIRQQSDEVLTVIKAKAQWPAWQPLNVRAAPSAEFSVDRTRHLMSFLTMMGPSPDWNVGLSAEDLCTKECGWVQKVVQDLIPWDAGTDSGVTYESPNKPTIPQEKIRPLTSLDHPQSPFYDPEGGSITQVARVVIERIARKGEQCNIVPDNVDDIVADLAPEEKDEDDTPETCIYSNWSPWSACSSSTCDKGKRMRQRMLKAQLDLSVPCPDTQDFQPCMGPGCSDEDGSTCTMSEWITWSPCSISCGMGMRSRERYVKQFPEDGSVCTLPTEETEKCTVNEECSPSSCLMTEWGEWDECSATCGMGMKKRHRMIKMNPADGSMCKAETSQAEKCMMPECHTIPCLLSPWSEWSDCSVTCGKGMRTRQRMLKSLAELGDCNEDLEQVEKCMLPECPIDCELTEWSQWSECNKSCGKGHVIRTRMIQMEPQFGGAPCPETVQRKKCRIRKCLRNPSIQKLRWREARESRRSEQLKEESEGEQFPGCRMRPWTAWSECTKLCGGGIQERYMTVKKRFKSSQFTSCKDKKEIRACNVHPC

SEQ ID NO: 208 - DNA, 1362 bp, Homo sapiens
atggcttcacccagcctcccgggcagtgactgctcccaaatcattgatcacagtcatgtccccgagtttgaggtggccacctggatcaaaatcacccttattctggtgtacctgatcatcttcgtgatgggccttctggggaacagcgccaccattcgggtcacccaggtgctgcagaagaaaggatacttgcagaaggaggtgacagaccacatggtgagtttggcttgctcggacatcttggtgttcctcatcggcatgcccatggagttctacagcatcatctggaatcccctgaccacgtccagctacaccctgtcctgcaagctgcacactttcctcttcgaggcctgcagctacgctacgctgctgcacgtgctgacactcagctttgagcgctacatcgccatctgtcaccccttcaggtacaaggctgtgtcgggaccttgccaggtgaagctgctgattggcttcgtctgggtcacctccgccctggtggcactgcccttgctgtttgccatgggtactgagtaccccctggtgaacgtgcccagccaccggggtctcacttgcaaccgctccagcacccgccaccacgagcagcccgagacctccaatatgtccatctgtaccaacctctccagccgctggaccgtgttccagtccagcatcttcggcgccttcgtggtctacctcgtggtcctgctctccgtagccttcatgtgctggaacatgatgcaggtgctcatgaaaagccagaagggctcgctggccgggggcacgcggcctccgcagctgaggaagtccgagagcgaagagagcaggaccgccaggaggcagaccatcatcttcctgaggctgattgttgtgacattggccgtatgctggatgcccaaccagattcggaggatcatggctgcggccaaacccaagcacgactggacgaggtcctacttccgggcgtacatgatcctcctccccttctcggagacgtttttctacctcagctcggtcatcaacccgctcctgtacacggtgtcctcgcagcagtttcggcgggtgttcgtgcaggtgctgtgctgccgcctgtcgctgcagcacgccaaccacgagaagcgcctgcgcgtacatgcgcactccaccaccgacagcgcccgctttgtgcagcgcccgttgctcttcgcgtcccggcgccagtcctctgcaaggagaactgagaagattttcttaagcacttttcagagcgaggccgagccccagtctaagtcccagtcattgagtctcgagtcactagagcccaactcaggcgcgaaaccagccaattctgctgcagagaatggttttcaggagcatgaagtttga

SEQ ID NO: 209 - protein, 453 aa, Homo sapiens
MASPSLPGSDCSQIIDHSHVPEFEVATWIKITLILVYLIIFVMGLLGNSATIRVTQVLQKKGYLQKEVTDHMVSLACSDILVFLIGMPMEFYSIIWNPLTTSSYTLSCKLHTFLFEACSYATLLHVLTLSFERYIAICHPFRYKAVSGPCQVKLLIGFVWVTSALVALPLLFAMGTEYPLVNVPSHRGLTCNRSSTRHHEQPETSNMSICTNLSSRWTVFQSSIFGAFVVYLVVLLSVAFMCWNMMQVLMKSQKGSLAGGTRPPQLRKSESEESRTARRQTIIFLRLIVVTLAVCWMPNQIRRIMAAAKPKHDWTRSYFRAYMILLPFSETFFYLSSVINPLLYTVSSQQFRRVFVQVLCCRLSLQHANHEKRLRVHAHSTTDSARFVQRPLLFASRRQSSARRTEKIFLSTFQSEAEPQSKSQSLSLESLEPNSGAKPANSAAENGFQEHEV

SEQ ID NO: 210 - DNA, 625 bp, Homo sapiens
  misc_feature 607: n = A,T,C or G
agttctccttgcagaggactggcgccgggacgcgaagagcaacgggcgctgcacaaagcgggcgctgtcggtggtggagtgcgcatgtacgcgcaggcgcttctcgtggttggcgtgctgcagcgacaggcggcagcacagcacctgcacgaacacccgccgaaactgctgcgaggacaccgtgtacaggagcgggttgatgaccgagctgaggtagaaaaacgtctccgagaaggggaggaggatcatgtacgcccggaagtaggacctcgtccagtcgtgcttgggtttggccgcagccatgatcctccgaatctggttgggcatccagcatacggccaatgtcacaacaatcagccctgggcagacacgagcaggagggagagacagagaaaagaaaaacacagcatgagaacacagtaaatgaataaaaccataaaatatttagcccctctgttctgtgcttactggccaggaaatggtaccaatttttcagtgttggacttgacagcttcttttgccacaagcaagagagaatttaacactgtttcaaacccgggggagttggctgtgttaaagaaagaccattaaatgctttagacagtgnaaaaaaaaaaaaaaaaaa

SEQ ID NO: 211 - DNA, 1619 bp, Homo sapiens; identical to SEQ ID NO: 205

SEQ ID NO: 212 - DNA, 1010 bp, Homo sapiens
ccgcagccgggagcccgagcgcgggcgatgcaggctccgcgagcggcacctgcggctcctctaagctacgaccgtcgtctccgctggcagcagctgcgggccccagcagcctcggcagccacagccgctgcagcctggggcagcctccgctgctgtcgcctcctctgatgcgcttgccctctccctggccccgggactccgggagaatgtgggtcctaggcatcgcggcaactttttgcggattgttcttgcttccaaggctttgcgctgcaaatccagtgctaccagtgtgaagaattccagctgaacaacgactgctcctcccccgagttcattgtgaattgcacggtgaacgttcaagacatgtgtcagaaagaagtgatggagcaaagtgccgggatcatgtaccgcaagtcctgtgcatcatcagcggcctgtctcatcgcctctgccgggtaccagtccttctgctccccagggaaactgaactcagtttgcatcagctgctgcaacacccctctttgtaaccgggccaaggcccaagaaaaggggaagttctgcctcggccctcaggccagggctccgaaccaccatcctgtccctcaaattaagccctacttctcggcacactgctggaagcttgaagggagaaggcacccactcctgcatagtccatccaggcctcgccccacacaccccactccctgagagagcacgcccagggagaccaaaaaccgggataggcaacggacccccagacaccacaagggacccgaggacaaagacgcagacaactcgcgaaagccacccacgaatacaacggcccgaacacagatataacgcacgagccccgaccgacaagagaagaagcagaagaaacacccacagacagaaacagacaccagcaacaagcgaaaacagcaaaacgacactagcgagacaccacctgcacacaacaccacagcccaacacagaggacacgacaacaaagagacagcaccaacgacgaa

SEQ ID NO: 213 - DNA, 480 bp, Homo sapiens
gccaactccggaggctctggtgctcggcccgggagcgcgagcgggaggagcagagacccgcagccgggagcccgagcgcgggcgatgcaggctccgcgagcggcacctgcggctcctctaagctacgaccgtcgtctccgcggcagcagcgcgggccccagcagcctcggcagccacagccgctgcagccggggcagcctccgctgctgtcgcctcctctgatgcgcttgccctctcccggccccgggactccgggagaatgtgggtcctaggcatcgcggcaactttttgcggattgttcttgcttccaggctttgcgctgcaaatccagtgctaccagtgtgaagaattccagctgaacaacgactgctcctcccccgagttcattgtgaattgcacggtgaacgttcaagacatgtgtgagaaagaagtgatggagcaaagtgccgggatcatgtaccgcaagtcctgtgcatgatc

SEQ ID NO: 214 - DNA, 1897 bp, Homo sapiens
gccaactccggaggctctggtgctcggcccgggagcgcgagcgggaggagcagagacccgcagccgggagcccgagcgcgggcgatgcaggctccgcgagcggcacctgcggctcctctaagctacgaccgtcgtctccgcggcagcagcgcgggccccagcagcctcggcagccacagccgctgcagccggggcagcctccgctgctgtcgcctcctctgatgcgcttgccctctcccggccccgggactccgggagaatgtgggtcctaggcatcgcggcaactttttgcggattgttcttgcttccaggctttgcgctgcaaatccagtgctaccagtgtgaagaattccagctgaacaacgactgctcctcccccgagttcattgtgaattgcacggtgaacgttcaagacatgtgtcagaaagaagtgatggagcaaagtgccgggatcatgtaccgcaagtcctgtgcatcatcagcggcctgtctcatcgcctctgccgggtaccagtccttctgctccccagggaaactgaactcagtttgcatcagctgctgcaacacccctctttgtaacgggccaaggcccaagaaaaggggaagttctgcctcggccctcaggccagggctccgcaccaccatcctgttcctcaaattagccctcttctcggcacactgctgaagctgaaggagatgccaccccctcctgcattgttcttccagccctcgcccccaaccccccacctccctgagtgagtttcttctgggtgtccttttattctgggtagggagcgggagtccgtgttctcttttgttcctgtgcaaataatgaaagagctcggtaaagcattctgaataaattcagcytgactgaattttcagtatgtacttgaaggaaggaggtggagtgaaagttcacccccatgtctgtgtaaccggagtcaaggccaggctggcagagtcwgtccttagaagtcactgaggtgggcatctgccttttgtaaagcctccagtgtccattccatccctgatgggggcatagtttgagactgcagagtgagagtgacgttttcttagggctggagggccagttcccactcaaggctccctcgcttgacattcaaacttcatgctcctgaaaaccattctctgcagcagaattggctggtttcgcgcctgagttgggctctagtgactcgagactcaatgactgggacttagactggggctcggcctcgctctgaaaagtgcttaagaaaatcttctcagttctccttgcagaggactggcgccgggacgcgaagagcaacgggcgctgcacaaagcgggcgctgtcggtggtggagtgcgcatgtacgcgcaggcgcttctcgtggttggcgtgctgcagcgacaggcggcagcacagcacctgcacgaacacccgccgaaactgctgcgaggacaccgtgtacaggagcgggttgatgaccgagctgaggtagaaaaacgtctccgagaaggggaggaggatcatgtacgcccggaagtaggacctcgtccagtcgtgcttgggtttggccgcagccatgatcctccgaatctggttgggcatccagcatacggccaatgtcacaacaatcagccctgggcagacacgagcaggagggagagacagagaaaagaaaaacacagcatgagaacacagtaaatgaataaaaccataaaatatttagcccctctgttctgtgcttactggccaggaaatggtaccaatttttcagtgttggacttgacagcttcttttgccacaagcaagagagaatttaacactgtttcaaacccgggggagttggctgtgttaaagaaagaccattaaatgctttagacagtgtaaaaaaaaaaaaaaaaaaaa

SEQ ID NO: 215 - protein, 141 aa, Homo sapiens
MWVLGIAATFCGLFLLPGFALQIQCYQCEEFQLNNDCSSPEFIVNCTVNVQDMCQKEVMEQSAGIMYRKSCASSAACLIASAGYQSFCSPGKLNSVCISCCNTPLCNGPRPKKRGSSASALRPGLRTTILFLKLALFSAHC

SEQ ID NO: 216 - DNA, 443 bp, Homo sapiens
  misc_feature 185,208,304,339,348,386,421,428: n = A,T,C or G
cctttttttttttttttctcagttattgactggctgggtgtgacttagtacataagtactcaatattataaaaacctcaaataattgacttgattttacacaacatccttcccttttctacaagttaatttttttacaaatcatttgggttatctcctaaataggttatattttattgcttctanaaacaatgtttcaaaatatatgngcattatcagtaataatttgtataaatatttcccacaacaattttcataattttcaaagactaatttcttgactgaagatattttgctagggaagngaaactttaaaattttgagattttaaaaaaattgngtgaatggnggcatgcaaaggatttatatagtggctcccctaactgngtgccgatcaggacacatatttttagacatctaantctggancttaaatggagggac

SEQ ID NO: 217 - DNA, 527 bp, Homo sapiens
  misc_feature 521,523: n = A,T,C or G
agacacaacagtctgactatgagtgaggaaaatatctgggtcttttcgtcagtttggtgcatttgctgctgctgttgctactgtttgcctcaaacgctgtgtttaaacaacgttaaactcttagcctacaaggtggctcttatgtacatagttgttaatacatccaattaatgatgtctgacatgctatttttgtagggagaaaatatgtgctaatgatattttgagttaaaatatcttttggggaggatttgctgaaaagttgcacttttgttacaatgcttatgcttggtacaagcttatgctgtcttaaattattttaaaaaaataaatactgtctgtgagaaaccagctggtttagaaaagtttagtatgtgacgataaactagaaattacctttatattctagtattttcagcactccataaattctattacctaaatattgccacactattttgtgatttaaaaattcttactaaggaataaaaactttaatatacaaaaaaaaaaaaaaggggngnccgc

SEQ ID NO: 218 - DNA, 896 bp, Homo sapiens
  misc_feature 531,587,589,592,619,636,649,662,663,694,723,729,735,737,741,752,783,816,817,819,820,822,826,828,830,833,834,839,841,842,869,892: n = A,T,C or G
gcagaacattattttacagacagcaaggatgcttctgagtgacacctaggaaattatttgaagaaattctttttatatctacacctgttgtgtaagaaactttaaaacattggttattttctcacctttttttctaattcactttgattgctaggggtcatgtatgcttcgaagttacaggactaaaagagcaaactgaccggcctaaaactaaaatgacatttattccctagctacaaacatcagcgttattatgttaattataccttgccctctatcattataaatggttgccatggtgtttctaaaaataagtgttttaccattaatgtgtagagggcaaacaaagcataaagtactaagggatcatgcttatcctagggtctcacagaagagaggacatatttaattaatcttgtgaattacagaacaggttgtggtccagacaccaagaatcataggggtttttttttaaaaaacctaatagaagtagggggacctctctctttggctaagagtctaaaggaaggnaggcatctgtttaattagttggttcaccctggctttacctctggttaatgctttgngntantaggaaggaaaaatcctttatcttttnttccaagccctccctgnctgacttacccanactgggattaccnngaaaccccagggggatttatggggggagaanggattttttcaccctttaaacctcttaanccccanggggananaaancctcttggganagcctatggccctattttttaatatccaggnccccttggaaaacttttttttttttaaaagccnntnnantttnantnaannaaaananncaaccttttggccccaaaaaaaaaggnccccccctaaggcccccaccctntttt

SEQ ID NO: 219 - DNA, 770 bp, Homo sapiens
  misc_feature 525,527,574,619,628,730,752: n = A,T,C or G
aaagaaggttcacttccattacagtatgagtggcaaaaattgtctgactcacagaaaatgcccacttcatggttagcagaaatgacttcatctgttatatctgtaaaaaatgcctcttctgagtactctgggacatacagctgtacagtcagaaacagagtgggctctgatcagtgcctgttgcgtctaaacgttgtccctccttcaaataaagctggactaattgcaggagccattataggaactttgcttgctctagcgctcattggtcttatcatcttttgctgtcgtaaaaagcgcagagaagaaaaatatgaaaaggaagttcatcacgatatcagggaagatgtgccacctccaaagagccgtacgtccactgccagaagctacatcggcagtaatcattcatccctggggtccatgtctccttccaacatggaaggatattccaagactcagtataaccaagtaccaagtgaagactttgaacgcactcctcagagtccgactctcccacctgctaangnagctgcccctaatctaagtcgaatgggtgcgattcctgtgatgattncagcacagagcaaggatgggtctatagtatagagcctccatatgnctcatctgngctctccggggtcctttcctttttttgatatatgaaaacctattctgggctaaattgggtactagcctcaaatcatcaaaaaataagttaatcaggaactgnaccgaaaatattttttaaaaanttttgtttgggtatattc

SEQ ID NO: 220 - DNA, 892 bp, Homo sapiens
  misc_feature 2,3,208,321,337,542,551,560,590,606,613,614,620,639,640,645,646,652,659,661,663,666,676,679,707,708,709,717,718,719,726,728,730,732,738,742,751,764,773,777,782,792,821,825,827,828,831,832,833,870,880: n = A,T,C or G
tnnacactcaccgccctcgccgccgcgccatggacgcccccaggcaggtggtcaactttgggcctggtcccgccaagctgccgcactcagtgttgttagagatacaaaaggaattattagactacaaaggagttggcattagtgttcttgaaatgagtcacaggtcatcagattttgccaagattattaacaatacagagaatcttgngcgggaattgctagctgttccagacaactataaggtgatttttctgcaaggaggtgggtgcggccagttcagtgctgtccccttaaacctcattggcttgaaagcaggaaggngtgcggactatgtggngacaggagcttggtcagctaaggccgcagaagaagccaagaagtttgggactataaatatcgttcaccctaaacttgggagttatacaaaaattccagatccaagcacctggaacctcacccagatgcctcctacgtgtattattgcgcaaatgagacggtgcatggtggtggagtttgactttatacccgatgtccagggagcnagtactggntttgtgacntgtcctcaaactttcctgtccaagccaggngggatgttttcccaantttgggnnggtganttttttgctggggggcccnnaaaannaaatgnttggggntncntgnctttgggggnccnaccccgggggggcggaaattggttcccnnncgggaatnnnaaccccntngncngggggngggntttttggcnccccttccccggnaaaaggcgnggcnccccnttcggggggncccttgggggaaaataaaccaaaggggtnggcncnnggnnntttgggggaaacacaccgagcgcttccctttttgttnacccaacaangggccctttcca

SEQ ID NO: 221 - DNA, 629 bp, Homo sapiens
  misc_feature 408,502,507,540,542,545,550,562,572,576,623,628: n = A,T,C or G
cctttttttttttttttggtacaaattatgtaaaacatttgtgctaagaacttttctccctccccaaaccaaaaagaaaataaaaaataaaaaaattaaaaaaattaaaaattgagtattctaactacagctcaacaattgaatcaaatgtcactgttttgtaaatactttatccataacgaaagatataaacatgcaaaaaacctgaatccatagtccaaataatacatacacatgttctgaagtttctgcacttctccatagactatgccaataaaacattatgtacacatactatttttacagtgaagtggaaaaatacagaaataaaaaagtgtacatggattaagaccaaaatgtgtctaacattctagtttatgaaaaaattcaattttgctacaaattggngatatgaaaactccctttatttgcaaccagctgagtaagttttaagattttagtgaaaaaaaaaaaaaacaaactaaagtctaaaactagaagnaatgngcattttccaatctcatgggctcatcccccaananaanaaaancgctccatgagnttttttggtnggtnaattttggattttaaaaaaagcaaatgcaatgtaacaaaagcggggntgaanc

SEQ ID NO: 222 - DNA, 763 bp, Homo sapiens
  misc_feature 626,628,634,661,748,751: n = A,T,C or G
ggaagtgctgaatggtgttggcaggggtattaaacgtgcatttttactcaactacctcaggtattcagtaatacaatgaaaagcaaaattgttccttttttttgaaaattttatatactttataatgatagaagtccaaccgttttttaaaaaataaatttaaaatttaacagcaatcagctaacaggcaaattaagatttttacttctggctggtgacagtaaagctggaaaattaatttcagggttttttgaggcttttgacacagttattagttaaatcaaatgttcaaaaatacggagcagtgcctagtatctggagagcagcactaccatttattctttcatttatagttgggaaagtttttgacggtactaacaaagtggtcgcaggagattttggaacggctggtttaaatggcttcaggagacttcagttttttgtttagctacatgattgaatgcataataaatgctttgtgcttctgactatcaatacctaaagaaagtgcatcagtgaagagatgcaagactttcaactgactggcaaaaagcaagctttagcttgtcttataggatgcttagtttgccactacacttcagaccaatgggacagtcatagatggngngacagngttaaacgcaacaaaaggctacatttncatggggccagcactggcatgagcctccctaagcttttttgaagaattttaagccctggtaaattaaaaaaaaaaaaaaaaaagggnggnccccctccaaat

SEQ ID NO: 223 - DNA, 885 bp, Homo sapiens
  misc_feature 21,571,599,653,714,717,746,755,756,761,762,781,782,790,814,849,884: n = A,T,C or G
tggagccgctgtggttgctgnccgcggagtggaagcgcgtgcttttgtttgtgtccctggccatggcgctgcagctctcccgggagcagggaatcaccctgcgcgggagcgccgaaatcgtggccgagttcttctcattcggcatcaacagcattttatatcagcgtggcatatatccatctgaaacctttactcgagtgcagaaatacggactcaccttgcttgtaactactgatcttgagctcataaaatacctaaataatgtggtggaacaactgaaagattggttatacaagtgttcagttcagaaactggttgtagttatctcaaatattgaaagtggtgaggtcctggaaagatggcagtttgatattgagtgtgacaagactgcaaaagatgacagtgcacccagagaaaagtctcagaaagctatccaggatgaaatccgttcagtgatcagacagatcacagctacggtgacatttctgccactgttggaagtttcttgtcatttgatctgctgatttatacagacaaagatttggttgtacctgaaaaatgggaagagtcnggaccacagtttattacccaattctgangaagtcccgccttcgttcatttactactacaatccacaaagtaaatagcatggngggctacaaaaaattcctgtcaatgactgaggatgacatgaaggaaaaaaatggaaattgnaanttttgaaaagggggtttcctgaaaacagncatctatanntggannttggtttatttcattgggnnaatttttncctgggggggaaaaaccccaaaanggatacctttactggaaccggggggggaaattggncctttttattttttttttgggcccccaattttggnc

SEQ ID NO: 224 - DNA, 541 bp, Homo sapiens
  misc_feature 300,311,350,422,490,508,526: n = A,T,C or G
cctttttttttttttttaaaacaaacttaactttatttcctcactttcacttaaaacttgattttataaaacacatgaaaaaacatttttaagagttctgtatcacagaacattaaacagtacaaatatccattgcttcataggttcaagttacataaattaaagtcaaataattggaaactgattcaatagggaaaactatacatgaaatgaaggtcaaaaggagctatacagcaatatttcattggttatagattatgagttactttcaggaccttaacaaagattctgaatatttanacttcctttgntggattttatacttaaatatctccctacctatactgagncaaactacttgaccaaaacatctgatttaggaaagcatctagctttatagcacaagtttttccatctacagntactatcttcaaaggaatatacatcacaatgttgacaaaaaaacctcctggttccttttgaacaatgngcaataaattcatgatgntaaccccatggggaaggncaaaaaggggaccca

SEQ ID NO: 225 - DNA, 543 bp, Homo sapiens
  misc_feature 23,226,295,316,327,345,428,445,476,479,521,522: n = A,T,C or G
cctttttttttttttttgtaagnttaaatttattttttaaaaatgcttgtcttcctcactagacaatcaactctatgagggcagagactatgtcaccactgtcccaccagcccctggcacacagtaggtactcaataaatatatgttggaaggatggatggaggtaatggatggaaagatggatggaaggatgaatggagggatggatgtgacccagctgaagtgngagtaggaacattctcttattatgggtggaggaaagagagaggagattgagaaaataagataaaatacnttgatgagcatcatttttggngttcgaaaagnaggattgaattaggactnataaatctagagaattttacctctttcaatgcccaagccacacttttctatcactttgaaaccgaaaaagaaatactttcccnacatttgctttgctggnaggaaatgctttaataaaaatgcaatctctnagntgccatggcatcattaaaagaaaggatgtcatgcccaggccnnaacttgaaggggggaggcccc

SEQ ID NO: 226 - DNA, 703 bp, Homo sapiens
  misc_feature 530,535,560,567,584,600,664,671: n = A,T,C or G
tgttaatgcaattatagaaatacatcggagacacaacatgatgtggccattacaggtttcataaaattacactgacttggctgttacttgatcttaggaaacagcacagtttaagatattgtgaattctgacttatactttattaaatgctataaatctaaatagatcctgttggatgtgatgggtctagtccagtttatttaagttcatgtttcactgtttgcactttgcattgaacaatgggtttattcgctgatgtaaacggttcgagtgaagaattaatgcagtaagtatgacaacacatacacacttgcctctccccatctccagaagaggggagcagagtccgagcttatctaaatatgaatgtggccacaaagctgtggaaggtgacaaagcttaaacacctttgccctggctctgcattgtcacctagagagcaagaggtctatagaaacatcatgtcacatgaaacgattctctgctttttggtctgaacttgaaggccctaaactgcaaaatctaagagntgggngggtattaaaatgcttttaaaaagntaactgnggcaccaattctaatgnaatcccacttgggacngggtttttttggtttggtttggttttgggggggggggggggggggccctgggaaaaggggaacnaacatgnttttgaaatacatattgggaaaaaaaatgggg

SEQ ID NO: 227 - DNA, 501 bp, Homo sapiens
  misc_feature 1,5,154,239,281,292,336,421,459,470: n = A,T,C or G
ngtgnccctggccatggcgctgcagctctcccgggagcagggaatcaccctgcgcgggagcgccgaaatcgtggccgagttcttctcattcggcatcaacagcattttatatcagcgtggcatatatccatctgaaacctttactcgagtgcanaaatacggactcaccttgcttgtaactactgatcttgagctcataaaatacctaaataatgtggtggaacaactgaaagattggntatacaagtgttcagttcagaaactggttgtagttatctcanatattgaaagnggtgaggtcctggaaagatggcagtttgatattgagtgtgacangactgcaaaagatgacagtgcacccagagaaaagtctcagaaagctatccaggatgaaatccgttcagtgatcagacagatcacngctacggggacatttctgccctgttggaaggttcttgntcatttgatcngctgatttatacagacaaaagatttggtttg

SEQ ID NO: 228 - DNA, 539 bp, Homo sapiens
  misc_feature 3,13,101,405,440,456,465,513,526: n = A,T,C or G
ggnttatactgcnaaagttatgcattacaccatattcagttggtaacataaaccgagatataagaatttatatattggcttctggttattttcttagcacnggagtgccttttccaaccattgagtgcatgatcagattacacaaatacaagcacatatcatgtgttctcccatgagacattattcacttaggattgtctacaataaaaaaagttaaagtacaagcaataataaattcataagaattttttgaatttaaaataaatgcatgtgtctttgagaacatttcttttgaaattcatatttttaaaaataacaagtttcttaaatcagtcttttagtcgtgttttcatatggtatttatcagtaggtggaaacacttcacatcatttaaccccaaaaggnataataattaaactgcaattaaagggaggaacagntgaatcattacaacantaatacggngtacaaatcagagttggccacacaatacacatgtgtaatactggaaanaaatacaatatcngaatcctggatgg

SEQ ID NO: 229 - DNA, 790 bp, Homo sapiens
  misc_feature 576,622,678,706,738,755,766: n = A,T,C or G
cagagagcatgatcagtgctgatactgacaagtacttttttaccttaaaatcaacttctatggaactacaagatcaatctagctcccgagtgacattttccattgtctgtaataatgccctcggatgagttgtgtctaaaattaagttcatctttatttatatgcgaacttaactgccatagtccctaatgtattgcgtttgtaacctgatcgtattatgtttacagctgaaagatttcatctagacatgtctttcgtccttattattcaaagtgtaattgaaagagatatttagtattaagacatgttccccaattgagaattttccagaatattctacttaagaagaagaagagcaattaactgcctttagtgtaagggcgagagtgcatagaaatatgcaatgtaaaatgtttgcatgaattatttcacatcatgtaagctttcccatattcataagatgaacactatagaagtctcatttctctgtgatcttctgccattaggaaagtaaggagattggtatctatatctagtctcctttccatattgaactgcatggctctaatcctcagnggatttttatcccttctccggttatttaaaatttgccctatttaanctggaagcctggataaactgctgagccccgaatattcctggggattgggagtttanttgctgggagaaccacttggttgaagancaccatttttttcccttttttttctttttccngaattttttccctcaanccattggtttnctcttaaatggaaaaacccccccg

SEQ ID NO: 230 - DNA, 744 bp, Homo sapiens
  misc_feature 603,618,636,723,724: n = A,T,C or G
aaattttatgggtgggtgccaaatactgctgtgaatctatttgtatagtatccatgaatgaatttatggaaatagatatttgtgcagctcaatttatgcagagattaaatgacatcataatactggatgaaaacttgcatagaattctgattaaatagtgggtctgtttcacatgtgcagtttgaagtatttaaataaccactcctttcacagtttattttcttctcaagcgttttcaagatctagcatgtggattttaaaagatttgccctcattaacaagaataacatttaaaggagattgtttcaaaatatttttgcaaattgagataaggacagaaagattgagaaacattgtatattttgcaaaaacaagatgtttgtagctgtttcagagagagtacggtatatttatggtaattttatccactagcaaatcttgatttagtttgatagtgtgtggaattttattttgaaggataagaccatgggaaaattgtggtaaagactgtttgacccttcatgaaataattctgaagttgccatcagttttactaatcttctgtgaaagcatagatatgcgcatggtcacttttattggggncttataattaaatgnaaaattgaaattcatttntgttcaaaggggatatcttccaatagcctttttagtagtattcaaatatcagtctatggataatgattttatttctttcttaggagnntcaatgtggactaattcagt

SEQ ID NO: 231 - DNA, 797 bp, Homo sapiens
  misc_feature 429,446,495,523,537,626,628,642,664,707,711,713,727,733,786,793: n = A,T,C or G
gtgccgcctccaaagagccgtacgtccgctgccagaagctgcataggcagtaatcattcatccctggaatccatgtctccttccaacatggagggatattccaagactcagtataaacaagtaccgagtgaagactttgaacgcactcctcagagtccaactctcccacctgctaaggtagctgcccctaatctaggtcgaatgggcgtgattcctgtgatgattcccgcacagagcaaggatgggtctatagtatagagcctccatacgtctcatctgtgctttccgtgttcctttccttttttgatatatgaaaacctattctggtctaaattttgttactagcctcaaaatgtatccaaaaataagttaatcaggagctgtaaggaatatattttttaaaatttttctttggttatatcgaaatangttacaggcattaaagntagtaaagacaagtttaccatctgaaaaagctggatttctttaagaggntgattataaagggttctaaatttatcantacctaagtaagangtagcacttttgaatatgaaatcataagtgaagacattggtgaacttacttgcatacccaagttgatactttgagtaaccatctgaaangngggacttggataanttttaccattatttttaagganggggatcttaattatttatgggcccccagtctcccccccaaantaantnccgaaaacattccnttgacnaaaattaccccctgggggggggttggacctttggttttcccaggtttcttggnaaaactntggg

SEQ ID NO: 232 - DNA, 635 bp, Homo sapiens
  misc_feature 501,531,556,623,633: n = A,T,C or G
tattattaggatggtaagagtattataaggattggtacaaggcatgatgagtccttttgcttttaggcttttgacttctggttttagactttctttagcttctgttgttagacaacattgtgcaagcttggtttttataagtttgcatggattaaactgaacttaatgaaattgtccctccccccaaattctcagcacaatttttaggcccacaaggagtcaagcacctcaaggagatcttcagtttgaacttggtgtagacacagggatactgatgaatcaatattcaaattagctgttacctacttaagaaagagaggagaccttggggatttcgaggaagggttcataagggagattttagctgagaaataccatttgcacagtcaatcacttctgaccaagttatcagaaaaaggagaaaagaatgtctccccactaaatgttctagggtggtgagaaatctagggtgggtatctaaatcacaatatttggatattncaatatctaaatattggtggaaatactctnctgaagtgtcattgactctaaaaanacacttgtgatcatggcaggggttaaggtcatttttattcctataatccttatattaacaattcctntgattaaganaa

SEQ ID NO: 233 - DNA, 663 bp, Homo sapiens
  misc_feature 429,432,437,475,485,491,493,535,550,555,571,612,640,653: n = A,T,C or G
cctctgtatagaaatctaaaagaattttaccattcagttaattcaatgtgaacactggcacactgctcttaagaaactatgaagatctgagatttttttgtgtatgtttttgactcttttgagtggtaatcatatgtgtctttatagatgtacatacctccttgcacaaatggaggggaattcattttcatcactgggagtgtccttagtgtatgaaaaccatgctggtatatggcttcaagttgtaaaaatgaaagtgactttaaaagaaaataggggatggtccaggatctccactgataagactgtttttaagtaacttaaggacctttgggtctacaagtatatgtgaaaaaaatgagacttactgggtgaggaaatccattgtttaaagatggtccgtgtgtgtgtgtgtgtgtgtgtgtgtgntgngttgngttttgttttttaagggagggaatttattatttaccgntgcttgaaantactgngnaaatatatgtctgataatgatttgctctttgacaactaaaantaggactgtataagncctanatgcctcctggggggntgatcttacaagatattggtgatacccctttaaaaattggncccccggcatttttcccctttgcttctncaaattaaaaggnctttttccca

SEQ ID NO: 234 - DNA, 873 bp, Homo sapiens
  misc_feature 20,29,58,603,630,652,678,711,715,745,752,756,766,774,789,820,823,840,873: n = A,T,C or G
acttggggattctcatgttnatggatacngtttggcaatcactacattgaatgtagtnttttaaaaaaattaacttatgctattagttgacccatcattgctaattttggcccacacagtgtttgcattacaaaaacctgttctttacttcctagtcttgtttcagtcttaatatcagaagttcttgagttcaaaataagcacaacatgtcatccagggatggctagcttgtttgggattcatctaaactgctggcaatatctagacaaaaacattccacagtccagctaatatggttgtcacaactcttgaaaagggcccaacatctggatggcaagtgaaaatgtgatcagggtttaagaactacccactaataaataaacatggagctatttccatgtcttgggtgttgtgtttctaagaagagacagcctttccatcagaaaatttctgggagggaagaaaaagaacagttttgatgaattcgctttgcaaatcatcatccaatgttctttgtaaccagaaaggttttcttctgctttcttgcagctggtatactttctgctgagtgccctggggcctgacggtctgtgtgctggccgnggcctttgcccgcccaccactattcgncagctcacaccagtttacctgngagaccccctccgacttttggccagngcaaactggccccttccttcgggagccggctcnagcnaggacccttttggttttacccggggatggngaccggnctgnacccagccgnccactggncccttttcaaacctngttcctttccctcatccccagaaggaatttnttnaaattttgggccttggnggcccttgggggggcctttgggttgggcccctn

SEQ ID NO: 235 - DNA, 51 bp, Homo sapiens
  misc_feature 26,48: n = A,T,C or G
tttttttttttttttttttaattttngttttttttttttttttttttnggg

SEQ ID NO: 236 - DNA, 765 bp, Homo sapiens
  misc_feature 540,555,590,593,670,685,708,711,714,733,760: n = A,T,C or G
ggagacctaatgtttcatatgcagcgacaaagaaaacttcctgaagaacatgccagattttactctgcagaaatcagtctagcattaaattatcttcatgagcgagggataatttatagagatttgaaactggacaatgtattactggactctgaaggccacattaaactcactgactacggcatgtgtaaggaaggattacggccaggagatacaaccagcactttctgtggtactcctaattacattgctcctgaaattttaagaggagaagattatggtttcagtgttgactggtgggctcttggagtgctcatgtttgagatgatggcaggaaggtctccatttgatattgttgggagctccgataaccctgaccagaacacagaggattatctcttccaagttattttggaaaaacaaattcgcataccacgttctctgtctgtaaaagctgcaagtgttctgaagagttttcttaataaggaccctaaggaacgattgggttgtcatcctcaaacaggatttgctgatattcanggaccccgtctttcnaaatgttgattgggatatgatggacaaaaacaggnggnaccttcctttaaccaaatatttctggggaatttgggtttggacacctttgattctcaatttactaatggaacctggnccagctcactcccanaatgacgaatgacctttgggganggnaanaattgggatcaagtctggnaattttggaaagggtttttggaggtantattc

SEQ ID NO: 237 - DNA, 739 bp, Homo sapiens
  misc_feature 460,478,485,509,527,529,554,573,575,578,603,607,609,616,621,643,651,674,675,689,696,729: n = A,T,C or G
ctctactggaagtttgaccctgtgaaggtgaaggctctggaaggcttcccccgtctcgtgggtcctgacttctttggctgtgccgagcctgccaacactttcctctgaccatggcttggatgccctcaggggtgctgacccctgccaggccacgaatatcaggctagagacccatggccatctttgtggctgtgggcaccaggcatgggactgagcccatgtctcctcagggggatggggtggggtacaaccaccatgacaactgccgggagggccacgcaggtcgtggtcacctgccagcgactgtctcagactgggcagggaggctttggcatgacttaagaggaagggcagtcttgggcccgctatgcaggtcctggcaaacctggctgcctgtctccatccctgtccctcagggtagcaccatggcaggactgggggaactggagtgccttgctgnatccctgttgggagggtnctttcangggctggcactgaaacaaaggggntgggggcccatgggcttnancctgggtgaacaactgggcttgtanggcaagggcactttctgangncanggcttgggaaggggcctgcatctgnctgncnttttggntgacnaatcctgggaaatctggttttnccaaaatnccaggccaaaaaagtttaccagnncaaaatgggggganggggganttttttttatggcaaggaaaaaacccccagggncccttgggaa

SEQ ID NO: 238 - DNA, 818 bp, Homo sapiens
  misc_feature 311,378,441,442,494,505,520,525,540,545,551,570,600,602,616,619,639,641,650,656,671,684,686,697,701,724,726,732,738,749,759,762,792,797: n = A,T,C or G
cctggtgatcgcttcagtagagatgtctggtgatatggtgaactgatcaggccctgacatggatgttcccctagaggatatcacttctgtcctggagacctcagtggtagcaccactgggcacttcagaaaggacagtgcttccctctgtggctgagctggtcccttcagagccgctggactccctcaatccaggggtcagggaggaagctagctctgtctgaatcctcctagtctcaaggaaggcaggagttgatgtgagaacacttgtatcccccatggtggaggtggtacacattggagatgagtcanctaggacagaggactgtgatttatatccagagctggtggttgccacattggtccctcctgtgtttgnggaaggatgcacggcttctgtatgtgcagtgtctttgtaagtggtaagtctctcatgggaggnngggctcaaacttgaagatgaactggttccaggttcttgtgcttgtacccaanatatctgtggntgtccccggccaganggganaaagtgaagtcacanggaangggaanagggggggatatgtgctangaatgtggtggaaaacaaggatgaagtgangncccggcaggtaaanacnagcgggggaaggaatggaangncttggtttnttttcncaaaagggaagggcntaggccaatgacncnccctcccggancttntgcccattgggaagggggcattntnttgggngggggnaaaatccctgnaattaactanaanaaagggggtttcccccccaaaaaggggggnggttncttggggttcaaaataaaggg

SEQ ID NO: 239 - DNA, 829 bp, Homo sapiens
  misc_feature 207,379,714,717,736,762,770: n = A,T,C or G
ctggtcttggactcctgacctcaagtgatccacccccctcagcctcccaaagtgctaggattacaggcatgagtcactgcgtcaggccaaaattctgtattttcaattagagtcaaagcccaaggatgtctctaccatcttgtagcccttgccaatagcctactcttgtcttccagggttcctccaaatctctctccaaatatttgntatctactcattcaatacccttcttcaaccatcctcttgctttggaattgacatgaaccaactaggcccgccttattggtaggaattcatttgccctgcctgccagcccccatagagacagaaccattgcctagtgaaagaagattttaatgacgtgatgaaaatattttanaaagcaccttgaagattagtatttttatgtaacttctgttggagagatgtcttcaggagactgaagtagaagagcgactgtcaaaatggaaagtcccagagacatccaatttatgtaaatcaacatcacctgaattcagaatctcatccagatttcaacaaagacttctgaatgccaaccaaagaagaggactgaatttacagactctcactctaacaatatatgctggtcaatttgaaaaacagaataaaattattttggcaagaaactggatttttaatggacatatattggtttaaaatggtaccaactttttatttttaccccattttggnggnaaaaaacccggggaataangggaaaagcaaaagggaaaatatatncaaatatngggaaggtttttacctttaattttggttcattaaacctaacccagaaggccaaacaatt

SEQ ID NO: 240 - DNA, 177 bp, Homo sapiens
cctttttttttttttttacatacaaaatgttttaattgagaaaaaaattcaaaacagtcacacatatccattatcatcatggttctctgaaatattttcttatacaaatgaaatatttaaaatggaaaaattacatttttcaaatctaattaactaattatttttgtcctggtcgac

SEQ ID NO: 241 - DNA, 591 bp, Homo sapiens
  misc_feature 96,152,212,246,280,403,436,491,494,501,519,568,579: n = A,T,C or G
ccttttttttttttttcattaaataatccatcatcacattagtacaatacaattttatattttttaaatatactatatatgttaaggataaggggngaagttttcttcctttgtaatacctgttcaagagtttaatggattaggagattagngttaaccttgaggataaaagtacaaatttgtctcattaggacacttctaccaagcatttnttaaggctatagtttaacatttggtttcaaaaanaaaaaaaaaggtttcatttaaaaaataatttagngaattacattctttcataacttccaccctaattagttacaaagataagtctaaagattcttagttttgggtactaatttacatttatatttaaagattaattttacttggatcttaaaacaanaattttatgttggaaaaaagagaactaaatacntttgtataaaggctgtaaatgtcccatggcaaatgctctgtctcaatattttctnccncaattanaaacagggctctgcaaanagagacttgggttgttcaggttcacctttcccgaggaattgggggctgncatctgaagancatagagaaaca

SEQ ID NO: 242 - DNA, 924 bp, Homo sapiens
  misc_feature 102,104,591,592,595,596,640,641,650,683,706,708,720,734,735,757,759,779,791,804,806,825,837,905,912: n = A,T,C or G
aacctttcaggaaaatccaaggaaatacagaagcaaggcagcaccatagtcttcccagccaaggtggaagtgcctctggttcctccagcaattcccactggngntatcataactcaacagtctgttgcaatcagttgtagaaaggcacagagtgacagctggaatgcaaagaaatgtgcacaacccagagctctgtcagccttgccaaaactcaagtgcccccatgggagggtcttgcaacatatgttctgttgagcaaagaggttgcaaaccaagcggttattgcaataaacaccacttgtgacaaacaaagtttgtaagtttaaatttattttttaaaaatgcttgtcttcctcactagacaatcaactctatgagggcagagactatgtcaccactgtcccaccagcccctggcacacagtaggtactcaataaatatatgttgggaaggatggatggaggtaatggatggaaagatggatggaaggatgaatggagggaatggatgtgacccagctgaagtgtgagtaggaacattctcttattatgggtggaggaaagagagaggagattgagaaaaataagatannatnncattggatgaagccatcattttttggggggttccgaaaaaagtnngggatttgnaaatttaagggaacttaaataaaaatccttaanaaaaaaaatttttttaacccctncntttttccaaaangggccccccaaaannccccaacccaactttttttttncnttatttccaccttttttggnaaaaaacccccnaaaaaaaaaaggntnaaaaattaccccttttttncccccaaacccntttttggcctttttgccttggggaaagggaaaaaaggctttttaaataaaaaaaatggccaattcttnttaaaanttggccaatggg

SEQ ID NO: 243 - DNA, 278 bp, Homo sapiens
  misc_feature 211,276,277: n = A,T,C or G
cctttttttttttttttaagatttaactctgaatacaaatgtatttttttcttcttctctccctacatatattctaaaccttctaaagtttttttatttttttaaggatcactttatcataaaataaaatatccttttcatataataaattacctaataaaaagtctttttttttcatattagcccaggttctttgctacatttatatggnaataaacgcctttattaaaatagaatattaaattataaagaactgctttttttttttttttttgnna

SEQ ID NO: 244 - DNA, 3072 bp, Homo sapiens
gcgcagacgccccagccccccaccgcccccaaaggggcgagcgacgccaagctctgcgctctctacaaagaggccgagctgcgcctgaagggcagcagcaacaccacggagtgtgttcccgtgcccacctccgagcacgtggccgagatcgtgggcaggcaaggctgcaagattaaggccttgagggccaagaccaacacctacatcaagacaccggtgaggggcgaggaaccagtgttcatggtgacagggcgacgggaggacgtggccacagcccggcgggaaatcatctcagcagcggagcacttctccatgatccgtgcctcccgcaacaagtcaggcgccgcctttggtgtggctcctgctctgcccggccaggtgaccatccgtgtgcgggtgccctaccgcgtggtggggctggtggtgggccccaaaggggcaaccatcaagcgcatccagcagcaaaccaacacatacattatcacaccaagccgtgaccgcgaccccgtgttcgagatcacgggtgccccaggcaacgtggagcgtgcgcgcgaggagatcgagacgcacatcgcggtgcgcactggcaagatcctcgagtacaacaatgaaaacgacttcctggcggggagccccgacgcagcaatcgatagccgctactccgacgcctggcgggtgcaccagcccggctgcaagcccctctccaccttccggcagaacagcctgggctgcatcggcgagtgcggagtggactctggctttgaggccccacgcctgggtgagcagggcggggactttggctacggcgggtacctctttccgggctatggcgtgggcaagcaggatgtgtactacggcgtggccgagactagccccccgctgtgggcgggccaggagaacgccacgcccacctccgtgctcttctcctctgcctcctcctcctcctcctcttccgccaaggcccgcgctgggcccccgggcgcacaccgctcccctgccacttccgcgggacccgagctggccggactcccgaggcgccccccgggagagccgctccagggcttctctaaacttggtgggggcggcctgcggagccccggcggcgggcgggattgcatggtctgctttgagagcgaagtgactgccgcccttgtgccctgcggacacaacctgttctgcatggagtgtgcagtacgcatctgcgagaggacggacccagagtgtcccgtctgccacatcacagccacgcaagccatccgaatattctcctaagccccgtgccccatgcctccggggcccactccactgggcccaccctggacctgttttccactaaagccttttggaaagcggtgatttgaggggcaaggtgcttagagatactcgctcgctggggaaggggggagggaggcagtggtggctggagggtgcgccactttcagagcctctggtcaccctgtcctggaaagattgggagggggccagactgaaaattttactagagttacaactctgatacctcaacacacccttaaatctggaagcagctaagagaaacttttgttttgccagaggtggccactaaggcattctgacgccctctgcccacctcccccgctgtgtgtcactccaccccttcttccgaggagggggtgggtaaaagggagagggagaattaccacctgtatctagaggtgctctttgcaatccctaagccctctggtcctgacctccgacctcccagctctgtcttgttccttgtctttgtctttcttcccttccccctgcccctgcccctaccagcccagctttggggacaccatccttctggggagaagtagggggaggaatatttggatggtccctccattcctcttcaggcatctggaggccctctcccccactcctccaaagaaacatctcaaattattgatggaatgtatccccattctcagtgaaaatgtgaggaggggactaatactggggtaaagggtcaaacccccaccttcatcactatgggcattatatttagggagtagttcttgggctggattttctggttgtggaagtgggggcgccagagtagtgtgtctgctatttaaaggagcaggaaagggcgtgaggcaggaggagagactggtggagggaagagctgctcctcccatgcagtgcccgactccctgcacccctctcaacctgacctgaacctttattgaatccttattagcttgaatccttattagcttgaatcctccatgcaaatcatggagtctgtgtcccacctgatgtggttgaggagaagccaggtcttcaaagaggggtcagcctggggcaaagcaggactggggggaggtgggcagcagggcctattctgagaatcacatattgttacaggccttgcaccccctttgctgcttccctcctgctcatttggggctgccaccagctctccaccctcctggttccgctggccgggccaagagaggatggagggatgggagtcccaggagatccttgtaaatagtggggtgggactgttctgagtgatcacccgagcacttaaagctccagagtcccattcttcctggatggagcaggtggaggtgcagaggggatttcctcctctccttcctcctgtcgagaattaacacctctccacagccttcccctccagaacaccagccagggaggggtggggaaggaggtcacagccaagaaaactgccctgtgacgacttccctccttcccgcctatgtgagccatcctgagatgtctgtacaatagaaaccaaaccaaatgggcaccctcggttgccggggggcaggtggggaggggggtgggaagaagggatgtctgtctgtcgtccccctccccctctccactctttacccacaaaggcagaagactgttacactagggggctcagcaaattcaatcccacccttaccaattgagccaaacctagaaacaaacacaaaacacgaatagtgagagacaaaatagaggagagaaagagagcatgagagggagcgagacaggcgaccaacacagaggagagaaaacaaaaatagc

SEQ ID NO: 245 - DNA, 2323 bp, Homo sapiens
ggactctggctttgaggccccacgcctgggtgagcagggcggggactttggctacggcgggtacctctttccgggctatggcgtgggcaagcaggatgtgtactacggcgtggccgagactagccccccgctgtgggcgggccaggagaacgccacgcccacctccgtgctcttctcctcctcctcctcctcctcctcttccgccaaggcccgcgctgggcccccgggcgcacaccgctcccctgccacttccgcgggacccgagctggccggactcccgaggcgccccccgggagagccgctccggggcttctctaaacttggtgggggcggcctgcggagccccgcagccggcgggcgggattgcatggtctgctttgagagcgaagtgactgccgcccttgtgccctgcggacacaacctgttctgcatggagtgtgcagtacgcatctgcgagaggacggacccagagtgtcccgtctgccacatcacagccacgcaagccatccgaatattctcctaagccccgtgccccatgcctccggggcccactccactgggcccaccctggacctgttttccactaaagccttttggaaagcggtgatttgaggggcaaggtgcttagagatactcgctcgctggggaaggggggagggaggcagtggtggctggagggtgcgccactttcagagcctctggtcaccctgtcctggaaagattgggagggggccagactgaaaattttactagagttacaactctgatacctcaacacacccttaaatctggaagcagctaagagaaacttttgttttgccagaggtggccactaaggcattctgacgccctctgcccacctcccccgctgtgtgtcactccaccccttcttccgaggagggggtgggtaaaagggagagggagaattaccacctgtatctagaggtgctctttgcaatccctaagccctctggtcctgacctccgacctcccagctctgtcttgttccttgtctttgtctttcttcccttccccctgcccctgcccctaccagcccagctttggggacaccatccttctggggagaagtagggggaggaatatttggatggtccctccattcctcttcaggcatctggaggccctctcccccactcctccaaagaaacatctcaaattattgatggaatgtatccccattctcagtgaaaatgtgaggaggggactaatactggggtaaagggtcaaacccccaccttcatcactatgggcattatatttagggagtagttcttgggctggattttctggttgtggaagtgggggcgccagagtagtgtgtctgctatttaaaggagcaggaaagggcgtgaggcaggaggagagactggtggagggaagagctgctcctcccatgcagtgcccgactccctgcacccctctcaacctgacctgaacctttattgaatccttattagcttgaatccttattagcttgaatcctccatgcaaatcatggagtctgtgtcccacctgatgtggttgaggagaagccaggtcttcaaagaggggtcagcctggggcaaagcaggactggggggaggtgggcagcagggcctattctgagaatcacatattgttacaggccttgcaccccctttgctgcttccctcctgctcatttggggctgccaccagctctccaccctcctggttccgctggccgggccaagagaggatggagggatgggagtcccaggagatccttgtaaatagtggggtgggactgttctgagtgatcacccgagcacttaaagctccagagtcccattcttcctggatggagcaggtggaggtgcagaggggatttcctcctctccttcctcctgtcgagaattaacacctctccacagccttcccctccagaacaccagccagggaggggtggggaaggaggtcacagccaagaaaactgccctgtgacgacttccctccttcccgcctatgtgagccatcctgagatgtctgtacaatagaaaccaaaccaaatgggcaccctcggttgccggggggcaggtggggaggggggtgggaagaagggatgtctgtctgtcgtccccctccccctctccactctttacccacaaaggcagaagactgttacactagggggctcagcaaattcaatcccacccttaccaattgagccaaacctagaaacaaacacaaaacacgaatagtgagagacaaaatagaggagagaaagagagcatgagagggagcgagacaggcgaccaacacagaggagagaaaacaaaaatagc

SEQ ID NO: 246 - DNA, 5882 bp, Homo sapiens
  misc_feature 1100,1975,4288,5859,5862,5863,5868: n = A,T,C or G
gcgcagacgccccagccccccaccgcccccaaaggggcgagcgacgccaagctctgcgctctctacaaagaggccgagctgcgcctgaagggcagcagcaacaccacggagtgtgttcccgtgcccacctccgagcacgtggccgagatcgtgggcaggcaaggctgcaagattaaggccttgagggccaagaccaacacctacatcaagacaccggtgaggggcgaggaaccagtgttcatggtgacagggcgacgggaggacgtggccacagcccggcgggaaatcatctcagcagcggagcacttctccatgatccgtgcctcccgcaacaagtcaggcgccgcctttggtgtggctcctgctctgcccggccaggtgaccatccgtgtgcgggtgccctaccgcgtggtggggctggtggtgggccccaaaggggcaaccatcaagcgcatccagcagcaaaccaacacatacattatcacaccaagccgtgaccgcgaccccgtgttcgagatcacgggtgccccaggcaacgtggagcgtgcgcgcgaggagatcgagacgcacatcgcggtgcgcactggcaagatcctcgagtacaacaatgaaaacgacttcctggcggggagccccgacgcagcaatcgatagccgctactccgacgcctggcgggtgcaccagcccggctgcaagcccctctccaccttccggcagaacagcctgggctgcatcggcgagtgcggagtggactctggctttgaggccccacgcctgggtgagcagggcggggactttggctacggcgggtacctctttccgggctatggcgtgggcaagcaggatgtgtactacggcgtggccgagactagccccccgctgtgggcgggccaggagaacgccacgcccacctccgtgctcttctcctctgcctcctcctcctcctcctcttccgccaaggcccgcgctgggcccccgggcgcacaccgctcccctgccacttccgcgggacccgagctggccggactcccgaggcgccccccgggagagccgctccagggcttctctaaacttggtgggggcggcctgcggagccccgscggcgggcgggattgcatggtctgctttgagagcgaagtgactgccgcccttgtgccctgcggacacaacctgttctgcatggagtgtgcagtacgcatctgcgagaggacggacccagagtgtcccgtctgccacatcacagccacgcaagccatccgaatattctcctaagccccgtgccccatgcctccggggcccactccactgggcccaccctggacctgttttccactaaagccttttggaaagcggtgatttgaggggcaaggtgcttagagatactcgctcgctggggaaggggggagggaggcagtggtggctggagggtgcgccactttcagagcctctggtcaccctgtcctggaaagattgggagggggccagactgaaaattttactagagttacaactctgatacctcaacacacccttaaatctggaagcagctaagagaaacttttgttttgccagaggtggccactaaggcattctgacgccctctgcccacctcccccgctgtgtgtcactccaccccttcttccgaggagggggtgggtaaaagggagagggagaattaccacctgtatctagaggtgctctttgcaatccctaagccctctggtcctgacctccgacctcctaacatgaccctttacctcccaccccacccccatatcctgtttgggaaactgtcaccagtttccagcagtgtaagggagttggagtcctatcagaagttgcatagatcttctaggggttggggagagaagcatgtcaatcgtttctgtggctgaaaggctcagaagccatctgtccccacaaagctgggctagaggaatctggagaggagtcctcctctctgcccctgtccccygcagtgtttcccttcactctctccgcctatcttcccttcctttgggatcttccctttcctcaactctttcctttccctccagctctttgctttgctttcttttggtggctgtcactcccagctctgtcttgttccttgtctttgtctttcttcccttccccctgcccctgcccctaccagcccagctttggggacaccatccttctggggagaagtagggggaggaatatttggatggtccctccattcctcttcaggcatctggaggccctctcccccactcctccaaagaaacatctcaaattattgatggaatgtatccccattctcagtgaaaatgtgaggaggggactaatactggggtaaagggtcaaacccccaccttcatcactatgggcattatatttagggagtagttcttgggctggattttctggttgtggaagtgggggcgccagagtagtgtgtctgctatttaaaggagcaggaaagggcgtgaggcaggaggagagactggtggagggaagagctgctcctcccatgcagtgcccgactccctgcacccctctcaacctgacctgaacctttattgaatccttattagcttgaatccttattagcttgaatcctccatgcaaatcatggagtctgtgtcccacctgatgtggttgaggagaagccaggtcttcaaagaggggtcagcctggggcaaagcaggactggggggaggtgggcagcagggcctattctgagaatcacatattgttacaggccttgcaccccctttgctgcttccctcctgctcatttggggctgccaccagctctccaccctcctggttccgctggccgggccaagagaggatggagggatgggagtcccaggagatccttgtaaatagtggggtgggactgttctgagtgatcacccgagcacttaaagctccagagtcccattcttcctggatggagcaggtggaggtgcagaggggatttcctcctctccttcctcctgtcgagaattaacacctctccacagccttcccctccagaacaccagccagggaggggtggggaaggaggtcacagccaagaaaactgccctgtgacgacttccctccttcccgcctatgtgagccatcctgagatgtctgtacaatagaaaccaaaccaaatgggcaccctcggttgccggggggcaggtggggaggggggtgggaagaagggatgtctgtctgtcgtccccctccccctctccactctttacccacaaaggcagaagactgttacactagggggctcagcaaattcaatcccacccttaccaattgagccaaacctagaaacaaacacaaaacacgaatagtgagagacaaaatagaggagagaaagagagcatgagagggagcgagacaggcgaccaacacagaggagagaaaacaaaaatagcaaaaaaaaaaaaaaaaaagcagttctttataatttaatattctattttaataaaggcgtttattaccatataaatgtagcaaagaacctgggctaatatgaaaaaaaaagactttttattaggtaatttattatatgaaaaggatattttattttatgataaagtgatccttaaaaaaataaaaaaactttagaaggtttagaatatatgtagggagagaagaagaaaaaaatacatttgtattcagagttaaatcttaaaaaaaaaaagtgtttttaatatatgtttgggtttacgttgcttttttcccccactttttttttggggaggaatgtcatttgcttttcttgggggagcatcccgggggtgaatggtggagagaggagctgggggaacccggtccctcctgggacccttccagtagattggatttcactccatggactcctcctcccctctccccctccccctcaggggagccggcagagccaaacaaagaaagggattaacaagaaaggaagaagctgtaggactaaggactgaggatcctggggtgtcccccaccactttcccctgccctgtcgcaggggcaagtgaggagggggaatccagaattaaggcctagcaggcctataggaaccctcagagatgtgtgagatttaagagatctagatttttttttaaccaaaaacaagagagaaagagaagaaaaagagaaaccgaggggtttaaaagaaaagaatactacaaaataataattattaataataataattcaaatttatttcatataatcctagagagagaaagaaacaattactagttacttagtagacaatattaagatagcttaaagtttagtascattgagggcccctgggtccagtagaatgtataaaagttgtaaggaaaagataaatagaggagggaagtggctgagtccaccctgagttgcccaatcttcagataccagggttggatcaggttgctagtttaagattgggagcttccagtctgctggggttgattctgagaatccttggatttttaaattgtaggacaaagaaatgaggggttcatttcccagggtcttggaaaggatgcacactgatcatctcaataagacaggggctgggttgggggcagcagaggaggccaagcacattcacctgcacccctagtacctgggcagcccatactccaatgtggtatgtcccctcctggggctcccagctcaaaccctcccatgcctgcttcccccaggcctaactgaggaagtccttcttgaagtgtgacctcggtccacttctctacagattgatttaagagcctgggaagtcattccacaaacagacacacatgcacacacgcttctcaccttcagagcttcaagagcactgaggcgatcagtcccctacccctgttcccatccagctttccacttagctttgacctccatggcagcagtagcagtaacaatctcagtaattgttctttaaagctgactcgttcttcacctacttgcaaagtgctttcttgtctcataaaagttagattccaagaaggacttcccacggagtggagtggaaacactgtccttgaaggcctgggagaaaggcatccccatgggcacagaggctggggaaaggcacagggactttgggtgaccctaaccctgaccctctgctccagttcacctccatctatatgtgttcaggtaggggtcatctactgtaccctggcctgggaacacattgccctccccacacaaaactggagggcttggcttctgcgtgtgagaaatcaacatttttaaagcacttgccttctaccaaccccagcttgcaatcactgggccttcccctcctatccaaggggttggaggggccccttggctctccttttggcaggaggagcctgcttcattacaccaatgactctgccatccccctccctggccctagaccccaaacacatctccctctacccaatttactcttctcgccccacctagggacagattccccctgctctttttgtcctagaaaccccgctagtttgggatggtagcgtctggggtggggagggcttccccttccccactcgagggtgcgggtggggaagggggggtgggtggagacagccctggggcagggaggatggtctctccactgtagaaagtagagtaggattgtggtcagacttaatttgaggcatctagtgaagacacgtacaaatccaccaaggaaaaagatttcaaaagcaaaataaaagcgggaaataaaacagacccaagaataatcaagtcaaagtgatgttgcacaaaatgcagagaaaccaagaagggggagggttaatgtattaaatgtgctattaagaacttaattttattaaaagtactattacttaaaaaaaaaaaaaaaaaaaaaaaaaawaarwagtcrtatcgaatcgatgt

SEQ ID NO: 247 - protein, 343 aa, Homo sapiens
MVTGRREDVATARREIISAAEHFSMIRASRNKSGAAFGVAPALPGQVTIRVRVPYRVVGLVVGPKGATIKRIQQQTNTYIITPSRDRDPVFEITGAPGNVERAREEIETHIAVRTGKILEYNNENDFLAGSPDAAIDSRYSDAWRVHQPGCKPLSTFRQNSLGCIGECGVDSGFEAPRLGEQGGDFGYGGYLFPGYGVGKQDVYYGVAETSPPLWAGQENATPTSVLFSSASSSSSSSAKARAGPPGAHRSPATSAGPELAGLPRRPPGEPLQGFSKLGGGGLRSPGGGRDCMVCFESEVTAALVPCGHNLFCMECAVRICERTDPECPVCHITATQAIRIFS

SEQ ID NO: 248 - protein, 343 aa, Homo sapiens; identical to SEQ ID NO: 247

SEQ ID NO: 249 - protein, 343 aa, Homo sapiens
  variant 287: Xaa = Any amino acid
MVTGRREDVATARREIISAAEHFSMIRASRNKSGAAFGVAPALPGQVTIRVRVPYRVVGLVVGPKGATIKRIQQQTNTYIITPSRDRDPVFEITGAPGNVERAREEIETHIAVRTGKILEYNNENDFLAGSPDAAIDSRYSDAWRVHQPGCKPLSTFRQNSLGCIGECGVDSGFEAPRLGEQGGDFGYGGYLFPGYGVGKQDVYYGVAETSPPLWAGQENATPTSVLFSSASSSSSSSAKARAGPPGAHRSPATSAGPELAGLPRRPPGEPLQGFSKLGGGGLRSPXGGRDCMVCFESEVTAALVPCGHNLFCMECAVRICERTDPECPVCHITATQAIRIFS

SEQ ID NO: 250 - DNA, 7993 bp, Homo sapiens
aaaaatgttttcctttatatttctgaggtgaaattcttccataggcatttcaggaggttttttgtcaaacattttaaaagcaaaattgataccatgtttctataaaatacgatatgcgaaatgatgcctattgctatttgctactgggctggaagttaggaaatagtgacggaaaaaccccaaatgcatacagagatcatgagtacagccagtgatgccagtgatgtattacgaagattacaaaaaaggccacaaaagttcaatgctaatcccttctgggtcgaacacagagtgacacattcggcagacaattatattttacttatgtaacgaataagtcatatttttctgtactgggcatttttagaggaacatataaagaaatggatagtgtcttaggggtctcattttctaatttagaaatgttttcactcccatgtgaaaagatttgctaatatataacagacaatctaacttggggcatcctattaaaataatctattttccataacttcaacctttttaaaaaataaagtcagtgggaatttctaatttccctgtgggttttatctatatgcattttttaggttttttttttccttatcatataccttccagattttatgtttaaataaataaatgatatttcaagataaagttagtctataaagggacactaaatcagcctacagatgtcaatctctaggtttaactacagagatagttccacataaatgccaaaaagaagtggttttgatgtcaatctttatgaaaatggttttatttaaccattgtatcaagtctaactatacttgggcagatttgagctttaaaaataaagctatgtatttcgtttttaaaaatgtgcttctctgtttcttcatttacttacaactgtggaacagaatcacgagtttgtggacgaacaagtctttgaagaaagctgcatggaagttgcaactgttaatcgtccttcaagtcacagtccttcactgtcttcacaacaaggagtcaccagcacctgctgttcacgacgacacaaaaaaacttttcgcatcccaaatgccaatgtatcaggaagccatcaaggtagtatacaagaactcagcacgattcagatcagatgtgtggagagaacacctctgtctaacaggtacctgagattaatctgtgttgtctacacacctgtgctggttcccagggtgtgtcttctgcactcatgttgtcacttacatggcatctaaatccctagctcctatggttcaggaagagacaaaaatggtagcgtaacaagtaggaaaatggttctgcttgcatatccattaagggttaaaaatagtggttatatcagtattaaaagagtcgaaagaagaagagatgattgagtgcacacaaatgtgtttttctctcttctgttccacaaccttttcttatttgggcaaggacttttatactcaggagtctcttatatattcaatagtctgaaatgatggtggacactactaaaagagtcaaatgaaattgagatcaacatggctaaaaattattgaacagcatgtaaaaatatacaaacacactgtctgtaacagtaaaaatgaaaactttgcctacataagtcatttctatcaatatataattttgatcagaaaggtatttttggccatatcaaacacaaaatcaacataatacaaaaaataagtgaagtactaaaattgtttgcctctggttagtttatgaacaaattaagtaaaacttccatattgatatatttcctttgcttttccttattcactgtttttaccacaaatgtgtaaaaataaaaagttgtacatttaaacaatatatgtcattaaaaatcagtttctgccaaaataattttattctgttttcaaattgaacagcatatattgttagagtgagagtctgtaaattcagtcctcttctttgttggcattcagaagtctttgttgaaatctggatgagattctgaattcaggtgatgttgatttacataaattggatgtctctgggactagaaaattaaaattaggagccataagacttctatcttcaagatattttagctttgcagttttatatcctttataaagtgaagtcgacaatggaaaattatctagcaagaaaatcttaggacataaacatcttaattatgttttccatgaaaataaaataatcagtttcaagcttctgtgtatatccttctctctcatttcccttttatccccttccccacatagagtatacaattcatccaataataccatttgggagcttgaagtgttagatagtaatagataatttttttatttctgtaatatgtgatcatcattaagttcagaggtttgagggcattatatatcccaaaaagacatgaaaataaaaatattctgtggtcaaaggaattaagaaatcaatgtgatatggctctttcatagtggtttctcaaacctctcagaggtctgtcttaaataaaacttctttgctcagcccaggctgtccaaatcttacttgaagctctttctttcttacatttttttataatacatttagtttgataaatacttgatggaaccaagtcaaactgtcttttagaaatttatttaatgttaattactatgttcatctttaacatagattaagatttggtgtttcatatttttaattataataactttccttagacaattaaaatatttttataagcattacaacacatattcttcagttgtatgaaaaacattttaagtaaacaacttactttcctaaatatttttttttctatcagccgatccagtttaaatgccaaaatggaagagtgtgttaaactaaactgtgaacaaccttatgtgactacagcaataataagcatcccaacacctccagtaaccacaccagaaggagacgataggccagaatcccctgagtactcaggaggaaatattgtcagagtttctgctttgtaagacaattggaataaggtctaagagaattcgagccctggctgtgaaaagaatctcaacatagaagaaagaagaaacaataaatattctgcagattaatgcagcaaagaaagaaggttggtagtgaaacacaaagcttccaatcttaaggatgtgaataaaaccaccaaatggcatttctagacagtttgacctgttatacagagtaatattctgtggccctttgactttgtgaatgagcacaatgaaatgccgcctactgatgcttcttatgatcagaactcttttttaataaaataaataacataaatcgttgaacataatgttccagttgaatgcaaaacaaaaaaaatatggaaaacattttgataaaattttttcctgttaaaaccatgaacattggctatgatgaagattattacatatgaaaaaaaaactcacacaacatatttgtattgactgaaggaaaccatcataatgcatgctagaattctttgaagcagtgatctcagtttccttatgttgtcttcagaataggcatgataaactataattgtagaaaggggtaatttctgtgcacttacaacaagctgagtgttcatgttccatggtgggctgtgcaaataaactccttttagacctgcagtatttctcatggggatgctcattagtaaatctaaagtgttcagatagttcagtattcattatcgtttaactttgcacctagatactgttacaactgcaataatttgttgtacaactgttgtatcaggaatcaggatttttttgttgttgtactttccagatccttatagatacggtaagagccacattcgtagaaaaacttctggtgtggccaggttttaggtaactttttaatccaaaactattgtgccataaatgtttttcagtaatattttttggtccactgtattcctgtgacacagtgcattatctgttcttgtatttctatagcacctctctattgggtttatcatcatcaacaagactactgtttactgtagttcaagtgactttcctacttttgtatttccaaaaaaaattatcttgtaagtagcttgtcatcaatccccttgtcgaaaactagaaaaaaaggagttgacccatataaattatctctaacgtctttgttgtttatggaaaagcccagatactggatatatcactatgtattttatgaacagaattgactgggactaatatcacaggatcaatcatctcagaatcttacttgatgcattatttattttgctttagatcttgaatacattttgagaataactaatgtggattgaaatgtagagatacactggagtgctttatttagcaatatttgatgaaagcatgctttctacgccattcaggaaggcagcacaaatttatctcagaaaggttcctgtgtattgcaaggtacaattttctccaataaatcaggagaacaggagtttgatgatgcaaagttgatctctgtacatttaagtgaaaagtctttataacttttcacccttaaaatatttcagcagacatgtctgcacatgacagtgtaaaaaagtttaatgtcaaatgcaaagtttttattcattccaagccaccactgtaaggaataaagcttagcttctgtacatggaaagagctaataattatccctctgtcagagatgagatttttaaatgcttatgatatttaatcataaaaagggattaatccaaccattttctagtaaagccagaaattcttgcttcccatttctagaatagtttctagaacagtgctatgcacatattagatcttaataaacatttgctgagtgaaagtaagataaactcaactatctcttgggaagaactggcttcattcctagtacatcttttaaaaagttactaattttccagcagtacaaatattaacaattatattaacacctgcctcatgtcagtttatgcttctagagcaatgtctagtgaaacttatctgatggcatttattgaaaaccttctaaaaagtagactaaggaaaccataatcagaattactatgtcttttgattcccaatgagaagttctattttcatgttcttaatattacatacaagaaaatgcagttaggttatttcaattgacaattctgcctcctcttttgatttatcacttacccaaaattattaattttattaggcttttggaaaagaaaaaaaactttttgatgttttaggtgatttaaaaatataccgtgttggtggtgaatgactattgatgactgtgttaagtgcatctgtattgtaagtgaaatgtaattatttctgtgtaccatatggagtaactaaggtcattgtttttgacaattttgtttgaaattcatatatcttatttcaaaggatagcataatatctgcattatgctggaaaaaaatagacctttggagaatacttaaataaaacatgtgcatgcttgaacaggacaaaatgttgactgttgccctattttcttagatttcattcctttcccaaaattaggatatgccacactcataatacacatgttggaggaccttgtgagacatacaactcaaaggacacagcaattgaaagtaatgcttaaatctcatctgaatgggtggagacagtagcttttgctagtaatgggaattaaggcagggactttaacagaaaagatagtatcaattaaggaaagccagtccctgaaccttatatacttcttaaacaccactacttgcattaagcagagaagctcaggggtaatggttttggtcagaacttaaataaattcttaatcaaaggctttattctacctaggaaagcggggtgatttatttgcctagccatttgtgtgcatgtatgtgtatatgtagatataaatttgttcatacacatatacatagattttcattcatttttaatatgcaaccactaatggtttctcttaatatcttgaaagggctaaaaagcaagaaaatgttaagagtttatagaagaaggaaaaagacacaaaggaaattatttagtagaaagactgattttaactggtgatatatatggtcctcttggtggatagtctctatcttttcttgttaattattttcatttatagcctttgatttattaggtatcaatcttgcattaaaaagttcaatatgcctccctattccttcaacttagtcacaatgttggcttagaaatagcctcttgggagctataatgtgtctgccagtaacattgctcaaaagaataaaaaagggttcttgaaagtaaattgataactccttagagtttcataagaaaggcatcttctcttccctacagtgtcattaaggtgtttgttttattaactcactggtacaagaatggttattactctgcactgtgtaaacatctgaattttcaacacaattgtgtaggcacacagtatttttttaatgaaggtttaaattgtacctacgaaggtttaagtcttatctacttcagctggttgtattaggatactaaaatattctttacagagtttgattttttacttctaaggaattactagctttgaacagcaagtttgcttaagataattataaaatataattttacaaaatatttttagttgaaaataatattaaatacatagcatacctttaatctttctctttactgcctgccctgccttttttctcctttatcttcaagcattaattattattgtagcagatctttgcctttccctaattacttttttctctagcttttctatggaaatcctttaggttacataactaatattcattctacatataatccagtttattaaatacagatgatgggccagacatggtgatagagaaatacagattaagaaaccagatcaaatcctttttaaggaattatctagtggaaaatatctcaactctcttctttacactactattcattatcttacacttcaaatcttcacctttccattttgacagtcgctcttctacttcagtctcctgaagacatctctccaacagaagttacataaaaatactaatcttcaaggtgctttctaaaatattttcatcacgtcattaaaatcttctttcactaggcaatggttctgtctctatgggggctggcaggcagggcaaatgaattcctacctgcccagagaaagaacaggaaacaataaaggtaaaacaaaacccaaaggaagaagagcttcatgtttatgcattacattaaagttaaaaggaaataaactttctcaagtatccactctaccttttcaactataatttcagagaatgtgaagaaagctattaaaatagttttgcaggaggactgatacacaatgcgtctgtgaatctgaacaccacacctcaaattctattatctgatgaatcctattgaaatatctgagtaattgggacaacagaaaggtaagtctgtaatcaggggcactcaccaataaggcgggcctagttggttcatgtcaattccaaagcaagaggatggttgaagaagggtattgaatgagtagataacaaatatttggagagagatttggaggaaccctggaagacaagagtaggctattggcaagggctacaagatggtagagacatccttgggctttgactctaattgaaaatacagaattttggcctgacgcagtgactcatgcctgtaatcctagcactttgggaggctgaggggggtggatcacttgaggtcaggagtccaagaccagcctcatcaactggtgaaaccccatctctactaaaaaaagaaaaaaaaaaaatagccaggtgtcctggtgcatgcctgtaatctcagctactcaggagtctgaggcaggagaattgcttgaactcgggaggtggaggttgcaatgagctgagattgtgccactgcattccagcctgggtgacagggggagactccgtcttaaaaaaagaaaaaagaaaaaccagaattttaaagttcaatttagatcaaattaattcctt

SEQ ID NO: 251 - DNA, 3254 bp, Homo sapiens
ggtatggtgacatggtgccaaaaaccatagcagggaagatttttggttctatctgttcgctgagtggggtcttggtcattgctctacctgttccggtgattgtatccaacttcagtcgcatctaccaccagaatcaacgagcagacaaacgaagggcacaaaagaaagctagactggccaggatccgggcagccaaaagcggaagcgcaaatgcttacatgcagagcaaacggaatggtttactcagtaatcagctgcagtcctcagaggatgagcaggcttttgttagcaaatccggctccagctttgaaacccagcaccaccacctgcttcactgcctggaaaaaaccacgaatcacgagtttgtggacgaacaagtctttgaagaaagctgcatggaagttgcaactgttaatcgtccttcaagtcacagtccttcactgtcttcacaacaaggagtcaccagcacctgctgttcacgacgacacaaaaaaacttttcgcatcccaaatgccaatgtatcaggaagccatcaaggtagtatacaagaactcagcacgattcagatcagatgtgtggagagaacacctctgtctaacagccgatccagtttaaatgccaaaatggaagagtgtgttaaactaaactgtgaacaaccttatgtgactacagcaataataagcatcccaacacctccagtaaccacaccagaaggagacgataggccagaatcccctgagtactcaggaggaaatattgtcagagtttctgctttgtaagacaattggaataaggtctaagagaattcgagccctggctgtgaaaagaatctcaacatagaagaaagaagaaacaataaatattctgcagattaatgcagcaaagaaagaaggttggtagtgaaacacaaagcttccaatcttaaggatgtgaataaaaccaccaaatggcatttctagacagtttgacctgttatacagagtaatattctgtggccctttgactttgtgaatgagcacaatgaaatgccgcctactgatgcttcttatgatcagaactcttttttaataaaataaataacataaatcgttgaacataatgttccagttgaatgcaaaacaaaaaaaatatggaaaacattttgataaaattttttcctgttaaaaccatgaacattggctatgatgaagattattacatatgaaaaaaaaactcacacaacatatttgtattgactgaaggaaaccatcataatgcatgctagaattctttgaagcagtgatctcagtttccttatgttgtcttcagaataggcatgataaactataattgtagaaaggggtaatttctgtgcacttacaacaagctgagtgttcatgttccatggtgggctgtgcaaataaactccttttagacctgcagtatttctcatggggatgctcattagtaaatctaaagtgttcagatagttcagtattcattatcgtttaactttgcacctagatactgttacaactgcaataatttgttgtacaactgttgtatcaggaatcaggatttttttgttgttgtactttccagatccttatagatacggtaagagccacattcgtagaaaaacttctggtgtggccaggttttaggtaactttttaatccaaaactattgtgccataaatgtttttcagtaatattttttggtccactgtattcctgtgacacagtgcattatctgttcttgtatttctatagcacctctctattgggtttatcatcatcaacaagactactgtttactgtagttcaagtgactttcctacttttgtatttccaaaaaaaattatcttgtaagtagcttgtcatcaatccccttgtcgaaaactagaaaaaaaggagttgacccatataaattatctctaacgtctttgttgtttatggaaaagcccagatactggatatatcactatgtattttatgaacagaattgactgggactaatatcacaggatcaatcatctcagaatcttacttgatgcattatttattttgctttagatcttgaatacattttgagaataactaatgtggattgaaatgtagagatacactggagtgctttatttagcaatatttgatgaaagcatgctttctacgccattcaggaaggcagcacaaatttatctcagaaaggttcctgtgtattgcaaggtacaattttctccaataaatcaggagaacaggagtttgatgatgcaaagttgatctctgtacatttaagtgaaaagtctttataacttttcacccttaaaatatttcagcagacatgtctgcacatgacagtgtaaaaaagtttaatgtcaaatgcaaagtttttattcattccaagccaccactgtaaggaataaagcttagcttctgtacatggaaagagctaataattatccctctgtcagagatgagatttttaaatgcttatgatatttaatcataaaaagggattaatccaaccattttctagtaaagccagaaattcttgcttcccatttctagaatagtttctagaacagtgctatgcacatattagatcttaataaacatttgctgagtgaaagtaagataaactcaactatctcttgggaagaactggcttcattcctagtacatcttttaaaaagttactaattttccagcagtacaaatattaacaattatattaacacctgcctcatgtcagtttatgcttctagagcaatgtctagtgaaacttatctgatggcatttattgaaaaccttctaaaaagtagactaaggaaaccataatcagaattactatgtcttttgattcccaatgagaagttctattttcatgttcttaatattacatacaagaaaatgcagttaggttatttcaattgacaattctgcctcctcttttgatttatcacttacccaaaattattaattttattaggcttttggaaaagaaaaaaaactttttgatgttttaggtgatttaaaaatataccgtgttggtggtgaatgactattgatgactgtgttaagtgcatctgtattgtaagtgaaatgtaattatttctgtgtaccatatggagtaactaaggtcattgtttttgacaattttgtttgaaattcatatatcttatttcaaaggatagcataatatctgcattatgctggaaaaaaatagacctttggagaatacttaaataaaacatgtgcatgcttgaacaggac

SEQ ID NO: 252 - DNA, 5333 bp, Homo sapiens
cggctgctcgcgagctgctttctctcctcttccctttccgggtgcacggcgaggagaaagtctctatgcaactaagccccggcgcgcacttggccaggtatgtaccgcgggagcggcgcgttctgcgcggaagcagatgctgctgccgccacggcggcggcggctgccagctcctgagctctgtaactgtcacactgcacctgagctgaacttgaaaagagagtgaaggggcgattgggcgaacgcttttggcagacacagagggtgtttgtagacgtgggggaggagaatctctattaacgccccccaccgtaaccactgcacatcacctccatctctgcaaatacagcccgaggagtagaggcagcagcagctggacccccaaagagagacgtggggcagcggctgtgaccgcatctcctgagctacaacaacaggtcgcctttttgagactcctttggcgggaagggctacttggaaaggaaggtttgaaagagtgagaagggtaggtgtaagggttccctaattcgtcgaaagaattctattgggtgactctcgttcgtcttctctatcctacactccacatactgaccctatattatccagactgtgccggggagaaatcaaaaacacctgtttgaagaaacggctgcacctgtgtgcttatttgtgccagagggtggcctagcccacctgcaggaagagatttggctgggttctgttgagggtgattgttaggacgttgtattttgttgccattattccaaatacctgtcttggagggaaagttgcccttctgagaactgtgactttaccaggagccctatcttggaataagagttacacctctggaccacgtttctcactagtactttgcttgactggaggaagtgggtgacttttggctgcttcggtgacccattgtagacgcctcgttacccttcttccttccgcttcaagtaatcatggcggcgggggtggcagcgtggctgccttttgcaagggcagcggctatcgggtggatgcctgtggcctcggggcctatgccggctcccccgaggcaggagaggaaaaggacccaagatgctctcattgtgctgaatgtgagtggcacccgcttccagacgtggcaggacaccctggaacgttacccagacactctactgggcagttctgagagggactttttctaccacccagaaactcagcagtatttctttgaccgtgacccagacatcttccgccacatcctgaatttctaccgcactgggaagctccactatcctcgccacgagtgcatctctgcttacgatgaagaactggccttctttggcctcatcccggaaatcatcggcgactgctgttatgaggagtacaaggatcgcaggcgagagaacgccgagcgcctgcaggacgacgcggataccgacaccgctggggagagcgccttgcccaccatgactgcaaggcagagggtctggagggccttcgagaacccccacaccagcacgatggccctggtgttctactatgtcacggggtttttcattgccgtctctgtcatcgcgaatgtggtggaaacagtgccgtgcggatcaagcccaggtcacattaaagaactgccctgtggagagcggtatgctgtggccttcttctgcttggacacggcctgcgtcatgatcttcacagttgagtatttgcttcgcctggctgcagcgcctagtcgttaccgttttgtgcgtagtgtcatgagtatcatcgacgtggtggccatcctgccttattacattgggctggtgatgacagacaatgaggacgtcagcggagcctttgtcacactccgagtcttccgggtcttcaggatctttaagttttcccgccactctcaaggcctgcgcatcctggggtacacactgaagagttgtgcctcagaattgggcttcttgcttttctcgctcaccatggctatcatcatcttcgctacagttatgttctacgcagagaaggggtcttcggctagcaagttcaccagcatccctgcagccttctggtataccatcgtcaccatgacaacactagggtatggtgacatggtgccaaaaaccatagcagggaagatttttggttctatctgttcgctgagtggggtcttggtcattgctctacctgttccggtgattgtatccaacttcagtcgcatctaccaccagaatcaacgagcagacaaacgaagggcacaaaagaaagctagactggccaggatccgggcagccaaaagcggaagcgcaaatgcttacatgcagagcaaacggaatggtttactcagtaatcagctgcagtcctcagaggatgagcaggcttttgttagcaaatccggctccagctttgaaacccagcaccaccacctgcttcactgcctggaaaaaaccacgaatcacgagtttgtggacgaacaagtctttgaagaaagctgcatggaagttgcaactgttaatcgtccttcaagtcacagtccttcactgtcttcacaacaaggagtcaccagcacctgctgttcacgacgacacaaaaaaacttttcgcatcccaaatgccaatgtatcaggaagccatcaaggtagtatacaagaactcagcacgattcagatcagatgtgtggagagaacacctctgtctaacagccgatccagtttaaatgccaaaatggaagagtgtgttaaactaaactgtgaacaaccttatgtgactacagcaataataagcatcccaacacctccagtaaccacaccagaaggagacgataggccagaatcccctgagtactcaggaggaaatattgtcagagtttctgctttgtaagacaattggaataaggtctaagagaattcgagccctggctgtgaaaagaatctcaacatagaagaaagaagaaacaataaatattctgcagattaatgcagcaaagaaagaaggttggtagtgaaacacaaagcttccaatcttaaggatgtgaataaaaccaccaaatggcatttctagacagtttgacctgttatacagagtaatattctgtggccctttgactttgtgaatgagcacaatgaaatgccgcctactgatgcttcttatgatcagaactcttttttaataaaataaataacataaatcgttgaacataatgttccagttgaatgcaaaacaaaaaaaatatggaaaacattttgataaaattttttcctgttaaaaccatgaacattggctatgatgaagattattacatatgaaaaaaaaactcacacaacatatttgtattgactgaaggaaaccatcataatgcatgctagaattctttgaagcagtgatctcagtttccttatgttgtcttcagaataggcatgataaactataattgtagaaaggggtaatttctgtgcacttacaacaagctgagtgttcatgttccatggtgggctgtgcaaataaactccttttagacctgcagtatttctcatggggatgctcattagtaaatctaaagtgttcagatagttcagtattcattatcgtttaactttgcacctagatactgttacaactgcaataatttgttgtacaactgttgtatcaggaatcaggatttttttgttgttgtactttccagatccttatagatacggtaagagccacattcgtagaaaaacttctggtgtggccaggttttaggtaactttttaatccaaaactattgtgccataaatgtttttcagtaatattttttggtccactgtattcctgtgacacagtgcattatctgttcttgtatttctatagcacctctctattgggtttatcatcatcaacaagactactgtttactgtagttcaagtgactttcctacttttgtatttccaaaaaaaattatcttgtaagtagcttgtcatcaatccccttgtcgaaaactagaaaaaaaggagttgacccatataaattatctctaacgtctttgttgtttatggaaaagcccagatactggatatatcactatgtattttatgaacagaattgactgggactaatatcacaggatcaatcatctcagaatcttacttgatgcattatttattttgctttagatcttgaatacattttgagaataactaatgtggattgaaatgtagagatacactggagtgctttatttagcaatatttgatgaaagcatgctttctacgccattcaggaaggcagcacaaatttatctcagaaaggttcctgtgtattgcaaggtacaattttctccaataaatcaggagaacaggagtttgatgatgcaaagttgatctctgtacatttaagtgaaaagtctttataacttttcacccttaaaatatttcagcagacatgtctgcacatgacagtgtaaaaaagtttaatgtcaaatgcaaagtttttattcattccaagccaccactgtaaggaataaagcttagcttctgtacatggaaagagctaataattatccctctgtcagagatgagatttttaaatgcttatgatatttaatcataaaaagggattaatccaaccattttcaagtaaagccagaaattcttgcttcccatttctagaatagtttctagaacagtgctatgcacatattagatcttaataaacatttgctgagtgaaagtaagataaactcaactatctcttgggaagaactggcttcattcctagtacatcttttaaaaagttactaattttccagcagtacaaatattaacaattatattaacacctgcctcatgtcagtttatgcttctagagcaatgtctagtgaaacttatctgatggcatttattgaaaaccttctaaaaagtagactaaggaaaccataatcagaattactatgtcttttgattcccaatgagaagttctattttcatgttcttaatattacatacaagaaaatgcagttaggttatttcaattgacaattctgcctcctcttttgatttatcacttacccaaaattattaattttattaggcttttggaaaagaaaaaaaactttttgatgttttaggtgatttaaaaatataccgtgttggtggtgaatgactattgatgactgtgttaagtgcatctgtattgtaagtgaaatgtaattatttctgtgtaccatatggagtaactaaggtcattgtttttgacaattttgtttgaaattcatatatcttatttcaaaggatagcataatatctgcattatgctggaaaaaaatagacctttggagaatacttaaataaaacatgtgcatgcttgaacaggac

SEQ ID NO: 253 - DNA, 2351 bp, Homo sapiens
gaattctattgggtgactctcgttcgtcttctctatcctacactccacatactgaccctatattatccagactgtgccggggagaaatcaaaaacacctgtttgaagaaacggctgcacctgtgtgcttatttgtgccagagggtggcctagcccacctgcaggaagagatttggctgggttctgttgagggtgattgttaggacgttgtattttgttgccattattccaaatacctgtcttggagggaaagttgcccttctgagaactgtgactttaccaggagccctatcttggaataagagttacacctctggaccacgtttctcactagtactttgcttgactggaggaagtgggtgacttttggctgcttcggtgacccattgtagacgcctcgttacccttcttccttccgcttcaagtaatcatggcggcgggggtggcagcgtggctgccttttgcaagggcagcggctatcgggtggatgcctgtggcctcggggcctatgccggctcccccgaggcaggagaggaaaaggacccaagatgctctcattgtgctgaatgtgagtggcacccgcttccagacgtggcaggacaccctggaacgttacccagacactctactgggcagttctgagagggactttttctaccacccagaaactcagcagtatttctttgaccgtgacccagacatcttccgccacatcctgaatttctaccgcactgggaagctccactatcctcgccacgagtgcatctctgcttacgatgaagaactggccttctttggcctcatcccggaaatcatcggcgactgctgttatgaggagtacaaggatcgcaggcgagagaacgccgagcgcctgcaggacgacgcggataccgacaccgctggggagagcgccttgcccaccatgactgcaaggcagagggtctggagggccttcgagaacccccacaccagcacgatggccctggtgttctactatgtcacggggtttttcattgccgtctctgtcatcgcgaatgtggtggaaacagtgccgtgcggatcaagcccaggtcacattaaagaactgccctgtggagagcggtatgctgtggccttcttctgcttggacacggcctgcgtcatgatcttcacagttgagtatttgcttcgcctggctgcagcgcctagtcgttaccgttttgtgcgtagtgtcatgagtatcatcgacgtggtggccatcctgccttattacattgggctggtgatgacagacaatgaggacgtcagcggagcctttgtcacactccgagtcttccgggtcttcaggatctttaagttttcccgccactctcaaggcctgcgcatcctggggtacacactgaagagttgtgcctcagaattgggcttcttgcttttctcgctcaccatggctatcatcatcttcgctacagttatgttctacgcagagaaggggtcttcggctagcaagttcaccagcatccctgcagccttctggtataccatcgtcaccatgacaacactagggtatggtgacatggtgccaaaaaccatagcagggaagatttttggttctatctgttcgctgagtggggtcttggtcattgctctacctgttccggtgattgtatccaacttcagtcgcatctaccaccagaatcaacgagcagacaaacgaagggcacaaaagaaagctagactggccaggatccgggcagccaaaagcggaagcgcaaatgcttacatgcagagcaaacggaatggtttactcagtaatcagctgcagtcctcagaggatgagcaggcttttgttagcaaatccggctccagctttgaaacccagcaccaccacctgcttcactgcctggaaaaaaccacgaatcacgagtttgtggacgaacaagtctttgaagaaagctgcatggaagttgcaactgttaatcgtccttcaagtcacagtccttcactgtcttcacaacaaggagtcaccagcacctgctgttcacgacgacacaaaaaaacttttcgcatcccaaatgccaatgtatcaggaagccatcaaggtagtatacaagaactcagcacgattcagatcagatgtgtggagagaacacctctgtctaacagccgatccagtttaaatgccaaaatggaagagtgtgttaaactaaactgtgaacaaccttatgtgactacagcaataataagcatcccaacacctccagtaaccacaccagaaggagacgataggccagaatcccctgagtactcaggaggaaatattgtcagagtttctgctttgtaagacaattggaataaggtctaagagaattc

SEQ ID NO: 254 - DNA, 5333 bp, Homo sapiens; identical to SEQ ID NO: 252

SEQ ID NO: 255 - DNA, 5404 bp, Homo sapiens
ggagatgcaggaccacccactggcggggaagcagctagcagccctcccgcgcccccgcgctgccgagcgccttctgcctccgcgctcggacgagagcccgtgccggccccggccccggccccaccgcgccaacgccgcccgcccggccgccccgcagccccgccgccccgcagccccgcaccgcgctggccaggctcccgcgacagtggccccgcagtaagttggcaggagcgagtcccctccgttctcgcctcccccgcaccttttgaacttgttgctgctgctctgctcgcctgcgcctggcttttggaaggtgaaaaggaggagggaggcacggagggatgggggaagggaaagaagagctcgcttgagctttatttatgctctctcggcgcatcggattcggctgctcgcgagctgctttctctcctcttccctttccgggtgcacggcgaggagaaagtctctatgcaactaagccccggcgcgcacttggccaggtatgtaccgcgggagcggcgcgttctgcgcggaagcagatgctgctgccgccacggcggcggcggctgccagctcctgagctctgtaactgtcacactgcacctgagctgaacttgaaaagagagtgaaggggcgattgggcgaacgcttttggcagacacagagggtgtttgtagacgtgggggaggagaatctctattaacgccccccaccgtaaccactgcacatcacctccatctctgcaaatacagcccgaggagtagaggcagcagcagctggacccccaaagagagacgtggggcagcggctgtgaccgcatctcctgagctacaacaacaggtcgcctttttgagactcctttggcgggaagggctacttggaaaggaaggtttgaaagagtgagaagggtaggtgtaagggttccctaattcgtcgaaagaattctattgggtgactctcgttcgtcttctctatcctacactccacatactgaccctatattatccagactgtgccggggagaaatcaaaaacacctgtttgaagaaacggctgcacctgtgtgcttatttgtgccagagggtggcctagcccacctgcaggaagagatttggctgggttctgttgagggtgattgttaggacgttgtattttgttgccattattccaaatacctgtcttggagggaaagttgcccttctgagaactgtgactttaccaggagccctatcttggaataagagttacacctctggaccacgtttctcactagtactttgcttgactggaggaagtgggtgacttttggctgcttcggtgacccattgtagacgcctcgttacccttcttccttccgcttcaagtaatcatggcggcgggggtggcagcgtggctgccttttgcaagggcagcggctatcgggtggatgcctgtggcctcggggcctatgccggctcccccgaggcaggagaggaaaaggacccaagatgctctcattgtgctgaatgtgagtggcacccgcttccagacgtggcaggacaccctggaacgttacccagacactctactgggcagttctgagagggactttttctaccacccagaaactcagcagtatttctttgaccgtgacccagacatcttccgccacatcctgaatttctaccgcactgggaagctccactatcctcgccacgagtgcatctctgcttacgatgaagaactggccttctttggcctcatcccggaaatcatcggcgactgctgttatgaggagtacaaggatcgcaggcgagagaacgccgagcgcctgcaggacgacgcggataccgacaccgctggggagagcgccttgcccaccatgactgcaaggcagagggtctggagggccttcgagaacccccacaccagcacgatggccctggtgttctactatgtcacggggtttttcattgccgtctctgtcatcgcgaatgtggtggaaacagtgccgtgcggatcaagcccaggtcacattaaagaactgccctgtggagagcggtatgctgtggccttcttctgcttggacacggcctgcgtcatgatcttcacagttgagtatttgcttcgcctggctgcagcgcctagtcgttaccgttttgtgcgtagtgtcatgagtatcatcgacgtggtggccatcctgccttattacattgggctggtgatgacagacaatgaggacgtcagcggagcctttgtcacactccgagtcttccgggtcttcaggatctttaagttttcccgccactctcaaggcctgcgcatcctggggtacacactgaagagttgtgcctcagaattgggcttcttgcttttctcgctcaccatggctatcatcatcttcgctacagttatgttctacgcagagaaggggtcttcggctagcaagttcaccagcatccctgcagccttctggtataccatcgtcaccatgacaacactagggtaggtgccataatgggaaatgggatggaggttgggtatgggtgaggcgattgtggacccatcgaggttacatggtaactccggggaaatcatttgttttctttcctgagtttaggaaagcattatctaaatggtttggcaaaactcttttcatctgtgaaatgggtataatacacacgttgaagtattaaggcattgctggcaaatgttgatgcctgaaagtgataaagatacaaagaaattttagaattcctgaatatatgaaagtagtagcaatatttatattaatatataaaaatatgacaatgaaaaacaaaatctatgccctaataaagacacaaatatatacaatgtatattgaaatgtctataaagtggttcaatgcatttaaatgaaaagtttccaggtatacttgaactattattttcatatgaatagatacttatggtgtacatttttcctctaagaaccataattcctattttacatcgtaatacatagattgtagatgtaattatcaaagtattttataatatatatgcacatattcatatgtatgcactttaattatggttgataggttattcagtcttttagaatatcagagctgaaactgatcaactctacaatatgcagtggaattaaatttgcaacatatttcaagctctaggttcatagtttcaaaaaaagaagcaaaagactgtcatccacacatttttttttagaatctacagatcccatcaggcaatgggtccacacccattaaatacacatagaagatagagcagtatctggtaagattgatggtcaccaaggctggctgtattataaaatttggggtcctaatttccttttagaatttttttgtgaattcttttttgggggtataatgaagttctgcaaccaaagggacaaatttctgaattcatgctgttgtttttaatatacttttagggctagaattaagtttttttaggtagtagagaaaaggaaaggaggcaaatgattaataatgttaaactgcagaaaagtagcagcattttacatattagagaatctaataaaataaaatggttagatctttttacttttatgagttcttagataagctggggagtgatgtgagtgccttttcttcagctttgtgctactattcttgcataggtaatcagtttagtgaggatttggtaatggctataagaaaaaagttattcccaaggctactctttaagctttgtgtttttggataatttaattggcttctttaaatgacattgtggttaaagtcaacttaattttattagacatattgtgttgtacagaatttctcatgtcatgtggccagctaatggaatagtttatatatgaagaattttaggctaggttaaacaagaaattggggtaaagaaaaatacaatgatttatgatttttatttagtctcatttttttaaagacctactggtacatttaaaaaatgaattagtgaaaatccatgttctacttcttatatttcctttttatcttgttggcaaatttgtgacagtttataaggataaggatgatgcagaatgccttcgcagtgtaggtgctgattctttatcaaagaggtttgtttttgttgtcttgtcattttgtagcaggagatccttattaaggacaaatgggtagtgcaaatcaccatcatgacgtcaaattagaagtacacttgaataaaaaagattctgttttaataggaagggagaactattaaaatgaaatacactttaaaaatttggtatatatggctgtgtttcttgaggtatcagtgaatcattttaatgctatatagtgtctatatgattgaatataggtataaaaagaatgttgatgagaattctaatttcatatagctaatagttctatactagacttgagaagttagcattattttaaaacttgtttctggaaatgactttccaatttttattttattttgaagcttatttttgtttcaaggaaaatatgagacaacattaaacattagtgacaattttttattatgtaaataaaatacttaaaagcacccatttttgaaaatatttaagaaattgaattatattgcctgagtaaaatctatgcagtggatttagttcacatgtttcataggtaagtaagtggattagaagcattgaatacacacgctttctcagaacttggaagctaagtggaagttactttaaagacttagcttcactgaatttgaacattttaattttcaggaaattaagctaattaatgatcctgatggaatagagcagtcatacttttaagatgtggaaatgtcgatgtaatcaaaatgagaacatatatggacacttgacaaatcatatgctttataatataacagaatttcatagaaaaatgtctttaatttctcatcaaaatgtattatgtattgtaccctgaagaaacagattcttataagccctgcctttttggtgccatctttgtgactcagttatttaataatacaaaatattcaataaaagccattgcctaactttattgtttaggctgtagttctatacttagaggatgaagtgtaaggtgcaaactttctgggaaataatagttgaagccaatatccaactatgtctgaatgattatcaagagttatctgagctctttttatggggctattaattttaatggagctaaatgttcttcaattagtgataatagaagtgaaaatgtgattgtaaacagtggttattgaaagttccatctcgtatgaacgttatctacatgagaataaataaccaagagctttgtcattcaggactggcagaatcatttgcagcttctagagtattttagacaatattcagttggttttatgatctaaagaactgagtgtgtctatcttagaaccaaggtgtat

SEQ ID NO: 256 - DNA, 1597 bp, Homo sapiens
cggctgctcgcgagctgctttctctcctcttccctttccgggtgcacggcgaggagaaagtctctatgcaactaagccccggcgcgcacttggccaggtatgtaccgcgggagcggcgcgttctgcgcggaagcagatgctgctgccgccacggcggcggcggctgccagctcctgagctctgtaactgtcacactgcacctgagctgaacttgaaaagagagtgaaggggcgattgggcgaacgcttttggcagacacagagggtgtttgtagacgtgggggaggagaatctctattaacgccccccaccgtaaccactgcacatcacctccatctctgcaaatacagcccgaggagtagaggcagcagcagctggacccccaaagagagacgtggggcagcggctgtgaccgcatctcctgagctacaacaacaggtcgcctttttgagactcctttggcgggaagggctacttggaaaggaaggtttgaaagagtgagaagggtaggtgtaagggttccctaattcgtcgaaagaattctattgggtgactctcgttcgtcttctctatcctacactccacatactgaccctatattatccagactgtgccggggagaaatcaaaaacacctgtttgaagaaacggctgcacctgtgtgcttatttgtgccagagggtggcctagcccacctgcaggaagagatttggctgggttctgttgagggtgattgttaggacgttgtattttgttgccattattccaaatacctgtcttggagggaaagttgcccttctgagaactgtgactttaccaggagccctatcttggaataagagttacacctctggaccacgtttctcactagtactttgcttgactggaggaagtgggtgacttttggctgcttcggtgacccattgtagacgcctcgttacccttcttccttccgcttcaagtaatcatggcggcgggggtggcagcgtggctgccttttgcaagggcagcggctatcgggtggatgcctgtggcctcggggcctatgccggctcccccgaggcaggagaggaaaaggacccaagatgctctcattgtgctgaatgtgagtggcacccgcttccagacgtggcaggacaccctggaacgttacccagacactctactgggcagttctgagagggactttttctaccacccagaaactcagcagtatttctttgaccgtgacccagacatcttccgccacatcctgaatttctaccgcactgggaagctccactatcctcgccacgagtgcatctctgcttacgatgaagaactggccttctttggcctcatcccggaaatcatcggcgactgctgttatgaggagtacaaggatcgcaggcgagagaacgccgagcgcctgcaggacgacgcggataccgacaccgctggggagagcgccttgcccaccatgactgcaaggcagagggtctggagggccttcgagaacccccacaccagcacgatggccctggtgttctactatgtcacggggtttttcattgccgtctctgtcatcgcgaatgtggtggaaacagtgccgtgcggatc

SEQ ID NO: 257 - protein, 255 aa, Homo sapiens
MVPKTIAGKIFGSICSLSGVLVIALPVPVIVSNFSRIYHQNQRADKRRAQKKARLARIRAAKSGSANAYMQSKRNGLLSNQLQSSEDEQAFVSKSGSSFETQHHHLLHCLEKTTNHEFVDEQVFEESCMEVATVNRPSSHSPSLSSQQGVTSTCCSRRHKKTFRIPNANVSGSHQGSIQELSTIQIRCVERTPLSNSRSSLNAKMEECVKLNCEQPYVTTAIISIPTPPVTTPEGDDRPESPEYSGGNIVRVSAL

SEQ ID NO: 258 - protein, 630 aa, Homo sapiens
MAAGVAAWLPFARAAAIGWMPVASGPMPAPPRQERKRTQDALIVLNVSGTRFQTWQDTLERYPDTLLGSSERDFFYHPETQQYFFDRDPDIFRHILNFYRTGKLHYPRHECISAYDEELAFFGLIPEIIGDCCYEEYKDRRRENAERLQDDADTDTAGESALPTMTARQRVWRAFENPHTSTMALVFYYVTGFFIAVSVIANVVETVPCGSSPGHIKELPCGERYAVAFFCLDTACVMIFTVEYLLRLAAAPSRYRFVRSVMSIIDVVAILPYYIGLVMTDNEDVSGAFVTLRVFRVFRIFKFSRHSQGLRILGYTLKSCASELGFLLFSLTMAIIIFATVMFYAEKGSSASKFTSIPAAFWYTIVTMTTLGYGDMVPKTIAGKIFGSICSLSGVLVIALPVPVIVSNFSRIYHQNQRADKRRAQKKARLARIRAAKSGSANAYMQSKRNGLLSNQLQSSEDEQAFVSKSGSSFETQHHHLLHCLEKTTNHEFVDEQVFEESCMEVATVNRPSSHSPSLSSQQGVTSTCCSRRHKKTFRIPNANVSGSHQGSIQELSTIQIRCVERTPLSNSRSSLNAKMEECVKLNCEQPYVTTAIISIPTPPVTTPEGDDRPESPEYSGGNIVRVSAL

SEQ ID NO: 259 - protein, 630 aa, Homo sapiens; identical to SEQ ID NO: 258

SEQ ID NO: 260 - protein, 630 aa, Homo sapiens; identical to SEQ ID NO: 258

SEQ ID NO: 261 - protein, 630 aa, Homo sapiens; identical to SEQ ID NO: 258

SEQ ID NO: 262 - DNA, 2707 bp, Homo sapiens
ggcaggccgagccagccgtgcgccgcgctccagggcccagggcgccgcacacgcacccacccacccacccagcctcgcagcgccatgggcaagaacaagcagccacgcggccagcagaggcaggggggcccgccggccgcggacgccgctgggcccgacgacatggagccgaagaagggcacgggggcccccaaggagtgcggggaggaggagccccggacctgctgcggctgccggttcccgctgctgctcgccctgctgcagctggccctgggcatcgccgtgaccgtggtgggcttcctcatggcgagcatcagctcctccctgctagtcagggacactccattttgggctgggatcattgtctgcttagtggcctatcttggcttgtttatgctttgtgtctcatatcaggttgacgaacggacatgtattcaattttctatgaaactgttatactttctgctgagtgccctgggcctgacggtctgtgtgctggccgtggcctttgccgcccaccactattcgcagctcacacagtttacctgtgagaccacactcgactcttgccagtgcaaactgccctcctcggagccgctcagcaggacctttgtttaccgggatgtgacggactgtaccagcgtcactggcactttcaaactgttcttactcatccagatgattcttaatttggtctgcggccttgtgtgcttgttggcctgctttgtgatgtggaaacataggtaccaggtcttctatgtgggtgtcaggatatgctccctcacggcttccgaaggcccccagcaaaagatctaacattcttgctcaaagttgcgagagaaagtagcacatggagtagctgaggttaaacaaacaaaaaaaaattttaaacaaagaaaggaaaaaaattgacaataaaagtcactcttctaattgaatatttttatatttttatgaaacaaaagagcatttcttcaggtttctattgtattttttttaacattcttgcagagaaagcaagatccaaattgattttgggatattaaaagttaacagaacactgaacaaggaaagaatggcatagatctatctttacagtctggagttaattcctgttaactcattttatccattccttacataatcttctttcctgttagtccagtttgatggtgtgaatggtgaatttcaggcccagttgctaaattttgtggcatcttcctctagtccttcccacctccagtcatcagccccactctgtcttggagacaggcaggaggtgggggaagagctgaatctctttattttccctggtagagacatcttcaaggcatgaaatagcttaaagagcagagtagaaatggaagaggctttgcaaaaggctagataactaacaacacctgggttggggcggcggcctcttctcttcagctcccttagcttggctccgtaagtggatcacttgccaaatgctttagatgattgcctctcaataattgaaaggtggtggtagttgtattctaaatgatgtagaaggtttaaaaataattacattatgcttctattctatcatctaaaacaaatcattaaaactaatttctagctaattgttaattataattatgctcagaagtctatttaatgagctctgactgtacttacgctgcactgtcggtgttaagagaaattactctcacaagagcagaggcctgaagattctttcttctgaaagccaagcaccacaaggaaaaaaaaattattaatagctcaggttaaaaacacccatttaaacaaaaacaagagcatttgtaataggaagtgtttatacaaacagcacatttgtgatatgttgaaaagcatctctcttggcaaccaatctatgtttgaggaagattgggtaatgctgatgtgttccattcatgaaactgtatttgatacataatcctattattaattcgtatgcttagtcaacctaggaaatcaaaataatgttttgaagttcttatttgagcaatatggccttgacttggagggtagttttagttgttttgtttttaagtgactgtggtttaaagcacaaatgccccaaggtggggagacttctctctgtgattattgttgctattaaattctgaactgtatccatattttaaggaaggagctaaaaatggaaattcatgaaacataaatggtatcaagaactttatcagtatgctttgttgaaagcagaaattaagataataattgagttcaattcgcctctccgcattgcctattgatacactttactaatcatgaaattctaacctaaaaggaaaacattttcctgcttgtcttagaagaaagtggaataattccactgattgtgataatggtttcaatttctacacaatataaatatccagtataaaggaaagcgttaagtcggtaagctagaggattgtaaatatcttttatgtcctctagataaaacacccgattaacagatgttaaaccttttaatgttttgatttgctttaaaaatggccttcctacacattagctccagctaaaaagacacattggagagcttagaggataagtctctggagcagaatttatcacacacaaaagttacaccaacagaataccaagcagaatgatgaggacctgtaaaataccttgtgccctattaaaaaaaaaaaaaaaaaaaaaaaaaaaaaaaaaa

SEQ ID NO: 263 - DNA, 2707 bp, Homo sapiens; identical to SEQ ID NO: 262

SEQ ID NO: 264 - DNA, 732 bp, Homo sapiens
atgggcaagaacaagcagccacgcggccagcagaggcaggggggcccgccggccgcggacgccgctgggcccgacgacatggagccgaagaagggcacgggggcccccaaggagtgcggggaggaggagccccggacctgctgcggctgccggttcccgctgctgctcgccctgctgcagctggccctgggcatcgccgtgaccgtggtgggcttcctcatggcgagcatcagctcctccctgctagtcagggacactccattttgggctgggatcattgtctgcttagtggcctatcttggcttgtttatgctttgtgtctcatatcaggttgacgaacggacatgtattcaattttctatgaaactgttatactttctgctgagtgccctgggcctgacggtctgtgtgctggccgtggcctttgccgcccaccactattcgcagctcacacagtttacctgtgagaccacactcgactcttgccagtgcaaactgccctcctcggagccgctcagcaggacctttgtttaccgggatgtgacggactgtaccagcgtcactggcactttcaaactgttcttactcatccagatgattcttaatttggtctgcggccttgtgtgcttgttggcctgctttgtgatgtggaaacataggtaccaggtcttctatgtgggtgtcaggatatgctccctcacggcttccgaaggcccccagcaaaagatctaa

SEQ ID NO: 265 - DNA, 681 bp, Homo sapiens
atggagccgaagaagggcacgggggcccccaaggagtgcggggaggaggagccccggacctgctgcggctgccggttcccgctgctgctcgccctgctgcagctggccctgggcatcgccgtgaccgtggtgggcttcctcatggcgagcatcagctcctccctgctagtcagggacactccattttgggctgggatcattgtctgcttagtggcctatcttggcttgtttatgctttgtgtctcatatcaggttgacgaacggacatgtattcaattttctatgaaactgttatactttctgctgagtgccctgggcctgacggtctgtgtgctggccgtggcctttgccgcccaccactattcgcagctcacacagtttacctgtgagaccacactcgactcttgccagtgcaaactgccctcctcggagccgctcagcaggacctttgtttaccgggatgtgacggactgtaccagcgtcactggcactttcaaactgttcttactcatccagatgattcttaatttggtctgcggccttgtgtgcttgttggcctgctttgtgatgtggaaacataggtaccaggtcttctatgtgggtgtcaggatatgctccctcacggcttccgaaggcccccagcaaaagatctaacattcttgctcaaagttgcgagagaaa

SEQ ID NO: 266 - DNA, 1502 bp, Homo sapiens
gtgaatttcaggcccagttgctaaattttgtggcatcttcctctagtccttcccacctccagtcatcagccccactctgtcttggagacaggcaggaggtgggggaagagctgaatctctttattttccctggtagagacatcttcaaggcatgaaatagcttaaagagcagagtagaaacggaagaggctttgcaaaaggctagataactaacaacacctgggttggggcggcggcctcttctcttcagctcccttagcttggctccgtaagtggatcacttgccaaatgctttagatgattgcctctcaataattgaaaggtggtggtagttgtattctaaatgatgtagaaggtttaaaaataattacattatgcttctattctatcatctaaaacaaatcattaaaactaatttctagctaattgttaattataattatgctcagaagtctatttaatgagctctgactgtacttacgctgcactgtcggtgttaagagaaattactctcacaagagcagaggcctgaagattctttcttctgaaagccaagcaccacaaggaaaaacaaattattaatagctcaggttaaaaacacccatttaaacaaaaacaagagcatttgtaataggaagtgtttatacaaatagcacatttgtgatatgttgaaaagcatctctcttggcaaccaatctatgtttgaggaagattgggtaatgctgatgtgttccattcatgaaactgtatttgatacataatcctattattaattcgtatgcttagtcaacctaggaaatcaaaataatgttttgaagttcttatttgagcaatatggccttgacttggagggtagttttagttgttttgtttttaagtgactgtggtttaaagcacaaatgccccaaggtggggagacttctctctgtgattattgttgctattaaattctgaactgtatccatattttaaggaaggagctaaaaatggaaattcatgaaacataaatggtatcaagaactttatcagtatgctttgttgaaagcagaaattaagataataattgagttcaattcgcctctccgcattgcctattgatacactttactaatcatgaaattctaacctaaaaggaaaacattttcctgcttgtcttagaagaaagtggaataattccactgattgtgataatggtttcaatttctacacaatataaatatccagtataaaggaaagcgttaagtcggtaagctagaggattgtaaatatcttttatgtcctctagataaaacacccgattaacagatgttaaaccttttaatgttttgatttgctttaaaaatggccttcctacacattagctccagctaaaaagacacattggagagcttagaggataagtctctggagcagaatttatcacacacaaaagttacaccaacagaataccaagcagaatgatgaggacctgtaaaataccttgtgccctattaaaaaaaaaa

SEQ ID NO: 267 - DNA, 4013 bp, Homo sapiens
tgtgtggaacacactcatttggaggacttttgtacacatattttgtagtgtcacatatatgttttaattttgaattatatataagggaaggtgggggaagggcatcatcttctcagagctactttcctctgaacctggaaatgactggaactaatattactttgtgaagtgtccatttaccagaattgttctctgtagagagcaacttttgactgtggtaatgtaattcttgcactaagaactatgtgtactagtctcaaaagctggggactctgagccttacctagagtctcagcaggtggaccattaagattaacatttctagtaggtgagttcaatcacaaaaatatttcttgttccatagattttattgtggccatgtcagtgaacacccacaagttttgctcagaatattttaggtgtaagctaaatccctaaattgttcagagttcccacagccctgtagcagcagagcgagaactttaaccagactttttcaatcccaaagctaatctggaggccaacagtgttcaaaaccttggtgactgaggaaccatttagagttttttcaggctcaggaatcacatggtcgttgttgggcttggggtaagtttcacaggcgatgaagctgacgttgagtcacttgacttctggagccataatttattttctcccagcaacctcctactggggattctcatgtttatggatacagtttggcaatcactacattgaatgtagtcttttaaaaaaattaacttatgctattagttgacccatcattgctaattttggcccacacagtgtttgcattacaaaaacctgttctttacttcctagtcttgtttcagtcttaatatcagaagttcttgagttcaaaataagcacaacatgtcatccagggatggctagcttgtttgggattcatctaaactgctggcaatatctagacaaaaacattccacagtccagctaatatggttgtcacaactcttgaaaagggcccaacatctggatggcaagtgaaaatgtgatcagggtttaagaactacccactaataaataaacatggagctatttccatgtcttgggtgttgtgtttctaagaagagacagcctttccatcagaaaatttctgggagggaagaaaaagaacagttttgatgaattcgctttgcaaatcatcatccaatgttctttgtaaccagaaaggttttcttctgctttcttgcagctgttatactttctgctgagtgccctgggcctgacggtctgtgtgctggccgtggcctttgccgcccaccactattcgcagctcacacagtttacctgtgagaccacactcgactcttgccagtgcaaactgccctcctcggagccgctcagcaggacctttgtttaccgggatgtgacggactgtaccagcgtcactggcactttcaaactgttcttactcatccagatgattcttaatttggtctgcggccttgtgtgcttgttggcctgctttgtgatgtggaaacataggtaccaggtcttctatgtgggtgtcaggatatgctccctcacggcttccgaaggcccccagcaaaagatctaacattcttgctcaaagttgcgagagaaagtagcacatggagtagctgaggttaaacaaacaaaaaaaaattttaaacaaagaaaggaaaaaaattgacaataaaagtcactcttctaattgaatatttttatatttttatgaaacaaaagagcatttcttcaggtttctattgtattttttttaacattcttgcagagaaagcaagatccaaattgattttgggatattaaaagttaacagaacactgaacaaggaaagaatggcatagatctatctttacagtctggagttaattcctgttaactcattttatccattccttacataatcttctttcctgttagtccagtttgatggtgtgaatggtgaatttcaggcccagttgctaaattttgtggcatcttcctctagtccttcccacctccagtcatcagccccactctgtcttggagacaggcaggaggtgggggaagagctgaatctctttattttccctggtagagacatcttcaaggcatgaaatagcttaaagagcagagtagaaacggaagaggctttgcaaaaggctagataactaacaacacctgggttggggcggcggcctcttctcttcagctcccttagcttggctccgtaagtggatcacttgccaaatgctttagatgattgcctctcaataattgaaaggtggtggtagttgtattctaaatgatgtagaaggtttaaaaataattacattatgcttctattctatcatctaaaacaaatcattaaaactaatttctagctaattgttaattataattatgctcagaagtctatttaatgagctctgactgtacttacgctgcactgtcggtgttaagagaaattactctcacaagagcagaggcctgaagattctttcttctgaaagccaagcaccacaaggaaaaaaaaaattattaatagctcaggttaaaaacacccatttaaacaaaaacaagagcatttgtaataggaagtgtttatacaaacagcacatttgtgatatgttgaaaagcatctctcttggcaaccaatctatgtttgaggaagattgggtaatgctgatgtgttccattcatgaaactgtatttgatacataatcctattattaattcgtatgcttagtcaacctaggaaatcaaaataatgttttgaagttcttatttgagcaatatggccttgacttggagggtagttttagttgttttgtttttaagtgactgtggtttaaagcacaaatgccccaaggtggggagacttctctctgtgattattgttgctattaaattctgaactgtatccatattttaaggaaggagctaaaaatggaaattcatgaaacataaatggtatcaagaactttatcagtatgctttgttgaaagcagaaattaagataataattgagttcaattcgcctctccgcattgcctattgatacactttactaatcatgaaattctaacctaaaaggaaaacattttcctgcttgtcttagaagaaagtggaataattccactgattgtgataatggtttcaatttctacacaatataaatatccagtataaaggaaagcgttaagtcggtaagctagaggattgtaaatatcttttatgtcctctagataaaacacccgattaacagatgttaaaccttttaatgttttgatttgctttaaaaatggccttcctacacattagctccagctaaaaagacacattggagagcttagaggataagtctctggagcagaatttatcacacacaaaagttacaccaacagaataccaagcagaatgatgaggacctgtaaaataccttgtgccctattaaaaaaaaaaaaaaaaaaaaagccagtaactgaatccattttgatttttggttgagtttcctacacaaagaagaaaataactgagaatctggaatgttgtagtccatcctttaaagagtaagaaagtagcagttaatgctagtaaccgtgaattaggcaccactgaaagcacatcccgaatttctttaacaacaacattttatagtgaacactacaagtttttatatttaaaaattaagactctgtatatccttaaggtgctctatgctttaccagtaattcacagggtatttcaaatggtagaatcattttagcttctgtgcttcctttttctaaataatgcaacttgtaagagttgacattgtaataagctttataatagtataaccgtcaggagatatatatatatatatatacacatacacacacacacacacatatatactatacatatataaaatggggatattactattgtatgattaaatcattcttaagtccccaaggaaaaaaaatcataaacaaatagaaagaactaaacagaaaagaaagaaa

SEQ ID NO: 268 - DNA, 4003 bp, Homo sapiens
  misc_feature 1,2,3,4,6,7,8,9,10,11,12,13,14,16,17,266,764,769,773,774,777,781,783,784,788,789,790,791,792,796,797,802,808,809,813,814,815,819,820,821,825,826,830,831,832,833,835,836,839,842,845,855,856,857,860,861,862,863,867,868,870,871,877,879,880,881,882,887,892,893,899,903,904,907,908,910,911,912,913,917,918,920,921,924,925,926,929,931,933,935,1011,1012,1015,1023,1026,1081,1098,1133,1136,1137,3472,3996,4000: n = A,T,C or G
yswktkkwwywywytywtttggaggacttttgtacacatattttgtagtgtcacatatatgttttaattttgaattatacataagggaaggtgggggaagggcatcatcttctcagagctactttcctctgaacctggaaatgactggaactaatattactttgtgaagtgtccatttaccagaattgttctctgtagagagcaacttttgactgtggtaatgtaattcttgcactaagaactatgtgtactagtctcaaaagctkggggactctgagccttacctagagtctcagcaggtggaccattaagattaacatttctagtaggtgagttcaatcacaaaaatatttcttgttccatagattttattgtggccatgtcagtgaacacccacaagttttgctcagaatattttaggtgtaagctaaatccctaaattgttcagagttcccacagccctgtagcagcagagcgagaactttaaccagactttttcaatcccaaagctaatctggaggccaacagtgttcaaaaccttggtgactgaggaaccatttagagttttttcaggctcaggaatcacatggtcgttgttgggcttggggtaagtttcacaggcgatgaagctgacgttgagtcacttgacttctggagccataatttattttctcccagcaacctcctactggggattctcatgtttatggatacagtttggcaatcactacattgaatgtagtcttttaaaaaaattaacttakgctaktagyygasccakcmktgckmmkyttgsyccacmcagtgkytgcmkyacamrmaccyrttcyywmcywccyagyctygtttcagtckymatrksmaagwwcwwgcagcmamryrgccarcagakscagggrggcymgcykgyyksggaykcmkctrrrccygrcrayatggagccgaagaagggcacgggggcccccaaggagtgcggggaggaggagccccggacctgctgcggctgccggkkccmgctgctgmtcmgccctgctgcagctggccctgggcatcgccgtgaccgtggtgggcttcctcatgkcgagcatcagctcctcyctgctagtcagggacactccattttgggctgggartcwytgtctgcttagtggcctatcttggcttgtttatgctttgtgtctcatatcaggttgacgaacggacatgtattcaattttctatgaaactgttatactttctgctgagtgccctgggcctgacggtctgtgtgctggccgtggcctttgccgcccaccactattcgcagctcacacagtttacctgtgagaccacactcgactcttgccagtgcaaactgccctcctcggagccgctcagcaggacctttgtttaccgggatgtgacggactgtaccagcgtcactggcactttcaaactgttcttactcatccagatgattcttaatttggtctgcggccttgtgtgcttgttggcctgctttgtgatgtggaaacataggtaccaggtcttctatgtgggtgtcaggatatgctccctcacggcttccgaaggcccccagcaaaagatctaacattcttgctcaaagttgcgagagaaagtagcacatggagtagctgaggttaaacaaacaaaaaaaaattttaaacaaagaaaggaaaaaaattgacaataaaagtcactcttctaattgaatatttttatatttttatgaaacaaaagagcatttcttcaggtttctattgtattttttttaacattcttgcagagaaagcaagatccaaattgattttgggatattaaaagttaacagaacactgaacaaggaaagaatggcatagatctatctttacagtctggagttaattcctgttaactcattttatccattccttacataatcttctttcctgttagtccagtttgatggtgtgaatggtgaatttcaggcccagttgctaaattttgtggcatcttcctctagtccttcccacctccagtcatcagccccactctgtcttggagacaggcaggaggtgggggaagagctgaatctctttattttccctggtagagacatcttcaaggcatgaaatagcttaaagagcagagtagaaatggaagaggctttgcaaaaggctagataactaacaacacctgggttggggcggcggcctcttctcttcagctcccttagcttggctccgtaagtggatcacttgccaaatgctttagatgattgcctctcaataattgaaaggtggtggtagttgtattctaaatgatgtagaaggtttaaaaataattacattatgcttctattctatcatctaaaacaaatcattaaaactaatttctagctaattgttaattataattatgctcagaagtctatttaatgagctctgactgtacttacgctgcactgtcggtgttaagagaaattactctcacaagagcagaggcctgaagattctttcttctgaaagccaagcaccacaaggaaaaaaaaattattaatagctcaggttaaaaacacccatttaaacaaaaacaagagcatttgtaataggaagtgtttatacaaacagcacatttgtgatatgttgaaaagcatctctcttggcaaccaatctatgtttgaggaagattgggtaatgctgatgtgttccattcatgaaactgtatttgatacataatcctattattaattcgtatgcttagtcaacctaggaaatcaaaataatgttttgaagttcttatttgagcaatatggccttgacttggagggtagttttagttgttttgtttttaagtgactgtggtttaaagcacaaatgccccaaggtggggagacttctctctgtgattattgttgctattaaattctgaactgtatcccatattttaaggaaggagctaaaaatggaaattcatgaaacataaatggtatcaagaactttatcagtatgctttgttgaaagcagaaattaagataataattgagttcaattcgcctctccgcattgcctattgatacactttactaatcatgaaattctaacctaaaaggaaaacattttcctgcttgtcttagaagaaagtggaataattccactgattgtgataatggtttcaatttctacacaatataaatatccagtataaaggaaagcgttaagtcggtaagctagaggattgtaaatatcttttatgtcctctagataaaacacccgattaacagatgttaaaccttttaatgttttgatttgctttaaaaatggccttcctacacattagctccagctaaaaagacacattggagagcttagaggataagtctctggagcagaatttatcacacacaaaagttacaccaacagaataccaagcagaatgatgaggacctgtaaaataccttgtgccctattaaaaaaaaaaaaaaaaaaaaaarccagtaactgaatccattttgatttttggttgagtttcctacacaaagaagaaaataactgagaatctggaatgttgtagtccatcctttaaagagtaagaaagtagcagttaatgctagtaaccgtgaattaggcaccactgaaagcacatcccgaatttctttaacaacaacattttatagtgaacactacaagtttttatatttaaaaattaagactctgtatatccttaaggtgctctatgctttaccagtaattcacagggtatttcaaatggtagaatcattttagcttctgtgcttcctttttctaaataatgcaacttgtaagagttgacattgtaataagctttataatagtataaccgtcaggagatatatatatatatatacacatacacacacacacacacatatatactatacatatataaaatggggatattactattgtatgattaaatcattcttaagtccccaaggaaaaaaaatcataaacaaatagaaagaactaaacaaaaaaraaaraaa

SEQ ID NO: 269 - protein, 243 aa, Homo sapiens
MGKNKQPRGQQRQGGPPAADAAGPDDMEPKKGTGAPKECGEEEPRTCCGCRFPLLLALLQLALGIAVTVVGFLMASISSSLLVRDTPFWAGIIVCLVAYLGLFMLCVSYQVDERTCIQFSMKLLYFLLSALGLTVCVLAVAFAAHHYSQLTQFTCETTLDSCQCKLPSSEPLSRTFVYRDVTDCTSVTGTFKLFLLIQMILNLVCGLVCLLACFVMWKHRYQVFYVGVRICSLTASEGPQQKI

SEQ ID NO: 270 - protein, 243 aa, Homo sapiens; identical to SEQ ID NO: 269

SEQ ID NO: 271 - protein, 243 aa, Homo sapiens; identical to SEQ ID NO: 269

SEQ ID NO: 272 - protein, 217 aa, Homo sapiens
MEPKKGTGAPKECGEEEPRTCCGCRFPLLLALLQLALGIAVTVVGFLMASISSSLLVRDTPFWAGIIVCLVAYLGLFMLCVSYQVDERTCIQFSMKLLYFLLSALGLTVCVLAVAFAAHHYSQLTQFTCETTLDSCQCKLPSSEPLSRTFVYRDVTDCTSVTGTFKLFLLIQMILNLVCGLVCLLACFVMWKHRYQVFYVGVRICSLTASEGPQQKI

SEQ ID NO: 273 - DNA, 5738 bp, Homo sapiens
  misc_feature 356,357,358,396,627,691,1719,1722,1731,1739,3019,3228,3702,3946,3960,4004,4014,4015,4019,4024,4026,4038,4039,4044,4046,4047,4048,4050,4053,4083,4085,4087,4089,4090,4092,4094,4097,4098,4100,4105,4108,4322,4935,4937,5401,5514,5519,5531,5532,5534: n = A,T,C or G
atgttgccagaaatatccaccacaagaaaaatcattaagttccctacttcccccatcctggcagaatcatcagaaatgaccatcaagacccaaacaagtcctcctgggtctacatcagagagtacctttacattagacacatcaaccactccctccttggtaataacccattcgactatgactcagagattgccacactcagagataaccactcttgtgagtagaggtgctggggatgtgccacggcccagctctctccctgtggaagaaacaagccctccatcttcccagctgtctttatctgccatgatctcaccttctcctgtttcttccacattaccagcaagtagccactmskcttctgcttctgtgacttcacttctcacaccaggccamgtgaagactactgaggtgttggacgcaagtgcagaacctgaaaccagttcacctccaagtttgagcagcacctcagttgaaatactggccacctctgaagtcaccacagatacggagaaaattcatcctttctcaaacacggcagtaaccaaagttggaacttccagttctggacatgaatccccttcctctgtcctacctgactcagagacaaccaaagccacatcggcwatgggtaccatctccattatgggggatacaagtgtttctacattaactcctgccttatctaacrctaggaaaattcagtcagagccagcttcctcactgaccaccagattgagggagaccagcacctctgaagagaccagcttagccacagaagcaaacactgttctttctaaagtgtccactggtgctactactgaggtctccaggacagaagccatctcctttagcagaacatccatgtcaggccctgagcagtccacaatgtcacaagacatctccataggaaccatccccaggatttctgcctcctctgtcctgacagaatctgcaaaaatgaccatcacaacccaaacaggtccttcggagtctacactagaaagtacccttaatttgaacacagcaaccacaccctcttgggtggaaacccactctatagtaattcagggatttccacacccagagatgaccacttccatgggcagaggtcctggaggtgtgtcatggcctagccctccctttgtgaaagaaaccagccctccatcctccccgctgtctttacctgccgtgacctcacctcatcctgtttccaccacattcctagcacatatccccccctctccccttcctgtgacttcactttctcacctctggcccggcgacaaccacagatatcttgggtacaagcacagaacctggaaccagttcatcttcaagtttgagcaccacctcccatgagagactgaccacttacaaagacactgcacatacagaagccgtgcatccttccacaaacacaggagggaccaatgtggcaaccaccagctctggatataaatcacagtcctctgtcctagctgactcatctccaatgtgtaccacctccaccatgggggatacaagtgttctcacatcaactcctgccttccttgagactaggaggattcagacagagctagcttcctccctgacccctggattgagggagtccagcggctctgaagggaccagctcaggcaccaagatgagcactgtcctctctaaagtgcccactggtgctactactgagatctccaaggaagacgtcacctcggrgarcatccatchcaggtccygctcaatccacaatatcaccagacatctccacaagaaccgtcagctggttctctacatcccctgtcatgacagaatcagcagaaataaccatgaacacccatacaagtcctttaggggccacaacacaaggcaccagtactttggccacgtcaagcacaacctctttgacaatgacacactcaactatatctcaaggattttcacactcacagatgagcactcttatgaggaggggtcctgaggatgtatcatggatgagccctccccttctggaaaaaactagaccttccttttctctgatgtcttcaccagccacaacttcaccttctcctgtttcctccacattaccagagagcatctcttcctctcctcttcctgtgacttcactcctcacgtctggcttggcaaaaactacagatatgttgcacaaaagctcagaacctgtaaccaactcacctgcaaatttgagcagcacctcagttgaaatactggccacctctgaagtcaccacagatacagagaaaactcatccttcttcaaacagaacagtgaccgatgtggggacctccagttctggacatgaatccacttcctttgtcctagctgactcacagacatccaaagtcacatctccaatggttattacctccaccatggaggatacgagtgtctccacatcaactcctggcttttttgagactagcagaattcagacagaaccaacatcctccctgacccttggactgagaaagaccagcagctctgaggggaccagcttagccacagagatgagcactgtcctttctggagtgcccactggtgccactgctgaagtctccaggacagaagtcacctcctctagcagaacatccatctcaggctttgctcagctcacagtgtcaccagagacttccacagaaaccatcaccagactccctacctccagcataatgacagaatcagcagaaatgatgatcaagacacaaacagatcctcctgggtctacaccagagagtactcatactgtggacatatcaacaacacccaactgggtagaaacccactcgactgtgactcagagattttcacactcagagatgaccactcttgtgagcagaagccctggtgatatgttatggcctagtcaatcctctgtggaagaaaccagctctgcctcttccctgctgtctctgcctgccacgacctcaccttctcctgtttcctctacattagtagaggatttcccttccgcttctcttcctgtgacttctcttctcamccctggcctggtgataaccacagacaggatgggcataagcagagaacctggaaccagttccacttcaaatttgagcagcacctcccatgagagactgaccactttggaagacactgtagatacagaagacatgcagccttccacacacacagcagtgaccaacgtgaggacctccatttctggacatgaatcacaatcttctgtcctakctgactcagagacacccaaagccacatctccaatgggtaccacctacaccatgggggaaacgagtgtttccatatccacttctgacttctttgagaccagcagaattcagatagaaccaacatcctccctgacttctggattgagggagaccagcagctctgagaggatcagctcagccacagagggaagcactgtcctttctgaagtgcccagtggtgctaccactgaggtctccaggacagaagtgatatcctctaggggaacatccatgtcagggcctgatcagttcaccatatcaccagacatctctactgaagcgatcaccaggctttctacttcccccattatgacagaatcagcagaaagtgccatcactattgagacaggttctcctggggctacatcagagggtaccctcaccttggacacctcaacaacaaccttttggtcagggacccactcaactgcatctycaggattttcacactcagagatgaccactcttatgagtagaactcctggagatgtgccatggccgagccttccctctgtggaagaagccagctctgtctcttcctcactgtcttcacctgccatgacctcaacttcttttttctccacattaccagagagcatctcctcctctcctcatcctgtgactgcacttctcacccttggcccagtgaagaccacagacatgttgcgcacaagctcagracctgaaaccagtycacctccaaatttgagcagcacctcagctgaaatattagccacstctgaagtcrscaargatasakagaaaattcatmmctccycmmmcrcamctgtagtcaatgtagggactgtgatttatrawcwtmwaycmcckycmtctgwttyggctgacttagtgacaacaaaacccacatctccaatggctaccacctccactctggggaatacaagtgtttccacatcaactcctgccttcccagaaactatgatgacacagccaacttcctccctgacttctggattaagggagatcagtacctctcaagagaccagctcagcaacagagagaagtgcttctctttctggaatgcccactggygctactactaaggtctccagaacagaagccctctccttaggcagaacatccaccccaggtcctgctcaatccacaatatcaccagaaatctccacggaaaccatcactagaatttctactcccctcaccacgacaggatcagcagaaatgaccatcacccccaaaacaggtcattctggggcatcctcacaaggtacctttaccttggacacatcaagcagagcctcctggccaggaactcactcagctgcaactcacagatctccacactcagggatgaccactccttatgagcagaggtcctgaggatgtgtcatggccaagccgcccatcagtggaaaaaactagccctccatcttccctggtgtctttatctgcagtaacctcaccttcgccactttattccacaccatctgagagtagccactcatctcctctccgggtgacttctcttttcacccctgtcatgatgaagaccacagacatgttggacacaagcttggaacctgtgaccacttcacctcccagtatgaatatcacctcagatgagagtctggccacttctaaagccaccatggagacagaggcaattcagctttcagaaaacacagcwgygactcagatgggcaccatcagtgctagacaagaattctattcctcttatccaggcctcccagagccatccaaagtgacatctccagtggtcacctcttccaccataaaagacattgtttctacaaccatacctgcttcctctgagataacaagaattgagatggagtcaacatccaccctgacccccacaccaagggagaccagcacctcccaggagatccactcagccacaaagccaagcactgttccttacaaggcactcactagtgccacgattgaggactccatgacacaagtcatgtcctctagcagaggacctagccctgatcagtccacaatgtcacaagacatatccactgaagtgatcaccaggctctctacctcccccatcaagacagaatctacagaaatgacattaccacccaaacaggttctcctggggctacatcaaggggtacccttakccttggacacttcaacaacttttatgtcagggacccacttcaactgcatctcaaggattttcacactcacagatgaccgctcttatgagtagactcctggagatgtgccatgrctaasccatccctctgskgmagagcccgcctctgcctctttctcactggcttcacctgtcttgacctcatttttttcgttttttgcccattcccaaaaacctccaccttttttggttcctgggcaaactttttccctagggctggggaaacccaaaatgtggggccaacccagaactgaaacattccccccaatggacaacctttttgaaaagggcccctttgc

SEQ ID NO: 274 - DNA, 1024 bp, Homo sapiens
ccccacccgaaacacactcagcccttgcactgacctgccttctgattggaggctggttgcttcggataatgacctccaggaccccactgttggttacagcctgtttgtattattcttactgcaactcaagacacctgcagcagggcgtgagaaaaagtaaaagaccagtattttcacattgccaggtaccagaaacacagaagactgacacccgccacttaagtggggccagggctggtgtctgcccatgttgccatcctgatgggctgcttgccacaatgagggatcttcttcaatacatcgcttgcttctttgcctttttctctgctgggtttttgattgtggccacctggactgactgttggatggtgaatgctgatgactctctggaggtgagcacaaaatgccgaggcctctggtgggaatgcgtcacaaatgcttttgatgggattcgcacctgtgatgagtacgattccatacttgcggagcatcccttgaagctggtggtaactcgagcgttgatgattactgcagatattctagctgggtttggatttctcaccctgctccttggtcttgactgcgtgaaattcctccctgatgagccgtacattaaagtccgcatctgctttgttgctggagccacgttactaatagcaggtaccccaggaatcattggctctgtgtggtatgctgttgatgtgtatgtggaacgttctactttggttttgcacaatatatttcttggtatccaatataaatttggttggtcctgttggctcggaatggctgggtctctgggttgctttttggctggagctgttctcacctgctgcttatatctttttaaagatgttggacctgagagaaactatccttattccttgaggaaagcctattcagccgcgggtgtttccatggccaagtcatactcagcccctcgcacagagacggccaaaatgtatgctgtagacacaagggtgtaaaatgcacgtttcagggtgtgtttgcatatgatttaatc

SEQ ID NO: 275 - DNA, 1024 bp, Homo sapiens; identical to SEQ ID NO: 274

SEQ ID NO: 276 - DNA, 24110 bp, Homo sapiens
ccccacccgaaacacactcagcccttgcactgacctgccttctgattggaggctggttgcttcggataatgacctccaggaccccactgttggttacagcctgtttgtattattcttactgcaactcaagacacctgcagcagggcgtgagaaaaagtaaaagaccagtattttcacattgccaggtaccagaaacacagaagactgacacccgccacttaagtggggccagggctggtgtctgcccatgttgccatcctgatgggctgcttgccacaatgagggatcttcttcaatacatcgcttgcttctttgcctttttctctgctgggtttttgattgtggccacctggactgactgttggatggtgaatgctgatgactctctggaggtaagaagatagcagcttcttttcatgatccaggccagcccaaattttcgctaagtcccaactgccatgtacaacattcagtatctttactaaggctaatgataccaaaaataggcaacatggactatttattgagtctttacattattagctcatttaatcctcatagttaatttatgaggtaggtcttgttatcccattaaacagatgaagttactaaatagttcctccttttttcacaaggataaatttccacaagggtaattaagtgatctctgctactgagacctccagaaattcacgtcttccattgctgcatatatcatattgagtaacatttcagtaccacccttttttctaagataaattttttactcttgatgacagcattaagaatagtgtgatagactttttttaaggagtgttaataatctaaaacgttgagaaagaaaatgcaaggcatgcaaaacctacccaattaacatgcaagaggaaaaaacattatcttaatgatttccaagtaaaagaaaaaatgttgagggagaaaatgtctttccagtgcatcccaatgtacgggggacaggcatggatttaaatcctcccttaaaatgagttgctctagggaactgactactattcaaaagatgagtgagtgggttcacatttgaggattttatttttctcgctggagaagctcagaaagaagtaattttgaagttcaaaaccattacctgtggccataggaatctgagagaggcagaactgagtaaaaaatcaaatcttcagaattagctgctgttcattaatgaggcttaggaaaacacaggtaagaaaaagaaacaatatttcaagagctcaaaaaaaggagtatatagcaaaacaatttgctttttaatgtgcatcctgaagggaacaatttaccctagcaaatgctataatgtcacctctataaagtttaagaaagatactcgactgagtttatatatttttcttctaattttctttattaaactctcaaattggagttccaaatggaaagtaataatgattctattttgctgtgcattatttttgctgctcgctttttcttgcttttaatttgcctctgacttgaacatggcatttcaaaaccaatggagttaggaatacctctttaatgctagaaaattacatttccaaaaattgtgatagaattgaactactgtaaaggatgtctgctataagtgagcccagtgatgcattttatctggccatgaatatatgcaaagaatgaaataaatgccctttgaacagtgctcagggaaaagtgcagataaaacgttctgctgtcattagtttgccattatctagatggccagtggtaggtgatgaatacagaaatatgtttaacttgagcataattataattatgttttttaaaatacaaaaaaatgtaaaatcccatctaggggcattgttaaaatattttctaaaacaatttaaaagtctttctgcttaagctgacataattgctaacttcatttgataagaaatagttttagaaagggtcaaaccttgctgagagagagattgagagtcctggaatttaaagtgtcttctttcattttagtataaccaaccaatttgccatctgtcccatgaaagaatacttctagttaaaacgaatggaatgagcagtccaggttacacacctcaagtaaacccttgctaaccttgaaaaatagttaatatttcttagcttccttcttatttcccatacttaaaatgtattgctataatattcccaagaagccttcacatttaaaggaagaggctgggcatggtggcttatgcctgtaatcccaatactttggaaggccgaggcgggcagatcacaggtcaagagattgagaccatcctggccgacatagtgaaaccccatctctaacaaaaatacaaaaattagctgggtgtggtggcaggggcctgtagtcccagctactaaggaggctgaggcaggagaatcgcttgaactagggaggaggaggttgcactgagccgagattgtgccactgcactccagcctggcgacagagcgagactccatcttaaaaaagaagaagaagaagaagaaccctgtaacaaatccggctcccttctctttcaacaatctctttagttgtcaatatttttaaagagacaataatctcttataataattgctacttcaacaggccaggatagaaacttatattttccacaaatttgaaggttctgatgctagctcaattgctcttctcttttcttgccgccatcctcatttatatatcacctttggtccatataacaactcactttatgtttttatttttatttttttatagatttggtctcacgctgtcacccaggatagagtgtactggtgtgatcatatctcactgcagattcaaacttctaggctcaagtcatcctcccacctcagcctccctagtagctaggcctacaggtgcatgtccccacaactggctaattttaaaaattttttgtaagaacaaggactttctctgttctctaggctggtctccaactccaggcctcaagtgatcctcttgcctcggcctcctaaagtactgggattataggagtgaaccaccacagcagctcactttaatccattgttggacaaaagtcaacgaaacaagtgttttgttttgttttgattttttaagaaaaaaaaggaataccaatagacaatttaaataagaaggagtattatacttttccagtttttttttttttttttagtatatttggatgatgttggcacgggatttagaagaagagttttgtggttcaattcagtataaaaatatatacaattatatcatataaaaggaatgatgctactcctactagaagagacaaagataaagcaaaaattgctcctgcctctcaggagtgcacatttaatgaggggaaaacaaagatacacatgaaactataataaaaagcataaataaattcatattttgagcaagaaataaacgaagtatcattggggaagaaaatatgcgatgattacttcctatcagggccatcgaagcaggtctcatgaaagaaaaggcatttgagcaaagccttgaaacgatgtaaagcatttcaaattgtagaaatggcagcaggggcctttcagataaggggacagagtggcaaaagtgcaaagagaggagaagctcagggcttgttagaggactaaagtgggcaacacaaagaaggaggcagaaaaagctcagatattctttttgcctaccacactctacctatctcaaatgaagttcttcgtattaggtaaaaatgctaggaaagaaaatagcacacagaattacgatgtgcacaaccctacatcagtgactagaggccctcagctaccatcctgctcttgtcattattgattcatttgtgtctctgagaagactgatagggagaagagaatttgggttttgagatttttcaagcgtatgtttgcatcttaacccttattaactgccttctattaagcaagtcacatttcttgtctttggctcagttttttcaattgtatagttgaactcagttgtttctaaggtccttttcagtctttttttttttttttttaacatcatgtctccatgactgtctgaaacttcagagagttggacactcactaatggactggtggtgtctggcacttctccagacatttccattgggaatctagtggaaggatcctccatttttctgtagcttcatacactctcccttccttcattccaacttctctttttccttcatttccttgttccttccttcctcttctttattccccctctctcttttcctcccttttcttaactctctctctttccctcctatatccttctttttctttctttctctttcttcttcatctctttaccccttcatttctctctcttttctttctaccttttgtcatttcacaaataatctctgacaacttctgtatacttggcaccatggtgggtacatggttatgaagtaggatgagacctggagcctccctaggctactgccagtgtagtgagtgggacaaataggtgaaccgacagttatagcaccatgtagcacacactatgataatgaaaaccctaagcgagtggctcacttggtcttggataaccagtgaaagctacccaaaagaagagatatctaaactaggacttgaagaacaggtgagtgaagcaggagaagtagagagtattccaagctcaggaaatggcaggaactgaggtcaaggtacaagagcgttgtatgtttgcggtattttgtatatttcatcatagtaagaatttccagtttagagaaaagaatacaggggttgggtcattaagagttttgcataacatgtaaaatatagatcttataccataggcaagggtttgtcgctgtaagcaatatgtacatgtattttagaaagattaatcattctggctatctgtggggagagattggaagggtaaaatgaggtatggggagaacaactaggagacttttgttatagaccagaagggagacaatagtggtctgtacttcagtgacagcaagcatgaagaaatatgaaataagggaggctctaaaaatgtcaaattgatgagagttataattgactgcatgtggggaagtaggtgatgaagagacacagtcaaagatgaaatctgcctttctgatctggggcaattggaggggtggtgatgttactcaccaagagtttaaatgaatgaaaattagtatctatacagaaaccttcatggagcaatgtccctgaaccatcctactatttttttctgcttagcttattatacaatacttagaggtaggaacacaattctttacaatggagatgtttatagatttaactttatatcagagggacctgaggttaaatcccaccttaccaggcagtggctgtatgaatttagacagactcttaaatagctataaaccacatttcctattcagtaatgagagaataacaatacacacatcataagtttcttgtgagaattaaatgagcttgtgtatctaaagcatttgccacagcagcaggcagatatgaatcagccaataaatattagatacattattgtctatttgcagtttattggtttgtttttgttctggagaaggagatgacattttgcataatgttcgctgaacagagaaatataaggtttaaattctattcctatttttggtattgcttctcttggagaaattttgtttcctcctctaaagctgtcatttgcctttttttttttttttttttttaaatcagaatctctctctgtcgcccaagctggagtgcagtggcatgatcttggctcactgcagcctccacctcctgagttcaagcgattctcctgcctcagcctcctgagtagccgggactacaggcaagcaccaccacacccggctaatttttttgtattttttgtagagacggggtttcaccatattggccaggctcgtcttgaactcctgacctcaggtgatccgcccacctcatcctcccaaagtgctgggattacaggtgtgaaccagcatggctggccacccatttttatatttacatctatttcctttttgtctgaccagggaaataagacagataataagtcaaatagtgaaggttatttatcaaatgctcacatttacaaagaaaataaattgagataatagcattgttaatatgttaactacaagatatgcaattcttaattatttacactgaaatagtttcttcacacagtagttactgatctctcaattataaaaaggaaaaagtgttttcacaagaagatttcattttcagttcatctttgttaattatttattgagaacctgctatgtactgagcactagtatgattaaaattttattacctcaaaacaaagttgctcacattagtatttattttatctgtataatcaggttctcttctgggatttctatttgcattaatattacaattcttttaaatataaagtaaatattaaaattattatatccagcatgcccgttgattatatccatttttaaactttccaattgatttcaaactctttcagcagatgtttgaggctacaaatgttctcttatttatctcatgatttcctaagtacctagcactgatgtacattaatgtgaactcatgatgcatttgctcacatgagttgatagagcgcctagtaacacacttagcatacctgttgaaagaatgaatatattaatgatatgaggtgattattgaaaatctcacattgaaccttaaataagaagtatgtctgtaatgaaatgatcatttttttaaagcaagatttcgtatcttgctacaatttaaatattttcaggatatgtatttggttcatttttaaaaataaaattggaatacaaatctgattcttgggatatctaataaggttgatgaagagtatattctggactagagaatgtggctttttgcttagtgctttaaagagagaaataaagaaaacagagagaaaaaagaaattgataatttgtaataatttactcaacataaattgtgatctttatcactgggcaataatcattacaaagttgtaatgccatacttttaaaaggaagacattttaacgtatgtaaccatttaaaaatgttaaaatgaaaatatttagaaagttcagatatataacgtaggtcatatacctgtgaagagcacaaaattttatttttctactaagttggcattacccatttcttatgatttatacctttagccagtgctccacaaataaccagaactaaaacaaataaattttagcaaataaagtcatgtttttctttcgtatcttataaatgtaagatataaagtaaaaagaaaaggcaacattgattatgattattttagtcttgctccctcatctttattaaggcctgtattgactccatttaggccctgggatagtaaaaaatataatataaaatgtaaaggtaaaatgttcagagtcttggttggaaaatttaaatgcattttatgttaaaattgaaaatgtttccaattttagtagtcaaacgttatttactacaatattattaaaacttttgcgtcttaataaatataagtaagtactaagtaggtatctaagtaggaaagtcacatcttcaaggttaaaatatattagcacatggatagtaaagtggtgtgaaaattataaattcttacaatttgtatatgcaaggtggttttttaaaataatatatcttaatagcttattttcttttagttgtctgtacatttataatcttaatgcatattgacaaaaaataatcttgatagcggttaaccaacaataaacatttacaaaatctgctgtgtatatttactctctctctctctctgtagatagatacatagatagatagatagatagacacacacacagacataatttcctatgttactagagaagagataaatgccgaacattgttgaatgtctctaattctcaagtattttttagtgtttctaattctcaaaagatacataaaaagacaaggcaggcaaaattgttgctgcttatatttcaagattgataacaaaagagaaactgggaaaaactaggttagagaagtcttaatgggagactgctatagagtccagaataagaaatcccaggattaaggaactaatttatgtcactgtaactcaagttggaagagtcatcatctctatggtttcctaacacttttaagtgacatcctactcatttttagtactgtggtaaacactttccagagccagttgagtaaacaaggtccagaatgacaccaaatcaatgtaagctcttcagtctaaatcataattttttggcttctggatttagctgtttttgtttatttagtcaagtagaaattgcacttattaagtagcaactgtgcatataataaccattttctgctccaaacttccaatgagagtatatgaattgattgacaaattgagattttcttatcctcactaaacatttattaagcacctattatgtatcaagagtaagagactgtatgctccttgaagccagctgccatgtttgtctttattatcgctgtatctccagcattaatatagtcggcatgttgactagtaaatgacaggcatttaataaatatagtttgaagaataacagatgacctataattgcacatacaaaagataatgcaatattttaaatgctataataatatggacaaattcctgtgggatttcaggatagaaagtgataaatgccagctagagagacttgggtggtgggaggctaatgaaaaaacagaagtctttaaaatggggcttgaataataagtagaagtttgacaggttgagagcaaggagttattctcattagtataattagtatataccaggaagagaaaactcaaatgccttaaggaaccaagtagccagcgttcacgagagaggcaggttgggtaggatctgtggggaacctggtgagcctgagctccacctaaatgggagcagccactcctttcttgccagttgttgctttgtgagactggtgagttcaggtacccagaatgaccaagtttctaagggaaccctgaaatctgaactgttctgtaaaatctctacacatttttggcaactaattaagagattttttgctttcctcatgcttgtgacttctactttattattgtaccttaaataaacctacctctctccatttagcaggtaatccactcttcacttttgggaacaatagatattcattgaaacaatacaaattagcattgttttaacgttatttatcaatatataagttgcatgttagaaggagaaattttaaatttataatcctctatttcagacaactctgtcagattaaaagttattacttaacatttgcattttttaccctttaagaaaggttaactatgatatttgaaacatcagtctgcttttttaagaaccctgtcttaaaattttcaagaatttagatttgcttgctttttagtttctaataagccattttacaacagaggaataagtaaatgaagatgataaatcataccagagagcattcctaaatataataaaaaacatgaaaaattgtaaccttgtcttttgtgcacaaaggcacctttaagggtgtctccagtgagtgctacattaacacagaagtttagttaattacagccactattctcacgtaccttaactgagtgtgaataccaagccatctaatagtgtgcccctgagcattaatacctaatgaaattggattccttgttttctctaatgagctcattgcttttctaaatatggtcattgcaggtaaatgatcaatagccttgaaactgataccactactgaattattttggcaagatggaaatactcttatttgtgtaaaataagaatttttgaatatgcatttcagatcactttctaaataatgtcatgtatgacaggaatgaccatagtaggctagtttgtttcagtggctggcttatacagtaagaaattgtggagagtcgctgtctgatttacagcacagtgccttcaaacttgtatcacctagcttgagctaaagtgaactggatgcagcgtgttcctgttcattaagacactacagggcagtcagctttgagaagatctgttttctgttatgatatagcagttctgtacaaactgtctctaatatactaatttcctatagttgccgtaacaaatgaccataaactcggtggcttaacaaaagataactttattctctcacagttctggaggctggaagcttataatcaagaagttggcaaggctgcgctgcccctgaaagttctggaagaatccgttcttagcctcttccagcttctggtggctgtaggcattccttgacttgtagctttatccctccaatgtctctgcctcagaggtcacattgcatctttcttttgtctgtttctctcctgcatgtgtctcttataatgaaatttgtcagcccacctgtataacccaatatgatctcaaggtcctcagttacattttcaaagatcctttttccaaataaggtcatatactggtggtaagaatgtggacatatctttctgagggcctccatctttctccaccttcactgtggttagttagtaaagcctaacacagccactactcaagtcattatgatgtttaagcactttactaccactatttttatttattgagcatatcatttatattgcgtgtgtatttgtaatttttaattcttataaccatcctatgattatctcccgtatacagataaggagattgaggatcaaaaaaggtaagatcttccccaaggttacaacatagatagtaagagtttcaatctatatttaatatttaatgcatatataaatttaatttacgtgtaatgcacatataaatttagacgtccacattatttagaaatttatatgttgaatttcacaagatagctgtttatcattagattttttgatctctgtgttacacaggatgagataatcctccagaaagtccaagaattgtttccaacttaaacctaaggaggagcatgccaaggtgaagttcgcagaataatagccttgggatgagatccaagttagggcttacttcacccaaagctatcatccaatacccaattctggattactttattttaaaatggatttggaattctttttaaaaaaatgtttttaggctgggcacggtgcctcacgcctgtaatcccagcactttgggaggccgaggtgggcggatcacctgaggtcaggagttcgagatcagcctgaccaacatggggaaaccccgtctctactaaaaatacttaaaaaaaaaaagtagcctggcgtggtggcgcatgcctgtaatcccatctactcgggaggctgaggcaggagaatcgcttgaacccagaaggtggaggttgccgtgagccgatcgcgccattgcactccagcctggggaaaacagcgagactctgcctcaaaaaaaattgtttttaaacatttgtaactgtttaaacaattttttagcacatatgcatcttcttaaatggggtacctagtgatgttttgatacatataatgtatagtgatcccattagggtaattagcatacccatcatctcaaacatttattttttgttggaaacattaaatatccttttttctagctatttgaaattatatcattattaacaatagccatcctagagtgctatagaacaggggtccacaacccccaggccacagaccagtactagtccgtggcctgttagtaactgggctgtgcagtgggaggtgagcagtgagcaagtgagcattaccgcctaatggtggacagaagctccaccttctgtcggatcagcggcagtattcgattctcataggagtgcaaaccctgttgtgaactgcacatgcgagggttctgagttgcatgctccttacaagcacctaatgcctgatgatctgagctggaacagtttcatccaaaagcatccccaaccccctacccactggttccatggaaaaattgtcttgcacgaaaccggtccctggtgccaaaaaggttgaagaccactggtatagaacactggaacttattcctcttatctagctgcaattttgtatctcttaacaaatctctccttgttccttggcccctacccttcccagccttcagtatcctctgtcttattttttacctcgaggtttttttttctgtttgtttgtttagacggaatctcgctctgtcgccaggctgcagtgcagtggcgcgatctcggctcactgcaacatccgactcagtggttcaagcgatgctcctgcctcccgagtggctgggattacaggcacgcaccaccacgcctagctaatttttgtatttttagtagagacggggtttcaccatgttagccaggatgatcccgatctcctgacctcttgatccgtccgcctcagcctcccaaagtgctgggattacaggcgtgagccaccgtgcccgaccgagatcaacttcttatagcttccacatatgagtaaaaatatgcaatgtttaactttctattcctggcttatttcatttaacattattcagttccatccatgctgacttaaataaaagaatttcattttttaaattgttaaatagtattccattgtgtagatataccatattgtatttacccattgctctgtggttggatatctaggttgattccatgtcttggctattgtgaatagtgtcacaaagagcatggaggtgcgcacatactgatttcctttcctttgaataaatgcccagtagtgagatttgttggatcataaagaatgggttttaaacacactgcaatgctcaggagcacacccacacactgctgtgtttgagtcctatctcctccattaactatgctttcttggggttacttaactttcctgtgccccaatttcctcatttgtaaaatggatgataaataatatctcttaacgtcccttaagaaataagaaaaataataatatgctaaatagtaactgctttatggtatagactctgtattgaataattattaccaactataagtattttacatataaagtagtaatagaggtaaaacattcagaatcgcgatgaagttgcaagcagtagaattttatttggcacataacacggcctcaaaaaatagcatggggcagagagtttcatagtgcatgtattcctgaatattattttattttccaaagcaaagtgttcttatgtttttttttctccccacagcaatttaaccccctcctttgcattcctcatcccaccctgctctgttatttattctttcttggggaaaaaattaagtttttattttccaagataattcatagttaaactttactaaactattcccagatacagaaggtaatttgaatccataactgggtcagaggaaacaattgtattagctctgttccatatgcgtgagctctatcaagaacacctgaaactattttctgttggcatgtttacgattctaagaaatctattgtgacttacggtttgtagataaagtatgagaaggttcagggaactggtaccttctagctctaagtggattcttagagtcatctgcacatcatttccaagtaaaaatggattacagtcgtcaagttgtatgaaattaatgctcaatctgcttacatcctttacatagcttaagcatttataatatatcattggagcaataaataagacttggggcctttatatattttatttattggcatcttttcatttattggttttctttgctaattattttatatttataaacttcatatataaagataatatttttcttcatggaactcagtattcgtgataaagaaacaatatatatttttaatagactcaaggtgtcaacagttatcatctattgttatatatatatatattttttggtgagtaaatgtcagcacagtgacatgaaatgatgtttttccataaccataactcaatggtggaagcagccacagatatttaaatatatttagctctggttttatctcttccagacgtgacttatttctctacccccacccttcatgaggaagtggattcatttcctggcccagaaaggcttcaattgtcagtgcttaagggaaaatattctaatacgcattgtttgttgtaaatgaagttctgatcacatgtgtaaccacttactttgctatcaaacacaaccaccaacttctctttttgatcaaggggaactgaactgtgcctgcatgaattgtttcacacggtgtcttctctaacatctaggtgagcacaaaatgccgaggcctctggtgggaatgcgtcacaaatgcttttgatgggattcgcacctgtgatgagtacgattccatacttgcggagcatccctgtacgtatgccttagagctcactgcttgccaggaagggaaagggacagaaaactgagttcaggtttccattttgtgctttgttttctattgtactatattaaggttcggtccagtttgtaatggttagaaattgagctcatctcggaaatgtgaattgaaatatatacttcagcacattttctcttttctcatatatttgtaaattcattgagggaaaaatattatcttattaatctctgctgtccctcagagggccaggcatagtgcttcatactcagcaggcttacagtaaatggttttttaatcgaaataaactctttagggtgctgtaatttcattattaaactggacgggttagggggaaagcatttcagagatgttttaagctatgacttagttcaaatagaaagtttacagttatttcagttgaaggttagctaagaaagagagagtggcagaggcagaaagaggcagacagccagagagagagacatagactatggggatcagtggaagaaaaaaccaacacatgtgacgcactgttacaggcaatattgaagctggctcccttatcttccttacttttagctttaaattatagttaaagccaggtgcagtggctcacacttgtaatcctagcactttgggaggccaaggcgggtggatcactggaggtcaggagttcgaaaccagcctggccaacatggtgaaaccccatccctaataaaattaccaaaaaaagttagctcatcatggtggtgggagcctgtagttccagctagtcaggagactgaggcaggagaatcacttgaacccagaaggcagaggttacagtgagccgagatcacaccactgcactccagcctgggagacagagcaagacttcatctcaaaaataaaataataaataaataaattatagttacatgtcagcagagcccatgttgctatgaataaagaaatgtcttaaatttaaaaatcttactgttgattctctcaatccttctccatagtgtttatttgtttatttataagaacatcgaaccctgctaagaaacatctgttctgttatgaaagcaaagtggttttagtctctttcaaagcaaatgatgggtgatggcaccataaggcaacttcttttctcaagataaataaaaaattaagcccttggaatgtgacttttcccctgaacctctatttcagtccagaggaaagcacttaaaaacagcagcactctaaaattcttcatctgctatttaaaagttgggtgcgtggaacatttttaaaaacatagttcataaggtctttgttttatttttgttaaaaaggattttcttaattctttttcttttcccctctctgtgctaacctagttctacctacaaagaaacactatttgctgaataggaatggacattttgtctattctaaaaattcattaaaatggattttaattcataagctgataagaaaatgaaaaattaaaaaaaatttaaaacttttaaatactgttatatacatttatgcaagaagaaaatataatcactcataagctcactacgtagagaaaaccactgtttatatttaatatgttcctttttcatctttgacctatgtaacctattaacatatgggggtagagaaataagtcacgtctggaagagataaaacccatatggcctaatatgtaatattggccaagaagagtcatgatttaaatagctgaaagagaaaatgatctaatttccagaaattaccttctacttaatagcacaaactaactctccttcttctaaagatctccttatggcttcttctatcctgaactggcaaaaagaagtcttgaaatattttattctgcttccctgtgtcaaattttagccaattattatttttaaataaaaaaaaattaaagtgattatttattcaatatttattaagaaatttttgtaggacagatatgctaccttcgattcagcaatcggctacaatatttgtgaatgagacattttccagagtaggaggcaaaaaggaaaacatttatttagtttccactatctaccaggatgctctctgctagcataccaacaacaaaactaagtagtgaactgtggttaaacaagcaataatgtcaataatctcatattttttagttttatgaaaacattaggggtacttatgttcaagttcatacaaagtctgacttttaccggaggggtgtgttaatgttacctacttgtctgttttttttgctatgtctctgtgagttaatatggttccttcttctgactctgctttaaccatatgccctggtcttccagtgaagctggtggtaactcgagcgttgatgattactgcagatattctagctgggtttggatttctcaccctgctccttggtcttgactgcgtgaaattcctccctgatgagccgtacattaaagtccgcatctgctttgttgctggagccacgttactaatagcaggtaccggtctggctggactagcaacaggggtagggagactctgctaagggcttgaggtgaaggagagagttgtgctgaagctgctcattttcggattatatgtggcttccctttctagattgaaaaactaaaggtcacttctaccagccctgcatactttagctttgaagtcagctaattagtcttttgttaatatctcagaacaaaatatgaagctctcaggccgggtgtggtggcttatgcctatattcccagcactttgggaggccaaggcaggcagatcacttgaggccaggagtttgaaaccagctggccatcatggtgaaaccctatccccactaaaaatacaaatccaggcatggtggtgcacacctgtagtcccagctactcggcggggctgaggcaggagaatcgctcgaacccaggaggcggaggttgcagtgagcagagatcgcgccactgcactccagcctgggcaacagagcaagactccgtctcacggaaaaaaaaaaaaaaaaaaaggaaataaagaaaaaaaaaagctctaaaactatgttttggccatttaaaaagttacataacttcaatttttaaaataatttatcttgtgattattactgaagttaaaatcctaaagtaagccccaaacttctacctccttacctatacccaccaccaccaactccaccaattctttttaacaataaactaacaattgtgccaagtcctatgttaaacttgtcccacgtactaacccatttgttcataaatgtaacaataaacagatcatattgttatcctcacttaagatgcagataaataattgaagttctgaggactggtcaagcatatttattagtcaagcatgactaataaacaacatatcaaaaagcactttaagtagtatttattagtgaaacagcaaaaatgatactttattcaggtctgtcttcaacttcaaagcttagtcctcttcttttgcaacataatgtcttcttcttgtctgttagcaggaaaaatcttgtctgctaacaaagcgaatataagtggcagcctgaccaggcagtgtggggtagtacatcgatatggagtttggaactagaaacacttgtagatatgtatgtgtgatatattcacccgtgtctctgtttcctgatctgcaaagaggcatgaggctaaggtagtaacatgtagcctgaattgctatggtgaagatgcaatgtgggcacagcaaactgttagctgactgcctaaccctttgtatgctcagaacttgggcctccctgacttttgacacagaaatgttaagtcaacgtcctaataatcctcagattgtattataaagttacaaaaatttagaattcttcccttctgtaagtcatttatttaattatcccacctactgacagcatagaactttttaatatacaatgtaattcatttaacagatttaaacattatttaatctaattatttacggctatataattttgttcgagaatatttttgagctatcatcagtaaataacccatcttatgtaaaacaacaaaacaaatagcatttaaaaaataagtcactgaagaaaatcctgataggaatgactgaagaaataactaaattgaaagacaaagcatgtcctaagctttggaaactttagaattagtgtgctataaaatttatttttaaagtctataatctgttttgaaggtttagaaagggaatttctaactgaaaactgcagataatggcattatagcaatgctattgcaatatatactgcgttttctaaaggttatgtgtttattatctggcttttttttttttttttttttgagatggagtctcgctctgtcgcccaggctggagtgcagtggcgtgatctcggctcactgcaagctccacctcctgggttcacgccattctcctgcctcagcctcccaagtagctgggactacaggcgcccaccaccacgcctggctactttttgtatttttagtagagaaggggtttcaccatgttggccaggatggtctcaatctcttgacctcgtgatccgcccgcctcggccccccaaagcgctgggattacaggtgtgagccaatgtgcccggcctatctgctccttcttaaagttcttacattaaacaattaggagaagaatacagttaaatagtgatttaaatagatatcacagactatctagggaaaaaaatgtaaaattttttggagactacatattttattttatttttttagatttgggaaagacaaatatttctctcattagacagtaaaacaactctggaaagtaatctgaagagattgtttgtgaacacatgcatctaacttagcacagagtagcagaactttgaaatgaaggaaaagtaggatccagttatttgggtgttggtgggcaagatcttaacactaacgttgatacagcttcaggatatcagtaagcatacatttacaagtaaataactgaaaatccaactcaagcagacttagacaacatatagattactgatttcttgtaattgccttctgctaggcattgagcatgtggagagtacatattttaaaaacactcttttaattcagtgttttgtcctccaactcaccacatttcttattgcatctaggcttcaacatgcaatttatacctttaaaataacaggacactagtggcgtcatttcaaaccagttaattgtcagagaggctaagctgtggagatgtatttaaagggaataacatttcttggtccattcttatatggtgtgagggtagtagataaagatttatttgaaaataaaaacattttttacttcaattatttgtgtttgacctcaagacactgaaatcagtgactttaaaaacagttttcacatgggtgctgattacgtagctggcatagcttcaaaagggggtacagggagcattaaatacaatgatatttactcacaatttaaaaatcattacagaatgaacatatgctctatgttgtttgtgttagactacattctttttctgttttgtttggttttgttttagtattttcctttatacaatactaacatggcattggaaagacaggagaatcaaagaaaaccataacgatgaatttcgatttacacagataagcactgtgttatttcatttttgcattttctttatgtataaactgagataaaatttaaaaaagatacaagatggaaggcaaaaggaagagacagaagaagtgtccgaagttcgggttgcccatgaatccatgttactgtttttacctctctgaatcacgccagccattttgtgtagtaagcaggtatttttggatttaaattcagaaaatgtcccctattatttgtagcatcctccctttctttcaggtaccccaggaatcattggctctgtgtggtatgctgttgatgtgtatgtggaacgttctactttggttttgcacaatatatttcttggtatccaatataaatttggttggtcctgttggctcggaatggctgggtctctgggttgctttttggctggagctgttctcacctgctgcttatatctttttaaaggtaagaataaaataaaatagcaaatttccttgcctccactatcgtttttcccaatccagtggaaacaaatttcaaaaggaaaaaaatgttatttatttgaattcctacctattgccattaaaaattccaattgttcaagggcaattgaattgtaatactcaaacattattacccagttagttctatattaattgaaaaataaaatccacaactacaagcatgtccaatattcaaatgtataatagttatcttgatgtattacaattatacatatatacatatatacacacatatacataccgtatatatactatatatgtatatatactatatacatatatatacacatatagtatatatactatatatacatactgtatatatacccttgtatatatacgtatacatagtacatatgtatacacatatacacatatgtatatgcatatatgtatatgtatacatatatgtataattgtaatacatcaaaataactattgtacatttgaatattggacatagttgtagttgtggattttttcaattaatgtaacactaacttggtaataatgtttgagtattgtaattcagttgcccttgaacaattggaatttttaatggtaattggaatttttaatggtaacaggtaggaatacacccatgtatatgcatgtatatatacacacacgtatatgcatgtatatatgcacacacgtatatgcatgtatatatgcacacacgtatatgcatgtatatatgcacacatgtatatgtatgtatatatgcacacatgtatatgtatgtatatatgcacacatgtatatgtatattagaattatacatatatgtgtgtctatatatacaattatacctttataattgtatgcatatatgtagatatacatataattgtaatacattaaaataactattatacatttgaatattggacatggttgtagttgtgaattttctatatatatatattttgatgtattacaattatacatgcatatatatcttcacccactcaactaaatgtatatttagtgttaaactgagaagtggactaagatccagccaaatacttctttttaaagaatttaacatgttatgttgggtttctaaaaatatcacctaaaaaactaagggaatacctctcctgatgaagaaaaaaaaaataacaggaaatctacttggctgaattttaaacctaaaagaaactttcagaatgaaaatcttaaattgtcttctaggattcttcttagagttccaaaatgataccttctttgagtatctatattcttgttccttttgaggaagaacatataaaatggtattttataattttcccaagttcactgagttctacttatttttatatttctttcaaacagatgttggacctgagagaaactatccttattccttgaggaaagcctattcagccgcgggtgtttccatggccaagtcatactcagcccctcgcacagagacggccaaaatgtatgctgtagacacaagggtgtaaaatgcacgtttcagggtgtgtttgcatatgatttaatcaatcagtatggttacattgataaaatagtaagtcaatccaggaacagttatttagaattcatattgaattaaattaattgctagcttaatcaaaatgtttgattctcctatactttttctttctattactcttatattttcccgtcattctctctgctaaccttccaccttatgcacacactttccctatattttaagataagtctgctaggatgtagaaatatttgtttgtgatttctatatagctattagagattatgacatagtaatattaaaatgaaatgatacttaaacagaaagcaatttccaaagaggccagggaccctaatctttgaagagatgaagaaacttacttttctccctggcttttggttcactttttgtacttttaacaagtgggtgaattatttgataattttgaggaagattattcttttaaattcaaactagtatgtcaatgcctaccattactctgattatattaaaacagaaaaaggaaataacaacttcgtataccagccactggtgagagttaaagacaagagctgcccccccacccccaaatgtcaaaggcaaatgctaaattgatactggagctcgtggtgactttctacctcactaacaacataagggatctccatattatttcaccactattctagctttgctgatatattgccaaatgattagactacagaatagttcaaccagagaatttactcatttattgattaaacatccaaatactattgtaatatactatgttaaaattcatcaattcaagtgcccacacaccactgaatcatcagcaccaagcaatatattagacatatggcaaaattcaacaaatatattttgatataaataaataaacgttcacgactttacttaaaaaatcaatgttgcggctgggcacggtagctcgcgtctgtaatccccgcactttgggaggccaaggcgggtggatcacgaggtcaagagacggagaccatcctggctaacatggtgaaaccctgtctctactaaaaatacaaaaattagccgggcgtggtggcggtgcctgtagtcccagctactcgggaggctgaggcaggagaatcgtttgaacccaggaggtggaggttgcagtgagcggagatcgcaccattgcactccagtctggcaacagagcgagactccatctcaaaaaacaaaaataaataaataaataaatattcttcataaaatgtgggttttggggaaaatatagaattacatatacatttaacgaagtcgctaatgacatttcattcatattcataatgtaaccatcttgaatttttttaattgtagcgattttaaaaatgtttgtaaaatttaatttccagttttctaattacttgtcagtcacattaataacattagtacctttatggtacccttgcagtacctgaaaagaatatcaacctgaaaagaatatcaactcacccagaaattagttctttgaaaaaaaagaaattaagttgtgaatttctaaagaccttgaaataagtgtttcaaatttaaagaacaaagaatgatgtgaaaatgagattatgattcctactacatgaattaacgtttcgagattgctgtttattacttcccagagtatctttaacagtattctctgaagcagttccaatctagttggagaattaacagcaattgatttaactatctcatttttattaactgtaatttactttaaaaatatttgcaaatcatactcattagttatttgatcattgttctatgcattttaaaattaattttgtgttgttcctctcaatatttgtttttaacatttattcccatttttattttatactattgtctgtcatgctttatgtattccaataagtgtcttgaaatccttgtggggaaaggcaggacaaaaataattagttaattagatttgaaaaatgtaatttttccattttaaatatttcatttgtataagaaaatatttcagagaaccatgatgataatggatatgtgtgactgttttgaatttttttctcaattaaaacattttgtatgtaatgggaggaatgtcaagatttgtt

SEQ ID NO: 277 - DNA, 3111 bp, Homo sapiens
  misc_feature 2411,3089,3090,3091,3092,3094,3095,3098,3099,3100,3101: n = A,T,C or G
ccccacccgaaacacactcagcccttgcactgacctgccttctgattggaggctggttgcttcggataatgacctccaggaccccactgttggttacagcctgtttgtattattcttactgcaactcaagacacctgcagcagggcgtgagaaaaagtaaaagaccagtattttcacattgccaggtaccagaaacacagaagactgacacccgccacttaagtggggccagggctggtgtctgcccatgttgccatcctgatgggctgcttgccacaatgagggatcttcttcaatacatcgcttgcttctttgcctttttctctgctgggtttttgattgtggccacctggactgactgttggatggtgaatgctgatgactctctggaggtgagcacaaaatgccgaggcctctggtgggaatgcgtcacaaatgcttttgatgggattcgcacctgtgatgagtacgattccatacttgcggagcatcccttgaagctggtggtaactcgagcgttgatgattactgcagatattctagctgggtttggatttctcaccctgctccttggtcttgactgcgtgaaattcctccctgatgagccgtacattaaagtccgcatctgctttgttgctggagccacgttactaatagcaggtaccccaggaatcattggctctgtgtggtatgctgttgatgtgtatgtggaacgttctactttggttttgcacaatatatttcttggtatccaatataaatttggttggtcctgttggctcggaatggctgggtctctgggttgctttttggctggagctgttctcacctgctgcttatatctttttaaagatgttggacctgagagaaactatccttattccttgaggaaagcctattcagccgcgggtgtttccatggccaagtcatactcagcccctcgcacagagacggccaaaatgtatgctgtagacacaagggtgtaaaatgcacgtttcagggtgtgtttgcatatgatttaatcaatcagtatggttacattgataaaatagtaagtcaatccaggaacagttatttagaattcatattgaattaaattaattgctagcttaatcaaaatgtttgattctcctatactttttctttctattactcttatattttcccgtcattctctctgctaaccttccaccttatgcacacactttccctatattttaagataagtctgctaggatgtagaaatatttgtttgtgatttctatatagctattagagattatgacatagtaatattaaaatgaaatgatacttaaacagaaagcaatttccaaagaggccagggaccctaatctttgaagagatgaagaaacttacttttctccctggcttttggttcactttttgtacttttaacaagtgggtgaattatttgataattttgaggaagattattcttttaaattcaaactagtatgtcaatgcctaccattactctgattatattaaaacagaaaaaggaaataacaacttcgtataccagccactggtgagagttaaagacaagagctgcccccccacccccaaatgtcaaaggcaaatgctaaattgatactggagctcgtggtgactttctacctcactaacaacataagggatctccatattatttcaccactattctagctttgctgatatattgccaaatgattagactacagaatagttcaaccagagaatttactcatttattgattaaacatccaaatactattgtaatatactatgttaaaattcatcaattcaagtgcccacacaccactgaatcatcagcaccaagcaatatattagacatatggcaaaattcaacaaatatattttgatataaataaataaacgttcacgactttacttaaaaaatcaatgttgcggctgggcacggtagctcgcgtctgtaatccccgcactttgggaggccaaggcgggtggatcacgaggtcaagagacggagaccatcctggctaacatggtgaaaccctgtctctactaaaaatacaaaaattagccgggcgtggtggcggtgcctgtagtcccagctactcgggaggctgaggcaggagaatcgtttgaacccaggaggtggaggttgcagtgagcggagatcgcaccattgcactccagtctggcaacagagcgagactccatctcaaaaaacaaaaataaataaataaataaatattcttcataaaatgtgggttttggggaaaatatagaattacatatacatttaacgaagtcgctaatgacatttcattcatattcataatgtaaccatcttgaatttttttaattgtagcgattttaaaaatgtttgtaaaatttaatttccagttttctaattacttgtcagycacattaataacattagtacctttatggtacccttgcagtacctgaaaagaatatcaacctgaaaagaatatcaactcacccagaaattagttctttgaaaaaaaagaaattaagttgtgaatttctaaagaccttgaaataagtgtttcaaatttaaagaacaaagaatgatgtgaaaatgagattatgattcctactacatgaattaacgtttcgagattgctgtttattacttcccagagtatctttaacagtattctctgaagcagttccaatctagttggagaattaacagcaattgatttaactatctcatttttattaactgtaatttactttaaaaatatttgcaaatcatactcattagttatttgatcattgttctatgcattttaaaattaattttgtgttgttcctctcaatatttgtttttaacatttattcccatttttattttatactattgtctgtcatgctttatgtattccaataagtgtcttgaaatccttgtggggaaaggcaggacaaaaataattagttaattagatttgaaaaatgtaatttttccattttaaatatttcatttgtataagaaaatatttcagagaaccatgatgataatggatatgtgtgactgttttgaatttttttctcaattaaaacattttgtatgtaawrrrarraawrwmaagatttgtt

SEQ ID NO: 278 - protein, 305 aa, Homo sapiens
MTSRTPLLVTACLYYSYCNSRHLQQGVRKSKRPVFSHCQVPETQKTDTRHLSGARAGVCPCCHPDGLLATMRDLLQYIACFFAFFSAGFLIVATWTDCWMVNADDSLEVSTKCRGLWWECVTNAFDGIRTCDEYDSILAEHPLKLVVTRALMITADILAGFGFLTLLLGLDCVKFLPDEPYIKVRICFVAGATLLIAGTPGIIGSVWYAVDVYVERSTLVLHNIFLGIQYKFGWSCWLGMAGSLGCFLAGAVLTCCLYLFKDVGPERNYPYSLRKAYSAAGVSMAKSYSAPRTETAKMYAVDTRV

SEQ ID NO: 279 - protein, 305 aa, Homo sapiens; identical to SEQ ID NO: 278

SEQ ID NO: 280 - protein, 305 aa, Homo sapiens; identical to SEQ ID NO: 278

SEQ ID NO: 281 - protein, 58 aa, Homo sapiens
MIRLQNSSTREFTHLLIKHPNTIVIYYVKIHQFKCPHTTESSAPSNILDIWQNSTNIF

SEQ ID NO: 282 - protein, 75 aa, Homo sapiens
MNMNEMSLATSLNVYVILYFPQNPHFMKNIYLFIYFCFLRWSLALLPDWSAMVRSPLTATSTSWVQTILLPQPPE

SEQ ID NO: 283 - DNA, 414 bp, Homo sapiens
atgcagcatcaccaccatcaccaccacttcttgcttccaggctttgcgctgcaaatccagtgctaccagtgtgaagaattccagctgaacaacgactgctcctcccccgagttcattgtgaattgcacggtgaacgttcaagacatgtgtcagaaagaagtgatggagcaaagtgccgggatcatgtaccgcaagtcctgtgcatcatcagcggcctgtctcatcgcctctgccgggtaccagtccttctgctccccagggaaactgaactcagtttgcatcagctgctgcaacacccctctttgtaacgggccaaggcccaagaaaaggggaagttctgcctcggccctcaggccagggctccgcaccaccatcctgttcctcaaattagccctcttctcggcacactgctga

SEQ ID NO: 284 - protein, 137 aa, Homo sapiens
MQHHHHHHHFLLPGFALQIQCYQCEEFQLNNDCSSPEFIVNCTVNVQDMCQKEVMEQSAGIMYRKSCASSAACLIASAGYQSFCSPGKLNSVCISCCNTPLCNGPRPKKRGSSASALRPGLRTTILFLKLALFSAHC

SEQ ID NO: 285 - DNA, 1740 bp, Homo sapiens
  misc_feature 755,756,757,758,759,760,761,762,763,764,765,766,767,768,769,770,771,772,773,774,775,776,777,778,779,780,781,782,783,784,785,786,787,788,789,790,791,792,793,794,795,796,797,798,799,800,801,802,803,804,805,806,807,808,809,810,811,812,813,814,815,816,817,818,819,820,821,822,823,824,825,826,827,828,829,830,831,832,833,834,835,1605,1606,1607,1608,1609,1610,1611,1612,1613,1614,1615,1616,1617,1618,1619,1620,1621,1622,1623,1624,1625,1626,1629: n = A,T,C or G
ggaaaattcatgaagaggggactgaaatccacaactcaatcagcatagagcagaagtaagggggaagtggtaagaggtgcactatgaatgagctggagaatttaaagggaggctgaactcagagtcgaagtgaccttgagaagataaaccctctggaaattctcagaatctcaggatgggccccagagtatctaaagatgctacagttcaagggattgagccaattgtatataaatcttaatggataggttgacctcagcataaaacttgggtggaaattttaaacaggtttctttatttcagcacttctcagagccactcattgtataaggtactttgtgaatatccagatagtattcttcaaactctcttttatttccccagggggcatcccataggacaagaagcattctttgtgacactctgtgggaagagctggtttaaaggggtacctgtctgggcaacactgtcccacaggggcccccatgaccaaactaactctgcttctacccagaaagggtgcagagtagccactagacttttatgtggcaaatgggatggttatgcccagcctgaagccaagatgccctttctggttgccttgatttgtgtttaacagctccaaatgcttaatgaggcagtaagagacgtctctcttgggcagtacttcccaactaggggtgagtttgccacccttacccccatcccagtgaatatttgcaattcctaaagacgtgttttgattgtcacactgnnnnnnnnnnnnnnnnnnnnnnnnnnnnnnnnnnnnnnnnnnnnnnnnnnnnnnnnnnnnnnnnnnnnnnnnnnnnnnnnnacaaaagagaattatctagccccaaatgtccataacactgctgttgagaaaacctaccgcaggatcttactgggcttcataggtaagcttgccctttgttctggcttctgtagatatataaaataaagacactgcccagtccctccctcaacgtcccgagccagggctcaaggcaaattccaataacagtagaatgaacactaaatattgatttcaaaatctcagcaactagaagaatgaccaaccatcctggttggcctgggactgtcctagttttagcattgaaagtttcaggttccaggaaagccctcaggcctgggctgctggtcaccctagcagctgagggactcttcaatacagaattagtctttgcgcactggagatgaatatactttaatttgtaacatgtgaaaacatctataaacatctactggaagcctgttctgtctgcaccgacattttcattgaagtacggattcttcctgacctagatgacagctggctgctgacaactttgcgagggctcggtatataaactgagctttgtacctatttttaataattacatgatatagtatataacttggattaacccagtattcgggtattttcaatttccttggggagcttagagggacggacaaataaaaaagattatttcaacattcaaatatatgccattggtttacatatgaagataaccacatatatgtataaattcaccgttactttttagcaatactataaaatccaacagaaaaaaatagcannnnnnnnnnnnnnnnnnnnnnganttagtctttgtgggtttggggcaagcaactgcccttctcagttaggatgggggagttctggacatttctagctaaagcccaggggtcaagggaatgataaactcctcggtc

SEQ ID NO: 286 - protein, 116 aa, Homo sapiens [sequence omitted]

SEQ ID NO: 287 - DNA, 32 bp, Artificial Sequence
  PCR primer
cacttcttgcttccaggctttgcgctgcaaat

SEQ ID NO: 288 - DNA, 29 bp, Artificial Sequence
  PCR primer
actagctcgagtcagcagtgtgccgagaa

SEQ ID NO: 289 - protein, 114 aa, Homo sapiens
MWVLGIAATFCGLFLLPGFALQIQCYQCEEFQLNNDCSSPEFIVNCTVNVQDMCQKEVMEQSAGIMYRKSCASSAACLIASAGYQSFCSPGKLNSVCISCCNTPLCNGPRPKKR

SEQ ID NO: 290 - protein, 115 aa, Homo sapiens
MWVLGIAATFCGLFLLPGFALQIQCYQCEEFQLNNDCSSPEFIVNCTVNVQDMCQKEVMEQSAGIMYRKSCASSAACLIASAGYQSFCSPGKLNSVCISCCNTPLCNGPRPKKRG

SEQ ID NO: 291 - protein, 30 aa, Artificial Sequence
  Synthetic peptide used for generation of rabbit polyclonal anti-sera against O591s.
YQCEEFQLNNDCSSPEFIVNCTVNVQDMCQ

SEQ ID NO: 292 - protein, 25 aa, Artificial Sequence
  Synthetic peptide used for generation of rabbit polyclonal anti-sera against O591s.
CQKEVMEQSAGIMYRKSCASSAACL

SEQ ID NO: 293 - protein, 30 aa, Artificial Sequence
  Synthetic peptide used for generation of rabbit polyclonal anti-sera against O591s.
TPLCNGPRPKKRGSSASALRPGLRTTIGCG

(19) United States
(12) Patent Application Publication
(10) Pub. No.: US 2003/0206918 A1
(43) Pub. Date: Nov. 6, 2003

(54) Compositions and methods for the therapy and diagnosis of ovarian cancer

(75) Inventors: Gary R. Fanger, Mill Creek, WA (US); Steven P. Fling, Bainbridge Island, WA (US)

Correspondence Address: CORIXA CORPORATION, 1124 COLUMBIA STREET, SUITE 200, SEATTLE, WA, 98104, US

(73) Assignee: Corixa Corporation, Seattle, WA (US)

(21) Appl. No.: 10/361,811

(22) Filed: Feb. 5, 2003

Related U.S. Application Data
(63) Continuation-in-part of application No. 10/212,677, filed on Aug. 2, 2002.
Application No. 10/212,677 is a continuation-in-part of application No. 09/970,966, filed on Oct. 2, 2001.
Application No. 09/970,966 is a continuation-in-part of application No. 09/825,294, filed on Apr. 3, 2001.
Application No. 09/825,294 is a continuation-in-part of application No. 09/713,550, filed on Nov. 14, 2000, now Pat. No. 6,617,109.
Application No. 09/713,550 is a continuation-in-part of application No. 09/656,668, filed on Sep. 7, 2000.
Application No. 09/656,668 is a continuation-in-part of application No. 09/640,173, filed on Aug. 15, 2000, now Pat. No. 6,613,515.
Application No. 09/640,173 is a continuation-in-part of application No. 09/561,778, filed on May 1, 2000, now abandoned.
Application No. 09/561,778 is a continuation-in-part of application No. 09/394,374, filed on Sep. 10, 1999, now abandoned.

(51) Int. Cl. A61K 39/00; C07H 21/04; C12P 21/02; C12N 5/06; C07K 14/47

(52) U.S. Cl. 424/185.1; 435/69.3; 435/320.1; 435/325; 530/350; 536/23.5

(57) ABSTRACT

Compositions and methods for the therapy and diagnosis of cancer, particularly ovarian cancer, are disclosed. Illustrative compositions comprise one or more ovarian tumor polypeptides, immunogenic portions thereof, polynucleotides that encode such polypeptides, antigen presenting cell that expresses such polypeptides, and T cells that are specific for cells expressing such polypeptides. The disclosed compositions are useful, for example, in the diagnosis, prevention and/or treatment of diseases, particularly ovarian cancer.

### BACKGROUND OF THE INVENTION

1. Field of the Invention

The present invention relates generally to ovarian cancer therapy. The invention is more specifically related to polypeptides comprising at least a portion of an ovarian carcinoma protein, and to polynucleotides encoding such polypeptides, as well as antibodies and immune system cells that specifically recognize such polypeptides. Such polypeptides, polynucleotides, antibodies and cells may be used in vaccines and pharmaceutical compositions for treatment of ovarian cancer.

2. Description of Related Art

Ovarian cancer is a significant health problem for women in the United States and throughout the world. Although advances have been made in detection and therapy of this cancer, no vaccine or other universally successful method for prevention or treatment is currently available. Management of the disease currently relies on a combination of early diagnosis and aggressive treatment, which may include one or more of a variety of treatments such as surgery, radiotherapy, chemotherapy and hormone therapy. The course of treatment for a particular cancer is often selected based on a variety of prognostic parameters, including an analysis of specific tumor markers. However, the use of established markers often leads to a result that is difficult to interpret, and high mortality continues to be observed in many cancer patients.

Immunotherapies have the potential to substantially improve cancer treatment and survival. Such therapies may involve the generation or enhancement of an immune response to an ovarian carcinoma antigen. However, to date, relatively few ovarian carcinoma antigens are known and the generation of an immune response against such antigens has not been shown to be therapeutically beneficial.

Accordingly, there is a need in the art for improved methods for identifying ovarian tumor antigens and for using such antigens in the therapy of ovarian cancer. The present invention fulfills these needs and further provides other related advantages.

### BRIEF SUMMARY OF THE INVENTION

Briefly stated, this invention provides compositions and methods for the therapy of cancer, such as ovarian cancer.

In one aspect, the present invention provides polynucleotide compositions comprising a sequence selected from the group consisting of:

(a) sequences provided in SEQ ID NO:1, 2, 5, 9, 10, 13, 16, 19, 23, 27, 28, 32, 33, 35, 38, 41-50, 52, 53, 56, 57, 63, 65, 69-72, 75, 78, 80-82, 84, 86, 89-93, 95, 97-100, 103, 107, 111, 114, 117, 120, 121, 125, 128, 132-134, 136, 137, 140, 143-146, 148-151, 156, 158, 160-162, 166-168, 171, 174-183, 185, 193-199, 203-206, 208, 216-246, 250-256, 262-268, 273-277, 283, 285 and 287-288;

(b) complements of the sequences provided in SEQ ID NO:1, 2, 5, 9, 10, 13, 16, 19, 23, 27, 28, 32, 33, 35, 38, 41-50, 52, 53, 56, 57, 63, 65, 69-72, 75, 78, 80-82, 84, 86, 89-93, 95, 97-100, 103, 107, 111, 114, 117, 120, 121, 125, 128, 132-134, 136, 137, 140, 143-146, 148-151, 156, 158, 160-162, 166-168, 171, 174-183, 185, 193-199, 203-206, 208, 216-246, 250-256, 262-268, 273-277, 283, 285, and 287-288;

(c) sequences consisting of at least 20 contiguous residues of a sequence provided in SEQ ID NO:1, 2, 5, 9, 10, 13, 16, 19, 23, 27, 28, 32, 33, 35, 38, 41-50, 52, 53, 56, 57, 63, 65, 69-72, 75, 78, 80-82, 84, 86, 89-93, 95, 97-100, 103, 107, 111, 114, 117, 120, 121, 125, 128, 132-134, 136, 137, 140, 143-146, 148-151, 156, 158, 160-162, 166-168, 171, 174-183, 185, 193-199, 203-206, 208, 216-246, 250-256, 262-268, 273-277, 283, 285 and 287-288;

(d) sequences that hybridize to a sequence provided in SEQ ID NO:1, 2, 5, 9, 10, 13, 16, 19, 23, 27, 28, 32, 33, 35, 38, 41-50, 52, 53, 56, 57, 63, 65, 69-72, 75, 78, 80-82, 84, 86, 89-93, 95, 97-100, 103, 107, 111, 114, 117, 120, 121, 125, 128, 132-134, 136, 137, 140, 143-146, 148-151, 156, 158, 160-162, 166-168, 171, 174-183, 185, 193-199, 203-206, 208, 216-246, 250-256, 262-268, 273-277, 283, 285, and 287-288 under moderately stringent conditions;

(e) sequences having at least 75% identity to a sequence provided in SEQ ID NO:1, 2, 5, 9, 10, 13, 16, 19, 23, 27, 28, 32, 33, 35, 38, 41-50, 52, 53, 56, 57, 63, 65, 69-72, 75, 78, 80-82, 84, 86, 89-93, 95, 97-100, 103, 107, 111, 114, 117, 120, 121, 125, 128, 132-134, 136, 137, 140, 143-146, 148-151, 156, 158, 160-162, 166-168, 171, 174-183, 185, 193-199, 203-206, 208, 210-214, 216-246, 250-256, 262-268, 273-277, 283, 285, and 287-288;

(f) sequences having at least 90% identity to a sequence provided in SEQ ID NO:1, 2, 5, 9, 10, 13, 16, 19, 23, 27, 28, 32, 33, 35, 38, 41-50, 52, 53, 56, 57, 63, 65, 69-72, 75, 78, 80-82, 84, 86, 89-93, 95, 97-100, 103, 107, 111, 114, 117, 120, 121, 125, 128, 132-134, 136, 137, 140, 143-146, 148-151, 156, 158, 160-162, 166-168, 171, 174-183, 185, 193-199, 203-206, 208, 210-214, 216-246, 250-256, 262-268, 273-277, 283, 285, and 287-288; and

(g) degenerate variants of a sequence provided in SEQ ID NO:1, 2, 5, 9, 10, 13, 16, 19, 23, 27, 28, 32, 33, 35, 38, 41-50, 52, 53, 56, 57, 63, 65, 69-72, 75, 78, 80-82, 84, 86, 89-93, 95, 97-100, 103, 107, 111, 114, 117, 120, 121, 125, 128, 132-134, 136, 137, 140, 143-146, 148-151, 156, 158, 160-162, 166-168, 171, 174-183, 185, 193-199, 203-206, 208, 210-214, 216-246, 250-256, 262-268, 273-277, 283, 285, and 287-288.

In one preferred embodiment, the polynucleotide compositions of the invention are expressed in at least about 20%, more preferably in at least about 30%, and most preferably in at least about 50% of ovarian tumors samples tested, at a level that is at least about 2-fold, preferably at least about 5-fold, and most preferably at least about 10-fold higher than that for normal tissues.

In one aspect, the present invention provides polypeptides comprising an immunogenic portion of an ovarian carcinoma protein, or a variant thereof that differs in one or more substitutions, deletions, additions and/or insertions such that the ability of the variant to react with ovarian carcinoma protein-specific antisera is not substantially diminished. Within certain embodiments, the ovarian carcinoma protein comprises a sequence that is encoded by a polynucleotide sequence selected from the group consisting of SEQ ID NO:1, 2, 5, 9, 10, 13, 16, 19, 23, 27, 28, 32, 33, 35, 38, 41-50, 52, 53, 56, 57, 63, 65, 69-72, 75, 78, 80-82, 84, 86, 89-93, 95, 97-100, 103, 107, 111, 114, 117, 120, 121, 125, 128, 132-134, 136, 137, 140, 143-146, 148-151, 156, 158, 160-162, 166-168, 171, 174-183, 185, 193-199, 203-205, 208, 210-214, 216-246, 250-256, 262-268, 273-277, 283, 285, and 287-288, and complements of such polynucleotides.

The present invention further provides polynucleotides that encode a polypeptide as described above or a portion thereof, expression vectors comprising such polynucleotides and host cells transformed or transfected with such expression vectors.

The present invention further provides polypeptide compositions comprising an amino acid sequence selected from the group consisting of sequences recited in SEQ ID NO:200-202, 207, 209, 215, 247-249, 257-261, 269-272, 278-282, 284, 286 and 289-293.

In certain preferred embodiments, the polypeptides of the present invention are immunogenic, i.e., they are capable of eliciting an immune response, particularly a humoral and/or cellular immune response, as further described herein.

The present invention further provides fragments, variants and/or derivatives of the disclosed polypeptide sequences, wherein the fragments, variants and/or derivatives preferably have a level of immunogenic activity of at least about 50%, preferably at least about 70% and more preferably at least about 90% of the level of immunogenic activity of a the ovarian carcinoma protein comprises an amino acid sequence encoded by a polynucleotide that comprises a sequence recited in any one of SEQ ID NO:1-185, 187-199, 203-206, 208, 210-214, 216-246, 250-256, 262-268, 273-277, 283 and 285.

Within other aspects, the present invention provides pharmaceutical compositions comprising a polypeptide and/or polynucleotide as described above and a physiologically acceptable carrier.

Within a related aspect of the present invention, the pharmaceutical compositions, e.g., vaccine compositions, are provided for prophylactic or therapeutic applications. Such compositions generally comprise an immunogenic polypeptide or polynucleotide of the invention and an immunostimulant, such as an adjuvant.

The present invention further provides pharmaceutical compositions that comprise: (a) an antibody or antigen-binding fragment thereof that specifically binds to a polypeptide of the present invention, or a fragment thereof; and (b) a physiologically acceptable carrier.

Within further aspects, the present invention provides pharmaceutical compositions comprising: (a) an antigen presenting cell that expresses a polypeptide as described above and (b) a pharmaceutically acceptable carrier or excipient. Illustrative antigen presenting cells include dendritic cells, macrophages, monocytes, fibroblasts and B cells.

Within related aspects, pharmaceutical compositions are provided that comprise: (a) an antigen presenting cell that expresses a polypeptide as described above and (b) an immunostimulant.

The present invention further provides, in other aspects, fusion proteins that comprise at least one polypeptide as described above, as well as polynucleotides encoding such fusion proteins, typically in the form of pharmaceutical compositions, e.g., vaccine compositions, comprising a physiologically acceptable carrier and/or an immunostimulant. The fusions proteins may comprise multiple immunogenic polypeptides or portions/variants thereof, as described herein, and may further comprise one or more polypeptide segments for facilitating the expression, purification and/or immunogenicity of the polypeptide(s).

Within further aspects, the present invention provides methods for stimulating an immune response in a patient, preferably a T cell response in a human patient, comprising administering a pharmaceutical composition described herein. The patient may be afflicted with ovarian cancer, in which case the methods provide treatment for the disease, or patient considered at risk for such a disease may be treated prophylactically.

Within further aspects, the present invention provides methods for inhibiting the development of a cancer in a patient, comprising administering to a patient a pharmaceutical composition as recited above. The patient may be afflicted with ovarian cancer, in which case the methods provide treatment for the disease, or patient considered at risk for such a disease may be treated prophylactically.

The present invention further provides, within other aspects, methods for removing tumor cells from a biological sample, comprising contacting a biological sample with T cells that specifically react with a polypeptide of the present invention, wherein the step of contacting is performed under conditions and for a time sufficient to permit the removal of cells expressing the protein from the sample.

Within related aspects, methods are provided for inhibiting the development of a cancer in a patient, comprising administering to a patient a biological sample treated as described above.

Methods are further provided, within other aspects, for stimulating and/or expanding T cells specific for a polypeptide of the present invention, comprising contacting T cells with one or more of: (i) an ovarian carcinoma polypeptide as described above; (ii) a polynucleotide encoding such a polypeptide; and/or (iii) an antigen presenting cell that expresses such a polypeptide; under conditions and for a time sufficient to permit the stimulation and/or expansion of T cells. Isolated T cell populations comprising T cells prepared as described above are also provided.

Within further aspects, the present invention provides methods for inhibiting the development of a cancer in a patient, comprising administering to a patient an effective amount of a T cell population as described above.

The present invention further provides methods for inhibiting the development of a cancer in a patient, comprising the steps of: (a) incubating CD4⁺ and/or CD8⁺ T cells isolated from a patient with one or more of: (i) a polypeptide comprising at least an immunogenic portion of polypeptide disclosed herein; (ii) a polynucleotide encoding such a polypeptide; and (iii) an antigen-presenting cell that expressed such a polypeptide; and (b) administering to the patient an effective amount of the proliferated T cells, and thereby inhibiting the development of a cancer in the patient. Proliferated cells may, but need not, be cloned prior to administration to the patient.

Within further aspects, the present invention provides methods for determining the presence or absence of a cancer, preferably an ovarian cancer, in a patient comprising: (a) contacting a biological sample obtained from a patient with a binding agent that binds to a polypeptide as recited above; (b) detecting in the sample an amount of polypeptide that binds to the binding agent; and (c) comparing the amount of polypeptide with a predetermined cut-off value, and therefrom determining the presence or absence of a cancer in the patient. Within preferred embodiments, the binding agent is an antibody, more preferably a monoclonal antibody.

The present invention also provides, within other aspects, methods for monitoring the progression of a cancer in a patient. Such methods comprise the steps of: (a) contacting a biological sample obtained from a patient at a first point in time with a binding agent that binds to a polypeptide as recited above; (b) detecting in the sample an amount of polypeptide that binds to the binding agent; (c) repeating steps (a) and (b) using a biological sample obtained from the patient at a subsequent point in time; and (d) comparing the amount of polypeptide detected in step (c) with the amount detected in step (b) and therefrom monitoring the progression of the cancer in the patient.

The present invention further provides, within other aspects, methods for determining the presence or absence of a cancer in a patient, comprising the steps of: (a) contacting a biological sample obtained from a patient with an oligonucleotide that hybridizes to a polynucleotide that encodes a polypeptide of the present invention; (b) detecting in the sample a level of a polynucleotide, preferably mRNA, that hybridizes to the oligonucleotide; and (c) comparing the level of polynucleotide that hybridizes to the oligonucleotide with a predetermined cut-off value, and therefrom determining the presence or absence of a cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of a cancer in a patient, comprising the steps of: (a) contacting a biological sample obtained from a patient with an oligonucleotide that hybridizes to a polynucleotide that encodes a polypeptide of the present invention; (b) detecting in the sample an amount of a polynucleotide that hybridizes to the oligonucleotide; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Within further aspects, the present invention provides antibodies, such as monoclonal antibodies, that bind to a polypeptide as described above, as well as diagnostic kits comprising such antibodies. Diagnostic kits comprising one or more oligonucleotide probes or primers as described above are also provided.

These and other aspects of the present invention will become apparent upon reference to the following detailed description. All references disclosed herein are hereby incorporated by reference in their entirety as if each was incorporated individually.

### BRIEF DESCRIPTION OF THE SEQUENCE IDENTIFIERS

SEQ ID NO:1, 2, 5, 9, 10, 13, 16, 19, 23, 27, 28, 32, 33, 35, 38, 41-50, 52, 53, 56, 57, 63, 65, 69-72, 75, 78, 80-82, 84, 86, 89-93, 95, 97-100, 103, 107, 111, 114, 117, 120, 121, 125, 128, 132-134, 136, 137, 140, 143-146, 148-151, 156, 158, 160-162, 166-168, 171, 174-183, 185, and 193-199 are described in Tables III-VII below.

SEQ ID NO:200 is the amino acid sequence of a polypeptide encoded by the polynucleotide recited in SEQ ID NO:182;

SEQ ID NO:201 is the amino acid sequence of a polypeptide encoded by the polynucleotide recited in SEQ ID NO:182;

SEQ ID NO:202 is the amino acid sequence of a polypeptide encoded by the polynucleotide recited in SEQ ID NO:182.

SEQ ID NO:203 is the determined extended cDNA sequence for SEQ ID NO:197.

SEQ ID NO:204 is the determined extended cDNA sequence for SEQ ID NO:198.

SEQ ID NO:205 is the determined extended cDNA sequence for SEQ ID NO:199.

SEQ ID NO:206 is the determined cDNA sequence for the coding region of O568S fused to an N-terminal His tag.

SEQ ID NO:207 is the amino acid sequence of the polypeptide encoded by the polynucleotide recited in SEQ ID NO:206.

SEQ ID NO:208 is the determined cDNA sequence for the coding region of GPR39 as downloaded from the High Throughput Genomics Database.

SEQ ID NO:209 is the amino acid sequence encoded by the cDNA sequence recited in SEQ ID NO:208.

SEQ ID NO:210 is the nucleotide sequence of O1034C an ovary specific EST clone discovered using electronic subtraction.

SEQ ID NO:211 is the full length nucleotide sequence of O591 S.

SEQ ID NO:212 is the sequence BF345141 which shows sequence homology with O1034C/O591S allowing for the extension of O591 S.

SEQ ID NO:213 is the sequence BE336607 which shows sequence homology with O1034C/O591S allowing for the extension of O591S.

SEQ ID NO:214 is the consensus nucleotide sequence of O1034C/O591S containing 1897 base pairs.

SEQ ID NO:215 is the predicted translation of the open reading frame identified within SEQ ID NO:214 (nucleotides 260-682).

SEQ ID NO:216 is a determined 5′ DNA sequence of clone number 91226.5.

SEQ ID NO:217 is a determined 5′ DNA sequence of clone number 91227.2.

SEQ ID NO:218 is a determined 5′ DNA sequence of clone number 91230.2.

SEQ ID NO:219 is a determined 5′ DNA sequence of clone number 91231.2.

SEQ ID NO:220 is a determined 5′ DNA sequence of clone number 91238.3.

SEQ ID NO:221 is a determined 5′ DNA sequence of clone number 91239.6.

SEQ ID NO:222 is a determined 5′ DNA sequence of clone number 91240.2.

SEQ ID NO:223 is a determined 5′ DNA sequence of clone number 91241.2.

SEQ ID NO:224 is a determined 5′ DNA sequence of clone number 91242.5.

SEQ ID NO:225 is a determined 5′ DNA sequence of clone number 91243.6.

SEQ ID NO:226 is a determined 5′ DNA sequence of clone number 91245.2.

SEQ ID NO:227 is a determined 5′ DNA sequence of clone number 91246.4.

SEQ ID NO:228 is a determined 3′ DNA sequence of clone number 91247.3.

SEQ ID NO:229 is a determined 5′ DNA sequence of clone number 91247.4.

SEQ ID NO:230 is a determined 5′ DNA sequence of clone number 91249.2.

SEQ ID NO:231 is a determined 5′ DNA sequence of clone number 91253.2.

SEQ ID NO:232 is a determined 5′ DNA sequence of clone number 91254.2.

SEQ ID NO:233 is a determined 5′ DNA sequence of clone number 91259.2.

SEQ ID NO:234 is a determined 3′ DNA sequence of clone number 91261.3.

SEQ ID NO:235 is a determined 5′ DNA sequence of clone number 91261.4.

SEQ ID NO:236 is a determined 5′ DNA sequence of clone number 91262.2.

SEQ ID NO:237 is a determined 5′ DNA sequence of clone number 91263.2.

SEQ ID NO:238 is a determined 5′ DNA sequence of clone number 91264.2.

SEQ ID NO:239 is a determined 5′ DNA sequence of clone number 91268.2.

SEQ ID NO:240 is a determined 5′ DNA sequence of clone number 91269.5.

SEQ ID NO:241 is a determined 5′ DNA sequence of clone number 91271.5.

SEQ ID NO:242 is a determined 3′ DNA sequence of clone number 91273.3.

SEQ ID NO:243 is a determined 5′ DNA sequence of clone number 91274.6.

SEQ ID NO:244 is the DNA sequence of GenBank Accession Number 18549403, which shares homology to SEQ ID NO:246.

SEQ ID NO:245 is the DNA sequence of GenBank Accession Number 10436393_FLJ14035, which shares homology to SEQ ID NO:246.

SEQ ID NO:246, also referred to as O646SgenomicContig, is a DNA (contig) sequence assembled based on a search of the publicly available databases using SEQ ID NO:243 as a query.

SEQ ID NO:247 is a amino acid sequence corresponding to the DNA sequence of GenBank Accession Number 18549403, SEQ ID NO:244.

SEQ ID NO:248 is a amino acid sequence corresponding to the DNA sequence of GenBank Accession Number 10436393_FLJ14035, SEQ ID NO:245.

SEQ ID NO:249 is a amino acid sequence corresponding to a polypeptide encoded by SEQ ID NO:246, also referred to as O646GenomicContig_Major ORF.

SEQ ID NO:250 is the DNA sequence of GenBank Accession Number 3980529, which shares homology to SEQ ID NO:262.

SEQ ID NO:251 is the DNA sequence of GenBank Accession Number 13629915, which shares homology to SEQ ID NO:262.

SEQ ID NO:252 is the DNA sequence of GenBank Accession Number 9789986, which shares homology to SEQ ID NO:262.

SEQ ID NO:253 is the DNA sequence of GenBank Accession Number 6006516, which shares homology to SEQ ID NO:262.

SEQ ID NO:254 is the DNA sequence of GenBank Accession Number 5689424, which shares homology to SEQ ID NO:262.

SEQ ID NO:255 is the DNA sequence of GenBank Accession Number 15638833, which shares homology to SEQ ID NO:262.

SEQ ID NO:256, also referred to as O646SGenomicContig, is a DNA (contig) sequence assembled based on a search of the publicly available databases using SEQ ID NO:243 as a query.

SEQ ID NO:257 is an amino acid sequence corresponding to the DNA sequence of GenBank Accession Number 13629915, SEQ ID NO:251.

SEQ ID NO:258 is an amino acid sequence corresponding to the DNA sequence of GenBank Accession Number 9789986, SEQ ID NO:252.

SEQ ID NO:259 is an amino acid sequence corresponding to the DNA sequence of GenBank Accession Number 6006516, SEQ ID NO:253.

SEQ ID NO:260 is an amino acid sequence corresponding to the DNA sequence of GenBank Accession Number 5689424, SEQ ID NO:254.

SEQ ID NO:261, also referred to as O648S_GenomicContig_ORF, is a amino acid sequence corresponding to a polypeptide encoded by SEQ ID NO:262.

SEQ ID NO:262 is the DNA sequence of GenBank Accession Number 16933560, which shares homology to SEQ ID NO:268.

SEQ ID NO:263 is the DNA sequence of GenBank Accession Number 12053028, which shares homology to SEQ ID NO:268.

SEQ ID NO:264 is the DNA sequence of GenBank Accession Number 7638812, which shares homology to SEQ ID NO:268.

SEQ ID NO:265 is the DNA sequence of GenBank Accession Number 939922, which shares homology to SEQ ID NO:268.

SEQ ID NO:266 is the DNA sequence of GenBank Accession Number 6093230, which shares homology to SEQ ID NO:268.

SEQ ID NO:267 is the DNA sequence of GenBank Accession Number 11465000, which shares homology to SEQ ID NO:268.

SEQ ID NO:268 also referred to as O647SgenomicContig3, is a DNA (contig) sequence assembled based on a search of the publicly available databases using SEQ ID NO:234 as a query.

SEQ ID NO:269 is an amino acid sequence corresponding to the DNA sequence of GenBank Accession Number 16933560, SEQ ID NO:262.

SEQ ID NO:270 is an amino acid sequence corresponding to the DNA sequence of GenBank Accession Number 12053028, SEQ ID NO:263.

SEQ ID NO:271 is an amino acid sequence corresponding to the DNA sequence of GenBank Accession Number 7638812, SEQ ID NO:264.

SEQ ID NO:272 is an amino acid sequence corresponding to the DNA sequence of GenBank Accession Number 939922, SEQ ID NO:265.

SEQ ID NO:273 also referred to as O645SgenomicContig2, is a DNA (contig) sequence assembled based on a search of the publicly available databases using SEQ ID NO:238 as a query.

SEQ ID NO:274 is the DNA sequence of GenBank Accession Number NM006580, also referred to as Claudin16, which shares homology to SEQ ID NO:277.

SEQ ID NO:275 is the DNA sequence of GenBank Accession NumberAF152101.1, also referred to as Paracellin-1, which shares homology to SEQ ID NO:277.

SEQ IN NO:276 is the DNA sequence of GenBank Accession Number 18425237, which shares homology to SEQ ID NO:277.

SEQ ID NO:277 also referred to as O644SgenomicContig2, is a DNA (contig) sequence assembled based on a search of the publicly available databases using SEQ ID NO:240 as a query.

SEQ ID NO:278 is an amino acid sequence corresponding to the DNA sequence of GenBank Accession Number NM006580, SEQ ID NO:277.

SEQ ID NO:279 is an amino acid sequence corresponding to the DNA sequence of GenBank Accession Number AF152101.1, SEQ ID NO:275.

SEQ ID NO:280 also referred to as O644S_GenomicContig2_ORF1, is a amino acid sequence corresponding to an open reading frame of SEQ ID NO:277.

SEQ ID NO:281 also referred to as O644S_GenomicContig2_ORF2, is a amino acid sequence corresponding to an open reading frame of SEQ ID NO:277.

SEQ ID NO:282 also referred to as O644S_GenomicContig2_ORF3, is a amino acid sequence corresponding to an open reading frame of SEQ ID NO:277.

SEQ ID NO:283 is a DNA sequence of a signal peptide minus O591S fusion protein containing a N-terminal histidine tag.

SEQ ID NO:284 is a corresponding amino acid sequence of a signal peptide minus O591S fusion protein containing a N-terminal histidine tag.

SEQ ID NO:285 is a 1740 bp DNA sequence identified by BlastN search of a LifeSeq Gold database using SEQ ID NO:198 as a query.

SEQ ID NO:286 is an amino acid sequence encode by the DNA sequence set forth in SEQ ID NO:285.

SEQ ID NO:287 is the sequence for the forward primer, CBH-005, used in the amplification of O591S-A.

SEQ ID NO:288 is the sequence for the reverse primer, CBH-003, used in the amplification of O591S-A.

SEQ ID NO:289 corresponds to the amino acid sequence corresponding to residue 1-114 of SEQ ID NO:215.

SEQ ID NO:290 corresponds to the amino acid sequence corresponding to residue 1-115 of SEQ ID NO:215 (O591S).

SEQ ID NO: 291 corresponds to amino acid residues 26-55 of SEQ ID NO:215 (O591S).

SEQ ID NO:292 corresponds to amino acid residues 53-78 of SEQ ID NO:215 (O591S).

SEQ ID NO:293 corresponds to amino acid residues 103-129 of SEQ ID NO:215 (O591S).

### DETAILED DESCRIPTION OF THE INVENTION

U.S. patents, U.S. patent application publications, U.S. patent applications, foreign patents, foreign patent applications and non-patent publications referred to in this specification and/or listed in the Application Data Sheet, are incorporated herein by reference, in their entirety.

The present invention is directed generally to compositions and their use in the therapy and diagnosis of cancer, particularly ovarian cancer. As described further below, illustrative compositions of the present invention include, but are not restricted to, polypeptides, particularly immunogenic polypeptides, polynucleotides encoding such polypeptides, antibodies and other binding agents, antigen presenting cells (APCS) and immune system cells (e.g., T cells).

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Polypeptide Compositions

As used herein, the term “polypeptide” “is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising epitopes, i.e., antigenic determinants substantially responsible for the immunogenic properties of a polypeptide and being capable of evoking an immune response.

Particularly illustrative polypeptides of the present invention comprise those encoded by a polynucleotide sequence set forth in any one of SEQ ID NO:1, 2, 5, 9, 10, 13, 16, 19, 23, 27, 28, 32, 33, 35, 38, 41-50, 52, 53, 56, 57, 63, 65, 69-72, 75, 78, 80-82, 84, 86, 89-93, 95, 97-100, 103, 107, 111, 114, 117, 120, 121, 125, 128, 132-134, 136, 137, 140, 143-146, 148-151, 156, 158, 160-162, 166-168, 171, 174-183, 185, 193-199, 203-206, 208, 210-214, 216-246, 250-256, 262-268, 273-277, 283, 285, and 287-288 or a sequence that hybridizes under moderately stringent conditions, or, alternatively, under highly stringent conditions, to a polynucleotide sequence identified above. Certain other illustrative polypeptides of the invention comprise amino acid sequences as set forth in any one of SEQ ID NO:200-202, 207, 209, 215, 247-249, 257-261, 269-272, 278-282, 284, 286, and 289-293.

The polypeptides of the present invention are sometimes herein referred to as ovarian tumor proteins or ovarian tumor polypeptides, as an indication that their identification has been based at least in part upon their increased levels of expression in ovarian tumor samples. Thus, a “ovarian tumor polypeptide” or “ovarian tumor protein,” refers generally to a polypeptide sequence of the present invention, or a polynucleotide sequence encoding such a polypeptide, that is expressed in a substantial proportion of ovarian tumor samples, for example preferably greater than about 20%, more preferably greater than about 30%, and most preferably greater than about 50% or more of ovarian tumor samples tested, at a level that is at least two fold, and preferably at least five fold, greater than the level of expression in normal tissues, as determined using a representative assay provided herein. An ovarian tumor polypeptide sequence of the invention, based upon its increased level of expression in tumor cells, has particular utility both as a diagnostic marker as well as a therapeutic target, as further described below.

In certain preferred embodiments, the polypeptides of the invention are immunogenic, i.e., they react detectably within an immunoassay (such as an ELISA or T-cell stimulation assay) with antisera and/or T-cells from a patient with ovarian cancer. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, immunogenic portions of the polypeptides disclosed herein are also encompassed by the present invention. An “immunogenic portion,” as used herein, is a fragment of an immunogenic polypeptide of the invention that itself is immunologically reactive (i.e., specifically binds) with the B-cells and/or T-cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques.

In one preferred embodiment, an immunogenic portion of a polypeptide of the present invention is a portion that reacts with antisera and/or T-cells at a level that is not substantially less than the reactivity of the full-length polypeptide (e.g., in an ELISA and/or T-cell reactivity assay). Preferably, the level of immunogenic activity of the immunogenic portion is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

In certain other embodiments, illustrative immunogenic portions may include peptides in which an N-terminal leader sequence and/or transmembrane domain have been deleted. Other illustrative immunogenic portions will contain a small N- and/or C-terminal deletion (e.g., 1-30 amino acids, preferably 5-15 amino acids), relative to the mature protein.

In another embodiment, a polypeptide composition of the invention may also comprise one or more polypeptides that are immunologically reactive with T cells and/or antibodies generated against a polypeptide of the invention, particularly a polypeptide having an amino acid sequence disclosed herein, or to an immunogenic fragment or variant thereof.

In another embodiment of the invention, polypeptides are provided that comprise one or more polypeptides that are capable of eliciting T cells and/or antibodies that are immunologically reactive with one or more polypeptides described herein, or one or more polypeptides encoded by contiguous nucleic acid sequences contained in the polynucleotide sequences disclosed herein, or immunogenic fragments or variants thereof, or to one or more nucleic acid sequences which hybridize to one or more of these sequences under conditions of moderate to high stringency.

The present invention, in another aspect, provides polypeptide fragments comprising at least about 5, 10, 15, 20, 25, 50, or 100 contiguous amino acids, or more, including all intermediate lengths, of a polypeptide compositions set forth herein, such as those set forth in SEQ ID NO:200-202, 207, 209, 215, 247-249, 257-261, 269-272, 278-282, 284, 286, and 289-293 or those encoded by a polynucleotide sequence set forth in any one of SEQ ID NO:1, 2, 5, 9, 10, 13, 16, 19, 23, 27, 28, 32, 33, 35, 38, 41-50, 52, 53, 56, 57, 63, 65, 69-72, 75, 78, 80-82, 84, 86, 89-93, 95, 97-100, 103, 107, 111, 114, 117, 120, 121, 125, 128, 132-134, 136, 137, 140, 143-146, 148-151, 156, 158, 160-162, 166-168, 171, 174-183, 185, 193-199, 203-206, 208, 210-214, 216-246, 250-256, 262-268, 273-277, 283, 285, 287-288.

In another aspect, the present invention provides variants of the polypeptide compositions described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequences set forth herein.

In one preferred embodiment, the polypeptide fragments and variants provide by the present invention are immunologically reactive with an antibody and/or T-cell that reacts with a full-length polypeptide specifically set for the herein.

In another preferred embodiment, the polypeptide fragments and variants provided by the present invention exhibit a level of immunogenic activity of at least about 50%, preferably at least about 70%, and most preferably at least about 90% or more of that exhibited by a full-length polypeptide sequence specifically set forth herein.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their immunogenic activity as described herein and/or using any of a number of techniques well known in the art.

For example, certain illustrative variants of the polypeptides of the invention include those in which one or more portions, such as an N-terminal leader sequence or transmembrane domain, have been removed. Other illustrative variants include variants in which a small portion (e.g., 1-30 amino acids, preferably 5-15 amino acids) has been removed from the N- and/or C-terminal of the mature protein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics. When it is desired to alter the amino acid sequence of a polypeptide to create an equivalent, or even an improved, immunogenic variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

TABLE I

| Amino Acids | | Codons | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | GAG | | | | |
| Arginine | Arg | R | AGA | AGG | CGA | CGC | CGG | CGU |
| Serine | Ser | S | AGC | AGU | UCA | UCC | UCG | UGU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gIn, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

Within other illustrative embodiments, a polypeptide may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al. New Engl. J. Med., 336:86-91, 1997).

In one preferred embodiment, the immunological fusion partner is derived from a Mycobacterium sp., such as a Mycobacterium tuberculosis-derived Ra12 fragment. Ra12 compositions and methods for their use in enhancing the expression and/or immunogenicity of heterologous polynucleotide/polypeptide sequences is described in U.S. Patent Application No. 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. Patent Application No. 60/158,585; see also, Skeiky et al., Infection and Immun. (1999) 67:3998-4007, incorporated herein by reference). C-terminal fragments of the MTB32A coding sequence express at high levels and remain as a soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class 11 molecules and thereby provide enhanced in vivo stimulation of CD4⁺ T-cells specific for the polypeptide.

Polypeptides of the invention are prepared using any of a variety of well known synthetic and/or recombinant techniques, the latter of which are further described below. Polypeptides, portions and other variants generally less than about 150 amino acids can be generated by synthetic means, using techniques well known to those of ordinary skill in the art. In one illustrative example, such polypeptides are synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

In general, polypeptide compositions (including fusion polypeptides) of the invention are isolated. An “isolated” polypeptide is one that is removed from its original environment. For example, a naturally-occurring protein or polypeptide is isolated if it is separated from some or all of the coexisting materials in the natural system. Preferably, such polypeptides are also purified, e.g., are at least about 90% pure, more preferably at least about 95% pure and most preferably at least about 99% pure.

Polynucleotide Compositions

The present invention, in other aspects, provides polynucleotide compositions. The terms “DNA” and “polynucleotide” are used essentially interchangeably herein to refer to a DNA molecule that has been isolated free of total genomic DNA of a particular species. “Isolated,” as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

As will be understood by those skilled in the art, the polynucleotide compositions of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be also recognized by the skilled artisan, polynucleotides of the invention may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a polypeptide/protein of the invention or a portion thereof or may comprise a sequence that encodes a variant or derivative, preferably and immunogenic variant or derivative, of such a sequence.

Therefore, according to another aspect of the present invention, polynucleotide compositions are provided that comprise some or all of a polynucleotide sequence set forth in any one of SEQ ID NO:1, 2, 5, 9, 10, 13, 16, 19, 23, 27, 28, 32, 33, 35, 38, 41-50, 52, 53, 56, 57, 63, 65, 69-72, 75, 78, 80-82, 84, 86, 89-93, 95, 97-100, 103, 107, 111, 114, 117, 120, 121, 125, 128, 132-134, 136, 137, 140, 143-146, 148-151, 156, 158, 160-162, 166-168, 171, 174-183, 185, 193-199, 203-206, 208, 210-214, 216-246, 250-256, 262-268, 273-277, 283, 285, 287-288, complements of a polynucleotide sequence set forth as described above, and degenerate variants of a polynucleotide sequence set forth as described above. In certain preferred embodiments, the polynucleotide sequences set forth herein encode immunogenic polypeptides, as described above.

In other related embodiments, the present invention provides polynucleotide variants having substantial identity to the sequences disclosed herein in SEQ ID NO:1, 2, 5, 9, 10, 13, 16, 19, 23, 27, 28, 32, 33, 35, 38, 41-50, 52, 53, 56, 57, 63, 65, 69-72, 75, 78, 80-82, 84, 86, 89-93, 95, 97-100, 103, 107, 111, 114, 117, 120, 121, 125, 128, 132-134, 136, 137, 140, 143-146, 148-151, 156, 158, 160-162, 166-168, 171, 174-183, 185, 193-199, 203-206, 208, 210-214, 216-246, 250-256, 262-268, 273-277, 283, 285, 287-288, for example those comprising at least 70% sequence identity, preferably at least 75%, 80%, 85%, 90%, 95%, 96%, 97%, 98%, or 99% or higher, sequence identity compared to a polynucleotide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the immunogenicity of the polypeptide encoded by the variant polynucleotide is not substantially diminished relative to a polypeptide encoded by a polynucleotide sequence specifically set forth herein). The term “variants” should also be understood to encompasses homologous genes of xenogenic origin.

In additional embodiments, the present invention provides polynucleotide fragments comprising various lengths of contiguous stretches of sequence identical to or complementary to one or more of the sequences disclosed herein. For example, polynucleotides are provided by this invention that comprise at least about 10, 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

In another embodiment of the invention, polynucleotide compositions are provided that are capable of hybridizing under moderate to high stringency conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5×and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60-65° C. or 65-70° C.

In certain preferred embodiments, the polynucleotides described above, e.g., polynucleotide variants, fragments and hybridizing sequences, encode polypeptides that are immunologically cross-reactive with a polypeptide sequence specifically set forth herein. In other preferred embodiments, such polynucleotides encode polypeptides that have a level of immunogenic activity of at least about 50%, preferably at least about 70%, and more preferably at least about 90% of that for a polypeptide sequence specifically set forth herein.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=-4 and a comparison of both strands.

Preferably, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Therefore, in another embodiment of the invention, a mutagenesis approach, such as site-specific mutagenesis, is employed for the preparation of immunogenic variants and/or derivatives of the polypeptides described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments of the present invention, the inventors contemplate the mutagenesis of the disclosed polynucleotide sequences to alter one or more properties of the encoded polypeptide, such as the immunogenicity of a polypeptide vaccine. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et a., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants of the present invention, recursive sequence recombination, as described in U.S. Pat. No. 5,837,458, may be employed. In this approach, iterative cycles of recombination and screening or selection are performed to “evolve” individual polynucleotide variants of the invention having, for example, enhanced immunogenic activity.

In other embodiments of the present invention, the polynucleotide sequences provided herein can be advantageously used as probes or primers for nucleic acid hybridization. As such, it is contemplated that nucleic acid segments that comprise a sequence region of at least about 15 nucleotide long contiguous sequence that has the same sequence as, or is complementary to, a 15 nucleotide long contiguous sequence disclosed herein will find particular utility. Longer contiguous identical or complementary sequences, e.g., those of about 20, 30, 40, 50, 100, 200, 500, 1000 (including all intermediate lengths) and even up to full length sequences will also be of use in certain embodiments.

The ability of such nucleic acid probes to specifically hybridize to a sequence of interest will enable them to be of use in detecting the presence of complementary sequences in a given sample. However, other uses are also envisioned, such as the use of the sequence information for the preparation of mutant species primers, or primers for use in preparing other genetic constructions.

Polynucleotide molecules having sequence regions consisting of contiguous nucleotide stretches of 10-14, 15-20, 30, 50, or even of 100-200 nucleotides or so (including intermediate lengths as well), identical or complementary to a polynucleotide sequence disclosed herein, are particularly contemplated as hybridization probes for use in, e.g., Southern and Northern blotting. This would allow a gene product, or fragment thereof, to be analyzed, both in diverse cell types and also in various bacterial cells. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15 and about 100 nucleotides, but larger contiguous complementarity stretches may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design nucleic acid molecules having gene-complementary stretches of 15 to 25 contiguous nucleotides, or even longer where desired.

Hybridization probes may be selected from any portion of any of the sequences disclosed herein. All that is required is to review the sequences set forth herein, or to any continuous portion of the sequences, from about 15-25 nucleotides in length up to and including the full length sequence, that one wishes to utilize as a probe or primer. The choice of probe and primer sequences may be governed by various factors. For example, one may wish to employ primers from towards the termini of the total sequence.

Small polynucleotide segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the PCRTM technology of U.S. Pat. No. 4,683,202 (incorporated herein by reference), by introducing selected sequences into recombinant vectors for recombinant production, and by other recombinant DNA techniques generally known to those of skill in the art of molecular biology.

The nucleotide sequences of the invention may be used for their ability to selectively form duplex molecules with complementary stretches of the entire gene or gene fragments of interest. Depending on the application envisioned, one will typically desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by a salt concentration of from about 0.02 M to about 0.15 M salt at temperatures of from about 50° C. to about 70° C. Such selective conditions tolerate little, if any, mismatch between the probe and the template or target strand, and would be particularly suitable for isolating related sequences.

Of course, for some applications, for example, where one desires to prepare mutants employing a mutant primer strand hybridized to an underlying template, less stringent (reduced stringency) hybridization conditions will typically be needed in order to allow formation of the heteroduplex. In these circumstances, one may desire to employ salt conditions such as those of from about 0.15 M to about 0.9 M salt, at temperatures ranging from about 20° C. to about 55° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

According to another embodiment of the present invention, polynucleotide compositions comprising antisense oligonucleotides are provided. Antisense oligonucleotides have been demonstrated to be effective and targeted inhibitors of protein synthesis, and, consequently, provide a therapeutic approach by which a disease can be treated by inhibiting the synthesis of proteins that contribute to the disease. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (U.S. Pat. No. 5,739,119 and U.S. Pat. No. 5,759,829). Further, examples of antisense inhibition have been demonstrated with the nuclear protein cyclin, the multiple drug resistance gene (MDG1), ICAM-1, E-selectin, STK-1, striatal GABAA receptor and human EGF (Jaskulski et al., Science. Jun. 10, 1988;240(4858):1544-6; Vasanthakumar and Ahmed, Cancer Commun. 1989;1(4):225-32; Peris et al., Brain Res Mol Brain Res. Jun. 15, 1998;57(2):310-20; U.S. Pat. No. 5,801,154; U.S. Pat. No. 5,789,573; U.S. Pat. No. 5,718,709 and U.S. Pat. No. 5,610,288). Antisense constructs have also been described that inhibit and can be used to treat a variety of abnormal cellular proliferations, e.g., cancer (U.S. Pat. No. 5,747,470; U.S. Pat. No. 5,591,317 and U.S. Pat. No. 5,783,683).

Therefore, in certain embodiments, the present invention provides oligonucleotide sequences that comprise all, or a portion of, any sequence that is capable of specifically binding to polynucleotide sequence described herein, or a complement thereof. In one embodiment, the antisense oligonucleotides comprise DNA or derivatives thereof. In another embodiment, the oligonucleotides comprise RNA or derivatives thereof. In a third embodiment, the oligonucleotides are modified DNAs comprising a phosphorothioated modified backbone. In a fourth embodiment, the oligonucleotide sequences comprise peptide nucleic acids or derivatives thereof. In each case, preferred compositions comprise a sequence region that is complementary, and more preferably substantially-complementary, and even more preferably, completely complementary to one or more portions of polynucleotides disclosed herein. Selection of antisense compositions specific for a given gene sequence is based upon analysis of the chosen target sequence and determination of secondary structure, Tm, binding energy, and relative stability. Antisense compositions may be selected based upon their relative inability to form dimers, hairpins, or other secondary structures that would reduce or prohibit specific binding to the target mRNA in a host cell. Highly preferred target regions of the mRNA, are those which are at or near the AUG translation initiation codon, and those sequences which are substantially complementary to 5′ regions of the mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17):3389-402).

The use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen (Morris et al., Nucleic Acids Res. Jul. 15, 1997;25(14):2730-6). It has been demonstrated that several molecules of the MPG peptide coat the antisense oligonucleotides and can be delivered into cultured mammalian cells in less than 1 hour with relatively high efficiency (90%). Further, the interaction with MPG strongly increases both the stability of the oligonucleotide to nuclease and the ability to cross the plasma membrane.

According to another embodiment of the invention, the polynucleotide compositions described herein are used in the design and preparation of ribozyme molecules for inhibiting expression of the tumor polypeptides and proteins of the present invention in tumor cells. Ribozymes are RNA-protein complexes that cleave nucleic acids in a site-specific fashion. Ribozymes have specific catalytic domains that possess endonuclease activity (Kim and Cech, Proc Natl Acad Sci USA. 1987 December;84(24):8788-92; Forster and Symons, Cell. Apr. 24, 1987;49(2):211-20). For example, a large number of ribozymes accelerate phosphoester transfer reactions with a high degree of specificity, often cleaving only one of several phosphoesters in an oligonucleotide substrate (Cech et ai., Cell. 1981 Dec;27(3 Pt 2):487-96; Michel and Westhof, J. Mol. Biol. Dec. 5, 1990;216(3):585-610; Reinhold-Hurek and Shub, Nature. May 14, 1992;357(6374):173-6). This specificity has been attributed to the requirement that the substrate bind via specific base-pairing interactions to the internal guide sequence (“IGS”) of the ribozyme prior to chemical reaction.

Six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., Proc Natl Acad Sci USA. Aug. 15, 1992;89(16):7305-9). Thus, the specificity of action of a ribozyme is greater than that of an antisense oligonucleotide binding the same RNA site.

The enzymatic nucleic acid molecule may be formed in a hammerhead, hairpin, a hepatitis 8 virus, group I intron or RNaseP RNA (in association with an RNA guide sequence) or Neurospora VS RNA motif. Examples of hammerhead motifs are described by Rossi et al. Nucleic Acids Res. Sep. 11, 1992;20(17):4559-65. Examples of hairpin motifs are described by Hampel et al. (Eur. Pat. Appl. Publ. No. EP 0360257), Hampel and Tritz, Biochemistry Jun. 13, 1989;28(12):4929-33; Hampel et al., Nucleic Acids Res. Jan. 25, 1990;18(2):299-304 and U.S. Pat. No. 5,631,359. An example of the hepatitis 8 virus motif is described by Perrotta and Been, Biochemistry. Dec. 1, 1992; 31 (47): 11843-52; an example of the RNaseP motif is described by Guerrier-Takada et al., Cell. 1983 December;35(3 Pt 2):849-57; Neurospora VS RNA ribozyme motif is described by Collins (Saville and Collins, Cell. May 18, 1990;61(4):685-96; Saville and Collins, Proc Natl Acad Sci USA. Oct. 1, 1991;88(19):8826-30; Collins and Olive, Biochemistry. Mar. 23, 1993;32(11):2795-9); and an example of the Group I intron is described in (U.S. Pat. No. 4,987,071). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

Ribozymes may be designed as described in Int. Pat. Appl. Publ. No. WO 93/23569 and Int. Pat. Appl. Publ. No. WO 94/02595, each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Ribozyme activity can be optimized by altering the length of the ribozyme binding arms, or chemically synthesizing ribozymes with modifications that prevent their degradation by serum ribonucleases (see, e.g., Int. Pat. Appl. PubI. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Sullivan et al. (Int. Pat. Appl. Publ. No. WO 94/02595) describes the general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Int. Pat. Appl. Publ. No. WO 94/02595 and Int. Pat. Appl. Publ. No. WO 93/23569, each specifically incorporated herein by reference.

Another means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells Ribozymes expressed from such promoters have been shown to function in mammalian cells. Such transcription units can be incorporated into a variety of vectors for introduction into mammalian cells, including but not restricted to, plasmid DNA vectors, viral DNA vectors (such as adenovirus or adeno-associated vectors), or viral RNA vectors (such as retroviral, semliki forest virus, sindbis virus vectors).

In another embodiment of the invention, peptide nucleic acids (PNAs) compositions are provided. PNA is a DNA mimic in which the nucleobases are attached to a pseudopeptide backbone (Good and Nielsen, Antisense Nucleic Acid Drug Dev. 1997 7(4) 431-37). PNA is able to be utilized in a number methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol 1997 June;15(6):224-9). As such, in certain embodiments, one may prepare PNA sequences that are complementary to one or more portions of the ACE mRNA sequence, and such PNA compositions may be used to regulate, alter, decrease, or reduce the translation of ACE-specific mRNA, and thereby alter the level of ACE activity in a host cell to which such PNA compositions have been administered.

PNAs have 2-aminoethyl-glycine linkages replacing the normal phosphodiester backbone of DNA (Nielsen et al., Science Dec. 6, 1991;254(5037):1497-500; Hanvey et al., Science. Nov. 27, 1992;258(5087):1481-5; Hyrup and Nielsen, Bioorg Med Chem. 1996 January;4(1):5-23). This chemistry has three important consequences: firstly, in contrast to DNA or phosphorothioate oligonucleotides, PNAs are neutral molecules; secondly, PNAs are achiral, which avoids the need to develop a stereoselective synthesis; and thirdly, PNA synthesis uses standard Boc or Fmoc protocols for solid-phase peptide synthesis, although other methods, including a modified Merrifield method, have been used.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg Med Chem. 1995 April;3(4):437-45). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography, providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (for example, Norton et al., Bioorg Med Chem. 1995 April;3(4):437-45; Petersen et al., J Pept Sci. 1995 May-Jun;1(3):175-83; Orum et al., Biotechniques. 1995 September; 119(3):472-80; Footer et al., Biochemistry. Aug. 20, 1996;35(33):10673-9; Griffith et al., Nucleic Acids Res. Aug. 11, 1995;23(15):3003-8; Pardridge et al., Proc Natl Acad Sci USA. Jun. 6, 1995;92(12):5592-6; Boffa et al., Proc Natl Acad Sci USA. Mar. 14, 1995;92(6):1901-5; Gambacorti-Passerini et al., Blood. Aug. 15, 1996;88(4):1411-7; Armitage et al., Proc Natl Acad Sci USA. Nov. 11, 1997; 94(23):12320-5; Seeger et al., Biotechniques. 1997 September;23(3):512-7). U.S. Pat. No. 5,700,922 discusses PNA-DNA-PNA chimeric molecules and their uses in diagnostics, modulating protein in organisms, and treatment of conditions susceptible to therapeutics.

Methods of characterizing the antisense binding properties of PNAs are discussed in Rose (Anal Chem. Dec. 15, 1993;65(24):3545-9) and Jensen et al. (Biochemistry. Apr. 22, 1997;36(16):5072-7). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs that have been described and will be apparent to the skilled artisan include use in DNA strand invasion, antisense inhibition, mutational analysis, enhancers of transcription, nucleic acid purification, isolation of transcriptionally active genes, blocking of transcription factor binding, genome cleavage, biosensors, in situ hybridization, and the like.

Polynucleotide Identification, Characterization and Expression

Polynucleotides compositions of the present invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references). For example, a polynucleotide may be identified, as described in more detail below, by screening a microarray of cDNAs for tumor-associated expression (i.e., expression that is at least two fold greater in a tumor than in normal tissue, as determined using a representative assay provided herein). Such screens may be performed, for example, using the microarray technology of Affymetrix, Inc. (Santa Clara, Calif.) according to the manufacturer's instructions (and essentially as described by Schena et al., Proc. Nat. Acad. Sci. USA 93:10614-10619, 1996 and Heller et al., Proc. Natl. Acad. Sci. USA 94:2150-2155, 1997). Alternatively, polynucleotides may be amplified from cDNA prepared from cells expressing the proteins described herein, such as tumor cells.

Many template dependent processes are available to amplify a target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Any of a number of other template dependent processes, many of which are variations of the PCR TM amplification technique, are readily known and available in the art. Illustratively, some such methods include the ligase chain reaction (referred to as LCR), described, for example, in Eur. Pat. Appl. Publ. No. 320,308 and U.S. Pat. No. 4,883,750; Qbeta Replicase, described in PCT Intl. Pat. Appl. Pubi. No. PCT/US87/00880; Strand Displacement Amplification (SDA) and Repair Chain Reaction (RCR). Still other amplification methods are described in Great Britain Pat. Appl. No. 2 202 328, and in PCT Intl. Pat. Appl. Pubi. No. PCT/US89/01025. Other nucleic acid amplification procedures include transcription-based amplification systems (TAS) (PCT Intl. Pat. Appl. Publ. No. WO 88/10315), including nucleic acid sequence based amplification (NASBA) and 3SR. Eur. Pat. Appl. Publ. No. 329,822 describes a nucleic acid amplification process involving cyclically synthesizing single-stranded RNA (“ssRNA”), ssDNA, and double-stranded DNA (dsDNA). PCT Intl. Pat. Appl. Publ. No. WO 89/06700 describes a nucleic acid sequence amplification scheme based on the hybridization of a promoter/primer sequence to a target single-stranded DNA (“ssDNA”) followed by transcription of many RNA copies of the sequence. Other amplification methods such as “RACE” (Frohman, 1990), and “one-sided PCR” (Ohara, 1989) are also well-known to those of skill in the art.

An amplified portion of a polynucleotide of the present invention may be used to isolate a full length gene from a suitable library (e.g., a tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²P) using well known techniques. A bacterial or bacteriophage library is then generally screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full length cDNA molecule can be generated by ligating suitable fragments, using well known techniques.

Alternatively, amplification techniques, such as those described above, can be useful for obtaining a full length coding sequence from a partial cDNA sequence. One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

In other embodiments of the invention, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences may be ligated to a heterologous sequence to encode a fusion protein. For example, to screen peptide libraries for inhibitors of polypeptide activity, it may be useful to encode a chimeric protein that can be recognized by a commercially available antibody. A fusion protein may also be engineered to contain a cleavage site located between the polypeptide-encoding sequence and the heterologous protein sequence, so that the polypeptide may be cleaved and purified away from the heterologous moiety.

Sequences encoding a desired polypeptide may be synthesized, in whole or in part, using chemical methods well known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the protein itself may be produced using chemical methods to synthesize the amino acid sequence of a polypeptide, or a portion thereof. For example, peptide synthesis can be performed using various solid-phase techniques (Roberge, J. Y. et al. (1995) Science 269:202-204) and automated synthesis may be achieved, for example, using the ABI 431A Peptide Synthesizer (Perkin Elmer, Palo Alto, Calif.).

A newly synthesized peptide may be substantially purified by preparative high performance liquid chromatography (e.g., Creighton, T. (1983) Proteins, Structures and Molecular Principles, W H Freeman and Co., New York, N.Y.) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In order to express a desired polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, any of a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of .beta.-galactosidase so that a hybrid protein is produced; pIN vectors (Van Heeke, G. and S. M. Schuster (1989) J. Biol. Chem. 264:5503-5509); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, N. (1987) EMBO J. 6:307-311. Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard, E. K. et al. (1994) Proc. Natl. Acad. Sci. 91:3224-3227).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, COS, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1990) Cell 22:817-23) genes which can be employed in tk.sup.- or aprt.sup.-cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides, neomycin and G-418 (Colbere-Garapin, F. et al (1981) J. Mol. Biol. 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). The use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif. 3:263-281) while the enterokinase cleavage site provides a means for purifying the desired polypeptide from the fusion protein. A discussion of vectors which contain fusion proteins is provided in Kroll, D. J. et al. (1993; DNA Cell Biol. 12:441-453).

In addition to recombinant production methods, polypeptides of the invention, and fragments thereof, may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

Antibody Compositions, Fragments thereof and Other Binding Agents

According to another aspect, the present invention further provides binding agents, such as antibodies and antigen-binding fragments thereof, that exhibit immunological binding to a tumor polypeptide disclosed herein, or to a portion, variant or derivative thereof. An antibody, or antigen-binding fragment thereof, is said to “specifically bind,” “immunogically bind,” and/or is “immunologically reactive” to a polypeptide of the invention if it reacts at a detectable level (within, for example, an ELISA assay) with the polypeptide, and does not react detectably with unrelated polypeptides under similar conditions.

Immunological binding, as used in this context, generally refers to the non-covalent interactions of the type which occur between an immunoglobulin molecule and an antigen for which the immunoglobulin is specific. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al. (1990) Annual Rev. Biochem. 59:439-473.

An “antigen-binding site,” or “binding portion” of an antibody refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Binding agents may be further capable of differentiating between patients with and without a cancer, such as ovarian cancer, using the representative assays provided herein. For example, antibodies or other binding agents that bind to a tumor protein will preferably generate a signal indicating the presence of a cancer in at least about 20% of patients with the disease, more preferably at least about 30% of patients. Alternatively, or in addition, the antibody will generate a negative signal indicating the absence of the disease in at least about 90% of individuals without the cancer. To determine whether a binding agent satisfies this requirement, biological samples (e.g., blood, sera, sputum, urine and/or tumor biopsies) from patients with and without a cancer (as determined using standard clinical tests) may be assayed as described herein for the presence of polypeptides that bind to the binding agent. Preferably, a statistically significant number of samples with and without the disease will be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of therapeutically useful molecules are known in the art which comprise antigen-binding sites that are capable of exhibiting immunological binding properties of an antibody molecule. The proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment which comprises both antigen-binding sites. An “Fv” fragment can be produced by preferential proteolytic cleavage of an IgM, and on rare occasions IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set which provide support to the CDRS and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J. Immunol. 138:4534-4538; and Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients.

As used herein, the terms “veneered FRs” and “recombinantly veneered FRs” refer to the selective replacement of FR residues from, e.g., a rodent heavy or light chain V region, with human FR residues in order to provide a xenogeneic molecule comprising an antigen-binding site which retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface. Davies et al. (1990) Ann. Rev. Biochem. 59:439-473. Thus, antigen binding specificity can be preserved in a humanized antibody only wherein the CDR structures, their interaction with each other, and their interaction with the rest of the V region domains are carefully maintained. By using veneering techniques, exterior (e.g., solvent-accessible) FR residues which are readily encountered by the immune system are selectively replaced with human residues to provide a hybrid molecule that comprises either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments. There are two general steps in veneering a murine antigen-binding site. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR which differ from the human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids.

In this manner, the resultant “veneered” murine antigen-binding sites are thus designed to retain the murine CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs which are believed to influence the “canonical” tertiary structures of the CDR loops. These design criteria are then used to prepare recombinant nucleotide sequences which combine the CDRs of both the heavy and light chain of a murine antigen-binding site into human-appearing FRs that can be used to transfect mammalian cells for the expression of recombinant human antibodies which exhibit the antigen specificity of the murine antibody molecule.

In another embodiment of the invention, monoclonal antibodies of the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

T Cell Compositions

The present invention, in another aspect, provides T cells specific for a tumor polypeptide disclosed herein, or for a variant or derivative thereof. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. No. 5,240,856; U.S. Pat. No. 5,215,926; WO 89/06280; WO 91/16116 and WO 92/07243). Alternatively, T cells may be derived from related or unrelated humans, non-human mammals, cell lines or cultures.

T cells may be stimulated with a polypeptide, polynucleotide encoding a polypeptide and/or an antigen presenting cell (APC) that expresses such a polypeptide. Such stimulation is performed under conditions and for a time sufficient to permit the generation of T cells that are specific for the polypeptide of interest. Preferably, a tumor polypeptide or polynucleotide of the invention is present within a delivery vehicle, such as a microsphere, to facilitate the generation of specific T cells.

T cells are considered to be specific for a polypeptide of the present invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a tumor polypeptide (100 ng/ml-100 μg/ml, preferably 200 ng/ml-25 μg/ml) for 3-7 days will typically result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). T cells that have been activated in response to a tumor polypeptide, polynucleotide or polypeptide-expressing APC may be CD4⁺ and/or CD8⁺. Tumor polypeptide-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺ T cells that proliferate in response to a tumor polypeptide, polynucleotide or APC can be expanded in number either in vitro or in vivo. Proliferation of such T cells in vitro may be accomplished in a variety of ways. For example, the T cells can be re-exposed to a tumor polypeptide, or a short peptide corresponding to an immunogenic portion of such a polypeptide, with or without the addition of T cell growth factors, such as interleukin-2, and/or stimulator cells that synthesize a tumor polypeptide. Alternatively, one or more T cells that proliferate in the presence of the tumor polypeptide can be expanded in number by cloning. Methods for cloning cells are well known in the art, and include limiting dilution.

Pharmaceutical Compositions

In additional embodiments, the present invention concerns formulation of one or more of the polynucleotide, polypeptide, T-cell and/or antibody compositions disclosed herein in pharmaceutically-acceptable carriers for administration to a cell or an animal, either alone, or in combination with one or more other modalities of therapy.

It will be understood that, if desired, a composition as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Therefore, in another aspect of the present invention, pharmaceutical compositions are provided comprising one or more of the polynucleotide, polypeptide, antibody, and/or T-cell compositions described herein in combination with a physiologically acceptable carrier. In certain preferred embodiments, the pharmaceutical compositions of the invention comprise immunogenic polynucleotide and/or polypeptide compositions of the invention for use in prophylactic and therapeutic vaccine applications. Vaccine preparation is generally described in, for example, M. F. Powell and M. J. Newman, eds., “Vaccine Design (the subunit and adjuvant approach),” Plenum Press (NY, 1995). Generally, such compositions will comprise one or more polynucleotide and/or polypeptide compositions of the present invention in combination with one or more immunostimulants.

It will be apparent that any of the pharmaceutical compositions described herein can contain pharmaceutically acceptable salts of the polynucleotides and polypeptides of the invention. Such salts can be prepared, for example, from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

In another embodiment, illustrative immunogenic compositions, e.g., vaccine compositions, of the present invention comprise DNA encoding one or more of the polypeptides as described above, such that the polypeptide is generated in situ. As noted above, the polynucleotide may be administered within any of a variety of delivery systems known to those of ordinary skill in the art. Indeed, numerous gene delivery techniques are well known in the art, such as those described by Rolland, Crit. Rev. Therap. Drug Carrier Systems 15:143-198, 1998, and references cited therein. Appropriate polynucleotide expression systems will, of course, contain the necessary regulatory DNA regulatory sequences for expression in a patient (such as a suitable promoter and terminating signal). Alternatively, bacterial delivery systems may involve the administration of a bacterium (such as Bacillus-Calmette-Guerrin) that expresses an immunogenic portion of the polypeptide on its cell surface or secretes such an epitope.

Therefore, in certain embodiments, polynucleotides encoding immunogenic polypeptides described herein are introduced into suitable mammalian host cells for expression using any of a number of known viral-based systems. In one illustrative embodiment, retroviruses provide a convenient and effective platform for gene delivery systems. A selected nucleotide sequence encoding a polypeptide of the present invention can be inserted into a vector and packaged in retroviral particles using techniques known in the art. The recombinant virus can then be isolated and delivered to a subject. A number of illustrative retroviral systems have been described (e.g., U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

In addition, a number of illustrative adenovirus-based systems have also been described. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476).

Various adeno-associated virus (MV) vector systems have also been developed for polynucleotide delivery. MV vectors can be readily constructed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors useful for delivering the polynucleotides encoding polypeptides of the present invention by gene transfer include those derived from the pox family of viruses, such as vaccinia virus and avian poxyirus. By way of example, vaccinia virus recombinants expressing the novel molecules can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK.sup.(−) recombinant can be selected by culturing the cells in the presence of 5-bromodeoxyuridine and picking viral plaques resistant thereto.

A vaccinia-based infection/transfection system can be conveniently used to provide for inducible, transient expression or coexpression of one or more polypeptides described herein in host cells of an organism. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide or polynucleotides of interest, driven by a T7 promoter. The polymerase expressed in the cytoplasm from the vaccinia virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al. Proc. Natl. Acad. Sci. USA (1986) 83:8122-8126.

Alternatively, avipoxyiruses, such as the fowlpox and canarypox viruses, can also be used to deliver the coding sequences of interest. Recombinant avipox viruses, expressing immunogens from mammalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxyiruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al. J. Biol. Chem. (1993) 268:6866-6869 and Wagner et al. Proc. Natl. Acad. Sci. USA (1992) 89:6099-6103, can also be used for gene delivery under the invention.

Additional illustrative information on these and other known viral-based delivery systems can be found, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993.

In certain embodiments, a polynucleotide may be integrated into the genome of a target cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronization with the host cell cycle. The manner in which the expression construct is delivered to a cell and where in the cell the polynucleotide remains is dependent on the type of expression construct employed.

In another embodiment of the invention, a polynucleotide is administered/delivered as “naked” DNA, for example as described in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

In still another embodiment, a composition of the present invention can be delivered via a particle bombardment approach, many of which have been described. In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

According to another embodiment, the pharmaceutical compositions described herein will comprise one or more immunostimulants in addition to the immunogenic polynucleotide, polypeptide, antibody, T-cell and/or APC compositions of this invention. An immunostimulant refers to essentially any substance that enhances or potentiates an immune response (antibody and/or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Within certain embodiments of the invention, the adjuvant composition is preferably one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Certain preferred adjuvants for eliciting a predominantly Th1-type response include, for example, a combination of monophosphoryl lipid A, preferably 3-de-O-acylated monophosphoryl lipid A, together with an aluminum salt. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other preferred formulations include more than one saponin in the adjuvant combinations of the present invention, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol_{R} to increase viscosity, or may be formulated in a dry powder form with a powder excipient such as lactose.

In one preferred embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other preferred formulations comprise an oil-in-water emulsion and tocopherol. Another particularly preferred adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 is disclosed in WO 00/09159. Preferably the formulation additionally comprises an oil in water emulsion and tocopherol.

Additional illustrative adjuvants for use in the pharmaceutical compositions of the invention include Montamide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Enhanzyn®) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other preferred adjuvants include adjuvant molecules of the general formula

HO(CH₂CH₂O)ₙ-A-R, (I):

wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₋₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described in the pending UK patent application GB 9820956.2.

According to another embodiment of this invention, an immunogenic composition described herein is delivered to a host via antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, fit3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcy receptor and mannose receptor. The mature phenotype is typically characterized by a lower expression of these markers, but a high expression of cell surface molecules responsible for T cell activation such as class I and class 11 MHC, adhesion molecules (e.g., CD54 and CD11) and costimulatory molecules (e.g., CD40, CD80, CD86 and 4-1BB).

APCs may generally be transfected with a polynucleotide of the invention (or portion or other variant thereof such that the encoded polypeptide, or an immunogenic portion thereof, is expressed on the cell surface. Such transfection may take place ex vivo, and a pharmaceutical composition comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

While any suitable carrier known to those of ordinary skill in the art may be employed in the pharmaceutical compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

In another illustrative embodiment, biodegradable microspheres (e.g., polylactate polyglycolate) are employed as carriers for the compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems such as described in WO/99 40934, and references cited therein, will also be useful for many applications. Another illustrative carrier/delivery system employs a carrier comprising particulate-protein complexes, such as those described in U.S. Pat. No. 5,928,647, which are capable of inducing a class I-restricted cytotoxic T lymphocyte responses in a host.

The pharmaceutical compositions of the invention will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The pharmaceutical compositions described herein may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles. Alternatively, a pharmaceutical composition may be stored in a freeze-dried condition requiring only the addition of a sterile liquid carrier immediately prior to use.

The development of suitable dosing and treatment regimens for using the particular compositions described herein in a variety of treatment regimens, including e.g., oral, parenteral, intravenous, intranasal, and intramuscular administration and formulation, is well known in the art, some of which are briefly discussed below for general purposes of illustration.

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (see, for example, Mathiowitz et al., Nature Mar. 27, 1997;386(6623):410-4; Hwang et al., Crit Rev Ther Drug Carrier Syst 1998;15(3):243-84; U.S. Pat. No. 5,641,515; U.S. Pat. No. 5,580,579 and U.S. Pat. No. 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparation and formulations.

Typically, these formulations will contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In certain circumstances it will be desirable to deliver the pharmaceutical compositions disclosed herein parenterally, intravenously, intramuscularly, or even intraperitoneally. Such approaches are well known to the skilled artisan, some of which are further described, for example, in U.S. Pat. No. 5,543,158; U.S. Pat. No. 5,641,515 and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. No. 5,756,353 and U.S. Pat. No. 5,804,212. Likewise, the delivery of drugs using intranasal microparticle resins (Takenaga et al., J Controlled Release Mar. 2, 1998;52(1-2):81-7) and lysophosphatidyl-glycerol compounds (U.S. Pat. No. 5,725,871) are also well-known in the pharmaceutical arts. Likewise, illustrative transmucosal drug delivery in the form of a polytetrafluoroetheylene support matrix is described in U.S. Pat. No. 5,780,045.

In certain embodiments, liposomes, nanocapsules, microparticles, lipid particles, vesicles, and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

The formation and use of liposome and liposome-like preparations as potential drug carriers is generally known to those of skill in the art (see for example, Lasic, Trends Biotechnol 1998 July;16(7):307-21; Takakura, Nippon Rinsho 1998 March;56(3):691-5; Chandran et al., Indian J Exp Biol. 1997 August;35(8):801-9; Margalit, Crit Rev Ther Drug Carrier Syst. 1995;12(2-3):233-61; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally difficult to transfect by other procedures, including T cell suspensions, primary hepatocyte cultures and PC 12 cells (Renneisen et al., J. Biol. Chem. Sep. 25, 1990;265(27):16337-42; Muller et al., DNA Cell Biol. 1990 April;9(3):221-9). In addition, liposomes are free of the DNA length constraints that are typical of viral-based delivery systems. Liposomes have been used effectively to introduce genes, various drugs, radiotherapeutic agents, enzymes, viruses, transcription factors, allosteric effectors and the like, into a variety of cultured cell lines and animals. Furthermore, he use of liposomes does not appear to be associated with autoimmune responses or unacceptable toxicity after systemic delivery.

In certain embodiments, liposomes are formed from phospholipids that are dispersed in an aqueous medium and spontaneously form multilamellar concentric bilayer vesicles (also termed multilamellar vesicles (MLVs).

Alternatively, in other embodiments, the invention provides for pharmaceutically-acceptable nanocapsule formulations of the compositions of the present invention. Nanocapsules can generally entrap compounds in a stable and reproducible way (see, for example, Quintanar-Guerrero et al., Drug Dev Ind Pharm. 1998 Dec;24(12):1113-28). To avoid side effects due to intracellular polymeric overloading, such ultrafine particles (sized around 0.1 μm) may be designed using polymers able to be degraded in vivo. Such particles can be made as described, for example, by Couvreur et al., Crit Rev Ther Drug Carrier Syst. 1988;5(1):1-20; zur Muhlen et al., EurJ Pharm Biopharm. 1998 March;45(2):149-55; Zambaux et al. J Controlled Release. Jan. 2, 1998;50(1-3):31-40; and U.S. Pat. No. 5,145,684.

Cancer Therapeutic Methods

In further aspects of the present invention, the pharmaceutical compositions described herein may be used for the treatment of cancer, particularly for the immunotherapy of ovarian cancer. Within such methods, the pharmaceutical compositions described herein are administered to a patient, typically a warm-blooded animal, preferably a human. A patient may or may not be afflicted with cancer. Accordingly, the above pharmaceutical compositions may be used to prevent the development of a cancer or to treat a patient afflicted with a cancer. Pharmaceutical compositions and vaccines may be administered either prior to or following surgical removal of primary tumors and/or treatment such as administration of radiotherapy or conventional chemotherapeutic drugs. As discussed above, administration of the pharmaceutical compositions may be by any suitable method, including administration by intravenous, intraperitoneal, intramuscular, subcutaneous, intranasal, intradermal, anal, vaginal, topical and oral routes.

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 25 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a tumor protein generally correlate with an improved clinical outcome. Such immune responses may generally be evaluated using standard proliferation, cytotoxicity or cytokine assays, which may be performed using samples obtained from a patient before and after treatment.

Cancer Detection and Diagnostic Compositions, Methods and Kits

In general, a cancer may be detected in a patient based on the presence of one or more ovarian tumor proteins and/or polynucleotides encoding such proteins in a biological sample (for example, blood, sera, sputum urine and/or tumor biopsies) obtained from the patient. In other words, such proteins may be used as markers to indicate the presence or absence of a cancer such as ovarian cancer. In addition, such proteins may be useful for the detection of other cancers. The binding agents provided herein generally permit detection of the level of antigen that binds to the agent in the biological sample. Polynucleotide primers and probes may be used to detect the level of mRNA encoding an ovarian tumor protein, which is also indicative of the presence or absence of a cancer. In general, a ovarian tumor sequence should be present at a level that is at least three fold higher in tumor tissue than in normal tissue

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length ovarian tumor proteins and polypeptide portions thereof to which the binding agent binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at Al 2-Al 3).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with ovarian cancer. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as ovarian cancer, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the tumor proteins or binding agents of the present invention. The above descriptions are intended to be exemplary only. For example, it will be apparent to those of ordinary skill in the art that the above protocols may be readily modified to use tumor polypeptides to detect antibodies that bind to such polypeptides in a biological sample. The detection of such tumor protein specific antibodies may correlate with the presence of a cancer.

A cancer may also, or alternatively, be detected based on the presence of T cells that specifically react with a tumor protein in a biological sample. Within certain methods, a biological sample comprising CD4⁺ and/or CD8⁺ T cells isolated from a patient is incubated with a tumor polypeptide, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with polypeptide (e.g., 5-25 μg/ml). It may be desirable to incubate another aliquot of a T cell sample in the absence of tumor polypeptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a cancer in the patient.

As noted above, a cancer may also, or alternatively, be detected based on the level of mRNA encoding a tumor protein in a biological sample. For example, at least two oligonucleotide primers may be employed in a polymerase chain reaction (PCR) based assay to amplify a portion of a tumor cDNA derived from a biological sample, wherein at least one of the oligonucleotide primers is specific for (i.e., hybridizes to) a polynucleotide encoding the tumor protein. The amplified cDNA is then separated and detected using techniques well known in the art, such as gel electrophoresis. Similarly, oligonucleotide probes that specifically hybridize to a polynucleotide encoding a tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a tumor protein of the invention that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence as disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

In another embodiment, the compositions described herein may be used as markers for the progression of cancer. In this embodiment, assays as described above for the diagnosis of a cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a tumor protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a tumor protein in a biological sample. Such kits generally comprise at least one oligonucleotide probe or primer, as described above, that hybridizes to a polynucleotide encoding a tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a tumor protein.

The following Examples are offered by way of illustration and not by way of limitation.

### EXAMPLES

### Example 1

### Identification of Representative Ovarian Carcinoma cDNA Sequences

Primary ovarian tumor and metastatic ovarian tumor cDNA libraries were each constructed in kanamycin resistant pZErO™-2 vector (Invitrogen) from pools of three different ovarian tumor RNA samples. For the primary ovarian tumor library, the following RNA samples were used: (1) a moderately differentiated papillary serous carcinoma of a 41 year old, (2) a stage IIIC ovarian tumor and (3) a papillary serous adenocarcinoma for a 50 year old Caucasian. For the metastatic ovarian tumor library, the RNA samples used were omentum tissue from: (1) a metastatic poorly differentiated papillary adenocarcinoma with psammoma bodies in a 73 year old, (2) a metastatic poorly differentiated adenocarcinoma in a 74 year old and (3) a metastatic poorly differentiated papillary adenocarcinoma in a 68 year old.

The number of clones in each library was estimated by plating serial dilutions of unamplified libraries. Insert data were determined from 32 primary ovarian tumor clones and 32 metastatic ovarian tumor clones. The library characterization results are shown in Table II.

TABLE II
CHARACTERIZATION OF cDNA LIBRARIES

| | # Clones | Clones with | Insert Size | Ave. Insert |
|---|---|---|---|---|
| Library | in Library | Insert (%) | Range (bp) | Size (bp) |
| Primary Ovarian | 1,258,000 | 97 | 175-8000 | 2356 |
| Tumor | | | | |
| Metastatic | 1,788,000 | 100 | 150-4300 | 1755 |
| Ovarian Tumor | | | | |

Four subtraction libraries were constructed in ampicillin resistant pcDNA3.1 vector (Invitrogen). Two of the libraries were from primary ovarian tumors and two were from metastatic ovarian tumors. In each case, the number of restriction enzyme cuts within inserts was minimized to generate full length subtraction libraries. The subtractions were each done with slightly different protocols, as described in more detail below.

A. POTS 2 Library: Primary Ovarian Tumor Subtraction Library

| | |
|---|---|
| Tracer: | 10 μg primary ovarian tumor library, digested with Not I |
| Driver: | 35 μg normal pancreas in pcDNA3.1(+) |
| | 20 μg normal PBMC in pcDNA3.1(+) |
| | 10 μg normal skin in pcDNA3.1(+) |
| | 35 μg normal bone marrow in pZErO ™-2 |
| | Digested with Bam HI/Xho I/Sca I |

Two hybridizations were performed, and Not 1-cut pcDNA3.1 (+) was the cloning vector for the subtracted library. Sequence results for previously unidentified clones that were randomly picked from the subtracted library are presented in Table III.

TABLE III
OVARIAN CARCINOMA SEQUENCES

| Sequence | SEQ ID NO |
|---|---|
| 21907 | 1 |
| 21909 | 2 |
| 21911 | 5 |
| 21920 | 9 |
| 21921 | 10 |
| 25099 | 143 |
| 25101 | 144 |
| 25103 | 145 |
| 25107 | 146 |
| 25111 | 148 |
| 25113 | 149 |
| 25115 | 150 |
| 25116 | 151 |
| 25752 | 156 |
| 25757 | 158 |
| 25763 | 160 |
| 25769 | 161 |
| 25770 | 162 |

B. POTS 7 Library: Primary Ovarian Tumor Subtraction Library

| | | |
|---|---|---|
| Tracer: | 10 μg | primary ovarian tumor library, digested with Not I |
| Driver | 35 μg | normal pancreas in pcDNA3.1(+) |
| | 20 μg | normal PBMC in pcDNA3.1(+) |
| | 10 μg | normal skin in pcDNA3.1(+) |
| | 35 μg | normal bone marrow in pZErO ™-2 |
| | | Digested with Bam HI/Xho I/Sca I |
| | ˜25 μg | pZErO ™-2, digested with Bam HI and Xho I |

Two hybridizations were performed, and Not 1-cut pcDNA3.1(+) was the cloning vector for the subtracted library. Sequence results for previously unidentified clones that were randomly picked from the subtracted library are presented in Table IV.

TABLE IV
OVARIAN CARCINOMA SEQUENCES

| Sequence | SEQ ID NO |
|---|---|
| 24937 | 125 |
| 24940 | 128 |
| 24946 | 132 |
| 24950 | 133 |
| 24951 | 134 |
| 24955 | 136 |
| 24956 | 137 |
| 25791 | 166 |
| 25796 | 167 |
| 25797 | 168 |
| 25804 | 171 |

C. OS1D Library: Metastatic Ovarian Tumor Subtraction Library

| | | |
|---|---|---|
| Tracer: | 10 μg | metastatic ovarian library in pZErO ™-2, digested |
| | | with Not I |
| Driver: | 24.5 μg | normal pancreas in pcDNA3.1 |
| | 14 μg | normal PBMC in pcDNA3.1 |
| | 14 μg | normal skin in pcDNA3.1 |
| | 24.5 μg | normal bone marrow in pZErO ™-2 |
| | 50 μg | pZErO ™-2, digested with Bam HI/Xho I/Sfu I |

Three hybridizations were performed, and the last two hybridizations were done with an additional 15 μg of biotinylated pZErO™-2 to remove contaminating pZErO™-2 vectors. The cloning vector for the subtracted library was pcDNA3.1/Not I cut. Sequence results for previously unidentified clones that were randomly picked from the subtracted library are presented in Table V.

TABLE V
Ovarian Carcinoma Sequences

| Sequence | SEQ ID NO |
|---|---|
| 23645.1 | 13 |
| 23660.1 | 16 |
| 23666.1 | 19 |
| 23679.1 | 23 |
| 24635 | 57 |
| 24647 | 63 |
| 24651 | 65 |
| 24661 | 69 |
| 24663 | 70 |
| 24664 | 71 |
| 24670 | 72 |
| 24675 | 75 |
| 24683 | 78 |

D. OS1F Library: Metastatic Ovarian Tumor Subtraction Library

| | | |
|---|---|---|
| Tracer: | 10 μg | metastatic ovarian tumor library, digested with Not I |
| Driver: | 12.8 μg | normal pancreas in pcDNA3.1 |
| | 7.3 μg | normal PBMC in pcDNA3.1 |
| | 7.3 μg | normal skin in pcDNA3.1 |
| | 12.8 μg | normal bone marrow in pZErO ™-2 |
| | 25 μg | pZErO ™-2, digested with Bam HI/Xho I/Sfu I |

One hybridization was performed. The cloning vector for the subtracted library was pcDNA3.1/Not I cut. Sequence results for previously unidentified clones that were randomly picked from the subtracted library are presented in Table VI.

TABLE VI
OVARIAN CARCINOMA SEQUENCES

| Sequence | SEQ ID NO |
|---|---|
| 24336 (79% with H. sapiens mitochondrial | 27 |
| genome (consensus sequence)) | |
| 24337 | 28 |
| 24341 (91% Homo sapiens chromosome 5, BAC | 32 |
| clone 249h5 (LBNL H149) | |
| 24344 | 33 |
| 24348 | 35 |
| 24351 | 38 |
| 24355 (91% Homo sapiens chromosome 17, | 41 |
| clone hCIT.91_J_4) | |
| 24356 | 42 |
| 24357 (87% S. scrofa mRNA for UDP glucose | 43 |
| pyrophosphorylase) | |
| 24358 | 44 |
| 24359 (78% Human mRNA for KIAA0111 gene, | 45 |
| complete cds) | |
| 24360 | 46 |
| 24361 | 47 |
| 24362 (88% Homo sapiens Chromosome 16 | 48 |
| BAC clone CIT987SK-A-233A7) | |
| 24363 (87% Homo sapiens eukaryotic translation | 49 |
| elongation factor 1 alpha 1 (EEF1A1) | |
| 24364 (89% Human DNA sequence from PAC | 50 |
| 27K14 on chromosome Xp11.3-Xp11.4) | |
| 24367 (89% Homo sapiens 12p13.3 BAC | 52 |
| RCPI11-935C2) | |
| 24368 | 53 |
| 24690 | 81 |
| 24692 | 82 |
| 24694 | 84 |
| 24696 | 86 |
| 24699 | 89 |
| 24701 | 90 |
| 24703 | 91 |
| 24704 (88% Homo sapiens chromosome 9, clone | 92 |
| hRPK.401_G_18) | |
| 24705 | 93 |
| 24707 | 95 |
| 24709 | 97 |
| 24711 | 98 |
| 24713 | 99 |
| 24714 (91% Human DNA sequence from clone | 100 |
| 125N5 on chromosome 6q26-27) | |
| 24717 (89% Homo sapiens proliferation- | 103 |
| associated gene A (natural killer-enhancing | |
| factor A) (PAGA) | |
| 24727 | 107 |
| 24732 | 111 |
| 24737 (84% Human ADP/ATP translocase | 114 |
| mRNA) | |
| 24741 | 117 |
| 24745 | 120 |
| 24746 | 121 |

The sequences in Table VII, which correspond to known sequences, were also identified in the above libraries.

TABLE VII
OVARIAN CARCINOMA SEQUENCES

| Identity | SEQ ID NO | Sequence | Library |
|---|---|---|---|
| H. sapiens DNA for muscle nicotinic | 3 | 21910 | POTS2 |
| acetylcholine receptor gene promotor, clone | | | |
| ICRFc105F02104 | | | |
| Homo sapiens complement Component 3 (C3) | 4 | 21913 | POTS2 |
| gene, exons 1-30. | | | |
| Homo sapiens SWI/SNF related, matrix | 6 | 21914 | POTS2 |
| associated, actin dependent regulator of | | | |
| chromatin, subfamily a, member 4 | | | |
| (SMARCA4) | | | |
| Human ferritin Heavy subunit mRNA, complete | 7 | 21915 | POTS2 |
| cds. | | | |
| Homo sapiens CGI-151 protein mRNA, | 8 | 21916 | POTS2 |
| complete cds | | | |
| Human BAC clone GS055K18 from 7p15-0p21 | 11 | 23636.1 | OS1D |
| HUMGFIBPA Human growth hormone- | 12 | 23637.1 | OS1D |
| dependent insulin-like growth factor-binding | | | |
| protein | | | |
| Homo sapiens ribosomal protein, large, P0 | 14 | 23647.1 | OS1D |
| (RPLP0) mRNA | | | |
| HUMTRPM2A Human TRPM-2 mRNA | 15 | 23657.1 | OS1D |
| HUMMTA Homo sapiens mitochondrial DNA | 17 | 23661.1 | OS1D |
| HSU78095 Homo sapiens placental bikunin | 18 | 23662.1 | OS1D |
| mRNA | | | |
| HUMTI227HC Human mRNA for TI-227H | 20 | 23669.1 | OS1D |
| HUMMTCG Human mitochondrion | 21 | 23673.1 | OS1D |
| Homo sapiens FK506-binding protein 1A | 22 | 23677.1 | OS1D |
| (12kD) (FKBP1A) mRNA | | | |
| Homo sapiens mRNA for zinc-finger DNA- | 24 | 24333 | OS1F |
| binding protein, complete cds | | | |
| Homo sapiens mRNA; cDNA DKFZp564E1962 | 25 | 24334 | OS1F |
| (from clone DKFZp564E1962) | | | |
| Homo sapiens tumor protein, translationally- | 26 | 24335 | OS1F |
| controlled 1 (TPT1) mRNA. | | | |
| Homo sapiens interleukin 1 receptor accessory | 29 | 24338 | OS1F |
| protein (IL1RAP) mRNA. | | | |
| Human mRNA for KIAA0026 gene | 30 | 24339 | OS1F |
| Homo sapiens K-Cl cotransporter KCC4 | 31 | 24340 | OS1F |
| mRNA, complete cds | | | |
| Homo sapiens nuclear chloride ion channel | 34 | 24345 | OS1F |
| protein (NCC27) mRNA | | | |
| Homo sapiens mRNA for DEPP (decidual | 36 | 24349 | OS1F |
| protein induced by progesterone) | | | |
| Homo sapiens atrophin-1 interacting protein 4 | 37 | 24350 | OS1F |
| (AIP4) mRNA | | | |
| Human collagenase type IV mRNA, 3′ end. | 39 | 24352 | OS1F |
| Human mRNA for T-cell cyclophilin | 40 | 24354 | OS1F |
| Homo sapiens tumor suppressing | 51 | 24366 | OS1F |
| subtransferable candidate 1 (TSSC1) | | | |
| Homo sapiens clone 24452 mRNA sequence | 54 | 24374 | OS1F |
| Homo sapiens eukaryotic translation | 55 | 24627 | OS1D |
| elongation factor 1 alpha 1 (EEF1A1) | | | |
| Genomic sequence from Human 9q34 | 56 | 24634 | OS1D |
| Human insulin-like growth factor-binding | 58 | 24636 | OS1D |
| protein-3 gene | | | |
| Human ribosomal protein L3 mRNA, 3′ end | 59 | 24638 | OS1D |
| Homo sapiens annexin II (lipocortin II) (ANX2) | 60 | 24640 | OS1D |
| mRNA | | | |
| Homo sapiens tubulin, alpha, ubiquitous (K- | 61 | 24642 | OS1D |
| ALPHA-1) | | | |
| Human non-histone chromosomal protein | 62 | 24645 | OS1D |
| HMG-14 mRNA | | | |
| Homo sapiens ferritin, heavy polypeptide 1 | 64 | 24648 | OS1D |
| (FTH1) | | | |
| Homo sapiens 12p13.3 PAC RPCI1-96H9 | 66 | 24653 | OS1D |
| (Roswell Park Cancer Institute Human | | | |
| PACLibrary) | | | |
| Homo sapiens T cell-specific tyrosine kinase | 67 | 24655 | OS1D |
| mRNA | | | |
| Homo sapiens keratin 18 (KRT18) mRNA | 68 | 24657 | OS1D |
| Homo sapiens growth arrest specific transcript | 73 | 24671 | OS1D |
| 5 gene | | | |
| Homo sapiens ribosomal protein S7 (RPS7) | 74 | 24673 | OS1D |
| Homo sapiens mRNA; cDNA DKFZp564H182 | 76 | 24677 | OS1D |
| Human TSC-22 protein mRNA | 77 | 24679 | OS1D |
| Human mRNA for ribosomal protein | 79 | 24687 | OS1D |
| Genomic sequence from Human 13 | 80 | 24689 | OS1F |
| Homo sapiens clone IMAGE 286356 | 83 | 24693 | OS1F |
| Homo sapiens v-fos FBJ murine osteosarcoma | 85 | 24695 | OS1F |
| viral oncogene homolog(FOS) mRNA | | | |
| Homo sapiens hypothetical 43.2 Kd protein | 87 | 24697 | OS1F |
| mRNA | | | |
| Human heat shock protein 27 (HSPB1) gene | 88 | 24698 | OS1F |
| exons 1-3 | | | |
| Homo sapiens senescence-associated | 94 | 24706 | OS1F |
| epithelial membrane protein (SEMP1) | | | |
| Human ferritin H chain mRNA | 96 | 24708 | OS1F |
| Homo sapiens mRNA for KIAA0287 gene | 101 | 24715 | OS1F |
| Homo sapiens CGI-08 protein mRNA | 102 | 24716 | OS1F |
| H. sapiens CpG island DNA genomic Mse1 | 104 | 24719 | OS1F |
| fragment, clone 84a5 | | | |
| Human clone 23722 mRNA | 105 | 24721 | OS1F |
| Homo sapiens zinc finger protein slug (SLUG) | 106 | 24722 | OS1F |
| gene | | | |
| Homo sapiens (clone L6) E-cadherin (CDH1) | 108 | 24728 | OS1F |
| gene | | | |
| Homo sapiens ribosomal protein L13 (RPL13) | 109 | 24729 | OS1F |
| H. sapiens RNA for snRNP protein B | 110 | 24730 | OS1F |
| Homo sapiens mRNA; cDNA DKFZp434K114 | 112 | 24734 | OS1F |
| Homo sapiens cornichon protein mRNA | 113 | 24735 | OS1F |
| Homo sapiens keratin 8 (KRT8) mRNA | 115 | 24739 | OS1F |
| Human DNA sequence from PAC 29K1 on | 116 | 24740 | OS1F |
| chromosome 6p21.3-22.2. | | | |
| Homo sapiens mRNA for KIAA0762 protein | 118 | 24742 | OS1F |
| Human clones 23667 and 23775 zinc finger | 119 | 24744 | OS1F |
| protein mRNA | | | |
| Human H19 RNA gene, complete cds. | 122 | 24933 | POTS7 |
| Human triosephosphate isomerase mRNA, | 123 | 24934 | POTS7 |
| complete cds. | | | |
| Human cyclooxygenase-1 (PTSG1) mRNA, | 124 | 24935 | POTS7 |
| partial cds | | | |
| Homo sapiens megakaryocyte potentiating | 126 | 24938 | POTS7 |
| factor (MPF) mRNA. | | | |
| Human mRNA for Apo1_Human (MER5(Aop1- | 127 | 24939 | POTS7 |
| Mouse)-like protein), complete cds | | | |
| Homo sapiens arylacetamide deacetylase | 129 | 24942 | POTS7 |
| (esterase) (AADAC) mRNA. | | | |
| Homo sapiens echinoderm microtubule- | 130 | 24943 | POTS7 |
| associated protein-like EMAP2 mRNA, | | | |
| complete cds | | | |
| Homo sapiens podocalyxin-like (PODXL) | 131 | 24944 | POTS7 |
| mRNA. | | | |
| Homo sapiens synaptogyrin 2 (SYNGR2) | 135 | 24952 | POTS7 |
| mRNA. | | | |
| Homo sapiens amyloid beta precursor protein- | 138 | 24959 | POIS7 |
| binding protein 1, 59kD (APPBP1) mRNA. | | | |
| Human aldose reductase mRNA, complete | 139 | 24969 | POTS7 |
| cds. | | | |
| Genomic sequence from Human 9q34, | 140 | 25092 | POTS2 |
| complete sequence [Homo sapiens] | | | |
| Human glyceraldehyde-3-phosphate | 141 | 25093 | POTS2 |
| dehydrogenase (GAPDH) mRNA, complete | | | |
| cds. | | | |
| Homo sapiens breast cancer suppressor | 142 | 25098 | POTS2 |
| candidate 1 (bcsc-1) mRNA, complete cds | | | |
| Homo sapiens SKB1 (S. cerevisiae) homolog | 147 | 25110 | POTS2 |
| (SKB1) mRNA. | | | |
| Homo sapiens prepro dipeptidyl peptidase I | 152 | 25117 | POTS2 |
| (DPP-I) gene, complete cds | | | |
| Homo sapiens preferentially expressed antigen | 153 | 25745 | POTS2 |
| of melanoma (PRAME) mRNA | | | |
| Human translocated t(8;14) c-myc (MYC) | 154 | 25746 | POTS2 |
| oncogene, exon 3 and complete cds | | | |
| Human 12S RNA induced by poly(rl), poly(rC) | 155 | 25749 | POTS2 |
| and Newcastle disease virus | | | |
| Human mRNA for fibronectin (FN precursor) | 157 | 25755 | POTS2 |
| Homo sapiens mRNA for hepatocyte growth | 159 | 25758 | POTS2 |
| factor activator inhibitor type 2,complete cds | | | |
| Homo sapiens mRNA for KIAA0552 protein, | 163 | 25771 | POTS7 |
| complete cds | | | |
| Homo sapiens IMP (inosine monophosphate) | 164 | 25775 | POTS7 |
| dehydrogenase 2 (IMPDH2) mRNA | | | |
| Homo sapiens clone 23942 alpha enolase | 165 | 25787 | POTS7 |
| mRNA, partial cds | | | |
| H. sapiens vegf gene, 3′UTR | 169 | 25799 | POTS7 |
| Homo sapiens 30S ribosomal protein S7 | 170 | 25802 | POTS7 |
| homolog mRNA, complete cds | | | |
| Homo sapiens acetyl-Coenzyme A | 172 | 25808 | POTS7 |
| acetyltransferase 2 (acetoacetyl Coenzyme A | | | |
| thiolase) (ACAT2) mRNA | | | |
| Homo sapiens Norrie disease protein (NDP) | 173 | 25809 | POTS7 |
| mRNA | | | |

Still further ovarian carcinoma polynucleotide and/or polypeptide sequences identified from the above libraries are provided below in Table VIII. Sequences O574S (SEQ ID NO:183 & 185), O584S (SEQ ID NO:193) and O585S (SEQ ID NO:194) represent novel sequences. The remaining sequences exhibited at least some homology with known genomic and/or EST sequences.

TABLE VIII

| SEQ ID: | Sequence | Library |
|---|---|---|
| 174: | O565S_CRABP | OS1D |
| 175: | O566S_Ceruloplasmin | POTS2 |
| 176: | O567S_41191.SEQ(1>487) | POTS2 |
| 177: | O568S_KIAA0762.seq(1>3999) | POTS7 |
| 178: | O569S_41220.seq(1>1069) | POTS7 |
| 179: | O570S_41215.seq(1>1817) | POTS2 |
| 180: | O571S_41213.seq(1>2382) | POTS2 |
| 181: | O572S_41208.seq(1>2377) | POTS2 |
| 182: | O573S_41177.seq(1>1370) | OS1F |
| 183: | O574S_47807.seq(1>2060) | n/a |
| 184: | O568S/VSGF DNA seq | n/a |
| 185: | O574S_47807.seq(1 >3000) | n/a |
| 186: | O568S/VSGF protein seq | n/a |
| 187: | 449H1(57581) | OS1D |
| 188: | 451E12(57582) | OS1D |
| 189: | 453C7_3′(57583.1)Osteonectin | OS1D |
| 190: | 453C7_5′(57583.2) | OS1D |
| 191: | 456G1_3′(57584.1)Neurotensin | OS1F |
| 192: | 456G1_5′(57584.2) | OS1F |
| 193: | O584S_465G5(57585) | OS1F |
| 194: | O585S_469B12(57586) | POTS2 |
| 195: | O569S_474C3(57587) | POTS7 |
| 196: | 483B1_3′(24934.1)Triosephosphate | POTS7 |
| 197: | 57885 Human preferentially expressed | POTS2 |
| | antigen of melanoma | |
| 198: | 57886 Chromosome 22q12.1 clone CTA- | POTS2 |
| | 723E4 | |
| 199: | 57887 Homologous to mouse brain cDNA | POTS2 |
| | clone MNCb-0671 | |

Further studies on the clone of SEQ ID NO:182 (also referred to as O573S) led to the identification of multiple open reading frames that encode the amino acid sequences of SEQ ID NO:200-202.

### Example 2

### Analysis of cDNA Expression Using Microarray Technology

In additional studies, sequences disclosed herein were found to be overexpressed in specific tumor tissues as determined by microarray analysis. Using this approach, cDNA sequences are PCR amplified and their mRNA expression profiles in tumor and normal tissues are examined using cDNA microarray technology essentially as described (Shena et al., 1995). In brief, the clones are arrayed onto glass slides as multiple replicas, with each location corresponding to a unique cDNA clone (as many as 5500 clones can be arrayed on a single slide or chip). Each chip is hybridized with a pair of cDNA probes that are fluorescence-labeled with Cy3 and Cy5 respectively. Typically, 1 μg of polyA⁺ RNA is used to generate each cDNA probe. After hybridization, the chips are scanned and the fluorescence intensity recorded for both Cy3 and Cy5 channels. There are multiple built-in quality control steps. First, the probe quality is monitored using a panel of ubiquitously expressed genes. Secondly, the control plate also can include yeast DNA fragments of which complementary RNA may be spiked into the probe synthesis for measuring the quality of the probe and the sensitivity of the analysis. Currently, the technology offers a sensitivity of 1 in 100,000 copies of mRNA. Finally, the reproducitility of this technology can be ensured by including duplicated control cDNA elements at different locations.

The microarray results for clones 57885 (SEQ ID NO: 197), 57886 (SEQ ID NO:198) and 57887 (SEQ ID NO:199) are as follows.

Clone 57885: 16/38 (42%) of ovarian tumors showed an expression signal value of >0.4. The mean value for all ovary tumors was 0.662 with a mean value of 0.187 for all normal tissues, which yields a 3.64 fold overexpression level in ovary tumor relative to essential normal tissues. Normal tissue expression was elevated (>0.4) in peritoneum, skin and thymus.

Clone 57886: 16/38 (42%) of ovarian tumors showed an expression signal value of >0.4. The mean value for all ovary tumors was 0.574 with a mean value of 0.166 for all normal tissues which yields a 3.46 fold overexpression level in ovary tumor relative to essential normal tissues. Normal tissue expression was elevated (>0.4) in heart, pancreas and small intestive.

Clone 57887: 17/38 (44%) of ovarian tumors showed an expression signal value of >0.4. The mean value for all ovary tumors is 0.744 with a mean value of 0.184 for all normal tissues which yields a 4.04 fold overexpression level in ovary tumor relative to essential normal tissues. Normal tissue expression was elevated (>0.4) in esophagus.

### Example 3

### Expression of Recombinant Antigen O568S in E. coli

This example describes the expression of recombinant antigen O568S (SEQ ID NO:177) in E. coli. This sequence was identified in Example 1 from the POTS 7 subtraction library using primary ovarian tumor cDNA as the tracer. PCR primers specific for the open reading frame of O568S were designed and used in the specific amplification of O568S. The PCR product was enzymatically digested with EcoRI and ligated into pPDM, a modified pET28 vector which had been cut with the restriction enzymes EcoRI and Eco72I. The construct sequence and orientation was confirmed through sequence analysis, the sequence of which is shown in SEQ ID NO:206. The vector was then transformed into the expression hosts, BLR (DE3) and HMS 174 (DE3) pLys S. Protein expression was confirmed, the sequence of which is provided in SEQ ID NO:207.

### Example 4

### Additional Sequence Obtained for Clone O591 S

The sequence of O591S (clone identifier 57887) was used to search public sequence databases. It was found that the reverse strand showed some degree of identity to the C-terminal end of GPR39. The cDNA for the coding region of GPR39 is disclosed in SEQ ID NO:208 and the corresponding amino acid sequence in SEQ ID NO:209. The GPR39 coding region contains two exons. Both O591 Sand GPR39, encoded by the complementary strand of O591S, are located on chromosome 2.

### Example 5

### Further Characterization of O591 S and Identification of Extended Sequence

O1034C is an ovary specific gene identified by electronic subtraction. Briefly, electronic subtraction involves an analysis of EST database sequences to identify ovarian-specific genes. In the electronic subtraction method used to identify O1034C, sequences of EST clones derived from ovary libraries (normal and tumor) were obtained from the GenBank public human EST database. Each ovary sequence was used as a “seed” query in a BLASTN search of the total human EST database to identify other EST clones that share sequence with the seed sequence (clones that potentially originated from the same mRNA). EST clones with shared sequence were grouped into clusters, and clusters that shared sequence with other clusters were grouped into superclusters. The tissue source of each EST within each supercluster was noted, and superclusters were ranked based on the distribution of the tissues from which the ESTs originated. Superclusters that comprise primarily, or solely, EST clones from ovary libraries were considered to represent genes that were differentially expressed in ovary tissue, relative to all other normal adult tissue.

This clone was identified from the public EST databases as Integrated Molecular Analysis of Genomics and their Expression (IMAGE) clone number 595449 (the IMAGE consortium is a repository of EST clones and cDNA clones) and is disclosed as SEQ ID NO:210. Accession numbers AA173739 and AA73383 represents the sequence of the identified EST in Genebank. This clone is part of Unigene cluster HS.85339 (Unigene is an experimental system for automatically partitioning Genbank sequences into a non-redundant set of gene-orientated clusters) and was annotated as encoding a neurotensin-like G protein coupled receptor (GRP39). However, the inventors have discovered that IMAGE#595449 encodes a novel protein derived from the complementary strand to that which encodes the potential GPR39.

Microarray analysis of the clone using a series of ovary tumor specific probes indicated that this clone was over expressed 4.95-fold in a group of ovary tumor and normal ovary samples as compared to a group of essential normal tissue samples.

IMAGE#59449 was subjected to a Blast A search of the EST database and Genbank and an electronic full length clone contig (O1034C) was generated by extending IMAGE#595449 and its resulting contigs to completion. This process was repeated to completion when no further EST sequences were identified to extend the consensus sequence. This electronically derived clone was identified as coding a previously described clone, O591 S, the sequence of which is disclosed in SEQ ID NO:211. The discovery of this ovary specific candidate is described in more detail in Example 4.

The consensus sequence for O1034C extended further 5′ than O591S due to the additional sequences derived from two EST clones, accession numbers BF345141 and BE336607, the sequences for which are disclosed in SEQ ID NO:212 and 213 respectively. Although BF345141 diverges from the O1034C/O591S consensus at its 3′-end (possibly representing a different splice form), and from BE336607 at several bases at its 5′-end, the two ESTs were compared to the available matching chromosome sequence. They were found on human chromosome 2, clone RP11-159N20:htgs database accession number AC010974. These sequences were used to extend O1034C/O591S to form a final consensus sequence for O1034C/O591S of 1897 base pairs, disclosed in SEQ ID NO:214.

An open reading frame (ORF) was identified within the O1034C/O591S consensus sequence (nucleotides 260-682), the predicted translation of which is disclosed in SEQ ID NO:215. A BLASTx database search against the Genbank database indicated that this ORF had no identity (E value <1e-25) with any known human protein. The only match was with the G protein-coupled receptors, including GPR39, which the inventors have shown to be encoded at the 3′-end of O1034C/O591 S on the complementary strand. However, the ORF did encode a protein that had 93% similarity (131/141 amino acids) and 91% identity (129/141 amino acids) with an un-named murine product (Accession #BAA95101), suggesting that this is a real translation product that represents a novel human ovary-specific antigen.

The novelty of O1034C/O591S was confirmed by Northern Blot analysis using single stranded probes that complement either GRP39 or O1034C/O591S. The strand-specific O1034C/O591S probe specifically hybridized to the ovary tumor samples probed on the Northern blot, whilst all samples were negative when probed with GPR39. In addition real-time PCR was performed using primers specific for either GPR39 or O1034C/O591S. These results further demonstrated the differential expression profiles of the two sequences. This protein is a putative membrane protein as determined from Corixa's Tmpred protein prediction algorithm.

### Example 6

### Expression Analysis and Further Characterization of Ovarian Sequence O568S

The ovarian sequence O568S was originally identified as cDNA clone 24742 (SEQ ID NO:118). Using clone 24742 as a query sequence to search public sequence databases, the sequence was found to have a high degree of homology with KIM0762 (SEQ ID NO:177) and with VSGF. The DNA sequence for VSGF is provided in SEQ ID 184 and the VSGF protein sequence is provided in SEQ ID NO:186.

Real-time PCR (see Gibson et al., Genome Research 6:995-1001, 1996; Heid et al., Genome Research 6:986-994, 1996) is a technique that evaluates the level of PCR product accumulation during amplification. This technique permits quantitative evaluation of mRNA levels in multiple samples. Briefly, mRNA is extracted from tumor and normal tissue and cDNA is prepared using standard techniques. Real-time PCR is performed, for example, using a Perkin Elmer/Applied Biosystems (Foster City, Calif.) 7700 Prism instrument. Matching primers and fluorescent probes are designed for genes of interest using, for example, the primer express program provided by Perkin Elmer/Applied Biosystems (Foster City, Calif.). Optimal concentrations of primers and probes are initially determined by those of ordinary skill in the art, and control (e.g., β-actin) primers and probes are obtained commercially from, for example, Perkin Elmer/Applied Biosystems (Foster City, Calif.). To quantitate the amount of specific RNA in a sample, a standard curve is generated using a plasmid containing the gene of interest. Standard curves are generated using the Ct values determined in the real-time PCR, which are related to the initial cDNA concentration used in the assay. Standard dilutions ranging from 10-10⁶ copies of the gene of interest are generally sufficient. In addition, a standard curve is generated for the control sequence. This permits standardization of initial RNA content of a tissue sample to the amount of control for comparison purposes.

By RealTime PCR analysis, O568 was highly overexpressed in the majority of ovary tumors and ovary tumor metastases tested relative to normal ovary tissue and relative to an extensive normal tissue panel. Little or no expression was observed in normal esophagus, spinal cord, bladder, colon, liver, PBMC (activated or resting), lung, skin, small intestine, stomach, skeletal muscle, pancreas, dendritic cells, heart, spleen bone marrow, thyroid, trachea, thymus, bronchia, cerebellum, ureter, uterus and peritoneum epithelium. Some low level expression was observed in normal breast, brain, bone, kidney, adrenal gland and salivary gland, but the expression levels in these normal tissues were generally at least several fold less than the levels observed in ovary tumors overexpressing O568S.

Moreover, a series of Northern blots was performed which also demonstrated that the ORF region of O568S is specifically overexpressed in ovary tumors. The initial blot contained RNA from a series of normal tissues as well as from ovary tumors. This blot was probed using, as a labeled probe, DNA from O568S that corresponded to the 3′UTR of the VSGF sequence disclosed in SEQ ID NO:184. This blot revealed an ovary tumor-specific 5.0 Kb message as well as a potential 3.5 Kb brain specific message and a ubiquitously expressed 1.35 Kb message.

Another Northern blot was performed with RNAs from a number of different brain tissues and probed with the 3′UTR region as above. Five of eleven brain samples showed overexpression of the 3.5 Kb message. In order to determine whether the ORF region of O568S was specifically overexpressed in ovary tumors, a series of three blots was carried out using three separate probes designed from within the VSGF ORF of O568S. Results from these experiments clearly indicated that only the 5.0 Kb message is expressed in ovary tumor.

### Example 7

### Synthesis of Polypeptides

Polypeptides are synthesized on a Perkin Elmer/Applied Biosystems Division 430A peptide synthesizer using FMOC chemistry with HPTU (O-Benzotriazole-N,N, N′,N′-tetramethyluronium hexafluorophosphate) activation. A Gly-Cys-Gly sequence is attached to the amino terminus of the peptide to provide a method of conjugation, binding to an immobilized surface, or labeling of the peptide. Cleavage of the peptides from the solid support is carried out using the following cleavage mixture: trifluoroacetic acid:ethanedithiol:thioanisole:water:phenol (40:1:2:2:3). After cleaving for 2 hours, the peptides are precipitated in cold methyl-t-butyl-ether. The peptide pellets are then dissolved in water containing 0.1% trifluoroacetic acid (TFA) and lyophilized prior to purification by C18 reverse phase HPLC. A gradient of 0%-60% acetonitrile (containing 0.1% TFA) in water (containing 0.1% TFA) is used to elute the peptides. Following lyophilization of the pure fractions, the peptides are characterized using electrospray or other types of mass spectrometry and by amino acid analysis.

### Example 8

### O568S Northern Blot Analysis

As described in Example 6, Northern blot analysis demonstrated that the ORF region of O568S was specifically over expressed in ovarian tumors. The original probe used corresponded to the 3′UTR of the VSGF sequence disclosed in SEQ ID NO:184. The results from these Northern blots revealed an ovarian tumor-specific 5.0 Kb message as well as a potential 3.5 Kb brain specific message. To confirm that the entire region covered by the ORF yields a single 5.0 Kb ovarian tumor-specific message, two additional probes were designed. The probes were located at the 5′ and 3′ regions of the ORF. Northern blot analysis using these two probes demonstrated that both probes hybridized to a 5.0 Kb product present only in ovarian tumor samples. Both probes failed to hybridize with RNA derived from multiple brain samples.

### Example 9

### Real Time PCR and Northern Blot Analysis of O590S

Real time PCR analysis of ovarian tumor antigen O590S was performed essentially as described in Example 6. O590S specific primers and probe were designed and quantitative Real Time PCR was performed on a panel of cDNAs prepared from a variety of tissues including ovarian tumor samples and a panel of normal tissues. This analysis revealed that O590S-specific mRNA was over expressed in approximately 65% of ovarian tumor samples tested, 100% tumor samples derived from SCID mice, and 100% ovarian tumor cell lines tested, when compared to normal ovarian tissue. No detectable expression was observed in normal tissues.

In addition to Real Time PCR, Northern blot analysis was performed to determine to transcript size of O590S. The Northern blot was probed with a 537 bp PCR product specific for O590S, which was designed to avoid regions of repeat sequences. This probe revealed a smeared band that was approximately 9.0 Kb in size, which was present in the majority of ovarian tumor samples tested.

### Example 10

### Analysis of cDNA Expression Using Microarray Technology

This example describes microarray expression analysis of ovary tumor-and tissue-specific cDNAs identified from OTCLS4, POTS2 and POTS7 (Subtraction libraries described in Example 1). Microarray analysis was performed essentially as described in Example 2. Sequence expression was determined by probing with a number of ovarian tumor samples, including papillary serous cystic carcinoma, papillary serous adenocarcinoma, papillary serous neoplasm, papillary serous carcinoma, papillary serous cytstadenocarcinoma, and a panel of normal tissues including adrenal gland, pituitary gland, thymus, bronchus, stomach, pancreas, skin, spinal cord, kidney, spleen, brain, breast, small intestine, thyroid, trachea, colon, PBMC resting, PBMC activated, lung, aorta, bone marrow, mammary epithelial tissue, esophagus, heart, and liver.

Clones showing an ovarian tumor mean or median value that was at least two fold greater than the normal tissue value were selected for further analysis. Further selection criteria was imposed on mean and median values as follows:

Mean tumor value ≧0.2 and mean normal value of <0.4 Median tumor value ≧0.2 and median normal value of <0.3.

Based on the selection criteria above, 26 clones were selected from the OTCLS4, POTS2 and POTS7 for sequencing. These sequences are disclosed herein in SEQ ID NOs:216-243. See Table IX for details.

TABLE IX

| SEQ | | GenBank | | | Ratio | Group | Group |
|---|---|---|---|---|---|---|---|
| ID NO | Clone ID | ID NO | GenBank Description | Ratio | 1/2 | 1 | 2 |
| 216 | 91226.5 | 15779016 | Homo sapiens, clone IMAGE:4047062, mRNA | Mean | 2.09 | 0.722 | 0.346 |
| 217 | 91227.2 | 14760620 | Homo sapiens bHLH protein DEC2 (DEC2), | Mean | 2.45 | 0.62 | 0.153 |
| | | | mRNA | | | | |
| 218 | 91230.2 | 13543043 | Homo sapiens, hypothetical protein dJ473B4, | Mean | 2.17 | 0.434 | 0.2 |
| | | | clone MGC:4987 IMAGE:3450155, mRNA, | | | | |
| | | | complete cds | | | | |
| 219 | 91231 | 13277551 | Homo sapiens, coxsackie virus and adenovirus | Mean | 2.16 | 0.545 | 0.253 |
| | | | receptor, clone MGC:5086 IMAGE:3463613, | | | | |
| | | | mRNA, complete cds | | | | |
| 220 | 91238.3 | 12804424 | Homo sapiens, similar to phosphoserine | Mean | 2.18 | 0.229 | 0.105 |
| | | | aminotransferase, clone MGC:1460 | | | | |
| | | | IMAGE:3544564, mRNA, complete cds | | | | |
| 221 | 91239.6 | 14589888 | Homo sapiens cadherin 2, type 1, N-cadherin | Media | 2.22 | 0.581 | 0.262 |
| | | | (neuronal) (CDH2), mRNA | n | | | |
| 222 | 91240.2 | 5729900 | Homo sapiens IGF-II mRNA-binding protein 3 | Mean | 2.08 | 0.236 | 0.114 |
| | | | (KOC1), mRNA | | | | |
| 223 | 91241.2 | 12653176 | Homo sapiens, MAD2 (mitotic arrest deficient, | Media | 2.13 | 0.316 | 0.148 |
| | | | yeast, homolog)-like 1, clone MGC:8662 | n | | | |
| | | | IMAGE:2964388, mRNA, complete cds | | | | |
| 224 | 91242.5 | 12653176 | Homo sapiens, MAD2 (mitotic arrest deficient, | Mean | 2.36 | 0.458 | 0.194 |
| | | | yeast, homolog)-like 1, clone MGC:8662 | | | | |
| | | | IMAGE:2964388, mRNA, complete cds | | | | |
| 225 | 91243.6 | 15297244 | Homo sapiens laminin, gamma 2 (nicein | Mean | 2.91 | 0.755 | 0.26 |
| | | | (100 kD), kalinin (105 kD), BM600 (100 kD), | | | | |
| | | | Herlitz junctional epidermolysis bullosa)) | | | | |
| | | | (LAMC2), mRNA | | | | |
| 226 | 91245.2 | 7022574 | Homo sapiens cDNA FLJ 10500 fis, clone | Mean | 2.1 | 0.571 | 0.272 |
| | | | NT2RP2000369 | | | | |
| 227 | 91246.4 | 1575533 | Human MAD2 (hsMAD2) mRNA, complete cds | Media | 2.51 | 0.292 | 0.116 |
| | | | | n | | | |
| 228 | 91247.3 | 5912166 | Homo sapiens mRNA; cDNA DKFZp564H1663 | Mean | 2.03 | 0.369 | 0.182 |
| | | | (from clone DKFZp564H1663) | | | | |
| 229 | 91247.4 | 5912166 | Homo sapiens mRNA; cDNA DKFZp564H1663 | Mean | 2.03 | 0.369 | 0.182 |
| | | | (from clone DKFZp564H1663) | | | | |
| 230 | 91249.2 | 14711935 | Homo sapiens, hypothetical protein FLJ10461, | Mean | 2.26 | 0.271 | 0.12 |
| | | | clone IMAGE:4102110, mRNA | | | | |
| 231 | 91253.2 | 14756011 | Homo sapiens similar to coxsackie virus and | Mean | 2.4 | 0.411 | 0.172 |
| | | | adenovirus receptor; 46 kD coxsackie and | | | | |
| | | | adenovirus receptor (CAR) protein (H. sapiens) | | | | |
| | | | (LOC93529), mRNA | | | | |
| 232 | 91254.2 | 11493240 | Human DNA sequence from clone RP11- | Mean | 5.15 | 1.396 | 0.271 |
| | | | 124N19 on chromosome 13, complete | | | | |
| | | | sequence [Homo sapiens] | | | | |
| 233 | 91259.2 | 14771329 | Homo sapiens Wilms tumor (WT1), mRNA | Mean | 3.87 | 0.406 | 0.105 |
| 234 | 91261.3 | 11465000 | Homo sapiens 12 BAC RP11-283G6 (Roswell | Mean | 2.57 | 0.34 | 0.132 |
| | | | Park Cancer Institute Human BAC library) | | | | |
| | | | complete sequence | | | | |
| 235 | 91261.4 | 11465000 | Homo sapiens 12 BAC RP11-283G6 (Roswell | Mean | 2.57 | 0.34 | 0.132 |
| | | | Park Cancer Institute Human BAG libray) | | | | |
| | | | complete sequence | | | | |
| 236 | 91262.2 | 4506070 | Homo sapiens protein kinase C, iota (PRKC1), | Mean | 2.46 | 0.695 | 0.282 |
| | | | mRNA | | | | |
| 237 | 91263.2 | 13647850 | Homo sapiens matrix metalloproteinase 11 | Mean | 2.63 | 0.254 | 0.097 |
| | | | (stromolysin 3) (MMP11), mRNA | | | | |
| 238 | 91264.2 | NA | NOVEL (no GENSEQ) | Mean | 15.6 | 2.058 | 0.132 |
| 239 | 91268.2 | 3980529 | Homo sapiens PAC clone RP4-797C5 from | Mean | 2.41 | 0.232 | 0.096 |
| | | | 7q31, complete sequence | | | | |
| 240 | 91269.5 | NA | NOVEL (no GENSEQ) | Mean | 3.04 | 0.226 | 0.074 |
| 241 | 91271.5 | 339440 | Homo sapiens transcriptional enhancer factor | Mean | 2.1 | 0.407 | 0.194 |
| | | | (TEF1) DNA, complete cds | | | | |
| 242 | 91273.3 | 15297244 | Homo sapiens laminin, gamma 2 (nicein | Mean | 2.5 | 0.625 | 0.25 |
| | | | (100 kD), kalinin (105 kD), BM600 (100 kD), | | | | |
| | | | Herlitz junctional epidermolysis bullosa)) | | | | |
| | | | (LAMC2), mRNA | | | | |
| 243 | 91274.6 | NA | NOVEL (GENSEQ”AAQ60336) | Mean | 2.58 | 0.204 | 0.079 |

### Example 11

### Expression Analysis and Further Characterization of Ovarian Sequence O646S

Ovarian tumor antigen O646S was originally described in Example 10 as clone 91274.6 (SEQ ID NO:243). Using SEQ ID NO:243 to search publicly available databases, a contig was generated, the DNA sequence of which is disclosed in SEQ ID NO:246, with a corresponding protein sequence disclosed in SEQ ID NO:249. This sequence was shown to share homology with Genbank Accession Number 18549403, the DNA and protein sequences of which are disclosed in SEQ ID NOs:244 and 247, respectively, and Genbank Accession Number FLJ14035, the DNA and protein sequences for which are disclosed in SEQ ID NOs:245 and 248, respectively.

### Example 12

### Further Characterization of Ovarian Sequence O648S

Ovarian tumor antigen O648S was originally described in Example 10 as clone 91268.2 (SEQ ID NO:239). Using SEQ ID NO:239 to search publicly available databases, a contig was generated, the DNA sequence of which is disclosed in SEQ ID NO:256, with a corresponding protein sequence disclosed in SEQ ID NO:261. This sequence was shown to share homology with several sequences including, Genbank Accession Number 3980529, the DNA sequence of which is disclosed in SEQ ID NOs:250, Genbank Accession Number 13629915, the DNA and protein sequences for which are disclosed in SEQ ID NOs:251 and 257, Genbank Accession Number 9789986, the DNA and protein sequences of which are disclosed in SEQ ID NOs:252 and 258, respectively, Genbank Accession Number 6006516, the DNA and protein sequences of which are disclosed in SEQ ID NOs:253 and 259, Genbank Accession Number 5689424, the DNA and protein sequences of which are disclosed in SEQ ID NOs:254 and 260, and Genbank Accession Number 15638833, the DNA sequence of which is disclosed in SEQ ID NO:255.

### Example 13

### Further Characterization of Ovarian Sequence O647S

Ovarian tumor antigen O647S was originally described in Example 10 as clone 91261.3 (SEQ ID NO:234). Using SEQ ID NO:234 to search publicly available databases, a contig was generated, the DNA sequence of which is disclosed in SEQ ID NO:268. This sequence was shown to share homology with several sequences, including Genbank Accession Number 16933560, the DNA and protein sequences of which are disclosed in SEQ ID NOs:262 and 269, Genbank Accession Number 12053028, the DNA and protein sequences for which are disclosed in SEQ ID NOs:263 and 270, Genbank Accession Number 7638812, the DNA and protein sequences of which are disclosed in SEQ ID NOs:264 and 271, Genbank Accession Number 939922, the DNA and protein sequences of which are disclosed in SEQ ID NOs:265 and 272, Genbank Accession Number 6093230, the DNA sequence of which are disclosed in SEQ ID NO:266 and Genbank Accession Number 11465000, the DNA sequence of which is disclosed in SEQ ID NO:267.

### Example 14

### Further Characterization of Ovarian Sequence O648S

Ovarian tumor antigen O645S was originally described in Example 10 as clone 91264.2 (SEQ ID NO:238). Using SEQ ID NO:238 to search publicly available databases, a contig was generated, the DNA sequence of which is disclosed in SEQ ID NO:273.

### Example 15

### Further Characterization of Ovarian Sequence O644S

Ovarian tumor antigen O644S was originally described in Example 10 as clone 91269.5 (SEQ ID NO:240). Using SEQ ID NO:240 to search publicly available databases, a contig was generated, the DNA sequence of which is disclosed in SEQ ID NO:277. This sequence was found to contain three open reading frames, the sequences of which are disclosed in SEQ ID NOs:280-282. These sequences were shown to share homology with Genbank Accession Number NM006580, the DNA and protein sequences of which are disclosed in SEQ ID NOs:274 and 278, Genbank Accession Number AF152101.1, the DNA and protein sequences for which are disclosed in SEQ ID NOs:275 and 279, and Genbank Accession Number 18425237, the DNA sequence of which is disclosed in SEQ ID NOs:276.

### Example 16

### O591S Expression in E. coli

The identification and characterization of O591S (SEQ ID NO: 214, encoding the protein of SEQ ID NO: 215) was described above (Example 1 and 4). For production and purification of O591 S protein used for antibody generation, a truncated form of O591 S, lacking the signal peptide sequence, was expressed in E. coli using a modified pET 28 vector with an N-terminal histidine tag.

The truncated coding region of O591 S-A was PCR amplified minus the signal sequence (amino acids 24-141) with the following primer pairs:

(SEQ ID NO:287)

| | |
|---|---|
| CBH-005 | 5′ cacttcttgcttccaggctttgcgctgcaaat 3′ |

(SEQ ID NO:288)

| | |
|---|---|
| CBH-003 | 5′ actagctcgagtcagcagtgtgccgagaa 3′ |

PCR amplification was performed under the following reaction conditions:

10 μl 10×Pfu buffer

1 μl 10 mM dNTPs 2 μl 10 μM of each primer

83 μl of sterile water

1.5 μl Pfu DNA polymerase (Stratagene, La Jolla, Calif.)

50ηg DNA

The reaction was amplified under the following conditions:

96° C. 2 minutes, followed by 40 cycles of

96° C. 20 seconds, 64° C. 15 seconds, and 72° C. 1 minute,

With a final extension step of 72° C. for 4 minutes.

The PCR product was digested with Xho I and cloned into pPDM His (a modified pET28 vector with a histidine tag in frame on the 5′ end) that has been digested with Eco721 and XhoI. Constructs were confirmed through nucleic acid sequence analysis, the corresponding DNA and protein sequence for which are disclosed in SEQ ID NOs:283 and 284, respectively. Following sequence analysis, the construct was then transformed into BLR (DE3) pLys S and HMS 174 (DE3) pLys S cells.

### Example 17

### The Generation of Rabbit Anti-O568S Polyclonal Antibodies and Expression Determination in Ovarian Tumors

The over-expression of O568S in ovarian tumor samples and normal ovary was verified using affinity purified rabbit polyclonal antibodies to O568S in the immunohistorchemical (1HC) analysis of ovarian tumors and normal tissues.

Rabbits were immunized with purified recombinant O568S protein and polyclonal antibodies prepared. Briefly, production and purification of the O568S antigen used for antibody generation was as follows:

The ovarian tumor protein antigen O568S (amino acids 29-808) was expressed in an E. coli recombinant expression system and grown overnight at 37° C. in LB Broth with the appropriate antibiotics in a shaking incubator. The next morning, 10 ml of the overnight culture was added to 500 ml of 2×YT plus the appropriate antibiotics in a 2L-baffled Erlenmeyer flask. When the Optical Density (at 560 nanometers) of the culture reached 0.4-0.6 the cells were induced with IPTG (1 mM) for 4 hours, and then harvested by centrifugation, washed with phosphate buffered saline and centrifuged again. The supernatant was discarded and the cells were either processed immediately or frozen for future use. When processed immediately, in order to break open the E. coli cells, twenty milliliters of lysis buffer was added to the cell pellets, followed by vortex mixing and French Press disruption at a pressure of 16,000 psi. This lysed cell suspension was then centrifuged, the resulting supernatant and pellet fractions of which were examined by SDS-PAGE for the presence of recombinant protein.

The pellet prepared as described above was resuspended in 10 mM Tris pH 8.0, 1% CHAPS, washed and centrifuged again. This step was repeated an additional two times. The washed pellet containing inclusion bodies was then solubilized with either 8 M urea or 6 M guanidine HCl containing 10 mM Tris pH 8.0 plus 10 mM imidazole (solubilization buffer). The solubilized protein was added to 5 ml of nickel-chelate resin (Qiagen Inc.) and incubated for 45 min to 1 hour at room temperature with continuous agitation. After incubation, the resin and protein mixture was added to a disposable column and the flow through containing unbound proteins was collected. The column containing resin with bound protein was then washed with 10-20 column volumes of solubilization buffer, and eluted using an elution buffer solution containing 8M urea, 10 mM tris pH 8.0 and 300 mM imidazole. Column fractions (amounting to 3 ml of elution buffer each) were collected and examined by SDS-PAGE for the presence of O568S protein. Fractions containing the desired protein were pooled for further characterization. As an additional purification step, a strong anion exchange resin such as Hi-Prep Q (Biorad) was equilibrated with the appropriate buffer and the pooled fractions containing O568S protein were loaded onto this column and eluted using an increasing salt gradient. Fractions were collected and again evaluated by SDS-PAGE for the presence of O568S protein. The appropriate fractions were identified, combined and dialyzed against 10 mM Tris pH 8.0. Purity was determined by SDS-PAGE or HPLC, the concentration of purified protein was determined by Lowry assay or Amino Acid Analysis, the amino terminal protein sequence was determined to confirm authenticity, and the level of endotoxin was determined using a standard Limulus (LAL) assay. Fractions containing purified O568S were pooled, sterilized by filtration using a 0.22 micron filter, aliquoted and frozen until needed.

For the generation of polyclonal antiserum, rabbits were immunized with 400 micrograms of purified O568S protein combined with 100 micrograms of muramyldipeptide (MDP) and an equal volume of Incomplete Freund's Adjuvant (IFA). Every four weeks thereafter, animals were boosted with 100 micrograms of O568S antigen mixed with an equal volume of IFA. Seven days following each boost a blood sample from each immunized animal was taken and a serum fraction therefrom prepared by incubating the blood sample at 4° C. for 12-24 hours, clarified by centrifugation.

In order to characterize the above-mentioned rabbit polyclonal anti-0568S antiserum, 96 well plates were coated with the appropriate antigen in 50 μl (typically 1 μg of protein), incubated at 4C for 20 hours, after which 250 μl of BSA blocking buffer was added followed by an additional 2 hours of incubation at room temperature (RT). Each well was then washed 6 times with PBS/0.01% tween. The rabbit anti-O568S antiserum to be tested was diluted in PBS, 50 μl of which was added to each well and incubated at RT for 30 minutes. Plates were washed as described above and then 50 μl of a 1:10000 dilution of goat anti-rabbit horse radish peroxidase (HRP) conjugated antibody was added and incubated at RT for 30 minutes. Next, plates were washed as described above and 100 μl of TMB containing microwell Peroxidase was added. Substrate was added to each well, incubated for 15 minutes in the dark at RT, the calorimetric reaction stopped with the addition of 100 μl of 1N H2SO4 and signal determined immediately at 450 nm.

For IHC analysis, paraffin embedded formalin-fixed tissue was sliced into 4 micron sections. Steam heat induced epitope retrieval (SHIER) in 0.1M sodium citrate buffer (pH 6.0) was used for optimal staining conditions. Sections were incubated with 10% serum/PBS for 5 minutes. Primary antibody (0.5 μg/ml rabbit affinity purified anti-O568S polyclonal antibody) was added to each section for 25 minutes at varying concentrations, followed by a 25 minute incubation with an anti-rabbit biotinylated antibody. Rabbit IgG was also tested on all tissues and served as a negative control. Endogenous peroxidase activity was blocked by three 1.5 minute incubations with hydrogen peroxidase. The avidin biotin complex/horse radish peroxidase (ABC/HRP) system was used along with DAB chromogen to visualize antigen expression. Slides were counterstained with hematoxylin.

The tissues tested and their expression profiles are described in detail in Table X. Of the ovarian cancer metastases tested, six were adenocarcinomas, five of which tested positive and one was marginal. The majority of the tumor samples stained positive with a strong membrane localized signal, demonstrating that O568S is expressed on the surface of the tumor cells.

TABLE X
Tissue Expression of O568S

| TISSUE | O568S EXPRESSION |
|---|---|
| Ovarian cancer | 3/5 |
| Ovarian cancer metastases | 8/12 |
| Normal Ovary | 3/4 |
| Normal lung (alveolar epithelium) | 0/1 |
| Normal lung (bronchiole epithelium) | 0/1 |
| Brain Cortex | 6/6 |
| | (marginal staining of selected |
| | neuronal populations) |
| Brain (spinal cord) | 6/6 |
| | (marginal staining of purkinje |
| | cells) |
| Stomach | 5/5 |
| | (marginal staining of selected |
| | neuronal populations) |
| Skin | 0/1 |
| Heart | 0/1 |
| Kidney | 0/1 |
| Liver | 0/1 |
| Colon | 0/1 |
| Tonsil | 0/1 |
| Vagina | 1/1 |
| | (squamous epithelium) |

### Example 18

### Real-Time PCR Analysis of Ovarian Tumor Antigens Identified from the OTCLS4, POTS2 and POTS7 Libraries

Clones identified as having a good expression profile by microarray analysis (as described in Example 10), were further analyzed by real-time PCR on an extended panel of ovarian tumor and normal tissue samples (including ovary, aorta, adrenal gland, bladder, bone, bronchus, brain, breast, CD34+ cells, dendritic cells, esophagus, heart, kidney, large intestine, liver, lung, lymph nodes, pancreas, peritoneum, bane marrow, skin, small intestine, spinal cord, spleen, stomach, thymus, thyroid, tonsil, trachea, ureter, uterus). Real time PCR was performed as described above in Example 6.

The first-strand cDNA used in the quantitative real-time PCR was synthesized from 20 μg of total RNA that was treated with DNase I (Amplification Grade, Gibco BRL Life Technology, Gaithersburg, Md.), using Superscript Reverse Transcriptase (RT) (Gibco BRL). Real-time PCR was performed with an ABIPRISM 7900 sequence detection system (PE Biosystems, Foster City, Calif.). The 7900 system uses SYBRTM green, a fluorescent dye that only intercalates into double stranded DNA, and a set of gene-specific forward and reverse primers. The increase in fluorescence was monitored during the whole amplification process. The optimal concentration of primers was determined using a checkerboard approach, and a pool of cDNAs from tumors was used in this process. The PCR reaction was performed in 12.5 μl volumes that included 2.5 μl of SYBR green buffer, 2 μl of cDNA template and 2.5 μl each of the forward and reverse primers for the gene of interest. The cDNAs used for RT reactions were diluted 1:10 for each gene of interest and 1:100 for the β-actin control. The expression of the gene of interest in various tissue samples was represented by comparative C_{T} (threshold cycle) method. C_{T} indicates the fractional cycle number at which the amount of amplified target reaches a fixed threshold. The C_{T} value of normal aorta, skin, peritoneum, thyroid gland, dendritic cells, or CD34⁺ cells was used as a comparative reference in order to evaluate the over-expression levels seen with each of the genes.

The following clones have been evaluated on the extended ovarian real-time panel. In some cases where expression was fairly ubiquitous, mean real-time expression values were determined for ovarian tumor (not including ovarian tumor cell line and SCID samples), normal ovarian, and other normal tissues (not including normal ovary). All clones were found to be over-expressed in ovarian tumor to some degree, demonstrating their use as tumor immunotherapeutics and/or diagnostic targets.

Ovarian tumor antigen O644S (SEQ ID NO:240) was shown to be over-expressed in ovarian tumor tissue samples compared to normal tissues. Expression of O644S was similar in ovarian tumor samples compared to normal ovary. Mean expression ratios for O644S were as follows: ovarian tumor/normal ovary was 0.6 and ovarian tumor/other normal tissues was 5.8. These results indicate that O644S may be used in developing tumor immunotherapeutic and/or diagnostic agents.

Ovarian tumor antigen O645S (SEQ ID NO:238) was found to be over-expressed in over 70% of the ovarian tumors tested, and 100% of ovarian tumor SCID samples. No expression was detected in the normal tissues tested. This finding further supports the use of ovarian tumor antigen O645S in the diagnosis and treatment of ovarian cancer. Based on the excellent expression profile of this ovarian candidate, SEQ ID NO:238 was also run on an the Ovarian Metastatic Extended Panel, which included 14 primary ovarian tumors and 13 metastatic ovarian tumors. O645S was determined to be elevated in 10/14 (71%) of primary tumors and 11/13 (85%) metastatic tumors.

Ovarian tumor antigen O646S (SEQ ID NO:243) was found to be over-expressed in 100% of the ovarian tumors tested, 1/1 ovarian tumor cell lines (SKOV3-HTB77) and 100% of ovarian tumor SCID samples. Low-level expression was observed in 2/2 normal ovary samples tested, but no expression was detected in any other normal tissues tested. This finding further supports the use of ovarian tumor antigen O646S in the diagnosis and treatment of ovarian cancer, especially metastatic ovarian cancer. Based on the excellent expression profile of this ovarian candidate, SEQ ID NO:243 was also run on an the Ovarian Metastatic Extended Panel, which included 14 primary ovarian tumors and 13 metastatic ovarian tumors. O646S was determined to be elevated in 14/14 (100%) of primary tumors and 13/13 (100%) metastatic tumors.

Ovarian tumor antigen O647S (SEQ ID NO:234 and 235) was found to be over-expressed in over 80% of the ovarian tumors tested, and 100% of ovarian tumor SCID samples. O647S was also found to have low level expression in normal ovary, bronchus, brain/cerebellum, and heart. No expression was detected in any other normal tissues tested. This finding further supports the use of ovarian tumor antigen O647S in the diagnosis and treatment of ovarian cancer.

Ovarian tumor antigen O648S (SEQ ID NO:239) was found to be over-expressed in over 50% of the ovarian tumors tested. O648S was not expressed in normal ovary. Very low-level expression was seen in normal liver and pancreas. This finding further supports the use of ovarian tumor antigen O648S in the diagnosis and treatment of ovarian cancer.

Ovarian tumor antigen O651 S (SEQ ID NO:232) was found to be over-expressed in over 60% of the ovarian tumors tested, 1/1 ovarian tumor cell lines (SKOV3-HTB77) and 100% of ovarian tumor SCID samples. No expression was detected in the normal tissues tested. This finding further supports the use of ovarian tumor antigen O651 S in the diagnosis and treatment of ovarian cancer.

Ovarian tumor antigen O645S (SEQ ID NO:238) was found to be over-expressed in over 70% of the ovarian tumors tested, and 100% of ovarian tumor SCID samples. No expression was detected in the normal tissues tested. This finding further supports the use of ovarian tumor antigen O645S in the diagnosis and treatment of ovarian cancer.

### Example 19

### LifeSeq Analysis of Ovarian Tumor Antigen O590S

In Example 1 (Table VII) the DNA insert of clone 57886 was identified, and disclosed in SEQ ID NO:198 (606 bps in length), also referred to as O590S. Characterization of SEQ ID NO:198 by microarray analysis (Examples 2 and 9) indicated that corresponding mRNA was overexpressed in ovarian tumor tissue relative to normal tissues. Additional characterization by Northern blot analysis detected an mRNA transcript approximately 9.0 kb in size (Example 9). In this example, the DNA sequence for the ovarian tumor antigen O590S (SEQ ID NO: 198) disclosed in Example 1 was used as a query to perform a BlastN search of the Incyte Genomics LifeSeq Gold database (LGtemplatesJan2001). This analysis identified an identical sequence match on template number 93744.1, corresponding to a 1740 base pair sequence, as is disclosed in SEQ ID NO:285. The gene bin, 93744, from which this match was identified contained 21 clones from various tumor libraries. Further analysis of the template 93744.1 sequence (SEQ ID NO:285), identified a −2 open reading frame that would translate a polypeptide with a predicted amino acid sequence disclosed in SEQ ID NO:286. In addition, this analysis confirmed that the open reading frame identified by SEQ ID NO:286 overlaps with and is contained within the nucleotide sequence of SEQ ID NO:198 corresponding to the ovarian tumor antigen O590S.

### Example 20

### Analysis of Ovarian Tumor Antigen O664S

O644S (initially described in example 10 as SEQ ID NO:240, with extended open reading frames disclosed in SEQ ID NOs:280-282) was previously identified as having a good expression profile by microarray (see Example 18 for details) and was further analyzed by real-time PCR.

The first strand cDNA used in the quantitative real-time PCR was synthesized from 20 μg of total RNA that was treated with DNase I (Amplification Grade, Gibco BRLLife Technology, Gaithersburg, Md.0, using Superscript Reverse Transcriptase (RT) (Gibco BRL). Real-time PCR was performed with an ABIPRISM 7900 sequence detection system (PE Biosystems, Foster City, Calif.). The 7900 system uses SYBR™ green, a fluorescent dye that only intercalates into double stranded DNA, and a set of O644S specific forward and reverse primers. The increase in fluorescence was monitored during the whole amplification process. The optimal concentration of primers was determined using a checkerboard approach, and a pool of cDNAs from tumors was used in this process. The PCR was performed in 12.5 μl volumes that included 2.5 μl of SYBR green buffer, 2 μl of cDNA template and 2.5 μl each of the forward and reverse primer. The cDNAs used for the RT reactions were diluted 1:10 for O644S and 1:100 for the β-actin control. The expression of O644S in each of the tissue samples was represented by the comparative CT (threshold cycle) method. CT indicates the fractional cycle number at which the amount of amplified target reaches a fixed threshold. The CT value of normal skin was used as a comparative reference in order to evaluate the over-expression levels seen with O644S.

O644S did not show over-expression in ovarian tumor tissue compared to normal tissue, however it did show higher expression in ovarian tumor tissue than in other normal tissue. As O644S is over-expressed in ovarian tumor tissue compared to normal tissues, it is a useful ovarian tumor antigen for the development of immunotherapeutic and/or diagnostic reagents. The high expression of O644S in both ovary tumor and normal ovary demonstrates that it would be a useful marker in the detection of metastatic cancer.

U.S. patents, U.S. patent application publications, U.S. patent applications, foreign patents, foreign patent applications and non-patent publications referred to in this specification and/or listed in the Application Data Sheet, including, but not limited to U.S. application Ser. No. 09/970,966, filed Oct. 2, 2001 which is a continuation in part of Ser. No. 09/825,294, filed Apr. 3, 2001 which is a continuation in part of Ser. No. 09/713,550, filed Nov. 14, 2000 which is a continuation in part of Ser. No. 09/656,668, filed Sep. 7, 2000 which is a continuation in part of Ser. No. 09/640,173, filed Aug. 15, 2000 which is a continuation in part of Ser. No. 09/561,778, filed May 1, 2000, which is a continuation in part of Ser. No. 09/394,374, filed Sep. 10, 1999 and are incorporated herein by reference, in their entirety. Also incorporated herein by reference is U.S. patent application Ser. No. 09/820,089 filed Mar. 27, 2001.

### Example 21

### Cell Surface Expression of the Ovarian Tumor Antigen, O591 S

The identification and characterization of O591S (SEQ ID NO: 214, encoding the protein of SEQ ID NO: 215) was described above (Example 1 and 4). To characterize the cell surface expression of O591 S, cell lines were either transfected with full-length O591S cDNA or infected with an adenoviral expression construct expressing O591S cDNAs. These cell lines were then stained using purified rabbit polyclonal anti-O591S antibodies raised against synthetic O591S peptides, and surface expression analyzed by FACS. The O591S polyclonal antibodies were raised against the following peptides; peptide 1 (SEQ ID NO:291) corresponding to amino acid positions 26-55 of the O591S protein sequence (SEQ ID NO:215), peptide 2 (SEQ ID NO:292) corresponding to amino acid positions 53-78 of the O591S protein sequence (SEQ ID NO:215), and peptide 3 (SEQ ID NO:293) corresponding to amino acid positions 103-129 of O591S protein sequence (SEQ ID NO:215). Polyclonal antibodies were generated essentially as described in Example 17 of the present application.

Cell surface expression of O591 S was determined as follows:

1. oNXA cells were transfected by CaPO₄ precipitation with (a) a negative control cDNA cloned into the expression vector pBIB, or (b) O591 S cDNA cloned into the expression vector pBIB. Seventy-two hours post-transfection, the cells were harvested and stained with either (i) control rabbit polyclonal antibody, (ii) rabbit polyclonal anti-O591S antibody, or (iii) secondary antibody (anti-rabbit-FITC) alone. All cells transfected with an expression vector containing O591S stained using the O591S specific polyclonal antibodies, demonstrating surface expression of O591 S.

2. oNXA cells were transfected by CaPO₄ precipitation with either; pBIB/O591S (O591S cDNA cloned into the expression vectors pBIB), pcDNA/O591 S (O591 S cDNA cloned into the expression vector, pcDNA3), or pCEP/O591S (O591S cDNA cloned into the expression vector pCEP4). Seventy-two hours post-transfection, cells were harvested and stained with either (i) control rabbit polyclonal antibody or (ii) rabbit polyclonal anti-O591S antibody. O591S was detected on the surface of all cells transfected with O591S specific sequences. O591 S expression levels were shown to be highest with the episomal replicating vector pcDNA4.

3. oNXA and 293 cells were transfected by CaPO₄ precipitation with pcDNA/O591 S (O591 S cDNA cloned into the expression vector pc DNA3). Seventy-two hours post-transfection, the cells were harvested and stained with either (i) control rabbit polyclonal antibodies, or (ii) rabbit polyclonal anti-O519S antibody. The cells were than analyzed using FACS analysis. Both ONXA and 293 cells transfected with O591 S demonstrated cell surface expression of O591 S.

4. VA13 cells and ONXA cells were infected (MOI of 10:1) with O591S/adenovirus (O591S cDNA cloned into the adenoviral expression vector). Seventy-two hours post-infection, the cells were harvested and stained with either, (i) control rabbit polyclonal antibody, or (ii) rabbit polyclonal anti-O591 S antibody. The cells were then analyzed using FACS. Cells infected with O591 S/adenovirus demonstrated cell surface staining specific for O591 S.

To further characterize that O591 S was a surface expressed protein, ONXA cells were transfected by CaPO₄ precipitation with pBIB/O591S (O591S cDNA cloned into the expression vector pBIB). Seventy-two hours post-transfection the cells were harvested and incubated for an additional one hour in either the presence or absence of phoshatidylinositol phospholipae C(PI-PLC), an enzyme known to cleave glycosyl-phosphatidylinositol (GPI)-linked proteins. GPI-linked proteins are known to be surface expressed proteins. Following incubation with PI-PLC, the cells were washed and either stained with (i) rabbit polyclonal anti-O591S antibody, or (ii) secondary antibody (anti-rabbit-FITC) alone, and analyzed by FACS for O591S cell surface expression. Analysis demonstrated that cells treated with PI-PLC were negative for the cell surface expression of O591 S, further demonstrating that this protein is a surface expressed protein. Analysis of the O591 S protein sequence (SEQ ID NO:215) revealed that the enzyme PI-PLC cleaved at either the Arg at position 114 of SEQ ID NO:215, resulting in the generation of a liberated 114 amino acid fragment, the sequence of which is disclosed in SEQ ID NO:289, and theoretically a 27 amino acid cell associated fragment (residues 115-141 of SEQ ID NO:215) or at the Gly at position 115 of SEQ ID NO:215, resulting in the generation of a 115 amino acid fragment, the sequence of which is disclosed in SEQ ID NO:290 and theoretically a 26 amino acid cell associated fragment (residues 116-141 of SEQ ID NO:215).

These data demonstrate that O591S is a surface expressed, GPI-linked protein, making the sequence a target for therapeutic antibodies.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. An isolated polynucleotide comprising a sequence selected from the group consisting of:
(a) SEQ ID NO: 214;
(b) the complement SEQ ID NO:214;
(c) a sequence consisting of at least 20 contiguous residues of SEQ ID NO:214;
(d) sequences that hybridize to a SEQ ID NO:214 under moderately stringent conditions;
(e) a sequence having at least 75% identity to SEQ ID NO:214;
(f) a sequence having at least 90% identity SEQ ID NO:214 and (g) a degenerate variant of SEQ ID NO:214.

2. An isolated polypeptide comprising an amino acid sequence of an ovarian tumor protein selected from the group consisting of:
(a) polynucleotides recited in any one of sequences encoded by a polynucleotide of claim 1;
(b) sequences having at least 70% identity to a sequence encoded by a polynucleotide of claim 1;
(c) sequences having at least 90% identity to a sequence encoded by a polynucleotide of claim 1;
(d) sequences provided in SEQ ID NOs: 289-293;
(e) sequences having at least 70% identity to a sequence provided in SEQ ID NOs: 289-293; and
(f) sequences having at least 90% identity to a sequence provided in SEQ ID NOs: 289-293.

3. An expression vector comprising a polynucleotide of claim 1 operably linked to an expression control sequence.

4. A host cell transformed or transfected with an expression vector according to claim 3.

5. An isolated antibody, or antigen-binding fragment thereof, that specifically binds to a polypeptide of claim 2.

6. A method for detecting the presence of an ovarian cancer in a patient, comprising the steps of:
(a) obtaining a biological sample from the patient;
(b) contacting the biological sample with a binding agent that binds to a polypeptide of claim 2;
(c) detecting in the sample an amount of polypeptide that binds to the binding agent; and
(d) comparing the amount of polypeptide to a predetermined cut-off value and therefrom determining the presence of a cancer in the patient.

7. A fusion protein comprising at least one polypeptide according to claim 2.

8. An oligonucleotide that hybridizes to SEQ ID NO:214 under moderately stringent conditions.

9. A method for stimulating and/or expanding T cells specific for a tumor protein, comprising contacting T cells with at least one component selected from the group consisting of:
(a) polypeptides according to claim 2; (b) polynucleotides according to claim 1; and
(c) antigen-presenting cells that express a polypeptide according to claim 1,
under conditions and for a time sufficient to permit the stimulation and/or expansion of T cells.

10. An isolated T cell population, comprising T cells prepared according to the method of claim 9.

11. A composition comprising a first component selected from the group consisting of physiologically acceptable carriers and immunostimulants, and a second component selected from the group consisting of:
(a) polypeptides according to claim 2;
(b) polynucleotides according to claim 1;
(c) antibodies according to claim 5;
(d) fusion proteins according to claim 7; and
(e) antigen presenting cells that express a polypeptide according to claim 2.

12. A method for stimulating an immune response in a patient, comprising administering to the patient a composition of claim 11.

13. A method for the treatment of a cancer in a patient, comprising administering to the patient a composition of claim 11.

14. A method for determining the presence of a cancer in a patient, comprising the steps of:
(a) obtaining a biological sample from the patient;
(b) contacting the biological sample with an oligonucleotide according to claim 8;
(c) detecting in the sample an amount of a polynucleotide that hybridizes to the oligonucleotide; and
(d) compare the amount of polynucleotide that hybridizes to the oligonucleotide to a predetermined cut-off value, and therefrom determining the presence of the cancer in the patient.

15. A diagnostic kit comprising at least one oligonucleotide according to claim 8.

16. A diagnostic kit comprising at least one antibody according to claim 5 and a detection reagent, wherein the detection reagent comprises a reporter group.

17. A method for inhibiting the development of a cancer in a patient, comprising the steps of:
(a) incubating CD4+ and/or CD8+ T cells isolated from a patient with at least one component selected from the group consisting of: (i) polypeptides according to claim 2; (ii) polynucleotides according to claim 1; and (iii) antigen presenting cells that express a polypeptide of claim 2, such that T cell proliferate;
(b) administering to the patient an effective amount of the proliferated T cells, and thereby inhibiting the development of a cancer in the patient.
